(19) United States
(12) Patent Application Publication
(10) Pub. No.: US 2025/0122222 A1
(43) Pub. Date: Apr. 17, 2025

(54) HETEROCYCLIC COMPOUNDS AND METHODS OF USE

(71) Applicant: AMGEN INC., Thousand Oaks, CA (US)

(72) Inventors: Michael M. YAMANO, Thousand Oaks, CA (US); Yunxiao LI, Thousand Oaks, CA (US); Primali Vasundera NAVARATNE, Thousand Oaks, CA (US); Jose M. MEDINA, Thousand Oaks, CA (US); Ning CHEN, Thousand Oaks, CA (US); Liping PETTUS, Thousand Oaks, CA (US); Rene RAHIMOFF, Santa Monica, CA (US); Xiaofen LI, Thousand Oaks, CA (US); John STELLWAGEN, Beverly, MA (US); Francesco MANONI, Newbury Park, CA (US); Kexue LI, Newbury Park, CA (US); Brian Alan LANMAN, Woodland Hills, CA (US); Ryan Paul Wurz, Newbury Park, CA (US); Wei ZHAO, Westford, MA (US); Huan RUI, Los Angeles, CA (US); Josephine ESHON, Medford, MA (US)

(73) Assignee: AMGEN INC., Thousand Oaks, CA (US)

(21) Appl. No.: 18/695,673

(22) PCT Filed: Aug. 10, 2022
(86) PCT No.: PCT/US2022/039968

§ 371 Date: Mar. 26, 2024

Related U.S. Application Data
(60) Provisional application No. 63/231,543, filed on Aug. 10, 2021.
(60) Provisional application No. 63/289,576, filed on Dec. 14, 2021.

(51) Int. Cl. C07D 519/00; A61K 31/519; A61K 31/5386; A61K 31/541; A61K 31/553; A61K 31/554; C07D 471/04

(52) U.S. Cl. CPC C07D 519/00; A61K 31/519; A61K 31/5386; A61K 31/541; A61K 31/553; A61K 31/554; C07D 471/04

(57) ABSTRACT

The present disclosure provides compounds useful for the inhibition of KRAS. The compounds have a general Formula I: wherein the variables of Formula I are defined herein. This disclosure also provides pharmaceutical compositions comprising the compounds, uses of the compounds, and compositions for treatment of, for example, cancer.

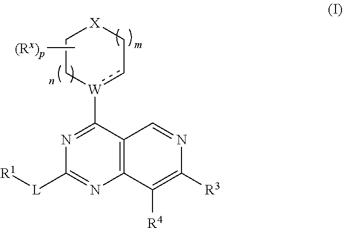

### CROSS-REFERENCE TO RELATED APPLICATION

This application claims the benefit of U.S. Provisional Patent Application No. 63/231,543, filed Aug. 10, 2021 and U.S. Provisional Patent Application No. 63/289,576, filed Dec. 14, 2021, each of which is incorporated by reference in its entirety.

### FIELD

The present disclosure provides compounds having activity as inhibitors of G12D mutant KRAS protein. This disclosure also provides pharmaceutical compositions comprising the compounds, uses and methods of treating certain disorders, such as cancer, including but not limited to Non-Small Cell Lung Cancer (NSCLC), colorectal cancer and/or pancreatic cancer.

### BACKGROUND

From its identification as one of the first human oncogenes in 1982 (Der et al., 1982), KRAS (the Kirsten rat sarcoma viral oncogene homologue) has been the focus of extensive academic and industrial research, as a key node in the MAPK signal transduction pathway, as a transforming factor in a network of parallel effector pathways (e.g., PI3K/AKT) (Vojtek et al., 1998) and as a potential target for anti-cancer agents (Malumbres et al., 2003). Despite progress in the development of inhibitors of upstream and downstream nodes in the MAPK pathway (e.g., EGFR (Sridhar et al., 2003), BRAF (Holderfield et al., 2014) and MOK (Caunt et al., 2015), the KRAS protein has historically proven resistant to direct inhibition.

KRAS is a G-protein that couples extracellular mitogenic signaling to intracellular, pro-proliferative responses. KRAS serves as an intracellular “on/off” switch. Mitogen stimulation induces the binding of GTP to KRAS, bringing about a conformational change which enables the interaction of KRAS with downstream effector proteins, leading to cellular proliferation. Normally, pro-proliferative signaling is regulated by the action of GTPase-activating proteins (GAPs), which return KRAS to its GDP-bound, non-proliferative state. Mutations in KRAS impair the regulated cycling of KRAS between these GDP- and GTP-bound states, leading to the accumulation of the GTP-bound active state and dysregulated cellular proliferation (Simanshu et al., 2017).

Attempts to develop inhibitors of mutated KRAS proteins have historically been thwarted by the absence of druggable pockets on the surface of the protein (Cox et al., 2014). In 2013, Shokat and colleagues identified covalent inhibitors of a common (O'Bryan, 2019) oncogenic mutant of KRAS, KRAS G12C, which bound to a previously unrecognized allosteric pocket on GDP-KRAS G12C and prevented its subsequent activation (Ostream et al., 2013). This discovery brought about significant new efforts in the KRAS inhibitor research, which have recently culminated in the entry of KRAS inhibitors in human clinical trials.

While some progress has been made on KRAS G12C inhibitors, there is a continued interest and effort to develop inhibitors of KRAS, particularly inhibitors of other KRAS such as KRAS G12D, G12V, G12A or G12S. Thus, there is a need to develop new inhibitors for KRAS G12D, G12V, G12A, G12S or G12C for the treatment of disorders, such as cancer.

### SUMMARY

In one aspect, the present application is directed to compound of formula (I):

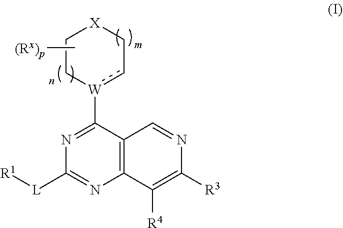

or a pharmaceutically acceptable salt of said compound, wherein;
[symbol] is a single bond or a double bond;
W is C, CH or N, wherein when W is CH or N, [symbol] is a single bond;
X is O, S, S(O), S(O)(NR^{z}) or S(O)₂;
n is 0, 1, 2, or 3;
m is 0, 1, 2 or 3;
p is 0, 1, 2 or 3;
each R^{x} is hydroxyl, halogen, oxo, cyano, —N(R^{z})₂, C₁₋₄ alkyl, C₁₋₄ alkoxy, C₁₋₄ haloalkyl, C₁₋₄ haloalkoxy, C₃₋₆ cycloalkyl, 5-7 membered heteroaryl, -T-R^{y} or two R^{x} taken together with adjacent carbon atoms can form a C₃₋₇ cycloalkyl or 5-7 membered heterocycloalkyl, wherein each C₃₋₇ cycloalkyl or 5-7 membered heterocycloalkyl is further substituted with 0-3 occurrences of R^{y} or two R^{x} taken together can form a bridged ring where the bridge is selected from one of the following: —C₁₋₄ alkylene, —C₁₋₄ alkylene-O—C₁₋₄ alkylene-, —O—, —S— or —C₁₋₄ alkylene-S—C₁₋₄ alkylene- and wherein each C₁₋₄ alkylene is further substituted with 0-2 occurrences of R^{y};
L is C₁₋₆ alkylene, —O—C₁₋₆ alkylene, —S—C₁₋₆ alkylene, NR^{z}, O or S, wherein each C₁₋₆ alkylene, —O—C₁₋₆ alkylene and —S—C₁₋₆ alkylene chain is substituted with 0-2 occurrences of R²;
R¹ is hydroxyl, aryl, heteroaryl, C₃₋₈ cycloalkyl or heterocycloalkyl substituted with 0-3 occurrences of R⁵;
R² is halogen, hydroxyl, C₁₋₄ alkyl or two R² on the same or adjacent carbon atoms can be taken together to form a C₃₋₇ cycloalkyl;
R³ is aryl or heteroaryl substituted with 0-3 occurrences of R⁶;
R⁴ is hydrogen, hydroxyl, halogen, C₁₋₄ alkyl, C₁₋₄ alkoxy, C₁₋₄ haloalkyl, C₂₋₄ alkenyl, C₂₋₄ alkynyl, C₃₋₇ cycloalkyl or cyano;
each R⁵ is halogen, oxo, hydroxyl, amino, cyano or C₁₋₄ alkyl;
each R⁶ is halogen, hydroxyl, cyano, —N(R)₂, C₁₋₄ alkyl, C₁₋₄ alkoxy, C₁₋₄ haloalkyl, C₁₋₄ haloalkoxy, C₂₋₄ alkynyl or C₃₋₆ cycloalkyl;
T is C₁₋₄ alkylene, —S(O)₂—, —C(O)—, —C₁₋₄ alkylene-C(O)—, C₁₋₄ alkylene-S(O)₂— or —S—;
R^{y} is halogen, C₁₋₄ alkyl, C₁₋₄ haloalkyl, C₁₋₄ alkoxy, hydroxyl, cyano or —N(R)₂; and
R^{z} is hydrogen or C₁₋₄ alkyl.

In a second aspect, provided herein is a pharmaceutical composition comprising a compound of Formula I or a pharmaceutically acceptable salt of said compound and a pharmaceutically acceptable excipient.

In a third aspect, provided herein is a compound of Formula I, or a pharmaceutically acceptable salt of said compound, or the pharmaceutical composition as described herein for use in treating cancer (e.g., NSCLC, colorectal cancer or pancreatic cancer).

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### DETAILED DESCRIPTION

Provided herein as embodiment 1 is a compound of formula (I):

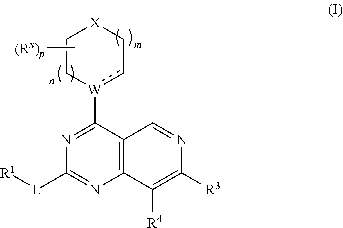

or a pharmaceutically acceptable salt of said compound, wherein;
[symbol] is a single bond or a double bond;
W is C, CH or N, wherein when W is CH or N, [symbol] is a single bond;
X is O, S, S(O), S(O)(NR^{z}) or S(O)₂;
n is 0, 1, 2, or 3;
m is 0, 1, 2 or 3;
p is 0, 1, 2 or 3;
each R^{x} is hydroxyl, halogen, oxo, cyano, —N(R^{z})₂, C₁₋₄ alkyl, C₁₋₄ alkoxy, C₁₋₄ haloalkyl, C₁₋₄ haloalkoxy, C₃₋₆ cycloalkyl, 5-7 membered heteroaryl, -T-R^{y} or two R^{x} taken together with adjacent carbon atoms can form a C₃₋₇ cycloalkyl or 5-7 membered heterocycloalkyl, wherein each C₃₋₇ cycloalkyl or 5-7 membered heterocycloalkyl is further substituted with 0-3 occurrences of R^{y} or two R^{x} taken together can form a bridged ring where the bridge is selected from one of the following: —C₁₋₄ alkylene, —C₁₋₄ alkylene-O—C₁₋₄ alkylene-, —O—, —S— or —C₁₋₄ alkylene-S—C₁₋₄ alkylene- and wherein each C₁₋₄ alkylene is further substituted with 0-2 occurrences of R^{y};
L is C₁₋₆ alkylene, —O—C₁₋₆ alkylene, —S—C₁₋₆ alkylene, NR^{z}, O or S, wherein each C₁₋₆ alkylene, —O—C₁₋₆ alkylene and —S—C₁₋₆ alkylene chain is substituted with 0-2 occurrences of R²;
R¹ is hydroxyl, aryl, heteroaryl, C₃₋ₛ cycloalkyl or heterocycloalkyl substituted with 0-3 occurrences of R⁵;
R² is halogen, hydroxyl, C₁₋₄ alkyl or two R² on the same or adjacent carbon atoms can be taken together to form a C₃₋₇ cycloalkyl;
R³ is aryl or heteroaryl substituted with 0-3 occurrences of R⁶;
R⁴ is hydrogen, hydroxyl, halogen, C₁₋₄ alkyl, C₁₋₄ alkoxy, C₁₋₄ haloalkyl, C₂₋₄ alkenyl, C₂₋₄ alkynyl, C₃₋₇ cycloalkyl or cyano;
each R⁵ is halogen, oxo, hydroxyl, amino, cyano or C₁₋₄ alkyl;
each R⁶ is halogen, hydroxyl, cyano, —N(R^{z})₂, C₁₋₄ alkyl, C₁₋₄ alkoxy, C₁₋₄ haloalkyl, C₁₋₄ haloalkoxy, C₂₋₄ alkynyl or C₃₋₆ cycloalkyl;
T is C₁₋₄ alkylene, —S(O)₂—, —C(O)—, —C₁₋₄ alkylene-C(O)—, C₁₋₄ alkylene-S(O)₂— or —S—;
R^{y} is halogen, C₁₋₄ alkyl, C₁₋₄ haloalkyl, C₁₋₄ alkoxy, hydroxyl, cyano or —N(R)₂; and
R^{z} is hydrogen or C₁₋₄ alkyl.

Provided herein as embodiment 2 is the compound according to embodiment 1, wherein L is C₁₋₆ alkylene (e.g., methylene or ethylene) substituted with 0-2 occurrences of R². Provided herein as embodiment 3 is the compound according to embodiment 1, wherein L is —O—C₁₋₆ alkylene (e.g., —O-methylene-, —O-ethylene- or —O-n-propylene) substituted with 0-2 occurrences of R². Provided herein as embodiment 4 is the compound according to embodiment 3, wherein L is —O-ethylene or —O-n-propylene substituted with 0-2 occurrences of R². Provided herein as embodiment 5 is the compound according to embodiment 4, wherein L is —O-ethylene substituted with 0 occurrences of R².

Provided herein as embodiment 6 is the compound according to any one of embodiments 1-5, wherein R¹ is heterocycloalkyl substituted with 0-3 occurrences of R⁵. Provided herein as embodiment 7 is the compound according to embodiment 6, wherein R¹ is 7-(hexahydro-1H-pyrrolizine) substituted with 0-3 occurrences of R⁵. Provided herein as embodiment 8 is the compound according to embodiment 7, wherein R¹ is 7-(hexahydro-1H-pyrrolizine) substituted with 0 occurrences of R⁵. Provided herein as embodiment 9 is the compound according to embodiment 7, wherein R¹ is 7-(hexahydro-1H-pyrrolizine) substituted with 1 occurrence of R⁵. Provided herein as embodiment 10 is the compound according to embodiment 9, wherein R⁵ is halogen (e.g., fluorine).

Provided herein as embodiment 11 is the compound according to embodiment 6, wherein R¹ is 2-pyrrolidine or 3-pyrrolidine substituted with 0-3 occurrences of R⁵. Provided herein as embodiment 12 is the compound according to embodiment 11, wherein R¹ is 3-pyrrolidine substituted with 1 occurrence of R⁵. Provided herein as embodiment 13 is the compound according to embodiment 12, wherein R⁵ is cyano.

Provided herein as embodiment 14 is the compound according to embodiment 11, wherein R¹ is 3-pyrrolidine substituted with 2 occurrences of R⁵. Provided herein as embodiment 15 is the compound according to embodiment 14, wherein one R⁵ is methyl and the other R⁵ is cyano.

Provided herein as embodiment 16 is the compound according to embodiment 11, wherein R¹ is 2-pyrrolidine substituted with 2 occurrences of R⁵. Provided herein as embodiment 17 is the compound according to embodiment 16, wherein R⁵ is C₁₋₄ alkyl (e.g., methyl), oxo, cyano or halogen (e.g., fluorine). Provided herein as embodiment 18 is the compound according to embodiment 17, wherein one R⁵ is methyl and the other R⁵ is fluorine. Provided herein as embodiment 19 is the compound according to embodiment 17, wherein one R⁵ is methyl and the other R⁵ is oxo.

Provided herein as embodiment 20 is the compound according to embodiment 3, wherein L is —O-n-propylene substituted with 2 occurrences of R². Provided herein as embodiment 21 is the compound according to embodiment 20, wherein the two R² are taken together with the same carbon atom to form a C₃₋₇ cycloalkyl (e.g., cyclopropyl). Provided herein as embodiment 22 is the compound according to embodiment 21, wherein R¹ is heterocycloalkyl (e.g., N-morpholinyl) substituted with 0-3 occurrences of R⁵. Provided herein as embodiment 23 is the compound according to embodiment 21, wherein R¹ is hydroxyl.

Provided herein as embodiment 24 is the compound according to any one of embodiments 1-23, wherein -L-R¹ is

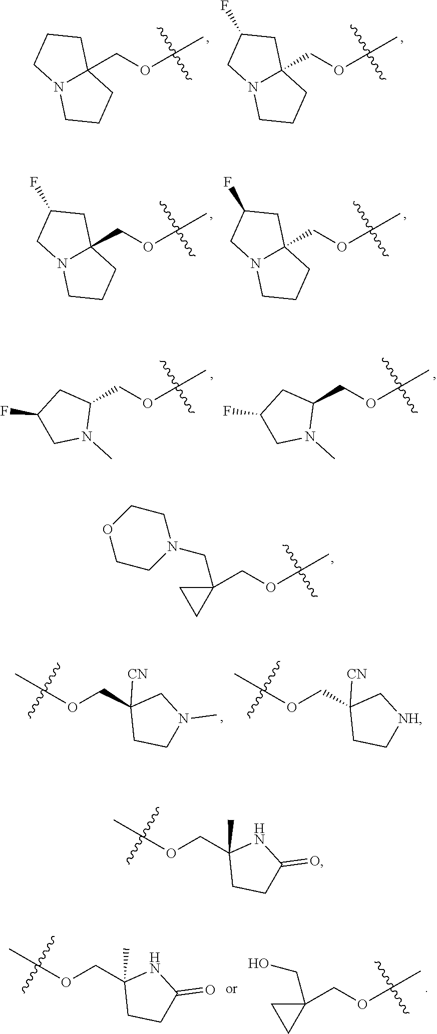

Provided herein as embodiment 25 is the compound according to embodiment 24, wherein -L-R¹ is or

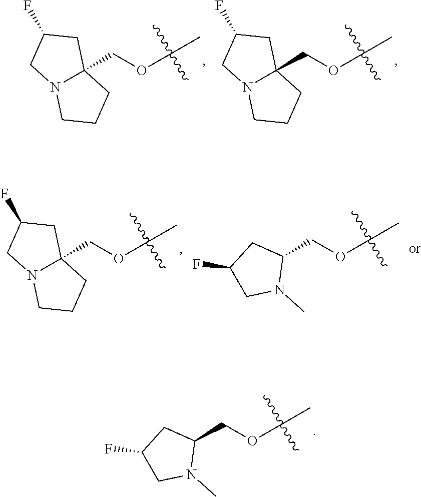

Provided herein as embodiment 26 is the compound according to embodiment 24, wherein -L-R¹ is

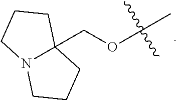 [SMILES: *OCC12CCCN1CCC2]

Provided herein as embodiment 27 is the compound according to embodiment 24, wherein -L-R¹ is

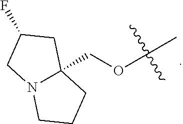 [SMILES: *OC[C@]12CCCN1C[C@H](F)C2]

Provided herein as embodiment 28 is the compound according to embodiment 24, wherein -L-R¹ is

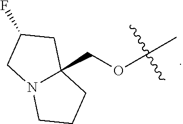 [SMILES: *OC[C@@]12CCCN1C[C@H](F)C2]

Provided herein as embodiment 29 is the compound according to embodiment 24, wherein -L-R¹ is

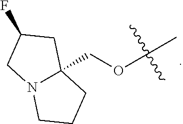 [SMILES: *OC[C@]12CCCN1C[C@@H](F)C2]

Provided herein as embodiment 30 is the compound according to embodiment 24, wherein -L-R¹ is

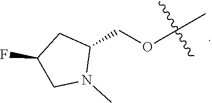 [SMILES: *OC[C@H]1C[C@H](F)CN1C]

Provided herein as embodiment 31 is the compound according to embodiment 24, wherein -L-R¹ is

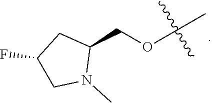 [SMILES: *OC[C@@H]1C[C@@H](F)CN1C]

Provided herein as embodiment 32 is the compound according to embodiment 24, wherein -L-R¹ is

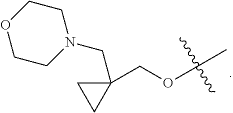 [SMILES: *OCC1(CN2CCOCC2)CC1]

Provided herein as embodiment 33 is the compound according to embodiment 24, wherein -L-R¹ is

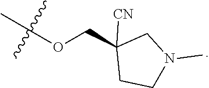 [SMILES: *OC[C@]1(C#N)CCN(C)C1]

Provided herein as embodiment 34 is the compound according to embodiment 24, wherein -L-R¹ is

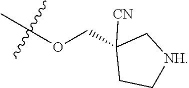 [SMILES: *OC[C@@]1(C#N)CCNC1]

Provided herein as embodiment 35 is the compound according to embodiment 24, wherein -L-R¹ is

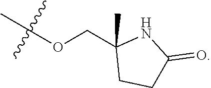 [SMILES: *OC[C@@]1(C)CCC(=O)N1]

Provided herein as embodiment 36 is the compound according to embodiment 24, wherein -L-R¹ is

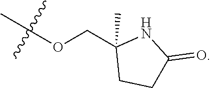 [SMILES: *OC[C@]1(C)CCC(=O)N1]

Provided herein as embodiment 37 is the compound according to embodiment 24, wherein -L-R¹ is

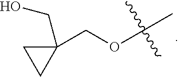 [SMILES: *OCC1(CO)CC1]

Provided herein as embodiment 38 is the compound according to any one of embodiments 1-37, wherein R³ is aryl (e.g., phenyl or naphthyl) substituted with 0-3 occurrences of R⁶.

Provided herein as embodiment 39 is the compound according to embodiment 38, wherein R³ is naphthyl substituted with 1 occurrence of R⁶. Provided herein as embodiment 40 is the compound according to embodiment 39, wherein R⁶ is halogen, amino, C₁₋₄ alkyl (e.g., methyl), C₁₋₄ haloalkyl (e.g., trifluoromethyl or difluoromethyl), hydroxyl or C₂₋₄ alkynyl (e.g., ethynyl). Provided herein as embodiment 41 is the compound according to embodiment 40, wherein R⁶ is hydroxyl.

Provided herein as embodiment 42 is the compound according to embodiment 40, wherein R³ is naphthyl substituted with 2 occurrences of R⁶. Provided herein as embodiment 43 is the compound according to embodiment 42, wherein R⁶ is C₁₋₄ alkyl, C₂₋₄ alkynyl, C₃_6 cycloalkyl, halogen, hydroxyl or —N(R^{z})₂. Provided herein as embodiment 44 is the compound according to embodiment 43, wherein R⁶ is ethyl, ethynyl, cyclopropyl, fluorine, chlorine, hydroxyl or —NH₂. Provided herein as embodiment 45 is the compound according to embodiment 42, wherein one R⁶ is ethynyl and the other R⁶ is hydroxyl. Provided herein as embodiment 46 is the compound according to embodiment 42, wherein one R⁶ is ethyl and the other R⁶ is hydroxyl. Provided herein as embodiment 47 is the compound according to embodiment 42, wherein one R⁶ is ethyl and the other R⁶ is fluorine. Provided herein as embodiment 48 is the compound according to embodiment 42, wherein both R⁶ are fluorine. Provided herein as embodiment 49 is the compound according to embodiment 42, wherein one R⁶ is cyclopropyl and the other R⁶ is hydroxyl. Provided herein as embodiment 50 is the compound according to embodiment 42, wherein one R⁶ is fluorine and the other R⁶ is hydroxyl. Provided herein as embodiment 51 is the compound according to embodiment 42, wherein one R⁶ is chlorine and the other R⁶ is —NH₂. Provided herein as embodiment 52 is the compound according to embodiment 42, wherein one R⁶ is ethynyl and the other R⁶ is fluorine.

Provided herein as embodiment 53 is the compound according to embodiment 40, wherein R³ is naphthyl substituted with 3 occurrences of R⁶. Provided herein as embodiment 54 is the compound according to embodiment 53, wherein R⁶ is C₁₋₄ alkyl, C₂₋₄ alkynyl, halogen or hydroxyl. Provided herein as embodiment 55 is the compound according to embodiment 54, wherein R⁶ is ethyl, ethynyl, fluorine or hydroxyl. Provided herein as embodiment 56 is the compound according to embodiment 53, wherein one R⁶ is hydroxyl, another R⁶ is ethyl and the final R⁶ is fluorine. Provided herein as embodiment 57 is the compound according to embodiment 53, wherein one R⁶ is hydroxyl, another R⁶ is ethynyl and the final R⁶ is fluorine. Provided herein as embodiment 58 is the compound according to embodiment 53, wherein two R⁶ are halogen (e.g., fluorine or chlorine) and the other R⁶ is hydroxy.

Provided herein as embodiment 59 is the compound according to embodiment 38, wherein R³ is phenyl substituted with 3 occurrences of R⁶. Provided herein as embodiment 60 is the compound according to embodiment 59, wherein one R⁶ is hydroxyl, another R⁶ is cyclopropyl and the final R⁶ is chlorine.

Provided herein as embodiment 61 is the compound according to any one of embodiments 1-37, wherein R³ is heteroaryl (e.g., 4-(1H-indazole) or 4-benzo[d]thiazolyl) substituted with 0-3 occurrences of R⁶. Provided herein as embodiment 62 is the compound according to embodiment 61, wherein R³ is 4-(1H-indazole) substituted with 2 occurrences of R⁶. Provided herein as embodiment 63 is the compound according to embodiment 62, wherein one R⁶ is methyl and the other R⁶ is chlorine. Provided herein as embodiment 64 is the compound according to embodiment 61, wherein R³ is 4-benzo[d]thiazolyl substituted with 2 occurrences of R⁶. Provided herein as embodiment 65 is the compound according to embodiment 64, wherein one R⁶ is fluorine and the other R⁶ is —NH₂.

Provided herein as embodiment 66 is the compound according to any one of embodiments 1-65, wherein R³ is

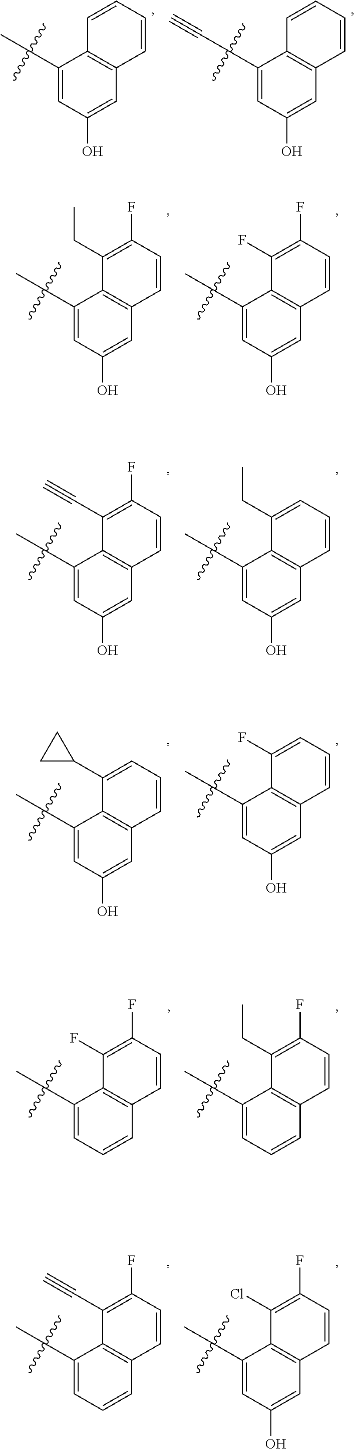

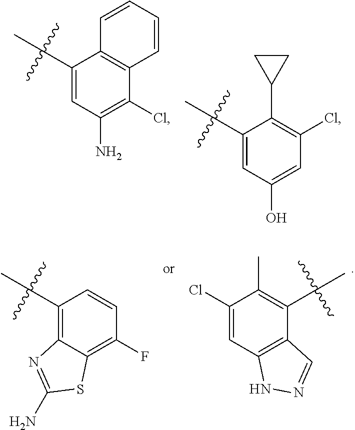

Provided herein as embodiment 67 is the compound according to embodiment 66, wherein R³ is

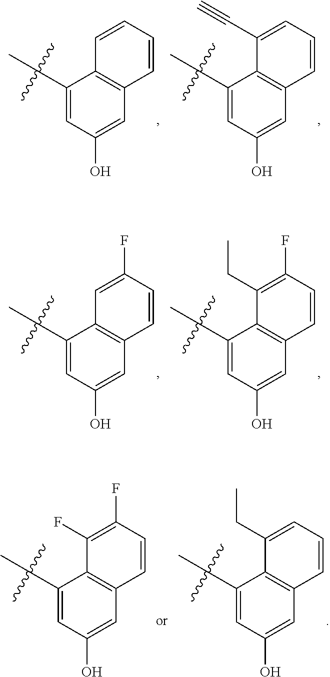

Provided herein as embodiment 68 is the compound according to embodiment 66, wherein R³ is

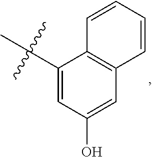 [SMILES: *c1cc(O)cc2ccccc12]

Provided herein as embodiment 69 is the compound according to embodiment 66, wherein R³ is

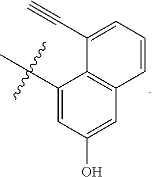 [SMILES: *c1cc(O)cc2cccc(C#C)c12]

Provided herein as embodiment 70 is the compound according to embodiment 66, wherein R³ is

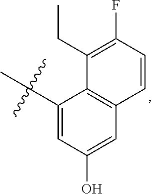 [SMILES: *c1cc(O)cc2ccc(F)c(CC)c12]

Provided herein as embodiment 71 is the compound according to embodiment 66, wherein R³ is

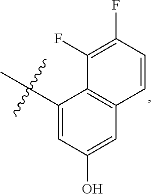 [SMILES: *c1cc(O)cc2ccc(F)c(F)c12]

Provided herein as embodiment 72 is the compound according to embodiment 66, wherein R³ is

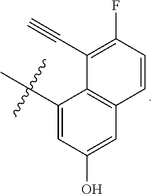 [SMILES: *c1cc(O)cc2ccc(F)c(C#C)c12]

Provided herein as embodiment 73 is the compound according to embodiment 66, wherein R³ is

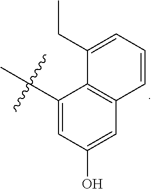 [SMILES: *c1cc(O)cc2cccc(CC)c12]

Provided herein as embodiment 74 is the compound according to embodiment 66, wherein R³ is

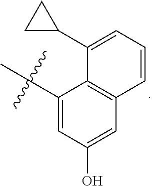 [SMILES: *c1cc(O)cc2cccc(C3CC3)c12]

Provided herein as embodiment 75 is the compound according to embodiment 66, wherein R³ is

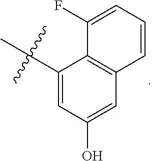 [SMILES: *c1cc(O)cc2cccc(F)c12]

Provided herein as embodiment 76 is the compound according to embodiment 66, wherein R³ is

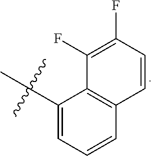 [SMILES: *c1cccc2ccc(F)c(F)c12]

Provided herein as embodiment 77 is the compound according to embodiment 66, wherein R³ is

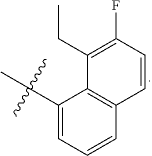 [SMILES: *c1cccc2ccc(F)c(CC)c12]

Provided herein as embodiment 78 is the compound according to embodiment 66, wherein R³ is

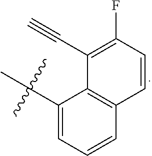 [SMILES: *c1cccc2ccc(F)c(C#C)c12]

Provided herein as embodiment 79 is the compound according to embodiment 66, wherein R³ is

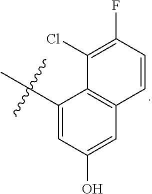 [SMILES: *c1cc(O)cc2ccc(F)c(Cl)c12]

Provided herein as embodiment 80 is the compound according to embodiment 66, wherein R³ is

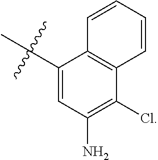 [SMILES: *c1cc(N)c(Cl)c2ccccc12]

Provided herein as embodiment 81 is the compound according to embodiment 66, wherein R₃ is

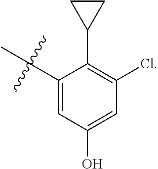 [SMILES: *c1cc(O)cc(Cl)c1C1CC1]

Provided herein as embodiment 82 is the compound according to embodiment 66, wherein R³ is

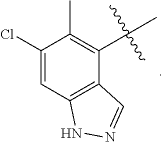 [SMILES: *c1c(C)c(Cl)cc2[nH]ncc12]

Provided herein as embodiment 83 is the compound according to embodiment 66, wherein R is

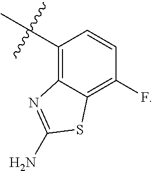 [SMILES: *c1ccc(F)c2sc(N)nc12]

Provided herein as embodiment 84 is the compound according to any one of embodiments 1-83, wherein W is N and [symbol] is a single bond.

Provided herein as embodiment 85 is the compound according to embodiment 84, wherein X is S. Provided herein as embodiment 86 is the compound according to embodiment 85, wherein n is 1 and m is 1. Provided herein as embodiment 87 is the compound according to embodiment 86, wherein p is 0. Provided herein as embodiment 88 is the compound according to embodiment 86, wherein p is 1. Provided herein as embodiment 89 is the compound according to embodiment 88, wherein R^{x} is -T-R^{y} (e.g., CH₂OH). Provided herein as embodiment 90 is the compound according to embodiment 85, wherein n is 1 and m is 2 or m is 1 and n is 2. Provided herein as embodiment 91 is the compound according to embodiment 90, wherein p is 0.

Provided herein as embodiment 92 is the compound according to embodiment 84, wherein X is S(O)₂. Provided herein as embodiment 93 is the compound according to embodiment 92, wherein n is 1 and m is 1. Provided herein as embodiment 94 is the compound according to embodiment 92, wherein n is 1 and m is 2. Provided herein as embodiment 95 is the compound according to embodiment 92, wherein n is 2 and m is 0. Provided herein as embodiment 96 is the compound according to embodiment 93, 94 or 95, wherein p is 0.

Provided herein as embodiment 97 is the compound according to embodiment 93, wherein p is 2. Provided herein as embodiment 98 is the compound according to embodiment 97, wherein two R^{x} taken together form a bridged ring wherein the bridge is —C₁₋₄ alkylene (e.g., methylene or ethylene) further substituted with 0-2 occurrences of R^{y}. Provided herein as embodiment 99 is the compound according to embodiment 98, wherein two R^{x} taken together form a bridged ring wherein the bridge is methylene or ethylene further substituted with 0 occurrences of R^{y}.

Provided herein as embodiment 100 is the compound according to embodiment 84, wherein x is S(O). Provided herein as embodiment 101 is the compound according to embodiment 100, wherein n is 1 and m is 1. Provided herein as embodiment 102 is the compound according to embodiment 100, wherein n is 1 and m is 2. Provided herein as embodiment 103 is the compound according to embodiment 101 or 102, wherein p is 0.

Provided herein as embodiment 104 is the compound according to embodiment 84, wherein X is S(O)(NR^{z}). Provided herein as embodiment 105 is the compound according to embodiment 104, wherein R^{z} is hydrogen. Provided herein as embodiment 106 is the compound according to embodiment 105, wherein n is 1 and m is 1. Provided herein as embodiment 107 is the compound according to embodiment 105, wherein n is 1 and m is 2. Provided herein as embodiment 108 is the compound according to embodiment 106 or 107, wherein p is 0.

Provided herein as embodiment 109 is the compound according to any one of embodiments 1-83, wherein

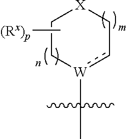

is

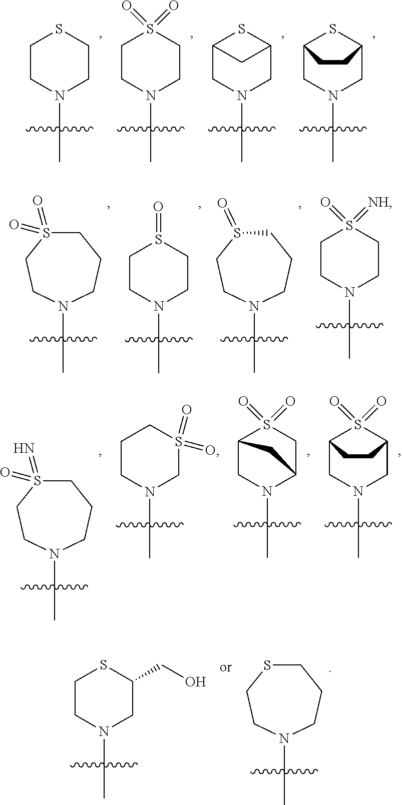

Provided herein as embodiment 110 is the compound according to embodiment 109, wherein

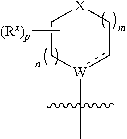

is

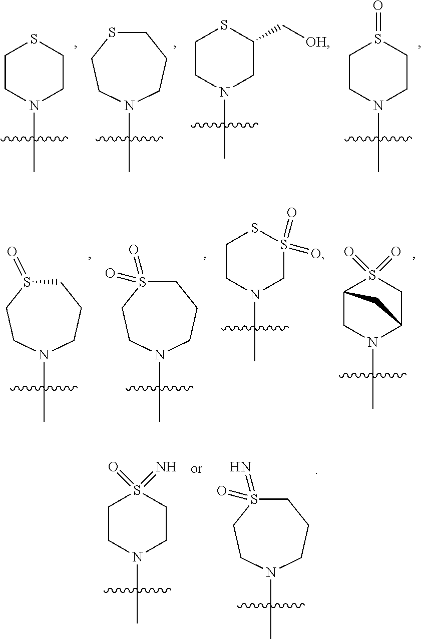

Provided herein as embodiment 111 is the compound according to embodiment 109, wherein

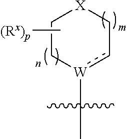

is

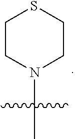 [SMILES: *N1CCSCC1]

Provided herein as embodiment 112 is the compound according to embodiment 109, wherein

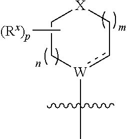

is

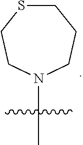 [SMILES: *N1CCCSCC1]

Provided herein as embodiment 113 is the compound according to embodiment 109, wherein

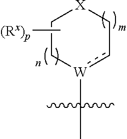

is

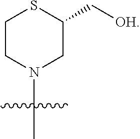 [SMILES: *N1CCS[C@H](CO)C1]

Provided herein as embodiment 114 is the compound according to embodiment 109, wherein

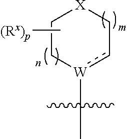

is

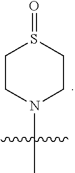 [SMILES: *N1CCS(=O)CC1]

Provided herein as embodiment 115 is the compound according to embodiment 109, wherein

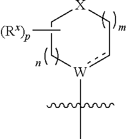

is

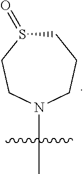 [SMILES: *N1CCC[S@@](=O)CC1]

Provided herein as embodiment 116 is the compound according to embodiment 109, wherein

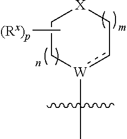

is

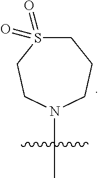 [SMILES: *N1CCCS(=O)(=O)CC1]

Provided herein as embodiment 117 is the compound according to embodiment 109, wherein

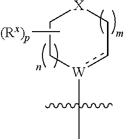

is

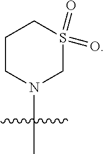 [SMILES: *N1CCCS(=O)(=O)C1]

Provided herein as embodiment 118 is the compound according to embodiment 109, wherein

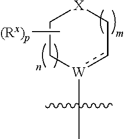

is

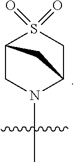 [SMILES: *N1C[C@H]2C[C@@H]1CS2(=O)=O]

Provided herein as embodiment 119 is the compound according to embodiment 109, wherein

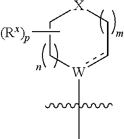

is

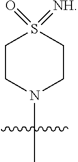 [SMILES: *N1CCS(=N)(=O)CC1]

Provided herein as embodiment 120 is the compound according to embodiment 109, wherein

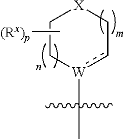

is

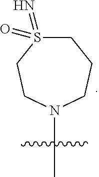 [SMILES: *N1CCCS(=N)(=O)CC1]

Provided herein as embodiment 121 is the compound according to embodiment 84, wherein x is O. Provided herein as embodiment 122 is the compound according to embodiment 121, wherein n is 1 and m is 1. Provided herein as embodiment 123 is the compound according to embodiment 122, wherein p is 0.

Provided herein as embodiment 124 is the compound according to embodiment 122, wherein p is 1. Provided herein as embodiment 125 is the compound according to embodiment 124, wherein R^{x} is C₁₋₄ alkyl, C₁₋₄ haloalkyl, oxo or -T-R^{y}. Provided herein as embodiment 126 is the compound according to embodiment 125, wherein -T-R^{y} is —CH₂CN, CH₂OH, —C(O)NH₂ or —CH₂OMe. Provided herein as embodiment 127 is the compound according to embodiment 125, wherein R^{x} is methyl, difluoromethyl, —CH₂CN, CH₂OH, —C(O)NH₂ or —CH₂OMe.

Provided herein as embodiment 128 is the compound according to embodiment 122, wherein p is 2. Provided herein as embodiment 129 is the compound according to embodiment 128, wherein two R^{x} taken together form a bridged ring wherein the bridge is selected from —C₁₋₄ alkylene (e.g., methylene or ethylene) further substituted with 0-2 occurrences of R^{y}. Provided herein as embodiment 130 is the compound according to embodiment 129, wherein two R^{x} taken together form a bridged ring wherein the bridge is methylene or ethylene further substituted with 0 occurrences of R^{y}.

Provided herein as embodiment 131 is the compound according to embodiment 128, wherein two R^{x} taken together with adjacent carbon atoms form a C₃₋₇ cycloalkyl further substituted with 0-3 occurrences of R^{y}. Provided herein as embodiment 132 is the compound according to embodiment 131, wherein two R^{x} taken together with adjacent carbon atoms form a cyclopropyl further substituted with 0 occurrences of R^{y}.

Provided herein as embodiment 133 is the compound according to embodiment 122, wherein p is 3. Provided herein as embodiment 134 is the compound according to embodiment 133, wherein two R^{x} taken together form a bridged ring wherein the bridge is selected from —C₄ alkylene (e.g., -n-propylene-) further substituted with 0-2 occurrences of R^{y}. Provided herein as embodiment 135 is the compound according to embodiment 134, wherein two R^{x} taken together form a bridged ring wherein the bridge is n-propylene further substituted with one occurrence of R^{y}. Provided herein as embodiment 136 is the compound according to embodiment 135, wherein R^{y} is cyano.

Provided herein as embodiment 137 is the compound according to any one of embodiments 1-83, wherein

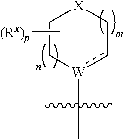

is

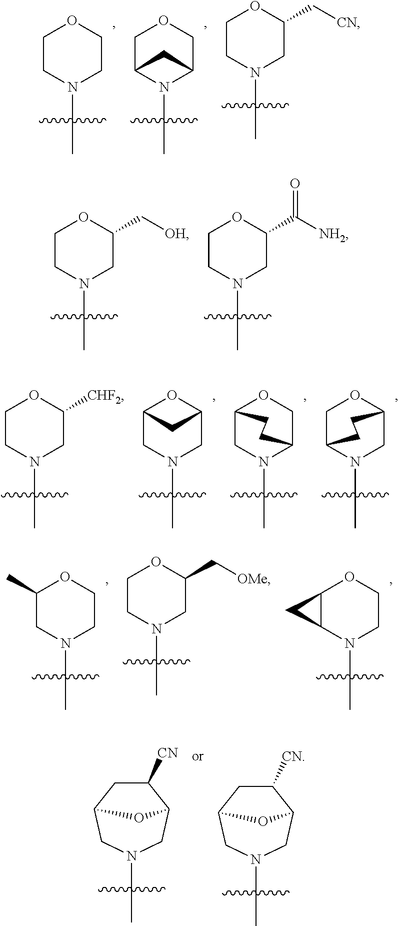

Provided herein as embodiment 138 is the compound according to embodiment 137, wherein

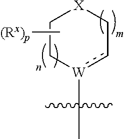

is

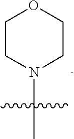 [SMILES: *N1CCOCC1]

Provided herein as embodiment 139 is the compound according to embodiment 137, wherein

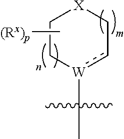

is

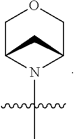 [SMILES: *N1[C@@H]2COC[C@H]1C2]

Provided herein as embodiment 140 is the compound according to embodiment 137, wherein

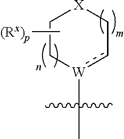

is

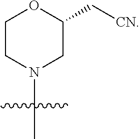 [SMILES: *N1CCO[C@H](CC#N)C1]

Provided herein as embodiment 141 is the compound according to embodiment 137, wherein

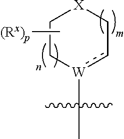

is

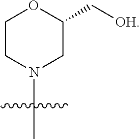 [SMILES: *N1CCO[C@H](CO)C1]

Provided herein as embodiment 142 is the compound according to embodiment 137, wherein

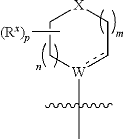

is

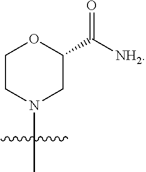 [SMILES: *N1CCO[C@H](C(N)=O)C1]

Provided herein as embodiment 143 is the compound according to embodiment 137, wherein

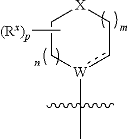

is

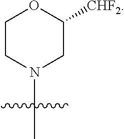 [SMILES: *N1CCO[C@H](C(F)F)C1]

Provided herein as embodiment 144 is the compound according to embodiment 137, wherein

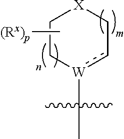

is

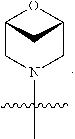 [SMILES: *N1C[C@H]2C[C@@H](C1)O2]

Provided herein as embodiment 145 is the compound according to embodiment 137, wherein

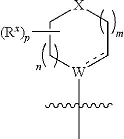

is

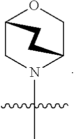

Provided herein as embodiment 146 is the compound according to embodiment 137, wherein

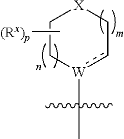

is

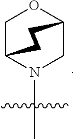

Provided herein as embodiment 147 is the compound according to embodiment 137, wherein

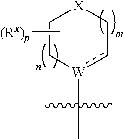

is

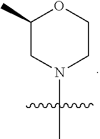 [SMILES: *N1CCO[C@H](C)C1]

Provided herein as embodiment 148 is the compound according to embodiment 137, wherein

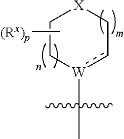

is

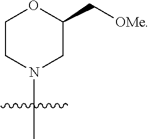 [SMILES: *N1CCO[C@@H](COC)C1]

Provided herein as embodiment 149 is the compound according to embodiment 137, wherein

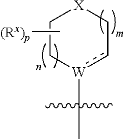

is

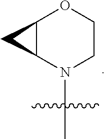 [SMILES: *N1CCO[C@@H]2C[C@@H]21]

Provided herein as embodiment 150 is the compound according to embodiment 137, wherein

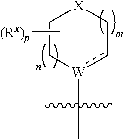

is

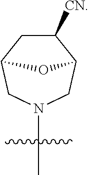 [SMILES: *N1C[C@H]2C[C@@H](C#N)[C@@H](C1)O2]

Provided herein as embodiment 151 is the compound according to embodiment 137, wherein

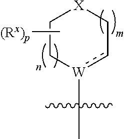

is

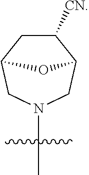 [SMILES: *N1C[C@H]2C[C@H](C#N)[C@@H](C1)O2]

Provided herein as embodiment 152 is the compound according to embodiment 121, wherein n is 1 and m is 2. Provided herein as embodiment 153 is the compound according to embodiment 152, wherein p is 0. Provided herein as embodiment 154 is the compound according to embodiment 152, wherein p is 1. Provided herein as embodiment 155 is the compound according to embodiment 154, wherein R^{x} is oxo, C₁₋₄ alkyl, C₁₋₄ alkoxy, hydroxy, halogen, cyano or -T-R^{y}. Provided herein as embodiment 156 is the compound according to embodiment 155, wherein R^{x} is methyl, cyano, oxo, hydroxy, methoxy, —C(O)N(H)(Me), —C(O)NH₂, —CH₂OH or —SO₂NH₂.

Provided herein as embodiment 157 is the compound according to embodiment 152, wherein p is 2. Provided herein as embodiment 158 is the compound according to embodiment 157, wherein R^{x} is hydroxy, halogen, C₁₋₄ alkyl, C₂₋₄ alkynyl, C₃₋₆ cycloalkyl or two R^{x} taken together form a bridged ring wherein the bridge is —C₁₋₄ alkylene (e.g., methylene). Provided herein as embodiment 159 is the compound according to embodiment 158, wherein R^{x} is hydroxy, methyl, ethyl, fluorine, ethynyl or cyclopropyl. Provided herein as embodiment 160 is the compound according to embodiment 158, wherein one R^{x} is hydroxy and the other R^{x} is methyl or ethyl. Provided herein as embodiment 161 is the compound according to embodiment 158, wherein one R^{x} is hydroxy and the other R^{x} is cyclopropyl. Provided herein as embodiment 162 is the compound according to embodiment 158, wherein one R^{x} is hydroxy and the other R^{x} is ethynyl. Provided herein as embodiment 163 is the compound according to embodiment 158, wherein both R^{x} are fluorine.

Provided herein as embodiment 164 is the compound according to embodiment 158, wherein two R^{x} taken together form a bridged ring wherein the bridge is methylene further substituted with 0-2 occurrences of R^{y}. Provided herein as embodiment 165 is the compound according to embodiment 164, wherein two R^{x} taken together form a bridged ring wherein the bridge is methylene further substituted with 0 occurrences of R^{y}. Provided herein as embodiment 166 is the compound according to embodiment 164, wherein two R^{x} taken together form a bridged ring wherein the bridge is methylene further substituted with 1 occurrence of R^{y}, wherein R^{y} is hydroxy.

Provided herein as embodiment 167 is the compound according to embodiment 121, wherein n is 2 and m is 2. Provided herein as embodiment 168 is the compound according to embodiment 167, wherein p is 2. Provided herein as embodiment 169 is the compound according to embodiment 168, wherein two R^{x} taken together form a bridged ring wherein the bridge is —O— or —C₁₋₄ alkylene wherein the —C₁₋₄ alkylene is further substituted with 0-2 occurrences of R^{y}. Provided herein as embodiment 170 is the compound according to embodiment 169, wherein two R^{x} taken together form a bridged ring wherein the bridge is —O—. Provided herein as embodiment 171 is the compound according to embodiment 169, wherein two R^{x} taken together form a bridged ring wherein the bridge is methylene further substituted with one occurrence of R^{y}. Provided herein as embodiment 172 is the compound according to embodiment 171, wherein R^{y} is hydroxy.

Provided herein as embodiment 173 is the compound according to any one of embodiments 1-83, wherein

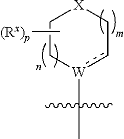

is

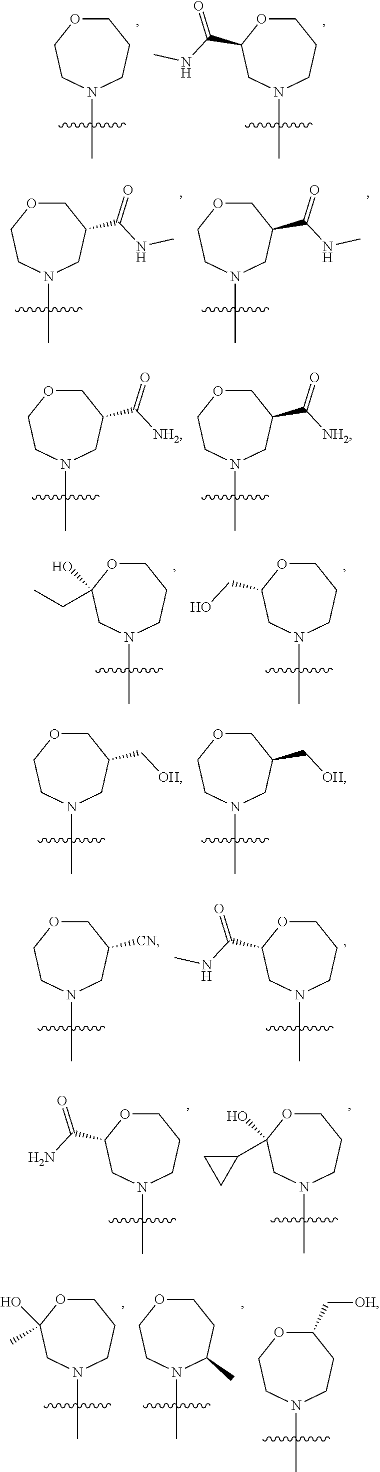

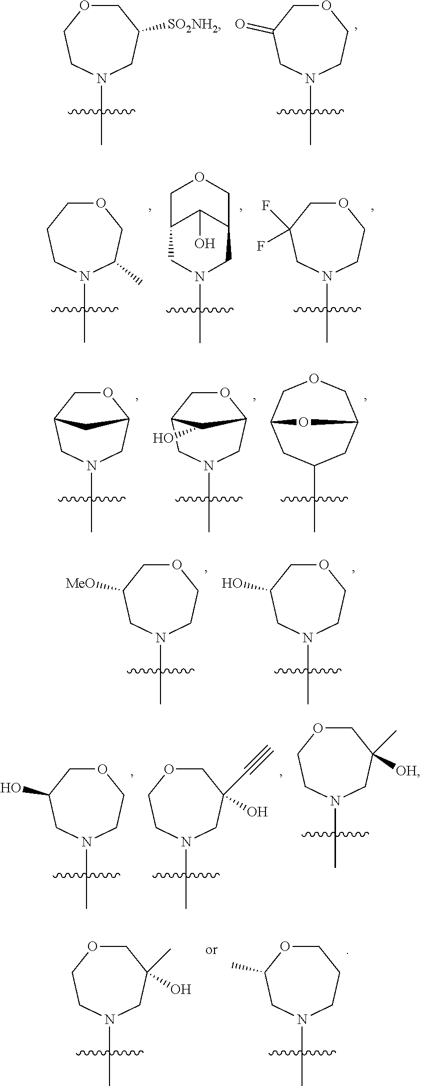

Provided herein as embodiment 174 is the compound according to embodiment 173, wherein

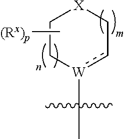

is

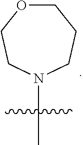 [SMILES: *N1CCCOCC1]

Provided herein as embodiment 175 is the compound according to embodiment 173, wherein

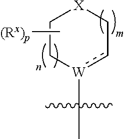

is

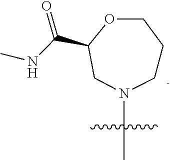 [SMILES: *N1CCCO[C@H](C(=O)NC)C1]

Provided herein as embodiment 176 is the compound according to embodiment 173, wherein

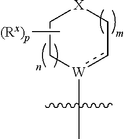

is

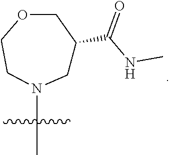 [SMILES: *N1CCOC[C@H](C(=O)NC)C1]

Provided herein as embodiment 177 is the compound according to embodiment 173, wherein

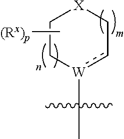

is

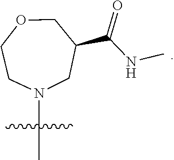 [SMILES: *N1CCOC[C@@H](C(=O)NC)C1]

Provided herein as embodiment 178 is the compound according to embodiment 173, wherein

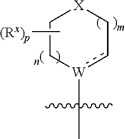

is

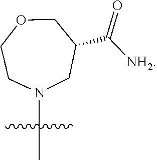 [SMILES: *N1CCOC[C@H](C(N)=O)C1]

Provided herein as embodiment 179 is the compound according to embodiment 173, wherein

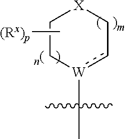

is

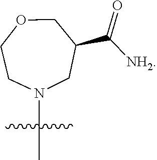 [SMILES: *N1CCOC[C@@H](C(N)=O)C1]

Provided herein as embodiment 180 is the compound according to embodiment 173, wherein

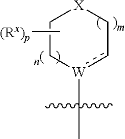

is

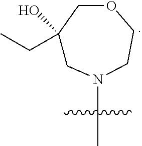 [SMILES: *N1CCOC[C@](O)(CC)C1]

Provided herein as embodiment 181 is the compound according to embodiment 173, wherein

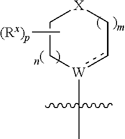

is

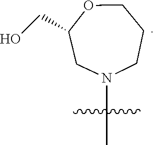 [SMILES: *N1CCCO[C@@H](CO)C1]

Provided herein as embodiment 182 is the compound according to embodiment 173, wherein

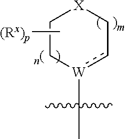

is

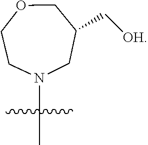 [SMILES: *N1CCOC[C@H](CO)C1]

Provided herein as embodiment 183 is the compound according to embodiment 173, wherein

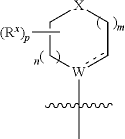

is

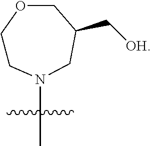 [SMILES: *N1CCOC[C@@H](CO)C1]

Provided herein as embodiment 184 is the compound according to embodiment 173, wherein

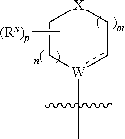

is

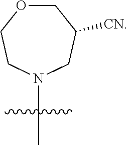 [SMILES: *N1CCOC[C@H](C#N)C1]

Provided herein as embodiment 185 is the compound according to embodiment 173, wherein

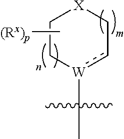

is

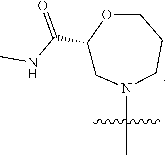 [SMILES: *N1CCCO[C@@H](C(=O)NC)C1]

Provided herein as embodiment 186 is the compound according to embodiment 173, wherein

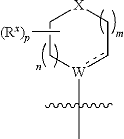

is

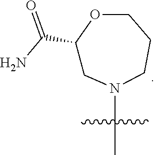 [SMILES: *N1CCCO[C@@H](C(N)=O)C1]

Provided herein as embodiment 187 is the compound according to embodiment 173, wherein

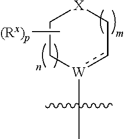

is

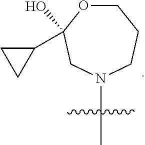 [SMILES: *N1CCCO[C@](O)(C2CC2)C1]

Provided herein as embodiment 188 is the compound according to embodiment 173, wherein

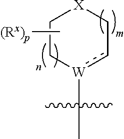

is

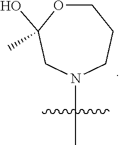 [SMILES: *N1CCCO[C@](C)(O)C1]

Provided herein as embodiment 189 is the compound according to embodiment 173, wherein

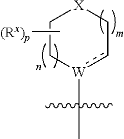

is

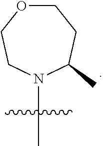 [SMILES: *N1CCOCC[C@H]1C]

Provided herein as embodiment 190 is the compound according to embodiment 173, wherein

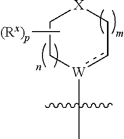

is

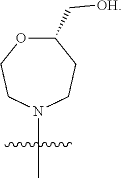 [SMILES: *N1CCO[C@H](CO)CC1]

Provided herein as embodiment 191 is the compound according to embodiment 173, wherein

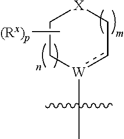

is

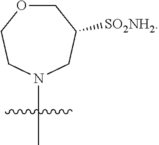 [SMILES: *N1CCOC[C@H](S(N)(=O)=O)C1]

Provided herein as embodiment 192 is the compound according to embodiment 173, wherein

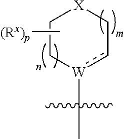

is

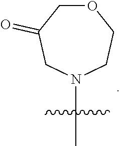 [SMILES: *N1CCOCC(=O)C1]

Provided herein as embodiment 193 is the compound according to embodiment 173, wherein

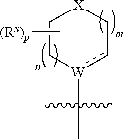

is

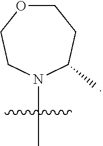 [SMILES: *N1CCOCC[C@@H]1C]

Provided herein as embodiment 194 is the compound according to embodiment 173, wherein

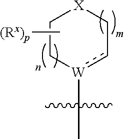

is

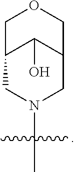 [SMILES: *N1CC2COC[C@@H](C1)C2O]

Provided herein as embodiment 195 is the compound according to embodiment 173, wherein

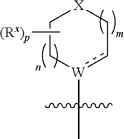

is

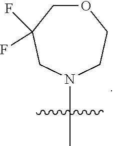 [SMILES: *N1CCOCC(F)(F)C1]

Provided herein as embodiment 196 is the compound according to embodiment 173, wherein

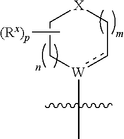

is

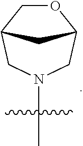 [SMILES: *N1C[C@@H]2CO[C@@H](C2)C1]

Provided herein as embodiment 197 is the compound according to embodiment 173, wherein

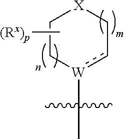

is

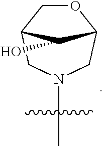 [SMILES: *N1C[C@@H]2CO[C@H](C1)[C@@H]2O]

Provided herein as embodiment 198 is the compound according to embodiment 173, wherein

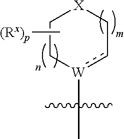

is

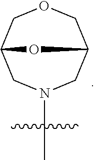 [SMILES: *N1C[C@H]2COC[C@@H](C1)O2]

Provided herein as embodiment 199 is the compound according to embodiment 173, wherein

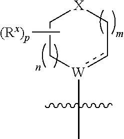

is

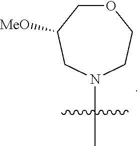 [SMILES: *N1CCOC[C@@H](OC)C1]

Provided herein as embodiment 200 is the compound according to embodiment 173, wherein

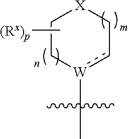

is

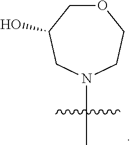 [SMILES: *N1CCOC[C@@H](O)C1]

Provided herein as embodiment 201 is the compound according to embodiment 173, wherein

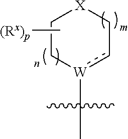

is

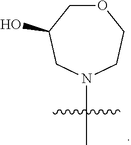 [SMILES: *N1CCOC[C@H](O)C1]

Provided herein as embodiment 202 is the compound according to embodiment 173, wherein

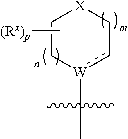

is

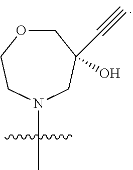 [SMILES: *N1CCOC[C@@](O)(C#C)C1]

Provided herein as embodiment 203 is the compound according to embodiment 173, wherein

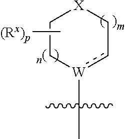

is

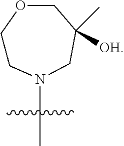 [SMILES: *N1CCOC[C@@](C)(O)C1]

Provided herein as embodiment 204 is the compound according to embodiment 173, wherein

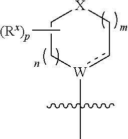

is

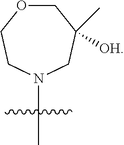 [SMILES: *N1CCOC[C@](C)(O)C1]

Provided herein as embodiment 205 is the compound according to embodiment 173, wherein

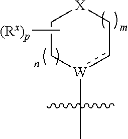

is

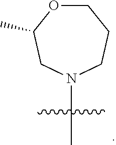 [SMILES: *N1CCCO[C@@H](C)C1]

Provided herein as embodiment 206 is the compound according to any one of embodiments 1-205, wherein R⁴ is C₁₋₄ alkyl, C₁₋₄ alkoxy, hydroxyl, halogen or C₁₋₄ haloalkyl. Provided herein as embodiment 207 is the compound according to embodiment 206, wherein R⁴ is C₁₋₄ alkyl, hydroxyl or halogen. Provided herein as embodiment 208 is the compound according to embodiment 207, wherein R⁴ is C₁₋₄ alkyl or halogen. Provided herein as embodiment 209 is the compound according to embodiment 208, wherein R⁴ halogen (e.g., fluorine or chlorine). Provided herein as embodiment 210 is the compound according to embodiment 209, wherein R⁴ is fluorine.

Provided herein as embodiment 211 is the compound according to embodiment 1, wherein is the compound is a compound of formula (II):

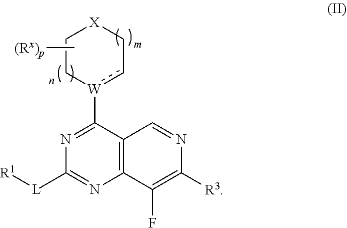

Provided herein as embodiment 212 is the compound according to embodiment 1, wherein is the compound is a compound of formula (III):

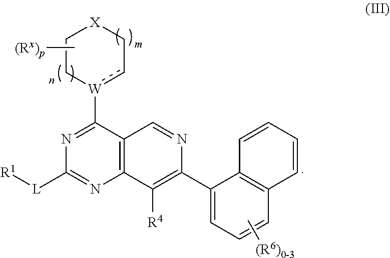

Provided herein as embodiment 213 is the compound according to embodiment 1, wherein is the compound is a compound of formula (IV):

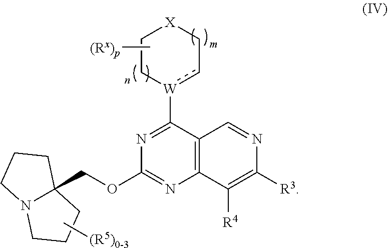

Provided herein as embodiment 214 is the compound according to embodiment 1, wherein is the compound is a compound of formula (V):

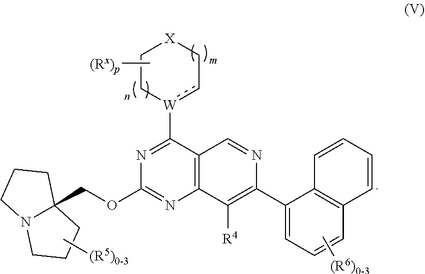

Provided herein as embodiment 215 is the compound according to any one of embodiments 1-214, wherein when R³ is

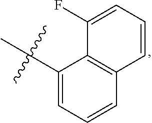 [SMILES: *c1cccc2cccc(F)c12]

then

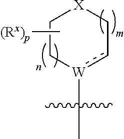

is not

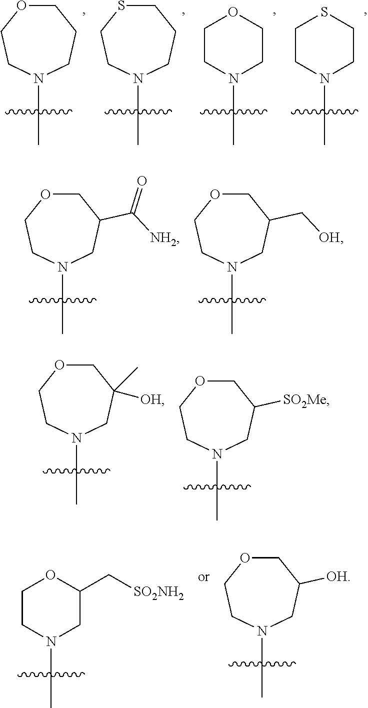

Provided herein as embodiment 216 is the compound according to any one of embodiments 1-214, wherein when R³ is

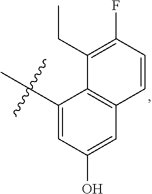 [SMILES: *c1cc(O)cc2ccc(F)c(CC)c12]

then

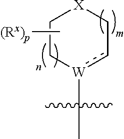

is not

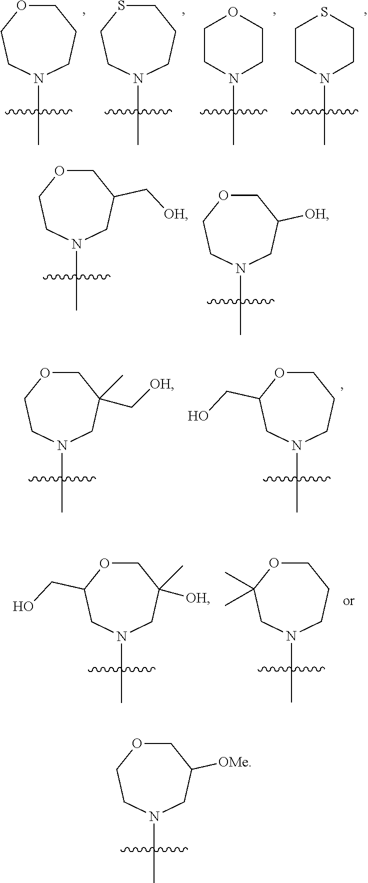

Provided herein as embodiment 217 is the compound according to any one of embodiments 1-214, wherein when R³ is

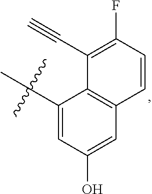 [SMILES: *c1cc(O)cc2ccc(F)c(C#C)c12]

then

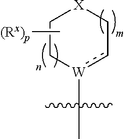

is not

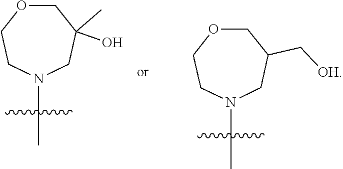

Provided herein as embodiment 218 is the compound according to any one of embodiments 1-214, wherein when R³ is

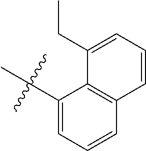 [SMILES: *c1cccc2cccc(CC)c12]

then

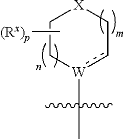

is not

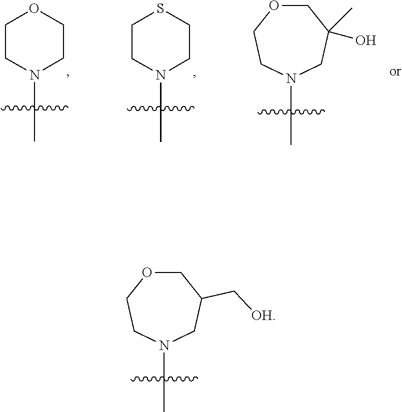

Provided herein as embodiment 219 is the compound according to any one of embodiments 1-214, wherein when R³ is

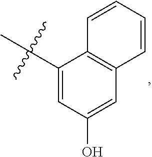 [SMILES: *c1cc(O)cc2ccccc12]

then

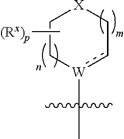

is not

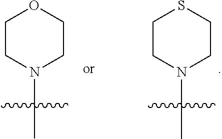 [SMILES: *N1CCOCC1 ; *N1CCSCC1]

Provided herein as embodiment 220 is the compound according to any one of embodiments 1-214, wherein when R³ is

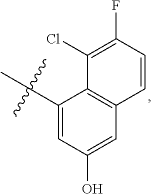 [SMILES: *c1cc(O)cc2ccc(F)c(Cl)c12]

then

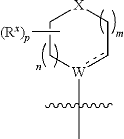

is not

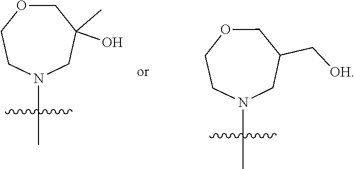

Provided herein as embodiment 221 is the compound according to any one of embodiments 1-214, wherein when R³ is

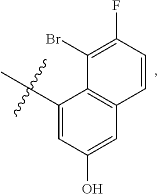 [SMILES: *c1cc(O)cc2ccc(F)c(Br)c12]

then

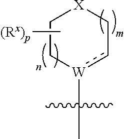

is not

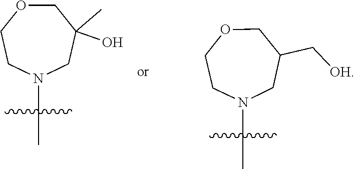

Provided herein as embodiment 222 is the compound according to any one of embodiments 1-214, wherein when R³ is

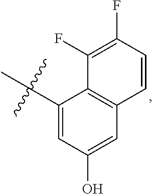 [SMILES: *c1cc(O)cc2ccc(F)c(F)c12]

then

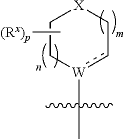

is not

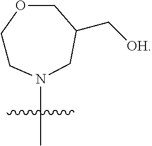 [SMILES: *N1CCOCC(CO)C1]

Provided herein as embodiment 223 is the compound according to any one of embodiments 1-214, wherein when R³ is

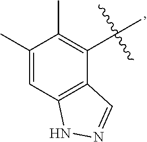

then

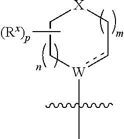

is not

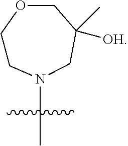 [SMILES: *N1CCOCC(C)(O)C1]

Provided herein as embodiment 224 is the compound according to embodiment 1, wherein the compound is not:
4-(8-fluoro-7-(8-fluoronaphthalen-1-yl)-2-((hexahydro-1H-pyrrolizin-7a-yl)methoxy)pyrido[4,3-d]pyrimidin-4-yl)-1,4-oxazepane;
4-(8-fluoro-7-(8-fluoronaphthalen-1-yl)-2-((hexahydro-1H-pyrrolizin-7a-yl)methoxy)pyrido[4,3-d]pyrimidin-4-yl)-1,4-thiazepane;
4-(8-fluoro-7-(8-fluoronaphthalen-1-yl)-2-((tetrahydro-1H-pyrrolizin-7a(5H)-yl)methoxy)pyrido[4,3-d]pyrimidin-4-yl))morpholine
4-(8-fluoro-7-(8-fluoronaphthalen-1-yl)-2-((hexahydro-1H-pyrrolizin-7a-yl)methoxy)pyrido[4,3-d]pyrimidin-4-yl)thiomorpholine;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
4-(8-fluoro-7-(8-fluoronaphthalen-1-yl)-2-((hexahydro-1H-pyrrolizin-7a-yl)methoxy)pyrido[4,3-d]pyrimidin-4-yl)-6-methyl-1,4-oxazepan-6-ol;
5-ethyl-6-fluoro-4-(8-fluoro-2-(((2R,7aS)-2-fluorotetrahydro-1H-pyrrolizin-7a(5H)-yl)methoxy)-4-(6-((hydroxymethyl)-1,4-oxazepan-4-yl)pyrido[4,3-d]pyrimidin-7-yl)naphthalen-2-ol;
4-(8-fluoro-7-(8-fluoronaphthalen-1-yl)-2-((hexahydro-1H-pyrrolizin-7a-yl)methoxy)pyrido[4,3-d]pyrimidin-4-yl)-1,4-oxazepan-6-yl)nethanol;
4-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-1,4-oxazepan-6-ol;
4-(7-(8-ethynyl-7-fluoro-3-hydroxynaphthalen-1-yl)-8-fluoro-2-(((2R,7aS)-2-fluorohexahydro-1H-pyrrolizin-7a-yl))methoxy)pyrido[4,3-d]pyrimidin-4-yl)-6-methyl-1,4-oxazepan-6-ol;
(S)-4-(8-fluoro-2-((1-methylpyrrolidin-2-yl)methoxy)-4-morpholinopyrido[4,3-d]pyrimidin-7-yl)naphthalen-2-ol;
(S)-4-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-6-methyl-1,4-oxazepan-6-ol;
(S)-4-(7-(S-ethynyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-6-methyl-1,4-oxazepan-6-ol;
1-(4-(8-fluoro-7-(8-fluoronaphthalen-1-yl)-2-((tetrahydro-H-pyrrolizin-7a(5-H)-yl)methoxy)pyrido[4,3-d]pyrimidin-4-yl)morpholin-2-yl)methanesulfonamide;
(4-(8-fluoro-7-(8-fluoronaphthalen-1-yl)-2-((hexahydro-1H-pyrrolizin-7a-yl)methoxy)pyrido[4,3-d]pyrimidin-4-yl)-1,4-oxazepan-2-yl)nethanol;
4-(8-fluoro-7-(8-fluoronaphthalen-1-yl)-2-((hexahydro-1H-pyrrolizin-7a-yl)methoxy)pyrido[4,3-d]pyrimidin-4-yl)-1,4-oxazepane-6-carboxamide;
4-(8-fluoro-7-(8-fluoronaphthalen-1-yl)-2-((hexahydro-1H-pyrrolizin-7a-yl)methoxy)pyrido[4,3-d]pyrimidin-4-yl)-6-(methylsulfonyl)-1,4-oxazepane;
(4-(7-(8-ethylnaphthalen-1-yl)-8-fluoro-2-((hexahydro-1-H-pyrrolizin-7a-yl)methyoxy)pyrido[4,3-d]pyrimidin-4-yl-1,4-oxazepan-6-yl)methanol;
4-(7-(8-ethylnaphthalen-1-yl)-8-fluoro-2-((hexahydro-1H-pyrrolizin-7a-yl)methoxy)pyrido[4,3-d]pyrimidin-4-yl)-6-methyl-1,4-oxazepan-6-ol;
5-ethyl-6-fluoro-4-(8-fluoro-2-(((2R,7aS)-2-fluorohexahydro-1H-pyrrolizin-7a-yl)methoxy)-4-(1,4-oxazepan-4-yl)pyrido[4,3-d]pyrimidin-7-yl)naphthalen-2-ol;
(S)-4-(7-(8-ethyl-7-fluoro-3-hydroxynaphthalen-1-yl)-8-fluoro-2-((1-(pyrrolidin-1-ylmethyl)cyclopropyl)methoxy)pyrido[4,3-d]pyrimidin-4-yl)-6-methyl-1,4-oxazepan-6-ol;
4-(7-(8-ethylnaphthalen-1-yl)-8-fluoro-2-((hexahydro-H-pyrrolizin-7a-yl)methoxy)pyrido[4,3-d]pyrimidin-4-yl)-1,4-oxazepane;
5-ethyl-6-fluoro-4-(8-fluoro-2-(((2R,7aS)-2-fluorohexahydro-1H-pyrrolizin-7a-yl)methoxy)-4-(1,4-thiazepan-4-yl)pyrido[4,3-d]pyrimidin-7-yl)naphthalen-2-ol;
5-ethyl-6-fluoro-4-(8-fluoro-2-(((2R,7aS)-2-fluorotetrahydro-1H-pyrrolizin-7a(5H)-yl)methoxy)-4-(2-(hydroxymethyl)-1,4-oxazepan-4-yl)pyrido[1,3d]pyrimidin-7-yl)naphthalen-2-ol;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
(S)-4-(2-((1-((dimethylamino)methyl)cyclopropyl)methoxy)-7-(8-ethyl-7-fluoro-3-hydroxynaphthalen-1-yl)-8-fluoropyrido[4,3-d]pyrimidin-4-yl)-6-ethyl-1,4-oxazepan-6-ol;
(R)-4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
5-ethynyl-6-fluoro-4-(8-fluoro-2-(((2R,7aS)-2-fluorohexahydro-1H-pyrrolizin-7a-yl)methoxy)-4-(6-(hydroxymethyl)-1,4-oxazepan-4-yl)pyrido[4,3-d]pyrimidin-7-yl)naphthalen-2-ol;
4-(7-(8-bromo-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-6-methyl-1,4-oxazepan-6-ol;
5-bromo-6-fluoro-4-(8-fluoro-2-(((2R,7aS)-2-fluorohexahydro-1H-pyrrolizin-7a-yl)methoxy)-4-(6-(hydroxymethyl)-1,4-oxazepan-4-yl)pyrido[4,3-d]pyrimidin-7-yl)naphthalen-2-ol;
4-(7-(8-chloro-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-6-methyl-1,4-oxazepan-6-ol;
5-chloro-6-fluoro-4-(8-fluoro-2-(((2R,7aS)-2-fluorohexahydro-1H-pyrrolizin-7a-yl)methoxy)-4-(6-(hydroxymethyl)-1,4-oxazepan-4-yl)pyrido[4,3-d]pyrimidin-7-yl)naphthalen-2-ol;
5,6-difluoro-4-(8-fluoro-2-((hexahydro-1H-pyrrolizin-7a-yl)methoxy)-4-(6-(hydroxymethyl)-1,4-oxazepan-4-yl)pyrido[4,3-d]pyrimidin-7-yl)naphthalen-2-ol;
5-ethyl-6-fluoro-4-(8-fluoro-2-(((2R,7aS)-2-fluorotetrahydro-1H-pyrrolizin-7a(5H)-yl)methoxy)-4-(6-(hydroxymethyl)-6-methyl-1,4-oxazepan-4-yl)pyrido[4,3-d]pyrimidin-7-yl)naphthalen-2-ol;
(2R,6R)-4-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-(hydroxymethyl)-6-methyl-1,4-oxazepan-6-ol;
5-ethyl-6-fluoro-4-(8-fluoro-2-(((2R,7aS)-2-fluorotetrahydro-1H-pyrrolizin-7a(5H)-yl)methoxy)-4-(6-methoxy-1,4-oxazepan-4-yl)pyrido[4,3-d]pyrimidin-7-yl)naphthalen-2-ol;
4-(4-(2,2-dimethyl-1,4-oxazepan-4-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol; or
4-(7-(5,6-dimethyl-1H-indazol-4-yl)-8-fluoro-2-((hexahydro-1H-pyrrolizin-7a-yl)methoxy)pyrido[4,3-d]pyrimidin-4-yl)-6-methyl-1,4-oxazepan-6-ol.

Provided herein as embodiment 225 is the compound according to embodiment 1, wherein the compound is not:
4-(8-fluoro-7-(8-fluoronaphthalen-1-yl)-2-((hexahydro-1H-pyrrolizin-7a-yl)methoxy)pyrido[4,3-d]pyrimidin-4-yl)-1,4-oxazepane;
4-(8-fluoro-7-(8-fluoronaphthalen-1-yl)-2-((hexahydro-1H-pyrrolizin-7a-yl)methoxy)pyrido[4,3-d]pyrimidin-4-yl)-1,4-thiazepane;
4-(8-fluoro-7-(8-fluoronaphthalen-1-yl)-2-((tetrahydro-1H-pyrrolizin-7a(5H)-yl)methoxy)pyrido[4,3-d]pyrimidin-4-yl)morpholine;
4-(8-fluoro-7-(8-fluoronaphthalen-1-yl)-2-((hexahydro-1H-pyrrolizin-7a-yl)methoxy)pyrido[4,3-d]pyrimidin-4-v)thiomorpholine;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
4-(8-fluoro-7-(8-fluoronaphthalen-1-yl)-2-((hexahydro-1H-pyrrolizin-7a-yl)methoxy)pyrido[4,3-d]pyrimidin-4-yl)-6-methyl-1,4-oxazepan-6-ol;
5-ethyl-6-fluoro-4-(8-fluoro-2-(((2R,7aS)-2-fluorotetrahydro-1-H-pyrrolizine-7a(5H)-yl)methoxy)-4-(6-(hydroxymethyl)-1,4-oxazepan-4-yl)pyrido[4,3-d]pyrimidin-7-yl)naphthalen-2-ol;
(4-(8-fluoro-7-(8-fluoronaphthalen-1-yl)-2-((hexahydro-1H-pyrrolizin-7a-yl)methoxy)pyrido[4,3-d]pyrimidin-4-yl)-1,4-oxazepan-6-yl)methanol;
4-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-1,4-oxazepan-6-ol;
4-(7-(8-ethynyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-6-methyl-1,4-oxazepan-6-ol;
(S)-4-(8-fluoro-2-((1-methylpyrrolidin-2-yl)methoxy)-4-morpholinopyrido[4,3-d]pyrimidin-7-yl)naphthalen-2-ol;
(S)-4-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-6-methyl-1,4-oxazepan-6-ol; or
(S)-4-(7-(8-ethynyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-6-methyl-1,4-oxazepan-6-ol.

Provided herein as embodiment 226 is the compound according to embodiment 1, wherein the compound is selected from one of the following compounds:
5-Ethyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-ethyl-6-fluoro-4-(8-fluoro-2-(((2R,7aR)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one;
5-Ethyl-6-fluoro-4-(8-fluoro-2-(((2R,7aS)-2-fluorotetrahydro-1H-pyrrolizin-7a(5H)-yl)methoxy)-4-(1,4-thiazepan-4-yl)pyrido[4,3-d]pyrimidin-7-yl)naphthalen-2-ol;
5,6-Difluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethynyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
3-Chloro-4-cyclopropyl-5-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)phenol;
5-Ethyl-6-fluoro-4-(8-fluoro-2-(((2R,7aS)-2-fluorotetrahydro-1H-pyrrolizin-7a(5H)-yl)methoxy)-4-((S)-6-methoxy-1,4-oxazepan-4-yl)pyrido[4,3-d]pyrimidin-7-yl)naphthalen-2-ol;
5-Ethyl-6-fluoro-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carbonitrile;
4-(4-(6-Oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol; or
(S)-4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol.

Provided herein as embodiment 227 is the compound according to embodiment 1, wherein the compound is selected from one of the following compounds:
5-Ethyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-ethyl-6-fluoro-4-(8-fluoro-2-(((2R,7aR)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide; or
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one.

Provided herein as embodiment 228 is the compound according to embodiment 1, wherein the compound is selected from one of the following:
(S)-4-(8-Chloro-7-(8-ethyl-7-fluoro-3-hydroxynaphthalen-1-yl)-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
(S)-4-(7-(8-Ethyl-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
5-Ethyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
(S)-6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-ethyl-6-fluoro-4-(8-fluoro-2-(((2R,7aR)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one;
5-Ethyl-6-fluoro-4-(8-fluoro-2-(((2R,7aS)-2-fluorotetrahydro-1H-pyrrolizin-7a(5H)-yl)methoxy)-4-(1,4-thiazepan-4-yl)pyrido[4,3-d]pyrimidin-7-yl)naphthalen-2-ol;
5,6-Difluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethynyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
3-Chloro-4-cyclopropyl-5-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)phenol;
5-Ethyl-6-fluoro-4-(8-fluoro-2-(((2R,7aS)-2-fluorotetrahydro-1H-pyrrolizin-7a(5H)-yl)methoxy)-4-((S)-6-methoxy-1,4-oxazepan-4-yl)pyrido[4,3-d]pyrimidin-7-yl)naphthalen-2-ol;
5-Ethyl-6-fluoro-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carbonitrile;
4-(4-(6-Oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
(S)-4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 2);
5-Ethyl-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-6-carboxamide (Isomer 2); or
5-Ethyl-6-fluoro-4-(8-fluoro-2-(((2R,7aS)-2-fluorotetrahydro-1H-pyrrolizin-7a(5H)-yl)methoxy)-4-morpholinopyrido[4,3-d]pyrimidin-7-yl)naphthalen-2-ol.

Provided herein as embodiment 229 is the compound according to embodiment 1, wherein the compound is selected from one of the following:
(S)-4-(8-Chloro-7-(8-ethyl-7-fluoro-3-hydroxynaphthalen-1-yl)-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
(S)-4-(7-(8-Ethyl-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
5-Ethyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
(S)-6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-ethyl-6-fluoro-4-(8-fluoro-2-(((2R,7aR)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol; or
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2).

Provided herein as embodiment 230 is the compound according to embodiment 1, wherein the compound is selected from one of the following:
5-Ethyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-thiomorpholinopyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-(1,4-thiazepan-4-yl)pyrido[4,3-d]pyrimidin-7-yl)naphthalen-2-ol;
4-(4-((1R,5S)-3-Oxa-8-azabicyclo[3.2.1]octan-8-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
4-(4-(6-Oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-oxa-3-azabicyclo[3.2.1]octan-8-ol;
5-Ethynyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
4-(8-Fluoro-4-(1,4-oxazepan-4-yl)-2-((tetrahydro-1H-pyrrolizin-7a(5H)-yl)methoxy)pyrido[4,3-d]pyrimidin-7-yl)naphthalen-2-ol;
4-(8-Fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(7-(6-Chloro-5-methyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane;
4-(8-Fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethyl-6-fluoro-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
3-Chloro-4-cyclopropyl-5-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)phenol;
4-(4-((1R,5S)-3,9-Dioxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5,6-difluoronaphthalen-2-ol;
5-Ethyl-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
4-(4-((1R,5S)-3,9-Dioxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
3-Chloro-4-cyclopropyl-5-(8-fluoro-2-((1-(morpholinomethyl)cyclopropyl)methoxy)-4-(1,4-oxazepan-4-yl)pyrido[4,3-d]pyrimidin-7-yl)phenol;
4-(4-((1R,5S)-3,9-Dioxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5-Ethyl-6-fluoro-4-(8-fluoro-2-(((2R,7aS)-2-fluorotetrahydro-1H-pyrrolizin-7a(5H)-yl)methoxy)-4-morpholinopyrido[4,3-d]pyrimidin-7-yl)naphthalen-2-ol;
(S)-4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
(R)-4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
4-(4-((1R,5S)-6-Oxa-3-azabicyclo[3.1.1]heptan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(4-((1S,4R)-2-Oxa-5-azabicyclo[2.2.2]octan-5-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(4-(3-oxa-6-azabicyclo[3.1.1]heptan-6-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
5-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)-5-methylpyrrolidin-2-one;
3-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)pyrrolidine-3-carbonitrile;
5-Ethyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
3-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)-1-methylpyrrolidine-3-carbonitrile;
(S)-4-(7-(8-Ethyl-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
(S)-6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
(S)-4-(8-Chloro-7-(8-ethyl-7-fluoro-3-hydroxynaphthalen-1-yl)-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholine 1-oxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1,6-thiomorpholine
1-oxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1-oxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1,1-dioxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1,6,4-thiazepane 1-oxide;
5-Ethyl-6-fluoro-4-(8-fluoro-2-(((2R,7aS)-2-fluorotetrahydro-1H-pyrrolizin-7a(5H)-yl)methoxy)-4-((S)-6-methoxy-1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-(2-(hydroxymethyl)thiomorpholino)pyrido[4,3-d]pyrimidin-7-yl)naphthalen-2-ol;
5-ethyl-6-fluoro-4-(8-fluoro-2-(((2R,7aR)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-2-carboxamide (Isomer 1);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-2-carboxamide (Isomer 2);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-6-carboxamide (Isomer 1);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-6-carboxamide (Isomer 2);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carboxamide (Isomer 1);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carboxamide (Isomer 2);
5-Ethyl-6-fluoro-4-(8-fluoro-2-(((2R,7aS)-2-fluorotetrahydro-1H-pyrrolizin-7a(5H)-yl)methoxy)-4-(2-(hydroxymethyl)-1,4-oxazepan-4-yl)pyrido[4,3-d]pyrimidin-7-yl)naphthalen-2-ol (Isomer 1);
5-Ethyl-6-fluoro-4-(8-fluoro-2-(((2R,7aS)-2-fluorotetrahydro-1H-pyrrolizin-7a(5H)-yl)methoxy)-4-((S)-6-(hydroxymethyl)-1,4-oxazepan-4-yl)pyrido[4,3-d]pyrimidin-7-yl)naphthalen-2-ol (Isomer 1);
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-2-carboxamide;
5-Ethyl-6-fluoro-4-(8-fluoro-2-(((2R,7aS)-2-fluorotetrahydro-1H-pyrrolizin-7a(5H)-yl)methoxy)-4-((R)-2-methylmorpholino)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-((R)-2-(methoxymethyl)morpholino)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-((R)-5-methyl-1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-((S)-5-methyl-1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-(7-(hydroxymethyl)-1,4-oxazepan-4-yl)pyrido[4,3-d]pyrimidin-7-yl)naphthalen-2-ol;
7-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-oxa-7-azabicyclo[3.3.1]nonan-9-ol;
5-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-thia-5-azabicyclo[2.2.1]heptane 2,2-dioxide;
5-Ethyl-6-fluoro-4-(8-fluoro-2-(((2R,7aS)-2-fluorotetrahydro-1H-pyrrolizin-7a(5H)-yl)methoxy)-4-(2-(hydroxymethyl)morpholino)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carbonitrile;
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-sulfonamide;
4-(4-((1R,5S)-9-oxa-3-azabicyclo[3.3.1]nonan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
2-(4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)morpholin-2-yl)acetonitrile;
4-(4-((1R,6S)-2-oxa-5-azabicyclo[4.1.0]heptan-5-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-ethynyl-1,4-oxazepan-6-ol;
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 1);
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 2);
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
6-Cyclopropyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
4-(4-(2-(Difluoromethyl)morpholino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)morpholine-2-carboxamide; or
5-Ethyl-6-fluoro-4-(8-fluoro-2-((1-(hydroxymethyl)cyclopropyl)methoxy)-4-(1,4-oxazepan-4-yl)pyrido[4,3-d]pyrimidin-7-yl)naphthalen-2-ol.

Provided herein as embodiment 231 is the compound according to embodiment 1, wherein the compound is selected from one of the following:
5-Ethyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-thiomorpholinopyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-(1,4-thiazepan-4-yl)pyrido[4,3-d]pyrimidin-7-yl)naphthalen-2-ol;
4-(4-((1R,5S)-3-Oxa-8-azabicyclo[3.2.1]octan-8-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
4-(4-(6-Oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-oxa-3-azabicyclo[3.2.1]octan-8-ol;
5-Ethynyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
4-(8-Fluoro-4-(1,4-oxazepan-4-yl)-2-((tetrahydro-1H-pyrrolizin-7a(5H)-yl)methoxy)pyrido[4,3-d]pyrimidin-7-yl)naphthalen-2-ol;
4-(8-Fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(7-(6-Chloro-5-methyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane;
4-(8-Fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethyl-6-fluoro-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
3-Chloro-4-cyclopropyl-5-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)phenol;
4-(4-((1R,5S)-3,9-Dioxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5,6-difluoronaphthalen-2-ol;
5-Ethyl-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
4-(4-((1R,5S)-3,9-Dioxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
3-Chloro-4-cyclopropyl-5-(8-fluoro-2-((1-(morpholinomethyl)cyclopropyl)methoxy)-4-(1,4-oxazepan-4-yl)pyrido[4,3-d]pyrimidin-7-yl)phenol;
4-(4-((1R,5S)-3,9-Dioxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5-Ethyl-6-fluoro-4-(8-fluoro-2-(((2R,7aS)-2-fluorotetrahydro-1H-pyrrolizin-7a(5H)-yl)methoxy)-4-morpholinopyrido[4,3-d]pyrimidin-7-yl)naphthalen-2-ol;
(S)-4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
(R)-4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
4-(4-((1R,5S)-6-Oxa-3-azabicyclo[3.1.1]heptan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(4-((1S,4R)-2-Oxa-5-azabicyclo[2.2.2]octan-5-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(4-(3-oxa-6-azabicyclo[3.1.1]heptan-6-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
5-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)-5-methylpyrrolidin-2-one;
3-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)pyrrolidine-3-carbonitrile;
5-Ethyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
3-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)-1-methylpyrrolidine-3-carbonitrile;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholine 1-oxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1,6-thiomorpholine 1-oxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1-oxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1,1-dioxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1,6,4-thiazepane 1-oxide;
5-Ethyl-6-fluoro-4-(8-fluoro-2-(((2R,7aS)-2-fluorotetrahydro-1H-pyrrolizin-7a(5H)-yl)methoxy)-4-((S)-6-methoxy-1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-(2-(hydroxymethyl)thiomorpholino)pyrido[4,3-d]pyrimidin-7-yl)naphthalen-2-ol;
5-ethyl-6-fluoro-4-(8-fluoro-2-(((2R,7aR)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-2-carboxamide (Isomer 1);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-2-carboxamide (Isomer 2);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-6-carboxamide (Isomer 1);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-6-carboxamide (Isomer 2);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carboxamide (Isomer 1);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carboxamide (Isomer 2);
5-Ethyl-6-fluoro-4-(8-fluoro-2-(((2R,7aS)-2-fluorotetrahydro-1H-pyrrolizin-7a(5H)-yl)methoxy)-4-(2-(hydroxymethyl)-1,4-oxazepan-4-yl)pyrido[4,3-d]pyrimidin-7-yl)naphthalen-2-ol (Isomer 1);
5-Ethyl-6-fluoro-4-(8-fluoro-2-(((2R,7aS)-2-fluorotetrahydro-1H-pyrrolizin-7a(5H)-yl)methoxy)-4-((S)-6-(hydroxymethyl)-1,4-oxazepan-4-yl)pyrido[4,3-d]pyrimidin-7-yl)naphthalen-2-ol (Isomer 1);
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-2-carboxamide;
5-Ethyl-6-fluoro-4-(8-fluoro-2-(((2R,7aS)-2-fluorotetrahydro-1H-pyrrolizin-7a(5H)-yl)methoxy)-4-((R)-2-methylmorpholino)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-((R)-2-(methoxymethyl)morpholino)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-((R)-5-methyl-1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-((S)-5-methyl-1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-(7-(hydroxymethyl)-1,4-oxazepan-4-yl)pyrido[4,3-d]pyrimidin-7-yl)naphthalen-2-ol;
7-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-oxa-7-azabicyclo[3.3.1]nonan-9-ol;
5-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-thia-5-azabicyclo[2.2.1]heptane 2,2-dioxide;
5-Ethyl-6-fluoro-4-(8-fluoro-2-(((2R,7aS)-2-fluorotetrahydro-1H-pyrrolizin-7a(5H)-yl)methoxy)-4-(2-(hydroxymethyl)morpholino)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carbonitrile;
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-sulfonamide;
4-(4-((1R,5S)-9-oxa-3-azabicyclo[3.3.1]nonan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
2-(4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)morpholin-2-yl)acetonitrile;
4-(4-((1R,6S)-2-oxa-5-azabicyclo[4.1.0]heptan-5-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-ethynyl-1,4-oxazepan-6-ol;
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 1);
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 2);
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
6-Cyclopropyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
4-(4-(2-(Difluoromethyl)morpholino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)morpholine-2-carboxamide; or
5-Ethyl-6-fluoro-4-(8-fluoro-2-((1-(hydroxymethyl)cyclopropyl)methoxy)-4-(1,4-oxazepan-4-yl)pyrido[4,3-d]pyrimidin-7-yl)naphthalen-2-ol.

Provided herein as embodiment 232 is the compound according to embodiment 1, wherein the compound is selected from one of the following:
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-thiomorpholinopyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-(1,4-thiazepan-4-yl)pyrido[4,3-d]pyrimidin-7-yl)naphthalen-2-ol;
4-(4-((1R,5S)-3-Oxa-8-azabicyclo[3.2.1]octan-8-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
4-(4-(6-Oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-oxa-3-azabicyclo[3.2.1]octan-8-ol;
5-Ethynyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
4-(8-Fluoro-4-(1,4-oxazepan-4-yl)-2-((tetrahydro-1H-pyrrolizin-7a(5H)-yl)methoxy)pyrido[4,3-d]pyrimidin-7-yl)naphthalen-2-ol;
4-(8-Fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(7-(6-Chloro-5-methyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane;
4-(8-Fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethyl-6-fluoro-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
3-Chloro-4-cyclopropyl-5-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)phenol;
4-(4-((1R,5S)-3,9-Dioxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5,6-difluoronaphthalen-2-ol;
5-Ethyl-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
4-(4-((1R,5S)-3,9-Dioxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
3-Chloro-4-cyclopropyl-5-(8-fluoro-2-((1-(morpholinomethyl)cyclopropyl)methoxy)-4-(1,4-oxazepan-4-yl)pyrido[4,3-d]pyrimidin-7-yl)phenol;
4-(4-((1R,5S)-3,9-Dioxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5-Ethyl-6-fluoro-4-(8-fluoro-2-(((2R,7aS)-2-fluorotetrahydro-1H-pyrrolizin-7a(5H)-yl)methoxy)-4-morpholinopyrido[4,3-d]pyrimidin-7-yl)naphthalen-2-ol;
4-(4-((1R,5S)-6-Oxa-3-azabicyclo[3.1.1]heptan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(4-((1S,4R)-2-Oxa-5-azabicyclo[2.2.2]octan-5-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(4-(3-oxa-6-azabicyclo[3.1.1]heptan-6-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
5-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)-5-methylpyrrolidin-2-one;
3-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)pyrrolidine-3-carbonitrile;
5-Ethyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
3-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)-1-methylpyrrolidine-3-carbonitrile;
(S)-4-(7-(8-Ethyl-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
(S)-6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
(S)-4-(8-Chloro-7-(8-ethyl-7-fluoro-3-hydroxynaphthalen-1-yl)-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholine 1-oxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1,6-thiomorpholine
1-oxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1-oxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1,1-dioxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1,6,4-thiazepane 1-oxide;
5-Ethyl-6-fluoro-4-(8-fluoro-2-(((2R,7aS)-2-fluorotetrahydro-1H-pyrrolizin-7a(5H)-yl)methoxy)-4-(2-(hydroxymethyl)thiomorpholino)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-2-carboxamide (Isomer 1);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-2-carboxamide (Isomer 2);
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-2-carboxamide;
5-Ethyl-6-fluoro-4-(8-fluoro-2-(((2R,7aS)-2-fluorotetrahydro-1H-pyrrolizin-7a(5H)-yl)methoxy)-4-((R)-2-methylmorpholino)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-((R)-2-(methoxymethyl)morpholino)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-((R)-5-methyl-1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-((S)-5-methyl-1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-(7-(hydroxymethyl)-1,4-oxazepan-4-yl)pyrido[4,3-d]pyrimidin-7-yl)naphthalen-2-ol;
7-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-oxa-7-azabicyclo[3.3.1]nonan-9-ol;
5-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-thia-5-azabicyclo[2.2.1]heptane 2,2-dioxide;
5-Ethyl-6-fluoro-4-(8-fluoro-2-(((2R,7aS)-2-fluorotetrahydro-1H-pyrrolizin-7a(5H)-yl)methoxy)-4-(2-(hydroxymethyl)morpholino)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carbonitrile;
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide;
4-(4-((1R,5S)-9-oxa-3-azabicyclo[3.3.1]nonan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
2-(4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)morpholin-2-yl)acetonitrile;
4-(4-((1R,6S)-2-oxa-5-azabicyclo[4.1.0]heptan-5-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-ethynyl-1,4-oxazepan-6-ol;
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 1);
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 2);
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
6-Cyclopropyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
4-(4-(2-(Difluoromethyl)morpholino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)morpholine-2-carboxamide; or
5-Ethyl-6-fluoro-4-(8-fluoro-2-((1-(hydroxymethyl)cyclopropyl)methoxy)-4-(1,4-oxazepan-4-yl)pyrido[4,3-d]pyrimidin-7-yl)naphthalen-2-ol.

Provided herein as embodiment 233 is the compound according to embodiment 1, wherein the compound is selected from one of the following:
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-thiomorpholinopyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-(1,4-thiazepan-4-yl)pyrido[4,3-d]pyrimidin-7-yl)naphthalen-2-ol;
4-(4-((1R,5S)-3-Oxa-8-azabicyclo[3.2.1]octan-8-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
4-(4-(6-Oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-oxa-3-azabicyclo[3.2.1]octan-8-ol;
5-Ethynyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
4-(8-Fluoro-4-(1,4-oxazepan-4-yl)-2-((tetrahydro-1H-pyrrolizin-7a(5H)-yl)methoxy)pyrido[4,3-d]pyrimidin-7-yl)naphthalen-2-ol;
4-(8-Fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(7-(6-Chloro-5-methyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane;
4-(8-Fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethyl-6-fluoro-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
3-Chloro-4-cyclopropyl-5-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)phenol;
4-(4-((1R,5S)-3,9-Dioxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5,6-difluoronaphthalen-2-ol;
5-Ethyl-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
4-(4-((1R,5S)-3,9-Dioxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
3-Chloro-4-cyclopropyl-5-(8-fluoro-2-((1-(morpholinomethyl)cyclopropyl)methoxy)-4-(1,4-oxazepan-4-yl)pyrido[4,3-d]pyrimidin-7-yl)phenol;
4-(4-((1R,5S)-3,9-Dioxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5-Ethyl-6-fluoro-4-(8-fluoro-2-(((2R,7aS)-2-fluorotetrahydro-1H-pyrrolizin-7a(5H)-yl)methoxy)-4-morpholinopyrido[4,3-d]pyrimidin-7-yl)naphthalen-2-ol;
4-(4-((1R,5S)-6-Oxa-3-azabicyclo[3.1.1]heptan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(4-((1S,4R)-2-Oxa-5-azabicyclo[2.2.2]octan-5-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(4-(3-oxa-6-azabicyclo[3.1.1]heptan-6-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
5-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)-5-methylpyrrolidin-2-one;
3-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)pyrrolidine-3-carbonitrile;
5-Ethyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
3-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)-1-methylpyrrolidine-3-carbonitrile;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholine 1-oxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1,6-thiomorpholine 1-oxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1-oxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1,1-dioxide;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1,6,4-thiazepane 1-oxide;
5-Ethyl-6-fluoro-4-(8-fluoro-2-(((2R,7aS)-2-fluorotetrahydro-1H-pyrrolizin-7a(5H)-yl)methoxy)-4-(2-(hydroxymethyl)thiomorpholino)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-2-carboxamide (Isomer 1);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-2-carboxamide (Isomer 2);
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-2-carboxamide;
5-Ethyl-6-fluoro-4-(8-fluoro-2-(((2R,7aS)-2-fluorotetrahydro-1H-pyrrolizin-7a(5H)-yl)methoxy)-4-((R)-2-methylmorpholino)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-((R)-2-(methoxymethyl)morpholino)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-((R)-5-methyl-1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-((S)-5-methyl-1,4-oxazepan-4-yl)pyrido[4,3-d]pyrimidin-7-yl)naphthalen-2-ol;
5-Ethyl-6-fluoro-4-(8-fluoro-2-(((2R,7aS)-2-fluorotetrahydro-1H-pyrrolizin-7a(5H)-yl)methoxy)-4-(7-(hydroxymethyl)-1,4-oxazepan-4-yl)pyrido[4,3-d]pyrimidin-7-yl)naphthalen-2-ol;
7-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-oxa-7-azabicyclo[3.3.1]nonan-9-ol;
5-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-thia-5-azabicyclo[2.2.1]heptane 2,2-dioxide;
5-Ethyl-6-fluoro-4-(8-fluoro-2-(((2R,7aS)-2-fluorotetrahydro-1H-pyrrolizin-7a(5H)-yl)methoxy)-4-(2-(hydroxymethyl)morpholino)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carbonitrile;
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide;
4-(4-((1R,5S)-9-oxa-3-azabicyclo[3.3.1]nonan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
2-(4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)morpholin-2-yl)acetonitrile;
4-(4-((1R,6S)-2-oxa-5-azabicyclo[4.1.0]heptan-5-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-ethynyl-1,4-oxazepan-6-ol;
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 1);
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 2);
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
6-Cyclopropyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
4-(4-(2-(Difluoromethyl)morpholino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)morpholine-2-carboxamide; or
5-Ethyl-6-fluoro-4-(8-fluoro-2-((1-(hydroxymethyl)cyclopropyl)methoxy)-4-(1,4-oxazepan-4-yl)pyrido[4,3-d]pyrimidin-7-yl)naphthalen-2-ol.

Provided herein as embodiment 234 is the compound according to embodiment 1, wherein the compound is selected from one of the following:
(S)-4-(8-Chloro-7-(8-ethyl-7-fluoro-3-hydroxynaphthalen-1-yl)-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
(S)-4-(7-(8-Ethyl-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
5-Ethyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
(S)-6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-ethyl-6-fluoro-4-(8-fluoro-2-(((2R,7aR)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one;
5-Ethyl-6-fluoro-4-(8-fluoro-2-(((2R,7aS)-2-fluorotetrahydro-1H-pyrrolizin-7a(5H)-yl)methoxy)-4-(1,4-thiazepan-4-yl)pyrido[4,3-d]pyrimidin-7-yl)naphthalen-2-ol;
5,6-Difluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethynyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
3-Chloro-4-cyclopropyl-5-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)phenol;
5-Ethyl-6-fluoro-4-(8-fluoro-2-(((2R,7aS)-2-fluorotetrahydro-1H-pyrrolizin-7a(5H)-yl)methoxy)-4-((S)-6-methoxy-1,4-oxazepan-4-yl)pyrido[4,3-d]pyrimidin-7-yl)naphthalen-2-ol;
5-Ethyl-6-fluoro-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carbonitrile;
4-(4-(6-Oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
(S)-4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 2);
5-Ethyl-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-6-carboxamide (Isomer 2); or
5-Ethyl-6-fluoro-4-(8-fluoro-2-(((2R,7aS)-2-fluorotetrahydro-1H-pyrrolizin-7a(5H)-yl)methoxy)-4-morpholinopyrido[4,3-d]pyrimidin-7-yl)naphthalen-2-ol.

Provided herein as embodiment 235 is the compound according to embodiment 1, wherein the compound is selected from one of the following:
5-Ethyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-ethyl-6-fluoro-4-(8-fluoro-2-(((2R,7aR)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one;
5-Ethyl-6-fluoro-4-(8-fluoro-2-(((2R,7aS)-2-fluorotetrahydro-1H-pyrrolizin-7a(5H)-yl)methoxy)-4-(1,4-thiazepan-4-yl)pyrido[4,3-d]pyrimidin-7-yl)naphthalen-2-ol;
5,6-Difluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethynyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
3-Chloro-4-cyclopropyl-5-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)phenol;
5-Ethyl-6-fluoro-4-(8-fluoro-2-(((2R,7aS)-2-fluorotetrahydro-1H-pyrrolizin-7a(5H)-yl)methoxy)-4-((S)-6-methoxy-1,4-oxazepan-4-yl)pyrido[4,3-d]pyrimidin-7-yl)naphthalen-2-ol;
5-Ethyl-6-fluoro-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carbonitrile;
4-(4-(6-Oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
(S)-4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 2);
5-Ethyl-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-1,4-oxazepane-6-carboxamide (Isomer 2); or
5-Ethyl-6-fluoro-4-(8-fluoro-2-(((2R,7aS)-2-fluorotetrahydro-1H-pyrrolizin-7a(5H)-yl)methoxy)-4-morpholinopyrido[4,3-d]pyrimidin-7-yl)naphthalen-2-ol.

Provided herein as embodiment 236 is the compound according to embodiment 1, wherein the compound is selected from one of the following:
(S)-4-(8-Chloro-7-(8-ethyl-7-fluoro-3-hydroxynaphthalen-1-yl)-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
(S)-4-(7-(8-Ethyl-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-1,4-oxazepan-6-ol;
(S)-6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one;
5,6-Difluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethynyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
3-Chloro-4-cyclopropyl-5-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)phenol;
5-Ethyl-6-fluoro-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carbonitrile;
4-(4-(6-Oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 2);
5-Ethyl-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol; or
5-Ethyl-6-fluoro-4-(8-fluoro-2-(((2R,7aS)-2-fluorotetrahydro-1H-pyrrolizin-7a(5H)-yl)methoxy)-4-morpholinopyrido[4,3-d]pyrimidin-7-yl)naphthalen-2-ol.

Provided herein as embodiment 237 is the compound according to embodiment 1, wherein the compound is selected from one of the following:
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one;
5,6-Difluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethynyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
3-Chloro-4-cyclopropyl-5-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)phenol;
5-Ethyl-6-fluoro-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carbonitrile;
4-(4-(6-Oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane-6-carbonitrile (Isomer 2);
5-Ethyl-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
5-Ethyl-6-fluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol; or
5-Ethyl-6-fluoro-4-(8-fluoro-2-(((2R,7aS)-2-fluorotetrahydro-1H-pyrrolizin-7a(5H)-yl)methoxy)-4-morpholinopyrido[4,3-d]pyrimidin-7-yl)naphthalen-2-ol.

Provided herein as embodiment 238 is the compound according to embodiment 1, wherein the compound is not example 42, 43, 49, 50, 63, 64, 74, 83, 111, 149, 195, 200, 201, 249, 269, 270, 271, 289, 291, 315, 321, 334, 342, 343, 345, 361, 386, 391, 400, 401, 419, 420, 457, 496, 497, 499, 501 or 522 from international publication No. WO 2022/132200 (International Application No. PCT/US2021/010065).

Provided herein as embodiment 239 is the compound according to embodiment 1, wherein the compound is not example 42, 43, 49, 50, 63, 64, 74, 83, 111, 149, 195, 200 or 201 from international publication No. WO 2022/132200 (International Application No. PCT/US2021/010065).

The foregoing merely summarizes certain aspects of this disclosure and is not intended, nor should it be construed, as limiting the disclosure in any way.

### Formulation, and Route of Administration

While it may be possible to administer a compound disclosed herein alone in the uses described, the compound administered normally will be present as an active ingredient in a pharmaceutical composition. Thus, in one embodiment, provided herein is a pharmaceutical composition comprising a compound disclosed herein in combination with one or more pharmaceutically acceptable excipients, such as diluents, carriers, adjuvants and the like, and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by G D Tovey, Royal Society of Chemistry, 2018. In one embodiment, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

Provided herein as embodiment 240 is a pharmaceutical composition comprising the compound according to any one of embodiments 1-239, or a tautomer thereof, or a pharmaceutically acceptable salt of said compound or said tautomer, and a pharmaceutically acceptable excipient.

Provided herein as embodiment 241 is a compound according to any one of Embodiments 1-239, or a tautomer thereof, or a pharmaceutically acceptable salt of said compound or said tautomer, or the pharmaceutical composition according to embodiment 240 for use as a medicament.

### Methods of Use

As discussed herein (see, section entitled “Definitions”), the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms.

Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

In one embodiment, the disclosure provides methods of using the compounds or pharmaceutical compositions of the present disclosure to treat disease conditions, including but not limited to conditions implicated by KRAS G12D, G12V, G12A, G12S or G12C mutation (e.g., cancer). The cancer types are non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

KRAS G12D mutations occur with the alteration frequencies shown in the table below (TCGA data sets;¹⁻³ For example, the table shows that 32.4% of subjects with pancreatic cancer have a cancer wherein one or more cells express KRAS G12D mutant protein. Accordingly, the compounds provided herein, which bind to KRAS^{G12D} (see Section entitled “Biological Evaluation” below) are useful for treatment of subjects having a cancer, including, but not limited to the cancers listed in the table below.

| | Alteration |
|---|---|
| Cancer Type | Frequency |
| Pancreatic Adenocarcinoma (PAAD) | 32.4 |
| Colon Adenocarcinoma (COAD) | 12.25 |
| Rectal adenocarcinoma (READ) | 8.03 |
| Uterine corpus endometrial carcinoma | 6.04 |
| (UCEC) | |
| Lung Adenocarcinoma (LUAD) | 3.53 |
| Plasma Cell Tumors | 2.92 |
| Stomach Adenocarcinoma (STAD) | 2.27 |
| Bladder urothelial carcinoma (BLCA) | 1.46 |
| Cervical Squamous carcinoma (CESC) | 1.38 |
| Kidney Adenocarcinoma | 1.07 |
| Thymic Cancer | 0.81 |
| Myeloid Leukemia (LAML) | 0.69 |
| Liver Hepatocellular Carcinoma (LIHC) | 0.55 |
| Glioblastoma multiforme (GBM) | 0.51 |
| Skin Cutaneous Melanoma (SKCM) | 0.43 |
| Bladder Cancer | 0.4 |
| Prostate Adenocarcinoma (PRAD) | 0.2 |
| Breast Invasive Carcinoma (BRCA) | 0.1 |

Provided herein as embodiment 242 is a compound according to any one of embodiments 1-239 or a pharmaceutically acceptable salt thereof, or the pharmaceutical composition according to embodiment 240 for use in treating cancer.

Provided herein as Embodiment 243 is a compound according to any one of Embodiments 1-239 or a pharmaceutically acceptable salt thereof, or the pharmaceutical composition according to Embodiment 240 for use in treating cancer, wherein one or more cells express KRAS G12D, G12V, G12A, G12S or G12C mutant protein.

Provided herein as Embodiment 244 is the compound or pharmaceutical composition for use of Embodiment 242 or 243, wherein the cancer is pancreatic cancer, colorectal cancer, non-small cell lung cancer, small bowel cancer, appendiceal cancer, cancer of unknown primary, endometrial cancer, mixed cancer types, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 245 is a use of the compound according to any one of Embodiments 1-239 or a pharmaceutically acceptable salt thereof, or the pharmaceutical composition according to Embodiment 240 in the preparation of a medicament for treating cancer.

Provided herein as Embodiment 246 is a use of the compound according to any one of Embodiments 1-239 or a pharmaceutically acceptable salt thereof, or the pharmaceutical composition according to Embodiment 240 in the preparation of a medicament for treating cancer, wherein one or more cells express KRAS G12D, G12V, G12A, G12S or G12C mutant protein.

Provided herein as Embodiment 247 is the use according to Embodiment 245 or 246, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 248 is a method of treating cancer in a subject in need thereof, the method comprising administering to the subject a therapeutically effective amount of the compound according to any one of to any one of Embodiments 1-239 or a pharmaceutically acceptable salt thereof.

Provided herein as Embodiment 249 is a method of treating cancer in a subject in need thereof, the method comprising administering to the subject a therapeutically effective amount of the compound according to any one of to any one of Embodiments 1-239 or a pharmaceutically acceptable salt thereof, wherein one or more cells express KRAS G12D, G12V, G12A, G12S or G12C mutant protein.

Provided herein as Embodiment 250 is the method according to Embodiment 248 or 249, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 251 is the method according to Embodiment 248 or 249, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

Provided herein as Embodiment 252 is the method according to Embodiment 251, wherein the cancer is non-small cell lung cancer.

Provided herein as Embodiment 253 is the method according to Embodiment 251, wherein the cancer is colorectal cancer.

Provided herein as Embodiment 254 is the method according to Embodiment 251, wherein the cancer is pancreatic cancer.

Provided herein as Embodiment 255 is the method according to anyone of Embodiments 248-254, wherein the subject has a cancer that was determined to have one or more cells expressing the KRAS G12D, G12V, G12A, G12S or G12C mutant protein prior to administration of the compound or a pharmaceutically acceptable salt thereof.

### Combination Therapy

The present disclosure also provides methods for combination therapies in which an agent known to modulate other pathways, or other components of the same pathway, or even overlapping sets of target enzymes are used in combination with a compound of the present disclosure or a pharmaceutically acceptable salt thereof. In one aspect, such therapy includes but is not limited to the combination of one or more compounds of the disclosure with chemotherapeutic agents, therapeutic antibodies, and radiation treatment, to provide a synergistic or additive therapeutic effect. See, e.g., U.S. Pat. No. 10,519,146 B2, issued Dec. 31, 2019; specifically, the sections from column 201 (line 37) to column 212 (line 46) and column 219 (line 64) to column 220 (line 39), which are herewith incorporated by reference.

Provided herein as Embodiment 256 is the method according to anyone of Embodiments 248-255, which further comprises simultaneous, separate, or sequential administration of an effective amount of a second compound, wherein the second compound is an Aurora kinase A inhibitor, AKT inhibitor, arginase inhibitor, CDK4/6 inhibitor, ErbB family inhibitor, ERK inhibitor, FAK inhibitor, FGFR inhibitor, glutaminase inhibitor, IGF-1R inhibitor, KIF18A inhibitor, MCL-1 inhibitor, MEK inhibitor, mTOR inhibitor, PD-1 inhibitor, PD-L1 inhibitor, PI3K inhibitor, Rafkinase inhibitor, SHP2 inhibitor, SOS1 inhibitor, Src kinase inhibitor, or one or more chemotherapeutic agent.

In one embodiment, the second compound is administered as a pharmaceutically acceptable salt. In another embodiment the second compound is administered as a pharmaceutical composition comprising the second compound or a pharmaceutically acceptable salt thereof and a pharmaceutically acceptable excipient.

### Aurora Kinase A Inhibitors

Provided herein is the method according to anyone of Embodiments 248-255, which further comprises simultaneous, separate, or sequential administration of an effective amount of a second compound, wherein the second compound is an Aurora kinase A inhibitor.

Exemplary Aurora kinase A inhibitors for use in the methods provided herein include, but are not limited to, alisertib, cenisertib, danusertib, tozasertib, LY3295668 ((2R,4R)-1-[(3-chloro-2-fluorophenyl)methyl]-4-[[3-fluoro-6-[(5-methyl-1H-pyrazol-3-yl)amino]pyridin-2-yl]methyl]-2-methylpiperidine-4-carboxylic acid), ENMD-2076 (6-(4-methylpiperazin-1-yl)-N-(5-methyl-1H-pyrazol-3-yl)-2-[(E)-2-phenylethenyl]pyrimidin-4-amine), TAK-901 (5-(3-ethylsulfonylphenyl)-3,8-dimethyl-N-(1-methylpiperidin-4-yl)-9H-pyrido[2,3-b]indole-7-carboxamide), TT-00420 (4-[9-(2-chlorophenyl)-6-methyl-2,4,5,8,12-pentazatricyclo[8.4.0,03,7]tetradeca-1(14),3,6,8,10,12-hexaen-13-yl]morpholine), AMG 900 (N-[4-[3-(2-aminopyrimidin-4-yl)pyridin-2-yl]oxyphenyl]-4-(4-methylthiophen-2-yl)phthalazin-1-amine), MLN8054 (4-[[9-chloro-7-(2,6-difluorophenyl)-5H-pyrimido[5,4-d][2]benzazepin-2-yl]amino]benzoic acid), PF-03814735 (N-[2-[(1R,8S)-4-[[4-(cyclobutylamino)-5-(trifluoromethyl)pyrimidin-2-yl]amino]-11-azatricyclo[6.2.1.02,7]undeca-2(7),3,5-trien-11-yl]-2-oxoethyl]acetamide), SNS-314 (1-(3-chlorophenyl)-3-[5-[2-(thieno[3,2-d]pyrimidin-4-ylamino)ethyl]-1,3-thiazol-2-yl]urea), CYC116 (4-methyl-5-[2-(4-morpholin-4-ylanilino)pyrimidin-4-yl]-1,3-thiazol-2-amine), TAS-119, BI 811283, and TTP607.

### AKT Inhibitors

Provided herein is the method according to anyone of Embodiments 248-255, which further comprises simultaneous, separate, or sequential administration of an effective amount of a second compound, wherein the second compound is an AKT inhibitor.

Exemplary AKT inhibitors for use in the methods provided herein include, but are not limited to, afuresertib, capivasertib, ipatasertib, uprosertib, BAY1125976 (2-[4-(1-aminocyclobutyl)phenyl]-3-phenylimidazo[1,2-b]pyridazine-6-carboxamide), ARQ 092 (3-[3-[4-(1-aminocyclobutyl)phenyl]-5-phenylimidazo[4,5-b]pyridin-2-yl]pyridin-2-amine), MK2206 (8-[4-(1-aminocyclobutyl)phenyl]-9-phenyl-2H-[1,2,4]triazolo[3,4-f][1,6]naphthyridin-3-one), SR13668 (indolo[2,3-b]carbazole-2,10-dicarboxylic acid, 5,7-dihydro-6-methoxy-, 2,10-diethyl ester), ONC201 (11-benzyl-7-[(2-methylphenyl)methyl]-2,5,7,11-tetrazatricyclo[7.4.0.02,6]trideca-1(9),5-dien-8-one), ARQ 751 (N-(3-aminopropyl)-N-[(1R)-1-(3-anilino-7-chloro-4-oxoquinazolin-2-yl)but-3-ynyl]-3-chloro-2-fluorobenzamide), RX-0201, and LY2780301.

### Arginase Inhibitors

Provided herein is the method according to anyone of Embodiments 248-255, which further comprises simultaneous, separate, or sequential administration of an effective amount of a second compound, wherein the second compound is an arginase inhibitor.

Exemplary arginase inhibitors for use in the methods provided herein include, but are not limited to, numidargistat and CB 280.

### CDK4/6 Inhibitors

Provided herein is the method according to anyone of Embodiments 248-255, which further comprises simultaneous, separate, or sequential administration of an effective amount of a second compound, wherein the second compound is a CDK4/6 inhibitor.

The term “CDK 4/6” as used herein refers to cyclin dependent kinases (“CDK”) 4 and 6, which are members of the mammalian serine/threonine protein kinases.

The term “CDK 4/6 inhibitor” as used herein refers to a compound that is capable of negatively modulating or inhibiting all or a portion of the enzymatic activity of CDK 4 and/or 6.

Exemplary CDK 4/6 inhibitors for use in the methods provided herein include, but are not limited to, abemaciclib, palbociclib, ribociclib, trilaciclib, and PF-06873600 ((pyrido[2,3-d]pyrimidin-7(8H)-one, 6-(difluoromethyl)-8-[(1R,2R)-2-hydroxy-2-methylcyclopentyl]-2-[[1-(methylsulfonyl)-4-piperidinyl]amino]).

In one embodiment, the CDK4/6 inhibitor is palbociclib.

### ErbB Family Inhibitors

Provided herein is the method according to anyone of Embodiments 248-255, which further comprises simultaneous, separate, or sequential administration of an effective amount of a second compound, wherein the second compound is an ErbB family inhibitor.

The term “ErbB family” as used herein refers to a member of a mammalian transmembrane protein tyrosine kinase family including: ErbB1 (EGFR HER1), ErbB2 (HER2), ErbB3 (HER3), and ErbB4 (HER4).

The term “ErbB family inhibitor” as used herein refers to an agent, e.g., a compound or antibody, that is capable of negatively modulating or inhibiting all or a portion of the activity of at least one member of the ErbB family. The modulation or inhibition of one or more ErbB tyrosine kinase may occur through modulating or inhibiting kinase enzymatic activity of one or more ErbB family member or by blocking homodimerization or heterodimerization of ErbB family members.

In one embodiment, the ErbB family inhibitor is an EGFR inhibitor, e.g., an anti-EGFR antibody. Exemplary anti-EGFR antibodies for use in the methods provided herein include, but are not limited to, zalutumumab, nimotuzumab, matuzumab, necitumumab, panitumumab, and cetuximab. In one embodiment, the anti-EGFR antibody is cetuximab. In one embodiment, the anti-EGFR antibody is panitumumab.

In another embodiment the ErbB family inhibitor is a HER2 inhibitor, e.g., an anti-HER2 antibody. Exemplary anti-HER-2 antibodies for use in the methods provided herein include, but are not limited to, pertuzumab, trastuzumab, and trastuzumab emtansine.

In yet another embodiment the ErbB family inhibitor is a HER3 inhibitor, e.g., an anti-HER3 antibody, such as HMBD-001 (Hummingbird Bioscience).

In one embodiment, the ErbB family inhibitor is a combination of an anti-EGFR antibody and anti-HER2 antibody.

In one embodiment, the ErbB family inhibitor is an irreversible inhibitor. Exemplary irreversible ErbB family inhibitors for use in the methods provided herein include, but are not limited to, afatinib, dacomitinib, canertinib, poziotinib, AV 412 ((N-[4-[(3-chloro-4-fluorophenyl)amino]-7-[3-methyl-3-(4-methyl-1-piperazinyl)-1-butyn-1-yl]-6-quinazolinyl]-2-propenamide)), PF 6274484 ((N-[4-[(3-chloro-4-fluorophenyl)amino]-7-methoxy-6-quinazolinyl]-2-propenamide), and HKI 357 ((E)-N-[4-[3-chloro-4-[(3-fluorophenyl)methoxy]anilino]-3-cyano-7-ethoxyquinolin-6-yl]-4-(dimethylamino)but-2-enamide).

In one embodiment, the irreversible ErbB family inhibitor is afatinib. In one embodiment, the irreversible ErbB family inhibitor is dacomitinib.

In one embodiment, the ErbB family inhibitor is a reversible inhibitor. Exemplary reversible ErbB family inhibitors for use in the methods provided herein include, but are not limited to erlotinib, gefitinib, sapitinib, varlitinib, tarloxotinib, TAK-285 (N-(2-(4-((3-chloro-4-(3-(trifluoromethyl)phenoxy)phenyl)amino)-5H-pyrrolo[3,2-d]pyrimidin-5-yl)ethyl)-3-hydroxy-3-methylbutanamide), AEE788 ((S)-6-(4-((4-ethylpiperazin-1-yl)methyl)phenyl)-N-(1-phenylethyl)-7H-pyrrolo[2,3-d]pyrimidin-4-amine), BMS 599626 ((3S)-3-morpholinylmethyl-[4-[[1-[(3-fluorophenyl)methyl]-1H-indazol-5-yl]amino]-5-methylpyrrolo[2,1-f][1,2,4]triazin-6-yl]-carbamate), and GW 583340 (N-[3-chloro-4-[(3-fluorophenyl)methoxy]phenyl]-6-[2-[(2-methylsulfonylethylamino)methyl]-1,3-thiazol-4-yl]quinazolin-4-amine).

In one embodiment, the reversible ErbB family inhibitor is sapitinib. In one embodiment, the reversible ErbB family inhibitor is tarloxotinib.

### ERK Inhibitors

Provided herein is the method according to anyone of Embodiments 248-255, which further comprises simultaneous, separate, or sequential administration of an effective amount of a second compound, wherein the second compound is an ERK inhibitor.

Exemplary ERK inhibitors for use in the methods provided herein include, but are not limited to, ulixertinib, ravoxertinib, CC-90003 (N-[2-[[2-[(2-methoxy-5-methylpyridin-4-yl)amino]-5-(trifluoromethyl)pyrimidin-4-yl]amino]-5-methylphenyl]prop-2-enamide), LY3214996 (6,6-dimethyl-2-[2-[(2-methylpyrazol-3-yl)amino]pyrimidin-4-yl]-5-(2-morpholin-4-ylethyl)thieno[2,3-c]pyrrol-4-one), KO-947 (1,5,6,8-tetrahydro-6-(phenylmethyl)-3-(4-pyridinyl)-7H-pyrazolo[4,3-g]quinazolin-7-one), ASTX029, LTT462, and JSI-1187.

### FAK Inhibitors

Provided herein is the method according to anyone of Embodiments 248-255, which further comprises simultaneous, separate, or sequential administration of an effective amount of a second compound, wherein the second compound is a FAK inhibitor.

Exemplary FAK inhibitors for use in the methods provided herein include, but are not limited to, GSK2256098 (2-[[5-chloro-2-[(5-methyl-2-propan-2-ylpyrazol-3-yl)amino]pyridin-4-yl]amino]-N-methoxybenzamide), PF-00562271 (N-methyl-N-[3-[[[2-[(2-oxo-1,3-dihydroindol-5-yl)amino]-5-(trifluoromethyl)pyrimidin-4-yl]amino]methyl]pyridin-2-yl]methanesulfonamide), VS-4718 (2-[[2-(2-methoxy-4-morpholin-4-ylanilino)-5-(trifluoromethyl)pyridin-4-yl]amino]-N-methylbenzamide), and APG-2449.

### FGFR Inhibitors

Provided herein is the method according to anyone of Embodiments 248-255, which further comprises simultaneous, separate, or sequential administration of an effective amount of a second compound, wherein the second compound is an FGFR inhibitor.

Exemplary FGFR inhibitors for use in the methods provided herein include, but are not limited to, futibatinib, pemigatinib, ASP5878 (2-[4-[[5-[(2,6-difluoro-3,5-dimethoxyphenyl)methoxy]pyrimidin-2-yl]amino]pyrazol-1-yl]ethanol), AZD4547 (N-[5-[2-(3,5-dimethoxyphenyl)ethyl]-1H-pyrazol-3-yl]-4-[(3S,5R)-3,5-dimethylpiperazin-1-yl]benzamide), debio 1347 ([5-amino-1-(2-methyl-3H-benzimidazol-5-yl)pyrazol-4-yl]-(1H-indol-2-yl)methanone), INCB062079, H3B-6527 (N-[2-[[6-[(2,6-dichloro-3,5-dimethoxyphenyl)carbamoyl-methylamino]pyrimidin-4-yl]amino]-5-(4-ethylpiperazin-1-yl)phenyl]prop-2-enamide), ICP-105, CPL304110, HMPL-453, and HGS1036.

### Glutaminase Inhibitors

Provided herein is the method according to anyone of Embodiments 248-255, which further comprises simultaneous, separate, or sequential administration of an effective amount of a second compound, wherein the second compound is a glutaminase inhibitor.

Exemplary glutaminase inhibitors for use in the methods provided herein include, but are not limited to, telaglenastat, IPN60090, and OP 330.

### IGF-1R Inhibitors

Provided herein is the method according to anyone of Embodiments 234-241, which further comprises simultaneous, separate, or sequential administration of an effective amount of a second compound, wherein the second compound is an IGF-1R inhibitor.

Exemplary IGF-1R inhibitors for use in the methods provided herein include, but are not limited to, cixutumumab, dalotuzumab, linsitinib, ganitumab, robatumumab, BMS-754807 ((2S)-1-[4-[(5-cyclopropyl-1H-pyrazol-3-yl)amino]pyrrolo[2,1-f][1,2,4]triazin-2-yl]-N-(6-fluoropyridin-3-yl)-2-methylpyrrolidine-2-carboxamide), KW-2450 (N-[5-[[4-(2-hydroxyacetyl)piperazin-1-yl]methyl]-2-[(E)-2-(1H-indazol-3-yl)ethenyl]phenyl]-3-methylthiophene-2-carboxamide), PL225B, AVE1642, and B11B022.

### KIF18A Inhibitors

Provided herein is the method according to anyone of Embodiments 248-255, which further comprises simultaneous, separate, or sequential administration of an effective amount of a second compound, wherein the second compound is a KIF18A inhibitor.

Exemplary KIF18A inhibitors for use in the methods provided herein include, but are not limited to, the inhibitors disclosed in US 2020/0239441, WO 2020/132649, WO 2020/132651, and WO 2020/132653, each of which is herewith incorporated by reference in its entirety.

### MCL-1 Inhibitors

Provided herein is the method according to anyone of Embodiments 248-255, which further comprises simultaneous, separate, or sequential administration of an effective amount of a second compound, wherein the second compound is an MCL-1 inhibitor.

Exemplary MEK inhibitors for use in the methods provided herein include, but are not limited to, murizatoclax, tapotoclax, AZD 5991 ((3aR)-5-chloro-2,11,12,24,27,29-hexahydro-2,3,24,33-tetramethyl-22H-9,4,8-(metheniminomethyno)-14,20:26,23-dimetheno-10H,20H-pyrazolo[4,3-1][2,15,22,18,19]benzoxadithiadiazacyclohexacosine-32-carboxylic acid), MIK 665 ((aR)-α-[[(5S)-5-[3-Chloro-2-methyl-4-[2-(4-methyl-1-piperazinyl)ethoxy]phenyl]-6-(4-fluorophenyl)thieno[2,3-d]pyrimidin-4-yl]oxy]-2-[[2-(2-methoxyphenyl)-4-pyrimidinyl]methoxy]benzenepropanoic acid), and ABBV-467.

In one embodiment, the MCL-1 inhibitor is murizatoclax. In another embodiment, the MCL-1 inhibitor is tapotoclax.

### MEK Inhibitors

Provided herein is the method according to anyone of Embodiments 248-255, which further comprises simultaneous, separate, or sequential administration of an effective amount of a second compound, wherein the second compound is MEK inhibitor.

Exemplary MEK inhibitors for use in the methods provided herein include, but are not limited to, trametinib, cobimetinib, selumetinib, pimasertib, refametinib, PD-325901 (N-[(2R)-2,3-dihydroxypropoxy]-3,4-difluoro-2-(2-fluoro-4-iodoanilino)benzamide), AZD8330 (2-(2-fluoro-4-iodoanilino)-N-(2-hydroxyethoxy)-1,5-dimethyl-6-oxopyridine-3-carboxamide), GDC-0623 (5-(2-fluoro-4-iodoanilino)-N-(2-hydroxyethoxy)imidazo[1,5-a]pyridine-6-carboxamide), RO4987655 (3,4-difluoro-2-(2-fluoro-4-iodoanilino)-N-(2-hydroxyethoxy)-5-[(3-oxooxazinan-2-yl)methyl]benzamide), TAK-733 (3-[(2R)-2,3-dihydroxypropyl]-6-fluoro-5-(2-fluoro-4-iodoanilino)-8-methylpyrido[2,3-d]pyrimidine-4,7-dione), PD0325901 (N-[(2R)-2,3-dihydroxypropoxy]-3,4-difluoro-2-(2-fluoro-4-iodoanilino)benzamide), CI-1040 (2-(2-chloro-4-iodophenylamino)-N-(cyclopropylmethoxy)-3,4-difluorobenzamide), PD318088 (5-bromo-N-(2,3-dihydroxypropoxy)-3,4-difluoro-2-(2-fluoro-4-iodophenylamino)benzamide), PD98059 (2-(2-amino-3-methoxyphenyl)-4H-chromen-4-one), PD334581 (N-[5-[3,4-Difluoro-2-[(2-fluoro-4-iodophenyl)amino]phenyl]-1,3,4-oxadiazol-2-yl]-4-morpholineethanamine), FCN-159, CS3006, HL-085, SHR 7390, and WX-554.

In one embodiment, the MEK inhibitor is trametinib.

mTOR Inhibitors

Provided herein is the method according to anyone of Embodiments 248-255, which further comprises simultaneous, separate, or sequential administration of an effective amount of a second compound, wherein the second compound is an mTOR inhibitor.

Exemplary mTOR inhibitors for use in the methods provided herein include, but are not limited to, everolimus, rapamycin, zotarolimus (ABT-578), ridaforolimus (deforolimus, MK-8669), sapanisertib, buparlisib, pictilisib, vistusertib, dactolisib, Torin-1 (1-(4-(4-propionylpiperazin-1-yl)-3-(trifluoromethyl)cyclohexyl)-9-(quinolin-3-yl)benzo[h][1,6]naphthyridin-2(1H)-one), GDC-0349 ((S)-1-ethyl-3-(4-(4-(3-methylmorpholino)-7-(oxetan-3-yl)-5,6,7,8-tetrahydropyrido[3,4-d]pyrimidin-2-yl)phenyl)urea), and VS-5584 (SB2343, (5-(8-methyl-2-morpholin-4-yl-9-propan-2-ylpurin-6-yl)pyrimidin-2-amine).

In one embodiment, the mTOR inhibitor is everolimus.

### PD-1 Inhibitors

Provided herein is the method according to anyone of Embodiments 248-255, which further comprises simultaneous, separate, or sequential administration of an effective amount of a second compound, wherein the second compound is a PD-1 inhibitor.

Exemplary PD-1 inhibitors for use in the methods provided herein include, but are not limited to, pembrolizumab, nivolumab, cemiplimab, spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514, and the anti-PD-1 antibody as described in U.S. Pat. No. 10,640,504 B2 (the “Anti-PD-1 Antibody A,” column 66, line 56 to column 67, line 24 and column 67, lines 54-57), which is incorporated herein by reference.

In one embodiment, the PD-1 inhibitor is pembrolizumab. In another embodiment the PD-1 inhibitor is the Anti-PD-1 Antibody A.

### PD-L1 Inhibitors

Provided herein is the method according to anyone of Embodiments 248-255, which further comprises simultaneous, separate, or sequential administration of an effective amount of a second compound, wherein the second compound is a PD-L1 inhibitor.

Exemplary PD-L1 inhibitors for use in the methods provided herein include, but are not limited to, atezolizumab, avelumab, durvalumab, ZKAB001, TG-1501, SHR-1316, MSB2311, MDX-1105, KN035, IMC-001, HLX20, FAZ053, CS1001, CK-301, CBT-502, BGB-A333, BCD-135, and A167.

In one embodiment, the PD-L1 inhibitor is atezolizumab.

### PI3K Inhibitors

Provided herein is the method according to anyone of Embodiments 248-255, which further comprises simultaneous, separate, or sequential administration of an effective amount of a second compound, wherein the second compound is a PI3K inhibitor.

Exemplary PI3K inhibitors for use in the methods provided herein include, but are not limited to, idelalisib, copanlisib, duvelisib, alpelisib, taselisib, perifosine, buparlisib, umbralisib, pictilisib, dactolisib, voxtalisib, sonolisib, tenalisib, serabelisib, acalisib, CUDC-907 (N-hydroxy-2-[[2-(6-methoxypyridin-3-yl)-4-morpholin-4-ylthieno[3,2-d]pyrimidin-6-yl]methyl-methylamino]pyrimidine-5-carboxamide), ME-401 (N-[2-methyl-1-[2-(1-methylpiperidin-4-yl)phenyl]propan-2-yl]-4-(2-methylsulfonylbenzimidazol-1-yl)-6-morpholin-4-yl-1,3,5-triazin-2-amine), IPI-549 (2-amino-N-[(1S)-1-[8-[2-(1-methylpyrazol-4-yl)ethynyl]-1-oxo-2-phenylisoquinolin-3-yl]ethyl]pyrazolo[1,5-a]pyrimidine-3-carboxamide), SF1126 ((2S)-2-[[(2S)-3-carboxy-2-[[2-[[(2S)-5-(diaminomethylideneamino)-2-[[4-oxo-4-[[4-(4-oxo-8-phenylchromen-2-yl)morpholin-4-ium-4-yl]methoxy]butanoyl]amino]pentanoyl]amino]acetyl]amino]propanoyl]amino]-3-hydroxypropanoate), XL147 (N-[3-(2,1,3-benzothiadiazol-5-ylamino)quinoxalin-2-yl]-4-methylbenzenesulfonamide), GSK1059615 ((5Z)-5-[(4-pyridin-4-ylquinolin-6-yl)methylidene]-1,3-thiazolidine-2,4-dione), and AMG 319 (N-[(1S)-1-(7-fluoro-2-pyridin-2-ylquinolin-3-yl)ethyl]-7H-purin-6-amine).

### Raf Kinase Inhibitors

Provided herein is the method according to anyone of Embodiments 248-255, which further comprises simultaneous, separate, or sequential administration of an effective amount of a second compound, wherein the second compound is a Raf kinase inhibitor.

The term “RAF kinase” as used herein refers to a member of a mammalian serine/threonine kinases composed of three isoforms (C-Raf, B-Raf and A-Raf) and includes homodimers of each isoform as well as heterodimers between isoforms, e.g., C-Raf/B-Raf heterodimers.

The term “Raf kinase inhibitor” as used herein refers to a compound that is capable of negatively modulating or inhibiting all or a portion of the enzymatic activity of one or more member of the Raf family kinases, or is capable of disrupting Raf homodimer or heterodimer formation to inhibit activity.

In one embodiment, the Raf kinase inhibitor includes, but is not limited to, encorafenib, sorafenib, lifirafenib, vemurafenib, dabrafenib, PLX-8394 (N-(3-(5-(2-cyclopropylpyrimidin-5-yl)-3a,7a-dihydro-1H-pyrrolo[2,3-b]pyridine-3-carbonyl)-2,4-difluorophenyl)-3-fluoropyrrolidine-1-sulfonamide), Raf-709 (N-(2-methyl-5,-morpholino-6′-((tetrahydro-2H-pyran-4-yl)oxy)-[3,3′-bipyridin]-5-yl)-3-(trifluoromethyl)benzamide), LXH254 (N-(3-(2-(2-hydroxyethoxy)-6-morpholinopyridin-4-yl)-4-methylphenyl)-2-(trifluoromethyl)isonicotinamide), LY3009120 (1-(3,3-dimethylbutyl)-3-(2-fluoro-4-methyl-5-(7-methyl-2-(methylamino)pyrido[2,3-d]pyrimidin-6-yl)phenyl)urea), Tak-632 (N-(7-cyano-6-(4-fluoro-3-(2-(3-(trifluoromethyl)phenyl)acetamido)phenoxy)benzo[d]thiazol-2-yl)cyclopropanecarboxamide), CEP-32496 (1-(3-((6,7-dimethoxyquinazolin-4-yl)oxy)phenyl)-3-(5-(1,1,1-trifluoro-2-methylpropan-2-yl)isoxazol-3-yl)urea), CCT196969 (1-(3-(tert-butyl)-1-phenyl-1H-pyrazol-5-yl)-3-(2-fluoro-4-((3-oxo-3,4-dihydropyrido[2,3-b]pyrazin-8-yl)oxy)phenyl)urea), and RO5126766 (N-[3-fluoro-4-[[4-methyl-2-oxo-7-(2-pyrimidinyloxy)-2H-1-benzopyran-3-yl]methyl]-2-pyridinyl]-N′-methyl-sulfamide).

In one embodiment, the Raf kinase inhibitor is encorafenib. In one embodiment, the Raf kinase inhibitor is sorafenib. In one embodiment, the Raf kinase inhibitor is lifirafenib.

### SHP2 Inhibitors

Provided herein is the method according to anyone of Embodiments 248-255, which further comprises simultaneous, separate, or sequential administration of an effective amount of a second compound, wherein the second compound is a SHP2 inhibitor.

Exemplary SHP2 inhibitors for use in the methods provided herein include, but are not limited to, SHP-099 (6-(4-amino-4-methylpiperidin-1-yl)-3-(2,3-dichlorophenyl)pyrazin-2-amine dihydrochloride), RMC-4550 ([3-[(3S,4S)-4-amino-3-methyl-2-oxa-8-azaspiro[4.5]decan-8-yl]-6-(2,3-dichlorophenyl)-5-methylpyrazin-2-yl]methanol), TNO155, (3S,4S)-8-[6-amino-5-(2-amino-3-chloropyridin-4-yl)sulfanylpyrazin-2-yl]-3-methyl-2-oxa-8-azaspiro[4.5]decan-4-amine), and RMC-4630 (Revolution Medicine). In one embodiment, the SHP inhibitor for use in the methods provided herein is RMC-4630 (Revolution Medicine).

In another embodiment, exemplary SHP2 inhibitors for use in the methods provided herein include, but are not limited to, 3-[(1R,3R)-1-amino-3-methoxy-8-azaspiro[4.5]dec-8-yl]-6-(2,3-dichlorophenyl)-5-methyl-2-pyrazinemethanol (CAS 2172651-08-8), 3-[(3S,4S)-4-amino-3-methyl-2-oxa-8-azaspiro[4.5]dec-8-yl]-6-[(2,3-dichlorophenyl)thio]-5-methyl-2-pyrazinemethanol (CAS 2172652-13-8), 3-[(3S,4S)-4-amino-3-methyl-2-oxa-8-azaspiro[4.5]dec-8-yl]-6-[[3-chloro-2-(3-hydroxy-1-azetidinyl)-4-pyridinyl]thio]-5-methyl-2-pyrazinemethanol (CAS 2172652-38-7), and 6-[(2-amino-3-chloro-4-pyridinyl)thio]-3-[(3S,4S)-4-amino-3-methyl-2-oxa-8-azaspiro[4.5]dec-8-yl]-5-methyl-2-pyrazinemethanol (CAS 2172652-48-9).

In another embodiment, exemplary SHP2 inhibitors for use in the methods provided herein include, but are not limited to, 1-[5-(2,3-dichlorophenyl)-6-methylimidazo[1,5-a]pyrazin-8-yl]-4-methyl-4-piperidinamine (CAS 2240981-75-1), (1R)-8-[5-(2,3-dichlorophenyl)-6-methylimidazo[1,5-a]pyrazin-8-yl]-8-azaspiro[4.5]decan-1-amine (CAS 2240981-78-4), (3S,4S)-8-[7-(2,3-dichlorophenyl)-6-methylpyrazolo[1,5-a]pyrazin-4-yl]-3-methyl-2-oxa-8-azaspiro[4.5]decan-4-amine (CAS 2240982-45-8), (3S,4S)-8-[7-[(2-amino-3-chloro-4-pyridinyl)thio]pyrazolo[1,5-a]pyrazin-4-yl]-3-methyl-2-oxa-8-azaspiro[4.5]decan-4-amine (CAS 2240982-57-2), 4-[(3S,4S)-4-amino-3-methyl-2-oxa-8-azaspiro[4.5]dec-8-yl]-7-(2,3-dichlorophenyl)-6-methyl-pyrazolo[1,5-a]pyrazine-2-methanol (CAS 2240982-69-6), 7-[(2-amino-3-chloro-4-pyridinyl)thio]-4-[(3S,4S)-4-amino-3-methyl-2-oxa-8-azaspiro[4.5]dec-8-yl]-6-methyl-pyrazolo[1,5-a]pyrazine-2-methanol (CAS 2240982-73-2), and (3S,4S)-8-[7-[(2-amino-3-chloro-4-pyridinyl)thio]-6-methylpyrazolo[1,5-a]pyrazin-4-yl]-3-methyl-2-oxa-8-azaspiro[4.5]decan-4-amine (CAS 2240982-77-6).

In one embodiment, the SHP inhibitor for use in the methods provided herein is (1R)-8-[5-(2,3-dichlorophenyl)-6-methylimidazo[1,5-a]pyrazin-8-yl]-8-azaspiro[4.5]decan-1-amine (CAS 2240981-78-4).

In another embodiment, exemplary SHP2 inhibitors for use in the methods provided herein include, but are not limited to 3-[(1R)-1-amino-8-azaspiro[4.5]dec-8-yl]-6-(2,3-dichlorophenyl)-5-hydroxy-2-pyridinemethanol (CAS 2238840-54-3), 3-[(1R)-1-amino-8-azaspiro[4.5]dec-8-yl]-6-[(2,3-dichlorophenyl)thio]-5-hydroxy-2-pyridinemethanol (CAS 2238840-56-5), 5-[(1R)-1-amino-8-azaspiro[4.5]dec-8-yl]-2-(2,3-dichlorophenyl)-3-pyridinol (CAS 2238840-58-7), 3-[(1R)-1-amino-8-azaspiro[4.5]dec-8-yl]-6-(2,3-dichlorophenyl)-5-methyl-2-pyridinemethanol (CAS 2238840-60-1), (1R)-8-[6-(2,3-dichlorophenyl)-5-methyl-3-pyridinyl]-8-azaspiro[4.5]decan-1-amine (CAS 2238840-62-3), 3-[(1R)-1-amino-8-azaspiro[4.5]dec-8-yl]-6-[(2,3-dichlorophenyl)thio]-5-methyl-2-pyridinemethanol (CAS 2238840-63-4), (1R)-8-[6-[(2,3-dichlorophenyl)thio]-5-methyl-3-pyridinyl]-8-azaspiro[4.5]decan-1-amine (CAS 2238840-64-5), 5-(4-amino-4-methyl-1-piperidinyl)-2-[(2,3-dichlorophenyl)thio]-3-pyridinol (CAS 2238840-65-6), 5-[(1R)-1-amino-8-azaspiro[4.5]dec-8-yl]-2-[(2,3-dichlorophenyl)thio]-3-pyridinol (CAS 2238840-66-7), 6-[(2-amino-3-chloro-4-pyridinyl)thio]-3-[(3S,4S)-4-amino-3-methyl-2-oxa-8-azaspiro[4.5]dec-8-yl]-5-hydroxy-2-pyridinemethanol (CAS 2238840-67-8), 3-(4-amino-4-methyl-1-piperidinyl)-6-(2,3-dichlorophenyl)-5-hydroxy-2-pyridinemethanol (CAS 2238840-68-9), 3-[(3S,4S)-4-amino-3-methyl-2-oxa-8-azaspiro[4.5]dec-8-yl]-6-(2,3-dichlorophenyl)-5-methyl-2-pyridinemethanol (CAS 2238840-69-0), 6-[(2-amino-3-chloro-4-pyridinyl)thio]-3-[(3S,4S)-4-amino-3-methyl-2-oxa-8-azaspiro[4.5]dec-8-yl]-5-methyl-2-pyridinemethanol (CAS 2238840-70-3), 3-(4-amino-4-methyl-1-piperidinyl)-6-(2,3-dichlorophenyl)-5-methyl-2-pyridinemethanol (CAS 2238840-71-4), 6-[(2-amino-3-chloro-4-pyridinyl)thio]-3-(4-amino-4-methyl-1-piperidinyl)-2-pyridinemethanol (CAS 2238840-72-5), 5-[(2-amino-3-chloro-4-pyridinyl)thio]-2-[(3S,4S)-4-amino-3-methyl-2-oxa-8-azaspiro[4.5]dec-8-yl]-6-methyl-3-pyridinemethanol (CAS 2238840-73-6), 2-[(3S,4S)-4-amino-3-methyl-2-oxa-8-azaspiro[4.5]dec-8-yl]-5-(2,3-dichlorophenyl)-6-methyl-3-pyridinemethanol (CAS 2238840-74-7), 3-[(3S,4S)-4-amino-3-methyl-2-oxa-8-azaspiro[4.5]dec-8-yl]-6-(2,3-dichlorophenyl)-5-hydroxy-2-pyridinemethanol (CAS 2238840-75-8), and 2-[(2-amino-3-chloro-4-pyridyl)sulfanyl]-5-[(3S,4S)-4-amino-3-methyl-2-oxa-8-azaspiro[4.5]decan-8-yl]-6-(hydroxymethyl)pyridin-3-ol.

In one embodiment, the SHP inhibitor for use in the methods provided herein is 3-[(1R)-1-amino-8-azaspiro[4.5]dec-8-yl]-6-[(2,3-dichlorophenyl)thio]-5-hydroxy-2-pyridinemethanol (CAS 2238840-56-5).

In one embodiment, the SHP2 inhibitor for use in the methods provided herein is an inhibitor disclosed in U.S. Pat. No. 10,590,090 B2, US 2020/017517 A1, US 2020/017511 A1, or WO 2019/075265 A1, each of which is herewith incorporated by reference in its entirety.

### SOS1 Inhibitors

Provided herein is the method according to anyone of Embodiments 248-255, which further comprises simultaneous, separate, or sequential administration of an effective amount of a second compound, wherein the second compound is an SOS1 inhibitor.

Exemplary SOS1 inhibitors for use in the methods provided herein include, but are not limited to, BI 3406 (N-[(1R)-1-[3-amino-5-(trifluoromethyl)phenyl]ethyl]-7-methoxy-2-methyl-6-[(3S)-oxolan-3-yl]oxyquinazolin-4-amine), and BI 1701963.

### Src Kinase Inhibitors

Provided herein is the method according to anyone of Embodiments 248-255, which further comprises simultaneous, separate, or sequential administration of an effective amount of a second compound, wherein the second compound is a Src kinase inhibitor.

The term “Src kinase” as used herein refers to a member of a mammalian nonreceptor tyrosine kinase family including: Src, Yes, Fyn, and Fgr (SrcA subfamily); Lck, Hck, Blk, and Lyn (SrcB subfamily), and Frk subfamily.

The term “Src kinase inhibitor” as used herein refers to a compound that is capable of negatively modulating or inhibiting all or a portion of the enzymatic activity of one or more member of the Src kinases.

Exemplary Src kinase inhibitors for use in the methods provided herein include, but are not limited to, dasatinib, ponatinib, vandetanib, bosutinib, saracatinib, KX2-391 (N-benzyl-2-(5-(4-(2-morpholinoethoxy)phenyl)pyridin-2-yl)acetamide), SU6656 ((Z)-N,N-dimethyl-2-oxo-3-((4,5,6,7-tetrahydro-1H-indol-2-yl)methylene)indoline-5-sulfonamide), PP 1 (1-(tert-butyl)-3-(p-tolyl)-1H-pyrazolo[3,4-d]pyrimidin-4-amine), WH-4-023 (2,6-dimethylphenyl(2,4-dimethoxyphenyl)(2-((4-(4-methylpiperazin-1-yl)phenyl)amino)pyrimidin-4-yl)carbamate), and KX-01 (N-benzyl-2-(5-(4-(2-morpholinoethoxy)phenyl)pyridin-2-yl)acetamide).

In one embodiment, the Src kinase inhibitor is dasatinib. In one embodiment, the Src kinase inhibitor is saracatinib. In one embodiment, the Src kinase inhibitor is ponatinib. In one embodiment, the Src kinase inhibitor is vandetanib. In one embodiment, the Src kinase inhibitor is KX-01.

### Chemotherapeutic Agents

Provided herein is the method according to anyone of Embodiments 248-255, which further comprises simultaneous, separate, or sequential administration of an effective amount of a second compound, wherein the second compound is one or more chemotherapeutic agent.

Exemplary chemotherapeutic agents for use in the methods provided herein include, but are not limited to, leucovorin calcium (calcium folinate), 5-fluorouracil, irinotecan, oxaliplatin, cisplatin, carboplatin, pemetrexed, docetaxel, paclitaxel, gemcitabine, vinorelbine, chlorambucil, cyclophosphamide, and methotrexate.

### Definitions

The following definitions are provided to assist in understanding the scope of this disclosure.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the instant disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated. A bond drawn with a wavy line indicates that both stereoisomers are encompassed. This is not to be confused with a wavy line drawn perpendicular to a bond which indicates the point of attachment of a group to the rest of the molecule.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of other enantiomers or diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. Further, this disclosure encompasses pharmaceutical compositions comprising mixtures of any of the compounds disclosed herein and one or more other active agents disclosed herein. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. Accordingly, the scope of the instant disclosure is to be understood to encompass all tautomeric forms of the compounds disclosed herein.

### Isotopically-Labelled Compounds

Further, the scope of the present disclosure includes all pharmaceutically acceptable isotopically-labelled compounds of the compounds disclosed herein, such as the compounds of Formula I, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ¹¹C, ¹³C and ¹⁴C, chlorine, such as ³⁶CI, fluorine, such as ⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula I, for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers, tautomers, and isotopically-labelled forms thereof or a pharmaceutically acceptable salt of any of the foregoing may exist in solvated or unsolvated forms.

The term “solvate” as used herein refers to a molecular complex comprising a compound or a pharmaceutically acceptable salt thereof as described herein and a stoichiometric or non-stoichiometric amount of one or more pharmaceutically acceptable solvent molecules. If the solvent is water, the solvate is referred to as a “hydrate.”

Accordingly, the scope of the instant disclosure is to be understood to encompass all solvents of the compounds disclosed herein and the stereoisomers, tautomers and isotopically-labelled forms thereof or a pharmaceutically acceptable salt of any of the foregoing.

### Miscellaneous Definitions

This section will define additional terms used to describe the scope of the compounds, compositions and uses disclosed herein.

The term “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together. Non-limiting examples include phenyl, naphthyl or tetrahydronaphthyl, each of which may optionally be substituted with 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—, aryl-S-nitro, cyano, carboxy, alkyl-O—C(O)—-, carbamoyl, alkyl-S(O)—, sulfonyl, sulfonamido, phenyl, and heterocycloalkyl.

The terms “C₁₋₄alkyl,” and “C₁₋₆alkyl” as used herein refer to a straight or branched chain hydrocarbon containing from 1 to 4, and 1 to 6 carbon atoms, respectively. Representative examples of C₁₋₄alkyl or C₁₋₆ alkyl include, but are not limited to, methyl, ethyl, n-propyl, iso-propyl, n-butyl, sec-butyl, iso-butyl, tert-butyl, pentyl and hexyl.

The terms “C₁₋₄alkylene” and “C₁₋₆alkylene” refer to a straight or branched divalent alkyl group as defined herein containing 1 to 4, and 1 to 6 carbon atoms, respectively. Representative examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene and the like.

The term “C₂₋₄alkenyl” as used herein refers to a saturated hydrocarbon containing 2 to 4 carbon atoms having at least one carbon-carbon double bond. Alkenyl groups include both straight and branched moieties. Representative examples of C₂₋₄alkenyl include, but are not limited to, 1-propenyl, 2-propenyl, 2-methyl-2-propenyl, and butenyl.

The term “C₂₋₄alkynyl” as used herein refers to a saturated hydrocarbon containing 2 to 4 carbon atoms having at least one carbon-carbon triple bond. The term includes both straight and branched moieties. Representative examples of C₃₋₆alkynyl include, but are not limited to, ethynyl, 1-propynyl, 2-propynyl, 2-butynyl and 3-butynyl.

The term “C₁₋₄alkoxy” or “C₁₋₆alkoxy” as used herein refers to —OR, wherein R represents a C₁₋₄alkyl group or C₁₋₆alkyl group, respectively, as defined herein.

Representative examples of C₁₋₄alkoxy include, but are not limited to, methoxy, ethoxy, propoxy, iso-propoxy, and butoxy. Representative examples of C₁₋₆alkoxy include, but are not limited to, ethoxy, propoxy, iso-propoxy, and butoxy.

The term “C₃₋₈cycloalkyl” as used herein refers to a saturated carbocyclic molecule wherein the cyclic framework has 3 to 8 carbons. Representative examples of C₃₋₈cycloalkyl include, but are not limited to, cyclopropyl and cyclobutyl.

The term “deutero” as used herein as a prefix to another term for a chemical group refers to a modification of the chemical group, wherein one or more hydrogen atoms are substituted with deuterium (“D” or “2H”). For example, the term “C₁₋₄deuteroalkyl” refers to a C₁₋₄alkyl as defined herein, wherein one or more hydrogen atoms are substituted with D. Representative examples of C₁₋₄deuteroalkyl include, but are not limited to, —CH₂D, —CHD₂, —CD₃, —CH₂CD₃, —CDHCD₃, —CD₂CD₃, —CH(CD₃)₂, —CD(CHD₂)₂, and —CH(CH₂D)(CD₃).

The term “halogen” as used herein refers to —F, —CI, —Br, or —I.

The term “halo” as used herein as a prefix to another term for a chemical group refers to a modification of the chemical group, wherein one or more hydrogen atoms are substituted with a halogen as defined herein. The halogen is independently selected at each occurrence. For example, the term “C₁₋₄haloalkyl” refers to a C₁₋₄alkyl as defined herein, wherein one or more hydrogen atoms are substituted with a halogen. Representative examples of C₁₋₄haloalkyl include, but are not limited to, —CH₂F, —CHF₂, —CF₃, —CHFCl, —CH₂CF₃, —CFHCF₃, —CF₂CF₃, —CH(CF₃)₂, —CF(CHF₂)₂, and —CH(CH₂F)(CF₃).

As used herein, the term “heteroaryl” refers to a 5-20 membered monocyclic- or bicyclic- or tricyclic-aromatic ring system, having 1 to 8 heteroatoms selected from N, O and S. In certain preferred aspects, the heteroaryl is a 5-10 membered ring system (e.g., 5-7 membered monocycle, an 8-10 membered bicycle or a 11-14 membered tricycle) or a 5-7 membered ring system. Exemplary monocyclic heteroaryl groups include 2- or 3-thienyl, 2- or 3-furyl, 2- or 3-pyrrolyl, 2, 4-, or 5-imidazolyl, 3, 4-, or 5-pyrazolyl, 2, 4-, or 5-thiazolyl, 3, 4-, or 5-isothiazolyl, 2, 4-, or 5-oxazolyl, 3, 4-, or 5-isoxazolyl, 3- or 5-1,2,4-triazolyl, 4- or 5-1,2,3-triazolyl, tetrazolyl, 2, 3-, or 4-pyridyl, 3- or 4-pyridazinyl, 3, 4-, or 5-pyrazinyl, 2-pyrazinyl, and 2, 4-, and 5-pyrimidinyl. Exemplary bicyclic heteroaryl groups include 1-, 3, 4-, 5-, 6-, 7-, or 8-isoquinolinyl, 2, 3-, 4-, 5-, 6-, 7-, or 8-quinolinyl, 1-, 3, 4-, 5-, 6-, 7-, or 8-isoquinolinyl, 1-, 2, 4-, 5-, 6-, 7-, or 8-benzimidazolyl and 1-, 2, 3-, 4-, 5-, 6-, 7-, or 8-indolyl.

The term “heteroaryl” also refers to a group in which a heteroaromatic ring is fused to one or more aryl, cycloaliphatic, or heterocycloalkyl rings.

As used herein, the term “heterocycle,” “heterocycloalkyl” or “heterocyclo” refers to a saturated or unsaturated non-aromatic ring or ring system, e.g., which is a 4-, 5-, 6-, or 7-membered monocyclic, 7-, 8-, 9-, 10-, 11-, or 12-membered bicyclic or 10-, 11-, 12-, 13-, 14- or 15-membered tricyclic ring system and contains at least one heteroatom selected from O, S and N, where the N and S can also optionally be oxidized to various oxidation states. The heterocyclic group can be attached at a heteroatom or a carbon atom. The heterocycloalkyl can include fused or bridged rings as well as spirocyclic rings. Examples of heterocycles include tetrahydrofuran, dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, azetidine, thiazolidine, morpholine, and the like.

The term “pharmaceutically acceptable” as used herein refers to generally recognized for use in subjects, particularly in humans.

The term “pharmaceutically acceptable salt” as used herein refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66(1):1-19 (1977). See also Stahl et al., Pharmaceutical Salts: Properties, Selection, and Use, 2^{nd} Revised Edition (2011).

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherents, glidants, disintegrants, flavoring agents, coatings, binders, sweeteners, lubricants, sorbents, preservatives, and the like.

The term “subject” as used herein refers to humans and mammals, including, but not limited to, primates, cows, sheep, goats, horses, dogs, cats, rabbits, rats, and mice. In one embodiment the subject is a human.

The term “therapeutically effective amount” as used herein refers to that amount of a compound disclosed herein that will elicit the biological or medical response of a tissue, a system, or subject that is being sought by a researcher, veterinarian, medical doctor or other clinician.

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula I can be synthesized according to the following schemes. Any variables used in the following schemes are the variables as defined for Formula I, unless otherwise noted. All starting materials are either commercially available, for example, from Merck Sigma-Aldrich Inc., Fluorochem Ltd, and Enamine Ltd. or known in the art and may be synthesized by employing known procedures using ordinary skill. Starting material may also be synthesized via the procedures disclosed herein. Suitable reaction conditions, such as, solvent, reaction temperature, and reagents, for the Schemes discussed in this section, may be found in the examples provided herein.

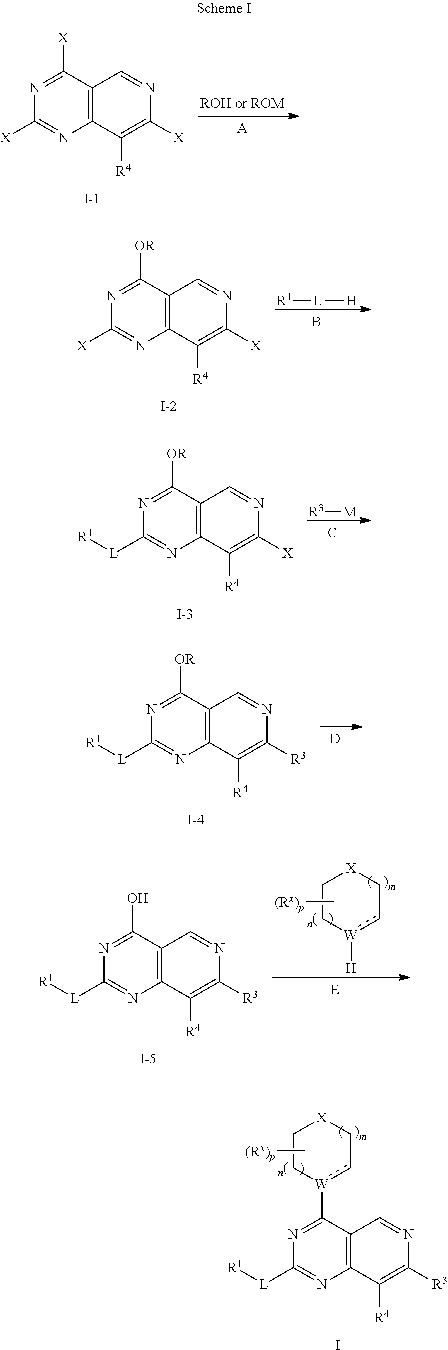

Compounds of Formula (I) can be prepared according to Scheme I. In step A, compound (I-1) is treated with an aliphatic alcohol, such as benzyl alcohol, and a base, such as Hunig's base, or metal alkoxide, such as potassium tert-butoxide, in a solvent such as 1,4-dioxane to give compound (I-2). In step B, compound (I-2) undergoes S_{N}Ar reaction with a nucleophile having the formula R¹-L-H in a solvent such as acetonitrile, in the presence of a base such as Hunig's base, to give compound (I-3). In step C, compound (I-3) is coupled with an organometallic reagent or a boronic acid (ester) to provide compound (I-4). This coupling reaction proceeds in a solvent or mixture of solvents such as 1,4-dioxane and water, and a catalyst such as cataCXium A Pd G3, with or without a base such as potassium phosphate. In step D, compound (I-4) is treated with a suitable set of reagents, such as Pd/C with H₂ to remove the alkyl group R, giving compound (I-5). In Step E, compound (I-5) is treated with an optionally substituted cyclic amine in the presence of coupling reagent such as HATU, and a base such as Hunig's base, in a solvent such as DMA to give compounds of Formula (I). In some cases, the species R³ will contain protecting group(s), which can be removed in step D or after step E in the synthetic sequence.

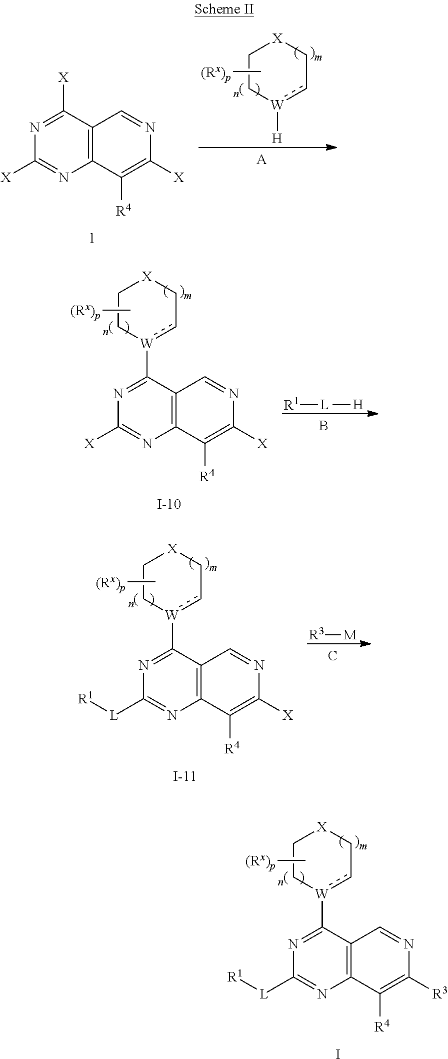

Compounds of Formula (I) can also be prepared according to Scheme II. In step A, compound (1) undergoes S_{N}Ar reaction with an optionally substituted cyclic amine in a solvent such as dichloromethane and in the presence of a base such as Hunig's base to give compound (I-10). In step B, compound (I-10) undergoes S_{N}Ar reaction with a nucleophile having the formula R¹-L-H in a solvent such as acetonitrile, in the presence of a base such as Hunig's base to give compound (I-11). In step C, compound (I-11) is coupled with an organometallic reagent or a boronic acid (ester) to provide compounds of formula (I). This coupling reaction proceeds in a solvent or mixture of solvents such as 1,4-dioxane and water, and a catalyst such as cataCXium A Pd G3, with or without a base such as potassium phosphate. In some cases, the species R³ will contain protecting group(s), which can be removed after step C in the synthetic sequence.

### EXAMPLES

This section provides specific examples of compounds of Formula I and methods of making the same.

### List of Abbreviations

TABLE 1

| | |
|---|---|
| Ac | acetyl |
| AcOH | acetic acid |
| aq or aq. | aqueous |
| Bn | benzyl |
| B₂pin₂ | bis(pinacolato)diboron |
| BOC or Boc | tert-butyloxycarbonyl |
| cataCXium A Pd G2 | chloro[(di(1-adamantyl)-N-butylphosphine)- |
| | 2-(2-aminobiphenyl)]palladium(II) |
| cataCXium A Pd G3 | mesylate[(di(1-adamantyl)-n-butylphosphine)- |
| | 2-(2′-amino-1,1′-biphenyl)]palladium(II) |
| COD or cod | 1,5-cyclooctadiene |
| DABCO | 1,4-diazabicyclo[2.2.2]octane |
| DBU | 1,8-diazabicyclo[5.4.0]undec-7-ene |
| DCE | 1,2-dichloroethane |
| DCM | dichloromethane |
| DMA | N,N-dimethylacetamide |
| DMF | N,N-dimethylformamide |
| DMSO | dimethyl sulfoxide |
| Dppf, DPPF or dppf | 1,1′-bis(diphenylphosphino)ferrocene |
| Dtbbpy | 4,4′-di-tert-butyl-2,2′-dipyridyl |
| eq or eq. or equiv. | equivalent |
| ESI or ES | electrospray ionization |
| Et | ethyl |
| EtOAc | ethyl acetate |
| G | gram(s) |
| H | hour(s) |
| HATU | 1-[bis(dimethylamino)methylene]-1H- |
| | 1,2,3-triazolo[4,5-b]pyridinium 3-oxid |
| | hexafluorophosphate |
| HBpin | 4,4,5,5-tetramethyl-1,3,2-dioxaborolane |
| HMPA | hexamethylphosphoramide |
| HOAc | acetic acid |
| HPLC | high pressure liquid chromatography |
| iPr | iso-propyl |
| iPr₂NEt or DIPEA | N-ethyl diisopropylamine (Hünig′s base) |
| KOAc | potassium acetate |
| LAH | lithium aluminum hydride |
| LC MS, LCMS, | liquid chromatography mass spectroscopy |
| LC-MS or LC/MS | |
| LHMDS or LiHMDS | lithium hexamethyldisilazide |
| m/z | mass divided by charge |
| mCPBA | meta-chloroperoxybenzoic acid |
| Me | methyl |
| MeCN | acetonitrile |
| MeOH | methanol |
| Mg | milligrams |
| Min | minutes |
| mL | milliliters |
| MS | mass spectra |
| NMR | nuclear magnetic resonance |
| Pd(dppf)Cl₂•DCM, | [1,1′- |
| Pd(dppf)Cl₂ | bis(diphenylphosphino)ferrocene]dichloro- |
| | palladium(II), complex with dichloromethane |
| Pd(dtbpf)Cl₂ | [1,1′-bis(di-tert-butyl- |
| | phosphino)ferrocene]dichloropalladium(II) |
| Pd(PPh₃)₄ | tetrakis(triphenylphosphine)palladium(0) |
| Ph | phenyl |
| PhMe | toluene |
| PIFA | (bis(trifluoroacetoxy)iodo)benzene |
| Pin | pinacolato |
| PMB | 4-methoxybenzyl |
| PMB-Cl | 4-methoxybenzyl chloride |
| Rbf | round-bottom flask |
| RP-HPLC | reverse phase high pressure liquid |
| | chromatography |
| RT or rt or r.t. | room temperature |
| sat. or satd. | saturated |
| SFC | supercritical fluid chromatography |
| TBAF | tetra-n-butylammonium fluoride |
| TBDPS | tert-butyldiphenylsilyl |
| TBDPSCl | tert-butyldiphenylsilyl chloride |
| TBS | tert-butyldimethylsilyl |
| TBSCl | tert-butyldimethylsilyl chloride |
| tBu | tert-butyl |
| TC | thiophene-2-carboxylate |
| TEA or Et₃N | triethylamine |
| TFA | trifluoroacetic acid |
| THF | tetrahydrofuran |
| TIPS | triisopropylsilyl |
| TMS | trimethylsilyl |
| UV | ultraviolet |

### General Analytical and Purification Methods

Provided in this section are descriptions of the general analytical and purification methods used to prepare the specific examples provided herein.

Chromatography: Unless otherwise indicated, crude product-containing residues were purified by passing the crude material or concentrate through either a Biotage or ISCO brand silica gel column pre-packed with flash silica (SiO₂) and eluting the product off the column with a solvent gradient as indicated.

Preparative HPLC Method: Where indicated, the compounds described herein were purified via reverse phase HPLC using Waters FractionLynx or Gilson semi-preparative HPLC-MS system utilizing one of the following two HPLC columns: (a) Phenomenex Gemini column (5 micron, C18, 150×30 mm) or (b) Waters X-select CSH column (5 micron, C18, 100×30 mm). A typical run through the instrument included: eluting at 45 mL/min with a linear gradient of 10% (v/v) to 100% MeCN (0.1% v/v formic acid) in water (0.1% formic acid) over 10 minutes; conditions can be varied to achieve optimal separations.

Proton NMR Spectra: Unless otherwise indicated, all ¹H NMR spectra were collected on a Bruker NMR instrument at 300, 400 or 500 MHz. All observed protons are reported as parts-per-million (ppm) downfield from tetramethylsilane (TMS) using the internal solvent peak as reference. Some ¹H signals may be missing due to exchange with D from MeOD, or due to signal suppression.

Mass Spectra (MS): Unless otherwise indicated, all mass spectral data for starting materials, intermediates and/or exemplary compounds are reported as mass/charge (m/z), having an [M+H]+ molecular ion. The molecular ion reported was obtained by electrospray detection method (commonly referred to as an ESI MS) utilizing a Waters Acquity UPLC/MS system. Compounds having an isotopic atom, such as bromine and the like, are generally reported according to the detected isotopic pattern, as appreciated by those skilled in the art.

### Preparation of Intermediates

### 7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (Intermediate A)

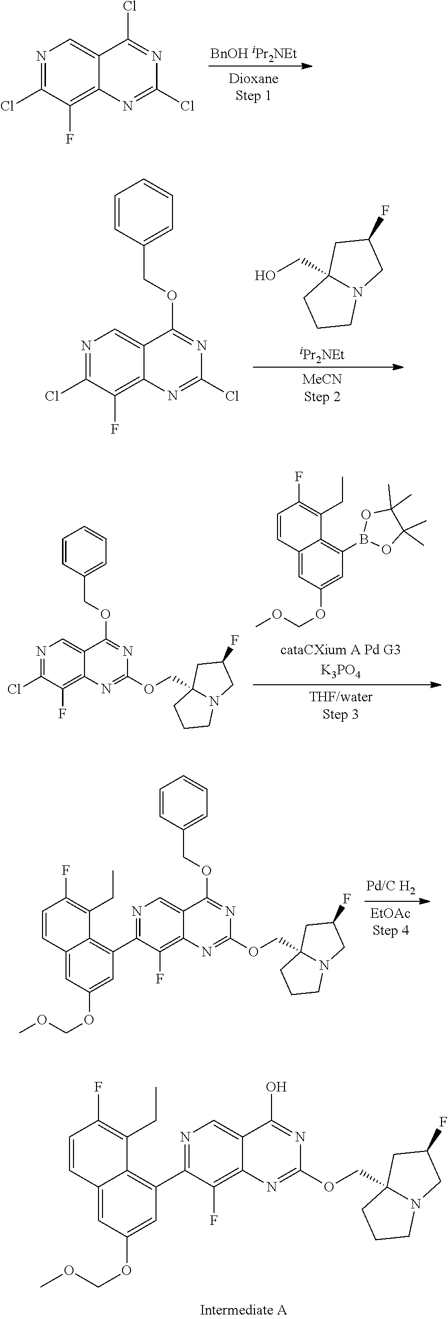

Step 1: 4-(Benzyloxy)-2,7-dichloro-8-fluoropyrido[4,3-d]pyrimidine. A 250-mL round-bottom flask charged with activated 3 Å molecular sieves was added 1,4-dioxane (48 mL), DIPEA (9.22 g, 12.5 mL, 71.3 mmol), benzyl alcohol (3.86 g, 3.7 mL, 35.7 mmol) and 2,4,7-trichloro-8-fluoropyrido[4,3-d]pyrimidine (6.00 g, 23.8 mmol). The mixture was stirred at 85° C. for 2 h. Volatiles were removed in vacuo and the residue was purified by column chromatography on silica gel, eluting with 0-100% 3:1 EtOAc/EtOH blend in heptane to yield 4-(benzyloxy)-2,7-dichloro-8-fluoropyrido[4,3-d]pyrimidine (3.30 g, 10.18 mmol, 43% yield). m/z (ESI): 325.9 (M+H)⁺.

Step 2: 4-(Benzyloxy)-7-chloro-8-fluoro-2-(((2S,7aR)-2-fluorotetrahydro-1H-pyrrolizin-7a(5H)-yl)methoxy)pyrido[4,3-d]pyrimidine. To a solution of 4-(benzyloxy)-2,7-dichloro-8-fluoropyrido[4,3-d]pyrimidine (3.30 g, 10.18 mmol) in acetonitrile (20 mL) were added ((2S,7aR)-2-fluorotetrahydro-1H-pyrrolizin-7a(5H)-yl)methanol (1.78 g, 11.20 mmol) and DIPEA (5.26 g, 7.1 mL, 40.7 mmol). The reaction was stirred at 80° C. for 1 h. Volatiles were removed under reduced pressure and the mixture was purified by column chromatography on silica gel, eluting with 0-100% 3:1 EtOAc/EtOH blend in heptane with 2% triethylamine additive to yield 4-(benzyloxy)-7-chloro-8-fluoro-2-(((2S,7aR)-2-fluorotetrahydro-1H-pyrrolizin-7a(5H)-yl)methoxy)pyrido[4,3-d]pyrimidine (2.60 g, 5.82 mmol, 57% yield). m/z (ESI): 447.0 (M+H)⁺.

Step 3: 4-(Benzyloxy)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine. To a solution of 4-(benzyloxy)-7-chloro-8-fluoro-2-(((2S,7aR)-2-fluorotetrahydro-1H-pyrrolizin-7a(5H)-yl)methoxy)pyrido[4,3-d]pyrimidine (2.60 g, 5.82 mmol) and 2-(8-ethyl-7-fluoro-3-(methoxymethoxy)naphthalen-1-yl)-4,4,5,5-tetramethyl-1,3,2-dioxaborolane (3.14 g, 8.73 mmol) in tetrahydrofuran (17 mL) and water (1.7 mL) were added potassium phosphate (3.70 g, 17.45 mmol) and cataCXium A Pd G3 (0.85 g, 1.16 mmol). The reaction mixture was stirred at 70° C. for 2 h. The reaction mixture was purified by column chromatography on silica gel, eluting with 0-50% 3:1 EtOAc/EtOH blend in heptane with 2% triethylamine additive to yield 4-(benzyloxy)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine (2.42 g, 3.75 mmol, 65% yield). m/z (ESI): 645.0 (M+H)⁺.

Step 4: 7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol. 4-(Benzyloxy)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine (2.42 g, 3.75 mmol) was dissolved in ethyl acetate (75 mL). Palladium on activated carbon (0.80 g, 0.75 mmol) was added and the mixture stirred at rt under an atmosphere of H₂ overnight. The mixture was filtered over celite and the filtercake washed with DCM:MeOH (2:1) until the filtrate ran clear. Volatiles were removed in vacuo to yield 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol as a slightly brownish foam which was used without further purification. m/z (ESI): 555.0 (M+H)⁺.

### 7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (Intermediate B)

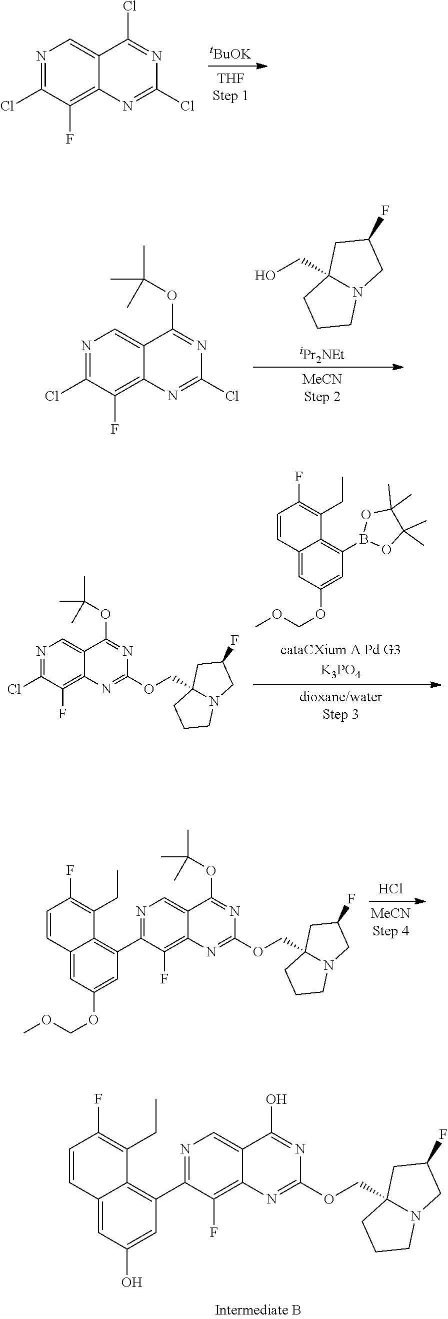

Step 1: 4-(tert-Butoxy)-2,7-dichloro-8-fluoropyrido[4,3-d]pyrimidine. To a stirring mixture of 2,4,7-trichloro-8-fluoropyrido[4,3-d]pyrimidine (2.50 g, 9.90 mmol) in THF (3.5 mL) at −40° C. was added slowly potassium tert-butoxide (1.0 M in THF, 14.9 mL, 14.85 mmol) over a period of 0.5 h. Additional 2-methyl-2-propano potassium salt, 1.0 M solution in THF (2.5 mL) was added after 1 h. The resulting mixture was stirred at −40° C. for 10 min before being poured onto ice and saturated aqueous ammonium hydroxide solution followed by extraction with EtOAc. The combined organics were dried over anhydrous sodium sulfate and concentrated under reduced pressure. The residue was purified by column chromatography on silica gel, eluting with a gradient of 0-20% EtOAc in heptane to give 4-(tert-butoxy)-2,7-dichloro-8-fluoropyrido[4,3-d]pyrimidine (1.12 g, 3.86 mmol, 39% yield). m/z (ESI): 234.0 (M-^{t}Bu+H)⁺.

Step 2: 4-(tert-Butoxy)-7-chloro-8-fluoro-2-(((2R,7aS)-2-fluorotetrahydro-1H-pyrrolizin-7a(5H)-yl)methoxy)pyrido[4,3-d]pyrimidine. A mixture of 4-(tert-butoxy)-2,7-dichloro-8-fluoropyrido[4,3-d]pyrimidine (0.58 g, 2.00 mmol), ((2R,7aS)-2-fluorotetrahydro-1H-pyrrolizin-7a(5H)-yl)methanol (0.45 g, 2.80 mmol) and 1,1′-dimethyltriethylamine (1.03 g, 1.4 mL, 8.00 mmol) in MeCN (6.0 mL) in a 10-mL microwave reaction vessel was subjected to microwave irradiation (16 h at 75° C.). Volatiles were removed under reduced pressure and the crude mixture was purified by column chromatography on silica gel, eluting with a gradient of 0-50% (20% MeOH in DCM) in DCM to give 4-(tert-butoxy)-7-chloro-8-fluoro-2-(((2R,7aS)-2-fluorotetrahydro-1H-pyrrolizin-7a(5H)-yl)methoxy)pyrido[4,3-d]pyrimidine (0.66 g, 1.60 mmol, 80% yield) as off-white solid. m/z (ESI): 413.2 (M+H)⁺.

Step 3: 4-(tert-Butoxy)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine. In a 5-mL microwave reaction vessel were placed 4-(tert-butoxy)-7-chloro-8-fluoro-2-(((2R,7aS)-2-fluorotetrahydro-1H-pyrrolizin-7a(5H)-yl)methoxy)pyrido[4,3-d]pyrimidine (0.66 g, 1.60 mmol), 2-(8-ethyl-7-fluoro-3-(methoxymethoxy)naphthalen-1-yl)-4,4,5,5-tetramethyl-1,3,2-dioxaborolane (0.92 g, 2.56 mmol), cataCXium A Pd G3 (0.23 g, 0.32 mmol), and potassium phosphate tribasic (0.85 g, 4.00 mmol) followed by 1,4-dioxane (10 mL) and water (1.8 mL). The resulting mixture was purged with nitrogen for 10 min before being sealed and irradiated under microwave at 85° C. for 3 h. Volatiles were removed under reduced pressure, and the crude residue was purified by column chromatography on silica gel, eluting with a gradient of 0-50% (20% MeOH in DCM) in DCM to give 4-(tert-butoxy)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine (0.84 g, 1.38 mmol, 86% yield) as colorless film. m/z (ESI): 611.2 (M+H)⁺.

Step 4: 7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol. To a stirred solution of 4-(tert-butoxy)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine (0.84 g, 1.38 mmol) in MeCN (2.0 mL) was added 4.0 M solution of HCl in dioxane (12 mL, 48.1 mmol) at rt. The resulting mixture was stirred at rt for 0.5 h. Volatiles were removed under reduced pressure. The crude residue was dissolved in MeOH/DCM, cooled in an ice bath, and neutralized with ammonium hydroxide before loading onto a silica gel precolumn and purified by column chromatography on silica gel, eluting with a gradient of 0-50% (20% MeOH in DCM) in DCM to give 7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (0.39 g, 0.76 mmol, 56% yield) as off-white solid. m/z (ESI): 511.0 (M+H)⁺.

### N-Methyl-1,4-oxazepane-2-carboxamide hydrochloride (Intermediate C1)

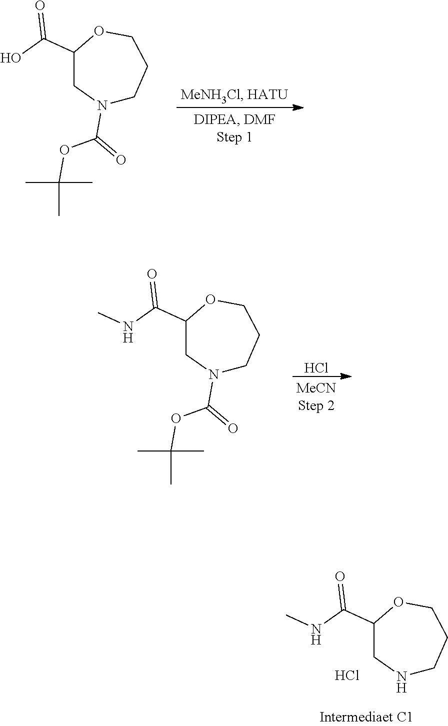 [SMILES: CC(C)(C)OC(=O)N1CCCOC(C(=O)O)C1 ; CNC(=O)C1CN(C(=O)OC(C)(C)C)CCCO1 ; Cl ; CNC(=O)C1CNCCCO1 ; Cl]

Step 1: tert-Butyl 2-(methylcarbamoyl)-1,4-oxazepane-4-carboxylate. 4-[(tert-Butoxy)carbonyl]-1,4-oxazepane-6-carboxylic acid (0.20 g, 0.82 mmol, AA Blocks), methylamine hydrochloride (0.10 g, 3.26 mmol, Spectrum Chemicals), DIPEA (0.43 mL, 2.45 mmol) and HATU (0.37 g, 0.98 mmol) were dissolved in DMF (4.0 mL). The reaction was stirred at rt for 16 h. Upon completion, the mixture was loaded onto a reverse phase column and purified by reverse phase HPLC to yield tert-butyl 2-(methylcarbamoyl)-1,4-oxazepane-4-carboxylate (0.21 g, 0.82 mmol, 100% yield) as colorless oil.

Step 2: N-Methyl-1,4-oxazepane-2-carboxamide hydrochloride. tert-Butyl 2-(methylcarbamoyl)-1,4-oxazepane-4-carboxylate (0.21 g, 0.82 mmol) was dissolved in 2.0 mL of MeCN and HCl (4.0 M in dioxane, 0.61 mL, 2.45 mmol). The reaction was stirred at rt for 30 min. Upon completion, the mixture was concentrated to give N-methyl-1,4-oxazepane-2-carboxamide (0.13 g, 0.82 mmol, 100% yield) as white solid, which was used in the following step without purification. m/z (ESI): 159.1 (M+H)⁺.

TABLE 2
Intermediates C2 to C4, prepared in an analogous manner to Intermediate C1.

| | | | | MS |
|---|---|---|---|---|
| | Chemical | | | m/z (ESI) |
| Intermediate# | Structure | Name | Reagent | (M + H)⁺ |
| C2 | 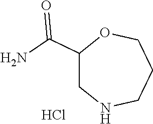 | 1,4-Oxazepane-2- carboxamide Hydrochloride | 4-(tert- butoxycarbonyl)- 1,4-oxazepane-2- carboxylic acid (CAS#: 1141669- 61-5, Combi Blocks) and NH₄Cl | 145.1 |
| C3 | 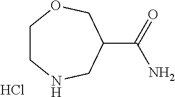 | 1,4-Oxazepane-6- carboxamide Hydrochloride | 1,4-oxazepane-6- carboxamide (CAS#: 1269755- 58-9, AA Blocks) and NH₄Cl | 145.1 |
| C4 | 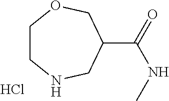 | N-Methyl-1,4- oxazepane-6- carboxamide Hydrochloride | 4-[(tert- butoxy)carbonyl]- 1,4-oxazepane-6- carboxylic acid (CAS#: 1269755- 58-9, AA Blocks) | 159.2 |

### (1,4-Oxazepan-2-yl)methanol Hydrochloride (Intermediate D1)

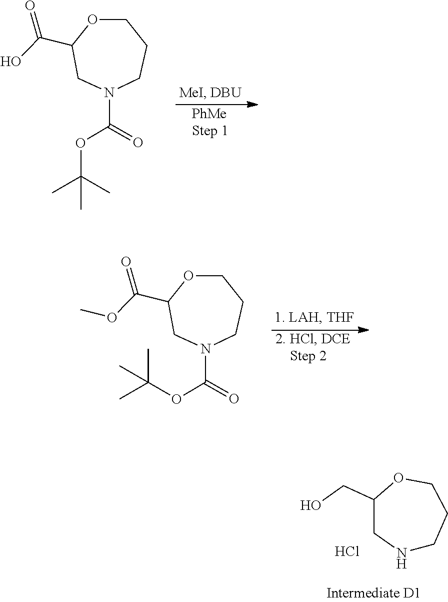

Step 1: 4-(tert-Butyl) 2-methyl 1,4-oxazepane-2,4-dicarboxylate. To a 100-mL round-bottomed flask was added 4-(tert-butoxycarbonyl)-1,4-oxazepane-2-carboxylic acid (0.50 g, 2.04 mmol, Enamine), toluene (10 mL) and DBU (0.31 g, 0.31 mL, 2.04 mmol). To the mixture was added Mel (0.38 mL, 6.12 mmol) and the reaction was allowed to stir at rt for 16 h. Upon completion, the reaction was concentrated under reduced pressure and purified by column chromatography on silica gel, eluting with a gradient of 0-100% EtOAc in heptanes to give 4-(tert-butyl) 2-methyl 1,4-oxazepane-2,4-dicarboxylate (0.48 g, 1.86 mmol, 91% yield) as yellow oil. ¹H NMR (400 MHz, CHLOROFORM-d): δ ppm 4.07-4.38 (m, 3 H), 3.79 (m, 4H), 3.55-3.72 (m, 1H), 3.07-3.36 (m, 2H), 1.86-2.06 (m, 2H), 1.49 (s, 9H).

Step 2: (1,4-Oxazepan-2-yl)methanol hydrochloride. To a 100-mL round-bottomed flask was added 4-(tert-butyl) 2-methyl 1,4-oxazepane-2,4-dicarboxylate (0.48 g, 1.86 mmol), THF (9.0 mL), and lithium aluminum hydride (2.0 M in THF, 1.88 mL, 3.77 mmol). The mixture was allowed to stir at rt for 2 h. Upon completion, the mixture was quenched with saturated aqueous Rochelle salt solution (0.5 mL) and stirred for an additional 1 h. The reaction mixture was then filtered through a celite plug and washed with EtOAc. The filtrate was concentrated under reduced pressure. The residue was redissolved in DCE (5 mL) and HCl (4.0 M in dioxane, 1.39 mL, 5.56 mmol). The reaction was stirred at rt for 2 h. Upon completion, the reaction was concentrated to give (1,4-oxazepan-2-yl)methanol hydrochloride (0.24 g, 1.85 mmol, 99% yield) as white solid, which was used in the following step without further purification. m/z (ESI): 132.2 (M+H)⁺.

### (1,4-Oxazepan-6-yl)methanol Hydrochloride (Intermediate D2)

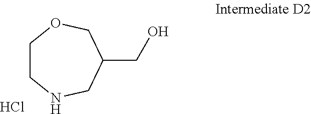

Synthesized in an analogous manner to Intermediate D1, using 4-(tert-butoxycarbonyl)-1,4-oxazepane-6-carboxylic acid (CAS #: 1269755-58-9, Enamine).

### Thiomorpholin-2-ylmethanol Hydrochloride (Intermediate E)

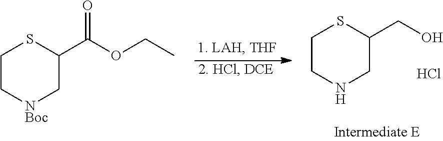

To a 100-mL round-bottomed flask was added 4-(tert-butyl) 2-ethyl thiomorpholine-2,4-dicarboxylate (0.50 g, 1.82 mmol, Accela), THF (9.0 mL), and lithium aluminum hydride (2.0 M in THF, 1.82 mL, 3.63 mmol). The mixture was allowed to stir at rt for 2 h. Upon completion, the mixture was quenched with saturated aqueous Rochelle salt solution (0.5 mL), and stirred for an additional 1 h. The reaction mixture was then filtered through a celite plug and washed with EtOAc. The filtrate was concentrated under reduced pressure. The residue was redissolved in DCE (5 mL) and HCl (4.0 M in dioxane, 1.36 mL, 5.45 mmol).

The reaction was stirred at rt for 2 h. Upon completion, the reaction was concentrated to give thiomorpholin-2-ylmethanol (0.24 g, 1.80 mmol, 99% yield) as white solid, which was used in the following step without further purification. ¹H NMR (400 MHz, METHANOL-d₄): δ ppm 3.73-3.82 (m, 1H), 3.64-3.73 (m, 2H), 3.53-3.61 (m, 1H), 3.17-3.26 (m, 2H), 3.14-3.30 (m, 1H), 2.92-3.05 (m, 2H).

### 3-Oxa-7-azabicyclo[3.3.1]nonan-9-ol (Intermediate F)

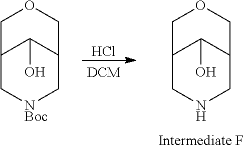 [SMILES: CC(C)(C)OC(=O)N1CC2COCC(C1)C2O ; Cl ; OC1C2CNCC1COC2]

To a 25-mL round-bottomed flask was added tert-butyl 9-hydroxy-3-oxa-7-azabicyclo[3.3.1]nonane-7-carboxylate (0.20 g, 0.82 mmol, Ambeed), DCM (4.0 mL), and HCl solution (4.0 M in dioxane, 0.62 mL, 2.47 mmol). The mixture was allowed to stir at rt for 4 h. Upon completion, the reaction was concentrated to give 3-oxa-7-azabicyclo[3.3.1]nonan-9-ol (0.12 g, 0.82 mmol, 100% yield), which was used in the following step without further purification. m/z (ESI): 144.1 (M+H)⁺.

### 2-(((tert-Butyldimethylsilyl)oxy)methyl)morpholine (Intermediate G)

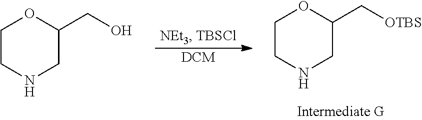

Morpholin-2-ylmethanol (0.38 g, 3.21 mmol, Combi Blocks) was dissolved in dichloromethane (16 mL). Triethylamine (0.49 g, 0.67 mL, 4.82 mmol) was added and the solution was cooled to 0° C. tert-Butylchlorodimethylsilane (0.53 g, 3.53 mmol) was added in one portion, and the mixture warmed to rt and stirred overnight. The crude mixtures were directly loaded onto silica gel and purified via column chromatography on silica gel, eluting with a gradient of 0-35% MeOH in DCM to yield 2-(((tert-butyldimethylsilyl)oxy)methyl)morpholine (0.45 mg, 1.94 mmol, 60% yield). m/z (ESI): 232.2 (M+H)⁺.

### 1,4-Oxazepane-6-carbonitrile (Intermediate H)

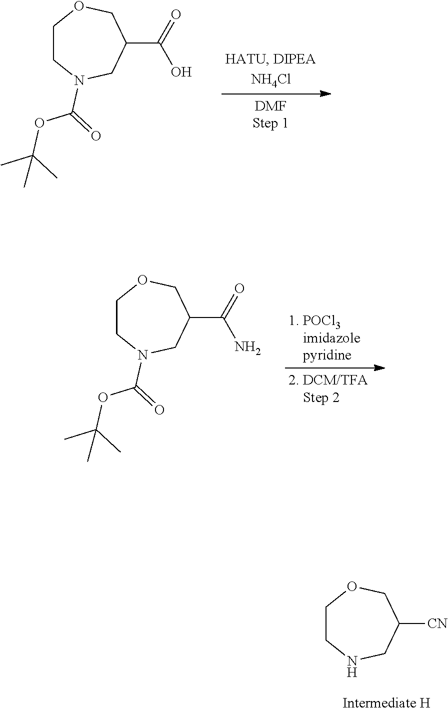 [SMILES: CC(C)(C)OC(=O)N1CCOCC(C(=O)O)C1 ; CC(C)(C)OC(=O)N1CCOCC(C(N)=O)C1 ; N#CC1CNCCOC1]

Step 1: tert-Butyl 6-carbamoyl-1,4-oxazepane-4-carboxylate. 4-[(tert-Butoxy)carbonyl]-1,4-oxazepane-6-carboxylic acid (0.20 g, 0.82 mmol), ammonium chloride (0.40 g, 7.48 mmol), DIPEA (0.32 g, 0.43 mL, 2.45 mmol) and HATU (0.37 g, 0.98 mmol) were dissolved in N,N-dimethylformamide (4.0 mL). The reaction was stirred at rt overnight. The mixture was loaded onto a reverse phase column and purified via reverse phase column chromatography (10-100% MeCN/H₂O+0.01% TFA) to yield tert-butyl 6-carbamoyl-1,4-oxazepane-4-carboxylate (0.16 g, 0.64 mmol, 78% yield) as colorless oil. m/z (ESI): 267.2 (M+H)⁺.

Step 2: 1,4-Oxazepane-6-carbonitrile. tert-Butyl 6-carbamoyl-1,4-oxazepane-4-carboxylate (0.15 g, 0.61 mmol) was dissolved in pyridine (1.5 mL) and 1H-imidazole (84 mg, 1.23 mmol) was added. The mixture was cooled to −30° C. before phosphorous oxychloride (0.38 g, 0.23 mL, 2.46 mmol) was added slowly dropwise. The mixture was stirred at the same temperature for 1 h. Saturated NH₄Cl (2 mL) was added to quench the reaction. The aqueous layer extracted with EtOAc (3×2 mL) and the combined organic layers were dried over Na₂SO₄. Volatiles were removed in vacuo and the crude residue was purified via column chromatography on silica gel, eluting with a gradient of 0-20% MeOH in DCM to yield tert-butyl 6-cyano-1,4-oxazepane-4-carboxylate (94 mg, 0.42 mmol, 68% yield as a colorless oil, which was then dissolved in DCM (2 mL) and TFA (200 uL). The mixture was stirred at rt for 1 h. Volatiles removed in vacuo and the residue was purified via column chromatography on silica gel, eluting with a gradient of 0-30% MeOH (with 0.5% 2 N NH₃ in MeOH) in DCM to yield 1,4-oxazepane-6-carbonitrile (45 mg, 0.36 mmol, 58% yield) as colorless oil. m/z (ESI): 127.2 (M+H)⁺.

### 8-Oxa-3-azabicyclo[3.2.1]octane-6-carbonitrile Hydrochloride (Intermediate I)

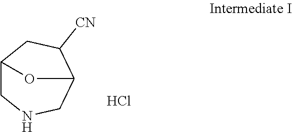

Synthesized in an analogous manner to Intermediate H, using 3-[(tert-butoxy)carbonyl]-8-oxa-3-azabicyclo[3.2.1]octane-6-carboxylic acid (CAS #: 1251010-77-1, Enamine). m/z (ESI): 151.2 (M+H)⁺.

### 6-Methyl-1,4-oxazepan-6-ol hydrochloride Isomer 1 (Intermediate J1) and Isomer 2 (Intermediate J2)

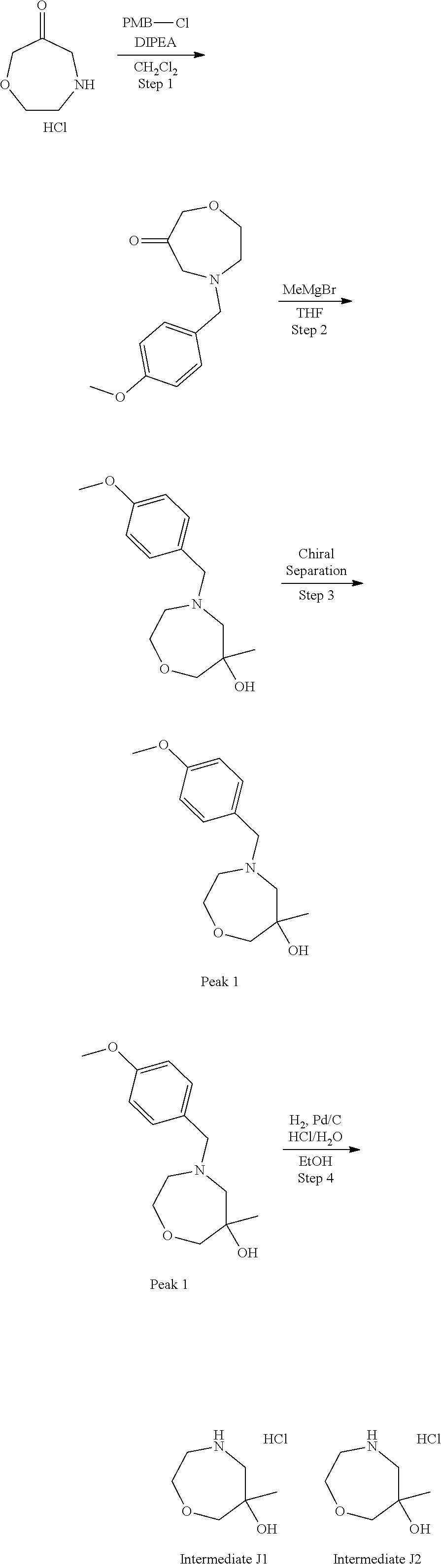

Step 1: 4-(4-Methoxybenzyl)-1,4-oxazepan-6-one. To a 20-mL vial was added 1,4-oxazepan-6-one hydrochloride (0.30 g, 1.98 mmol, AA BLOCKS LLC), 4-methoxybenzyl chloride (0.37 g, 0.32 mL, 2.38 mmol, TCI America), DIPEA (0.77 g, 1.0 mL, 5.94 mmol, Sigma-Aldrich Corporation) and DCM (10 mL). The reaction was stirred at rt overnight. The crude material was purified by column chromatography on silica gel column, eluting with a gradient of 0-80% 3:1 EtOAc/EtOH (with 1% TEA) in heptane to provide 4-(4-methoxybenzyl)-1,4-oxazepan-6-one (0.43 g, 1.83 mmol, 92% yield) as colorless oil. m/z (ESI): 236.2 (M+H)⁺.

Step 2: 4-(4-Methoxybenzyl)-6-methyl-1,4-oxazepan-6-ol. To a 100-mL round-bottomed flask was added 4-(4-methoxybenzyl)-1,4-oxazepan-6-one (0.87 g, 3.70 mmol) in THF (15 mL). The mixture was cooled to 0° C. before methylmagnesium bromide solution (3 M in Et₂O, 3.7 mL, 11.09 mmol, Sigma-Aldrich Corporation) was added. The reaction was stirred for 1 h. The reaction mixture was diluted with saturated NH₄Cl (15 mL) and extracted with EtOAc (2×15 mL). The organic extract was washed with saturated NaCl (15 mL) and dried over MgSO₄. The solution was filtered and concentrated in vacuo to give the crude material. The crude material was purified by column chromatography on silica gel, eluting with a gradient of 0-50% 3:1 EtOAc/EtOH in heptane to provide 4-(4-methoxybenzyl)-6-methyl-1,4-oxazepan-6-ol (0.65 g, 2.59 mmol, 70% yield) as yellow oil. m/z (ESI): 252.1 (M+H)⁺.

Step 3: Chiral separation. 4-(4-Methoxybenzyl)-6-methyl-1,4-oxazepan-6-ol (0.65 g, 2.59 mmol) was purified via SFC using a Chiralpak AD, 30×150 mm 5 μm, column with a mobile phase of 20% methanol with 0.2% triethylamine using a flowrate of 200 mL/min to generate 246 mg of peak 1 with an ee of >99% and 292 mg of peak 2 with an ee of >99%.

Step 4: 6-Methyl-1,4-oxazepan-6-ol hydrochloride. 4-(4-methoxybenzyl)-6-methyl-1,4-oxazepan-6-ol (0.24 g, 0.96 mmol, Peak 1) was dissolved in ethanol (5.8 mL). palladium on activated carbon (0.25 g, 0.23 mmol, Sigma-Aldrich Corporation) and aqueous HCl solution (2 N, 0.7 mL, 1.33 mmol, Sigma-Aldrich Corporation) were added and the mixture stirred at rt under an atmosphere of H₂ for 5 h. The catalyst was removed and the solution was concentrated to provide 6-methyl-1,4-oxazepan-6-ol hydrochloride (quant. yield, isomer 1, Intermediate J1). Isomer 2, Intermediate J2 was obtained by the same method.

### 6-((Trimethylsilyl)ethynyl)-1,4-oxazepan-6-ol hydrochloride (Intermediate K)

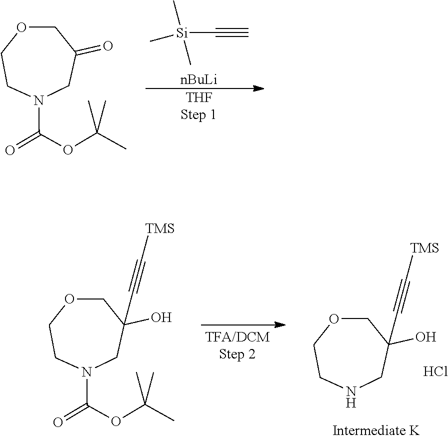

Step 1: tert-Butyl 6-hydroxy-6-((trimethylsilyl)ethynyl)-1,4-oxazepane-4-carboxylate. To a 50-mL round-bottomed flask was added (trimethylsilyl)acetylene (0.27 g, 0.27 mL, 2.79 mmol, Combi-Blocks Inc.) in THF (3.7 mL). The mixture was cooled to −78° C. before n-butyllithium solution in hexanes (2.5 M, 0.9 mL, 2.32 mmol, Sigma-Aldrich Corporation) was added. The reaction was stirred for 15 min before being warmed to 0° C. and tert-butyl 6-oxo-1,4-oxazepane-4-carboxylate (0.20 g, 0.2 mL, 0.93 mmol, Combi-Blocks Inc.) was added. The reaction was stirred at this temperature for 2 h. The reaction mixture was diluted with saturated. NH₄Cl (10 mL) and extracted with EtOAc (2×15 mL). The organic extract was washed with saturated. NaCl (15 mL) and dried over MgSO₄. The solution was filtered and concentrated in vacuo to give the crude material. The crude material was purified by column chromatography on silica gel, eluting with a gradient of 0-80% 3:1 EtOAc/EtOH in heptane to provide tert-butyl 6-hydroxy-6-((trimethylsilyl)ethynyl)-1,4-oxazepane-4-carboxylate (0.24 g, 0.77 mmol, 82% yield) as yellow oil.

Step 2: 6-((Trimethylsilyl)ethynyl)-1,4-oxazepan-6-ol hydrochloride. tert-Butyl 6-hydroxy-6-((trimethylsilyl)ethynyl)-1,4-oxazepane-4-carboxylate (0.12 g, 0.38 mmol) was dissolved in 2 mL DCM and 0.5 mL of TFA. The reaction was stirred for 1 h. The mixture was then concentrated in vacuo. To the residue was added 0.6 mL of 1 N HCl aq. solution and the mixture was lyophilized to provide 6-((trimethylsilyl)ethynyl)-1,4-oxazepan-6-ol hydrochloride (quant. yield). m/z (ESI): 214.2 (M+H)⁺.

### 4-(7-(8-Ethyl-7-fluoro-3-(methoxymethoxy)naphthalen-1-yl)-8-fluoro-2-(methylsulfinyl)pyrido[4,3-d]pyrimidin-4-yl)-1,4-oxazepane (Intermediate L)

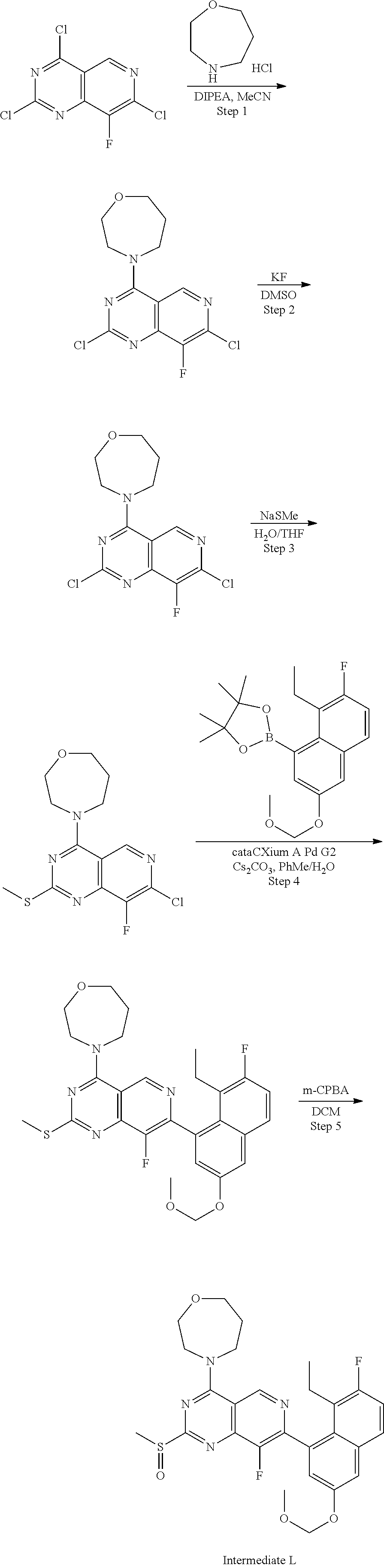

Step 1: 4-(2,7-Dichloro-8-fluoro-pyrido[4,3-d]pyrimidin-4-yl)-1,4-oxazepane. To a solution of 2,4,7-trichloro-8-fluoro-pyrido[4,3-d]pyrimidine (33 g, 0.13 mol, LabNetwork) and DIPEA (42 g, 57 mL, 0.33 mol) in MeCN (500 mL) was added 1,4-oxazepane hydrochloride (14 g, 0.10 mol) in portions at −40° C. The mixture was stirred at −40° C. for 1 h. The reaction mixture was diluted with H₂O (500 mL) and extracted with EtOAc (500 mL×3). The combined organic layers were washed with brine (250 mL×2), dried over Na₂SO₄, filtered and the filtrate was concentrated under reduced pressure to give a residue. The residue was purified by column chromatography on silica gel, eluting with 5:1 to 3:1 petroleum ether/EtOAc to give 4-(2,7-dichloro-8-fluoro-pyrido[4,3-d]pyrimidin-4-yl)-1,4-oxazepane (22 g, 70 mmol, 53% yield, 91% purity) as yellow solid. m/z (ESI): 317.1 (M+H)⁺.

Step 2: 4-(7-Chloro-2,8-difluoro-pyrido[4,3-d]pyrimidin-4-yl)-1,4-oxazepane. To a solution of 4-(2,7-dichloro-8-fluoro-pyrido[4,3-d]pyrimidin-4-yl)-1,4-oxazepane (32 g, 0.10 mol) in DMSO (330 mL) was added KF (59 g, 1.01 mol). The mixture was stirred at 80° C. for 12 h. The reaction mixture was diluted with H₂O (700 mL) and extracted with EtOAc (500 mL×3). The combined organic layers were washed with brine (350 mL×2), dried over Na₂SO₄, filtered and the filtrate was concentrated under reduced pressure to give a residue. The residue was purified by column chromatography on silica gel, eluting with 3:1 to 1:1 petroleum ether/EtOAc to give 4-(7-chloro-2,8-difluoro-pyrido[4,3-d]pyrimidin-4-yl)-1,4-oxazepane (20 g, 67 mmol, 66% yield) as yellow solid. m/z (ESI): 301.2 (M+H)⁺.

Step 3: 4-(7-Chloro-8-fluoro-2-methylsulfanyl-pyrido[4,3-d]pyrimidin-4-yl)-1,4-oxazepane. To a solution of 4-(7-chloro-2,8-difluoro-pyrido[4,3-d]pyrimidin-4-yl)-1,4-oxazepane (31 g, 0.10 mol) in THF (310 mL) was added dropwise NaSMe (20% purity in H₂O, 33 mL, 0.10 mol) at 0° C. The mixture was stirred at 20° C. for 2 h to give a yellow suspension. The reaction mixture was filtered and the cake was concentrated under reduced pressure to give a residue. The filtrate was concentrated under reduced pressure to give a residue, and the cake was triturated with 8:1 petroleum ether/EtOAc (250 mL) at 20° C. for 30 min. The suspension were filtered and the cake was washed with 8:1 petroleum ether/EtOAc (100 mL×3) and concentrated under reduced pressure to give 4-(7-chloro-8-fluoro-2-methylsulfanyl-pyrido[4,3-d]pyrimidin-4-yl)-1,4-oxazepane (37 g, 0.10 mol, 98% yield, 90% purity) as yellow solid. m/z (ESI): 329.0 (M+H)⁺.

Step 4: 4-(7-(8-Ethyl-7-fluoro-3-(methoxymethoxy)-1-naphthyl)-8-fluoro-2-methylsulfanyl-pyrido[4,3-d]pyrimidin-4-yl)-1,4-oxazepane. To a solution of 4-(7-chloro-8-fluoro-2-methylsulfanyl-pyrido[4,3-d]pyrimidin-4-yl)-1,4-oxazepane (10 g, 30 mmol) and 2-[8-ethyl-7-fluoro-3-(methoxymethoxy)-1-naphthyl]-4,4,5,5-tetramethyl-1,3,2-dioxaborolane (14 g, 40 mmol, PharmaBlock), Cs₂CO₃ (9.9 g, 30 mmol) in toluene (200 mL) and H₂O (25 mL) was added cataCXium A Pd G2 (2.0 g, 3.04 mmol) under N₂. The mixture was stirred at 100° C. for 12 h. The reaction mixture was diluted with H₂O (300 mL) and extracted with EtOAc (200 mL×3). The combined organic layers were washed with brine (150 mL×2), dried over Na₂SO₄, filtered and the filtrate was concentrated under reduced pressure to give a residue. The residue was purified by column chromatography on silica gel, eluting with 2:1 to 0:1 petroleum ether/EtOAc to give 4-(7-(8-ethyl-7-fluoro-3-(methoxymethoxy)-1-naphthyl)-8-fluoro-2-methylsulfanyl-pyrido[4,3-d]pyrimidin-4-yl)-1,4-oxazepane (11 g, 17 mmol, 57% yield, 91% purity) as yellow solid. m/z (ESI): 527.3 (M+H)⁺.

Step 5: 4-(7-(8-ethyl-7-fluoro-3-(methoxymethoxy)-1-naphthyl)-8-fluoro-2-methylsulfinyl-pyrido[4,3-d]pyrimidin-4-yl)-1,4-oxazepane. To a solution of 4-(7-(8-ethyl-7-fluoro-3-(methoxymethoxy)-1-naphthyl)-8-fluoro-2-methylsulfanyl-pyrido[4,3-d]pyrimidin-4-yl)-1,4-oxazepane (11 g, 22 mmol) in DCM (400 mL) at 0° C. was added m-CPBA (4.4 g, 22 mmol, 85% purity) in portions. Then the mixture was stirred at 0° C. for 1 h. The reaction mixture was quenched by addition aq. Na₂S₂O₃ (1000 mL) at 25° C., and then was diluted with H₂O (600 mL) and extracted with EtOAc (200 mL×3). The combined organic layers were washed with brine (200 mL×2), dried over Na₂SO₄, filtered and the filtrate was concentrated under reduced pressure to give a residue. The residue was purified by column chromatography on silica gel, eluting with 100% EtOAc to 10:1 EtOAc/MeOH to give 4-(7-(8-ethyl-7-fluoro-3-(methoxymethoxy)-1-naphthyl)-8-fluoro-2-methylsulfinyl-pyrido[4,3-d]pyrimidin-4-yl)-1,4-oxazepane (6.5 g, 11 mmol, 52% yield, 95% purity) as yellow solid. m/z (ESI): 543.1 (M+H)⁺.

### 6-Methyl-1,4-oxazepan-6-ol hydrochloride Isomer 1 (Intermediate M1) and Isomer 2 (Intermediate M2)

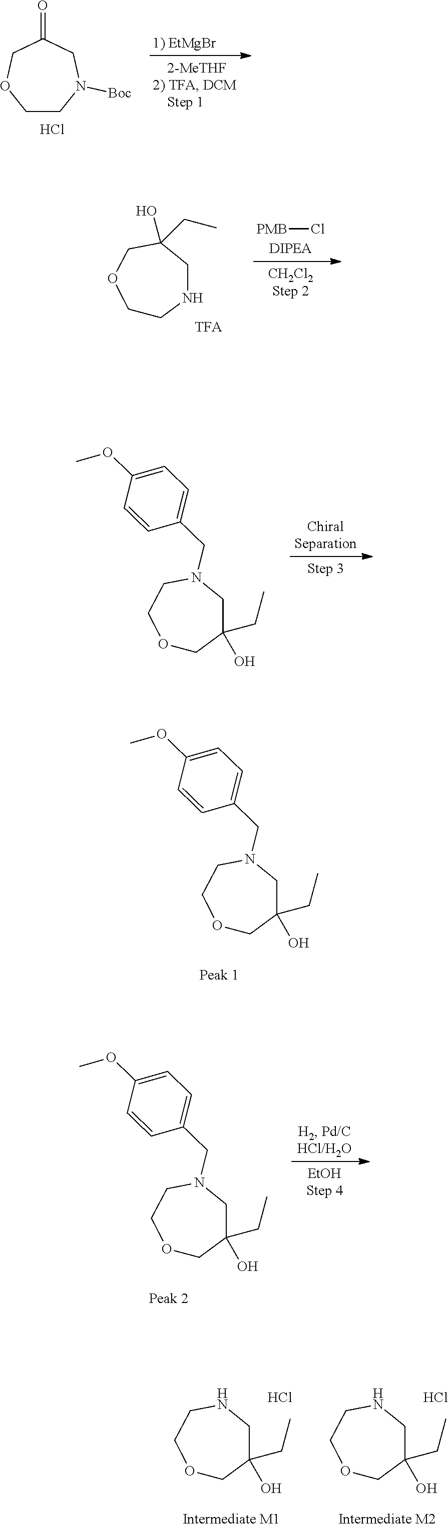

Step 1: 6-Ethyl-1,4-oxazepan-6-ol 2,2,2-trifluoroacetate. To a 100-mL round-bottomed flask was added tert-butyl 6-oxo-1,4-oxazepane-4-carboxylate (0.55 g, 2.56 mmol, Combi-Blocks Inc.) in 2-MeTHF (10 mL) at 0° C. Ethylmagnesium bromide solution, 1.0 M in tetrahydrofuran (6.4 mL, 6.40 mmol) was added dropwise. The reaction mixture was stirred at rt for 1 h and then diluted with saturated NH₄C₁ solution (15 mL) and extracted with EtOAc (2×15 mL). The organic extract was washed with saturated NaCl solution (15 mL) and dried over MgSO₄. The solution was filtered and concentrated in vacuo to give the crude material.

To the above material dissolved in 10 mL of DCM at 0° C. was added TFA (2 mL) dropwise. After stirring at 0° C. to rt for 2 h, the reaction mixture was fully concentrated and carried to the next step without further purification.

Step 2. 6-Ethyl-4-(4-methoxybenzyl)-1,4-oxazepan-6-ol. To a 100 mL round-bottomed flask was added 6-ethyl-1,4-oxazepan-6-ol 2,2,2-trifluoroacetate (0.66 g, 2.56 mmol), 4-methoxybenzyl chloride (0.48 g, 0.42 mL, 3.07 mmol), and N,N-diisopropylethylamine (0.99 g, 1.34 mL, 7.68 mmol) in DCM (10 mL). After stirring at rt overnight, the crude material was absorbed onto a plug of silica gel and purified by column chromatography on silica gel, eluting with a gradient of 0-80% 3:1 EtOAc/EtOH (with 1% TEA) in heptane, to provide 6-ethyl-4-(4-methoxybenzyl)-1,4-oxazepan-6-ol (0.40 g, 1.51 mmol, 59% yield) as colorless oil. m/z (ESI): 266.2 (M+H)⁺.

Step 3: Chiral separation. 6-Ethyl-4-(4-methoxybenzyl)-1,4-oxazepan-6-ol (0.4 g, 1.5 mmol) was purified via SFC using a Chiralpak AZ, 20×250 mm 5 m, column with a mobile phase of 15% methanol using a flowrate of 80 mL/min to generate 163 mg of peak 1 with an ee of 99% and 163 mg of peak 2 with an ee of 99%.

Step 4: 6-Ethyl-1,4-oxazepan-6-ol hydrochloride. 6-Ethyl-4-(4-methoxybenzyl)-1,4-oxazepan-6-ol (0.16 g, 0.61 mmol, Peak 1) was dissolved in ethanol (3.1 mL). Palladium on activated carbon (0.13 g, 0.12 mmol) and aqueous HCl solution (2 N, 0.35 mL, 0.7 mmol) were added and the mixture stirred at rt under an atmosphere of H₂ for 5 h. The catalyst was removed and the solution was concentrated to provide 6-ethyl-1,4-oxazepan-6-ol hydrochloride (quantitative yield, isomer 1, Intermediate M1). Isomer 2, Intermediate M2 was obtained by the same method.

### 5-Ethyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol

### Example 1

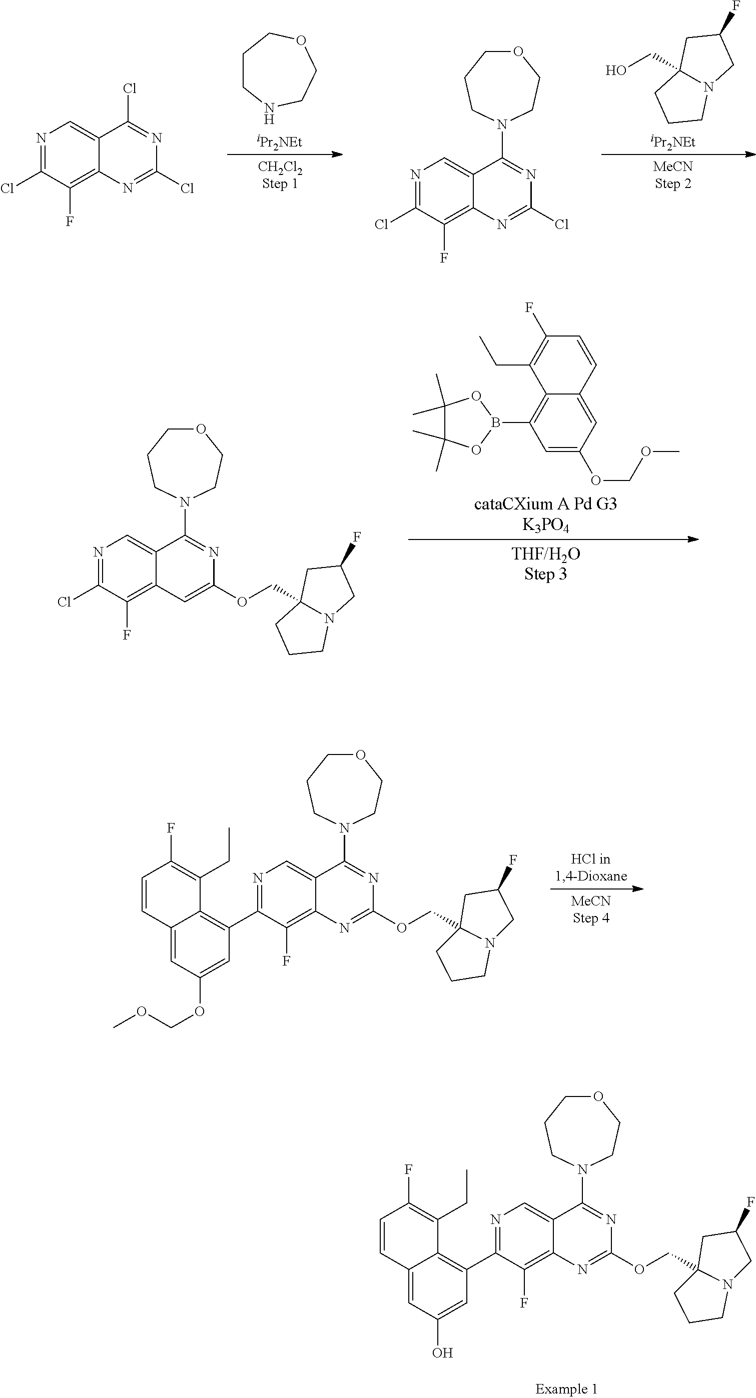

Step 1: 4-(2,7-Dichloro-8-fluoropyrido[4,3-d]pyrimidin-4-yl)-1,4-oxazepane. To a suspension of 2,4,7-trichloro-8-fluoropyrido[4,3-d]pyrimidine (2.00 g, 7.92 mmol) in dichloromethane (31.7 mL) at −40° C. was added 1,4-oxazepane (0.80 g, 7.92 mmol) followed by DIPEA (3.07 g, 4.2 mL, 23.77 mmol). The reaction was stirred at −40° C. for 1 h. The reaction mixture was diluted with DCM, washed with aqueous citric acid, and dried over anhydrous magnesium sulfate. The reaction mixture was filtered and concentrated to provide 4-(2,7-dichloro-8-fluoropyrido[4,3-d]pyrimidin-4-yl)-1,4-oxazepane (2.73 g, 8.61 mmol, 109% yield, 87% purity). The isolated product was used in the next reaction without further purification. m/z (ESI): 317.1 (M+H).

Step 2: 4-(7-Chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane. To a suspension of 4-(2,7-dichloro-8-fluoropyrido[4,3-d]pyrimidin-4-yl)-1,4-oxazepane (1.25 g, 3.94 mmol) and ((2R,7aS)-2-fluorotetrahydro-1H-pyrrolizin-7a(5H)-yl)methanol (0.75 g, 4.73 mmol) in acetonitrile (15.8 mL) was added DIEA (1.53 g, 2.1 mL, 11.82 mmol). The reaction was stirred at 75° C. overnight. The reaction mixture was concentrated, and the crude product was purified by column chromatography on silica gel, eluting with 0-100% 3:1 EtOAc/EtOH blend in heptane with 2% triethylamine additive to provide 4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane (0.67 g, 1.51 mmol, 38% yield) as light-yellow powder. m/z (ESI): 440.2 (M+H)⁺.

Step 3: 4-(7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane. To a solution of 4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane (0.12 g, 0.27 mmol) and 2-(8-ethyl-7-fluoro-3-(methoxymethoxy)naphthalen-1-yl)-4,4,5,5-tetramethyl-1,3,2-dioxaborolane (0.15 g, 0.41 mmol) in tetrahydrofuran (2.5 mL) and water (0.2 mL) were added potassium phosphate (0.17 g, 0.82 mmol) and cataCXium A Pd G3 (40 mg, 0.055 mmol). The reaction mixture was sparged with argon, capped, and stirred at 70° C. for 16 h. The reaction mixture was partitioned between water and ethyl acetate. The organic layer was concentrated. The crude product was purified by column chromatography on silica gel, eluting with 0-100% 3:1 EtOAc/EtOH blend in heptane with 2% triethylamine additive to provide 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane (0.17 g, 0.26 mmol, 95% yield) as off-white powder. m/z (ESI): 638.1 (M+H)⁺.

Step 4: 5-Ethyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol. To a solution of 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane (0.17 g, 0.26 mmol) in acetonitrile (5.2 mL) was added hydrogen chloride solution (4.0 M in dioxane, 1.3 mL, 5.17 mmol). The reaction was stirred at ambient temperature for 30 min. The reaction mixture was concentrated and the crude product was purified by reverse phase HPLC to provide 5-ethyl-6-fluoro-4-(8-fluoro-2-(((2R,7a,S)-2-fluorotetrahydro-H-pyrrolizin-7a(5H-yl)methoxy)-4-(1,4-oxazepan-4-yl)pyrido[4,3-d]pyrimidin-7-yl)naphthalen-2-ol (0.12 g, 0.14 mmol, 55% yield) as its TFA salt as light-yellow powder. m/z (ESI): 594.1 (M+H)⁺. ¹H NMR (400 MHz, DMSO-d₆) δ ppm 10.61-10.84 (br s, 1H) 9.78-10.08 (br s, 1H) 9.24 (s, 1H) 7.78 (dd, J=8.99, 6.06 Hz, 1H) 7.26-7.44 (m, 2H) 7.02 (d, J=2.51 Hz, 1H) 5.45-5.71 (m, 1H) 4.50-4.67 (m, 2H) 4.11-4.30 (m, 4H) 3.68-4.01 (m, 7H) 3.68-3.80 (m, 4H) 3.32 (brs, 1H) 1.90-2.43 (m, 10H) 0.74 (t, J=7.32 Hz, 3H).

TABLE 3
Examples 2 to 33, and 78-80, prepared in an analogous manner to Example 1.

| Ex. | | | Salt | | Method |
|---|---|---|---|---|---|
| # | Chemical Structure | Name | Form | Reagent | Change |
| 2 | 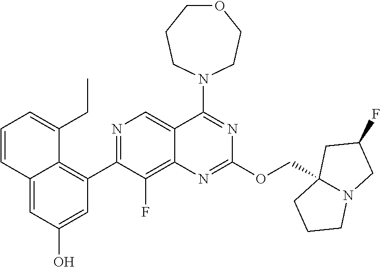 [SMILES: CCc1cccc2cc(O)cc(-c3ncc4c(N5CCCOCC5)nc(OC[C@@]56CCCN5C[C@H](F)C6)nc4c3F)c12] | 5-Ethyl-4-(8- fluoro-2- (((2R,7aS)-2- fluorotetrahydro- 1H-pyrrolizin- 7a(5H)- yl)methoxy)-4- (1,4-oxazepan-4- yl)pyrido[4,3- d]pyrimidin-7- yl)naphthalen-2- ol | bis (2,2,2- trifluoro- acetate) | Step 3: 2-(8- ethyl-3- (methoxymethoxy) naphthalen-1- yl)-4,4,5,5- tetramethyl- 1,3,2- dioxaborolane (CAS#: 2621932- 60-1, LabNetwork) | |
| 3 | 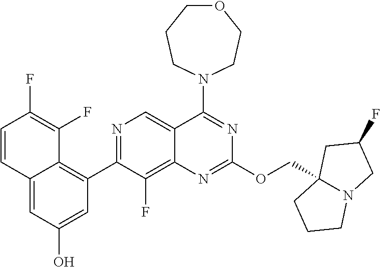 [SMILES: Oc1cc(-c2ncc3c(N4CCCOCC4)nc(OC[C@@]45CCCN4C[C@H](F)C5)nc3c2F)c2c(F)c(F)ccc2c1] | 5,6-Difluoro-4- (8-fluoro-2- (((2R,7aS)-2- fluorotetrahydro- 1H-pyrrolizin- 7a(5H)- yl)methoxy)-4- (1,4-oxazepan-4- yl)pyrido[4,3- d]pyrimidin-7- yl)naphthalen-2- ol | dihydro- chloride | Step 3: 2-(7,8- difluoro-3- (methoxymethoxy) naphthalen-1- yl)-4,4,5,5- tetramethyl- 1,3,2- dioxaborolane (CAS#: 2621935- 35-9, LabNetwork) | |
| 4 | 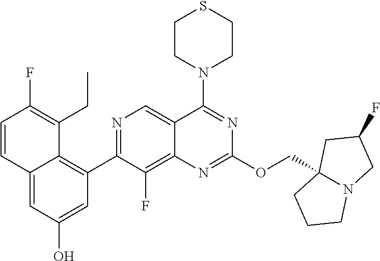 [SMILES: CCc1c(F)ccc2cc(O)cc(-c3ncc4c(N5CCSCC5)nc(OC[C@@]56CCCN5C[C@H](F)C6)nc4c3F)c12] | 5-Ethyl-6-fluoro- 4-(8-fluoro-2- (((2R,7aS)-2- fluorotetrahydro- 1H-pyrrolizin- 7a(5H)- yl)methoxy)-4- thiomorpholino- pyrido[4,3- d]pyrimidin-7- yl)naphthalen-2- ol | bis (2,2,2- trifluoro- acetate | Step 1: thiomorpholine (CAS#: 123-90-0, Combi Blocks) Step 3: Pd(dppf)Cl₂ was used | |
| 5 | 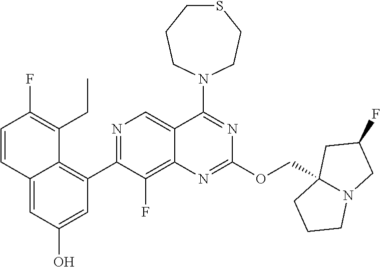 [SMILES: CCc1c(F)ccc2cc(O)cc(-c3ncc4c(N5CCCSCC5)nc(OC[C@@]56CCCN5C[C@H](F)C6)nc4c3F)c12] | 5-Ethyl-6-fluoro- 4-(8-fluoro-2- (((2R,7aS)-2- fluorotetrahydro- 1H-pyrrolizin- 7a(5H)- yl)methoxy)-4- (1,4-thiazepan-4- yl)pyrido[4,3- d]pyrimidin-7- yl)naphthalen-2- ol | bis (2,2,2- trifluoro- acetate) | Step 1: 1,4- thiazepane hydrochloride (CAS#: 108724- 14-7, Synthonix) | |
| 6 | 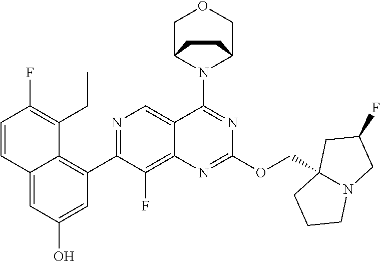 | 4-(4-((1R,5S)-3- Oxa-8- azabicyclo[3.2.1] octan-8-yl)-8- fluoro-2- (((2R,7aS)-2- fluorotetrahydro- 1H-pyrrolizin- 7a(5H)- yl)methoxy)pyrido [4,3- d]pyrimidin-7- yl)-5-ethyl-6- fluoronaphthalen- 2-ol | 2,2,2- trifluoro- acetate | Step 1: 3-oxa-8- azabicyclo[3.2.1] octane (CAS#: 280-07-9, Combi Blocks) | |
| 7 | 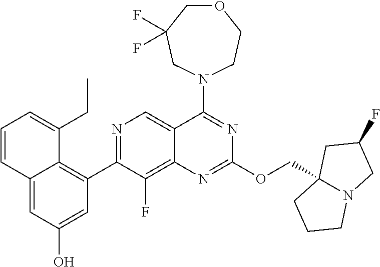 [SMILES: CCc1cccc2cc(O)cc(-c3ncc4c(N5CCOCC(F)(F)C5)nc(OC[C@@]56CCCN5C[C@H](F)C6)nc4c3F)c12] | 4-(4-(6,6- Difluoro-1,4- oxazepan-4-yl)- 8-fluoro-2- (((2R,7aS)-2- fluorotetrahydro- 1H-pyrrolizin- 7a(5H)- yl)methoxy)pyrido [4,3- d]pyrimidin-7- yl)-5- ethylnaphthalen- 2-ol | 2,2,2- trifluoro- acetate | Step 1: 6,6- difluoro-1,4- oxazepane hydrochloride (CAS#: 1341039- 23-3, Enamine) Step 3: 2-(8- ethyl-3- (methoxymethoxy) naphthalen-1- yl)-4,4,5,5- tetramethyl- 1,3,2- dioxaborolane (CAS#: 2621932- 60-1, LabNetwork) | Step 2: DBACO (0.2 eq.), Cs₂CO₃ (3 eq.) was used. |
| 8 | 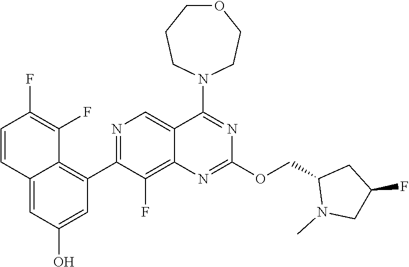 [SMILES: CN1C[C@H](F)C[C@H]1COc1nc(N2CCCOCC2)c2cnc(-c3cc(O)cc4ccc(F)c(F)c34)c(F)c2n1] | 5,6-Difluoro-4- (8-fluoro-2- (((2S,4R)-4- fluoro-1- methylpyrrolidin- 2-yl)methoxy)-4- (1,4-oxazepan-4- yl)pyrido[4,3- d]pyrimidin-7- yl)naphthalen-2- ol | bis (2,2,2- trifluoro- acetate | Step 2: ((2S,4R)- 4-fluoro-1- methylpyrrolidin- 2-yl)methanol (CAS#: 2206737- 78-0, eNovation) Step 3: 2-(7,8- difluoro-3- (methoxymethoxy) naphthalen-1- yl)-4,4,5,5- tetramethyl- 1,3,2- dioxaborolane (CAS#: 2621935- 35-9, LabNetwork) | |
| 9 | 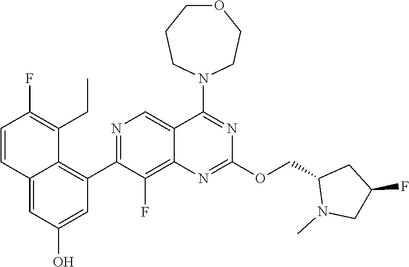 [SMILES: CCc1c(F)ccc2cc(O)cc(-c3ncc4c(N5CCCOCC5)nc(OC[C@@H]5C[C@@H](F)CN5C)nc4c3F)c12] | 5-Ethyl-6-fluoro- 4-(8-fluoro-2- (((2S,4R)-4- fluoro-1- methylpyrrolidin- 2-yl)methoxy)-4- (1,4-oxazepan-4- yl)pyrido[4,3- d]pyrimidin-7- yl)naphthalen-2- ol | bis (2,2,2- trifluoro- acetate) | Step 2: ((2S,4R)- 4-fluoro-1- methylpyrrolidin- 2-yl)methanol (CAS#: 2206737- 78-0, eNovation) | |
| 10 | 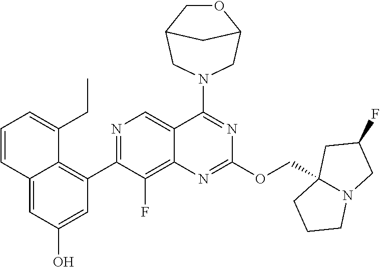 [SMILES: CCc1cccc2cc(O)cc(-c3ncc4c(N5CC6COC(C6)C5)nc(OC[C@@]56CCCN5C[C@H](F)C6)nc4c3F)c12] | 4-(4-(6-Oxa-3- azabicyclo[3.2.1] octan-3-yl)-8- fluoro-2- (((2R,7aS)-2- fluorotetrahydro- 1H-pyrrolizin- 7a(5H)- yl)methoxy)pyrido [4,3- d]pyrimidin-7- yl)-5- ethylnaphthalen- 2-ol | 2,2,2- trifluoro- acetate | Step 1: 6-oxa-3- azabicyclo[3.2.1] octane hydrochloride (CAS#: 1461708- 41-7, Enamine) Step 3: 2-(8- ethyl-3- (methoxymethoxy) naphthalen-1- yl)-4,4,5,5- tetramethyl- 1,3,2- dioxaborolane (CAS#: 2621932- 60-1, LabNetwork) | Step 2: DABCO (0.2 eq.), Cs₂CO₃ (3 eq.) was used. |
| 11 | 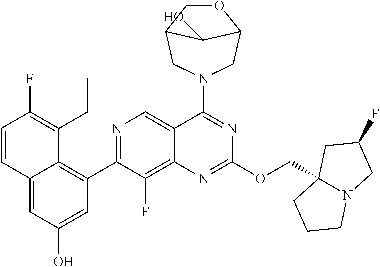 [SMILES: CCc1c(F)ccc2cc(O)cc(-c3ncc4c(N5CC6COC(C5)C6O)nc(OC[C@@]56CCCN5C[C@H](F)C6)nc4c3F)c12] | 3-(7-(8-Ethyl-7- fluoro-3- hydroxynaphthalen- 1-yl)-8-fluoro- 2-(((2R,7aS)-2- fluorotetrahydro- 1H-pyrrolizin- 7a(5H)- yl)methoxy)pyrido [4,3- d]pyrimidin-4- yl)-6-oxa-3- azabicyclo[3.2.1] octan-8-ol | 2,2,2- trifluoro- acetate | Step 1: (1R,5R,8R)-rel-6- oxa-3- azabicyclo[3.2.1] octan-8-ol (CAS#: 1408076- 41-4, PharmaBlock, Inc.) | |
| 12 | 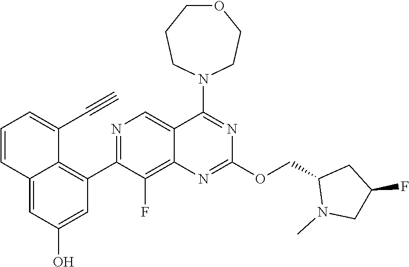 [SMILES: C#Cc1cccc2cc(O)cc(-c3ncc4c(N5CCCOCC5)nc(OC[C@@H]5C[C@@H](F)CN5C)nc4c3F)c12] | 5-Ethynyl-4-(8- fluoro-2- (((2S,4R)-4- fluoro-1- methylpyrrolidin- 2-yl)methoxy)-4- (1,4-oxazepan-4- yl)pyrido[4,3- d]pyrimidin-7- yl)naphthalen-2- ol | 2,2,2- trifluoro- acetate | Step 2: ((2S,4R)- 4-fluoro-1- methylpyrrolidin- 2-yl)methanol (CAS#: 2206737- 78-0, eNovation) Step 3: triisopropyl((6- (methoxymethoxy)- 8-(4,4,5,5- tetramethyl- 1,3,2- dioxaborolan-2- yl)naphthalen-1- yl)ethynyl)silane (CAS#: 2621932- 42-9, LabNetwork) | Step 3: Pd(PPh₃)₄ and Cs₂CO₃ were used. Additional step after Step 4. Details included below. |
| 13 | 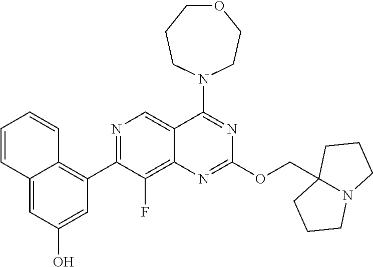 [SMILES: Oc1cc(-c2ncc3c(N4CCCOCC4)nc(OCC45CCCN4CCC5)nc3c2F)c2ccccc2c1] | 4-(8-Fluoro-4- (1,4-oxazepan-4- yl)-2- ((tetrahydro-1H- pyrrolizin- 7a(5H)- yl)methoxy)pyrido [4,3- d]pyrimidin-7- yl)naphthalen-2- ol | free base | Step 2: (tetrahydro-1H- pyrrolizin- 7a(5H)- yl)methanol (CAS#: 78449- 72-6, Enamine) Step 3: 4- (4,4,5,5- tetramethyl- 1,3,2- dioxaborolan-2- yl)-2-naphthol (CAS#: 2043962- 01-0, ChemBridge Corporation) | Step 3: Pd(PPh₃)₄ and Cs₂CO₃ were used. |
| 14 | 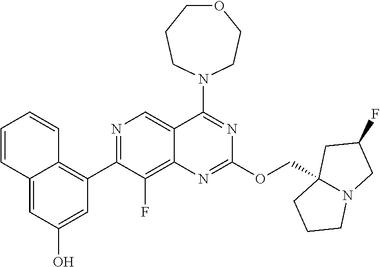 [SMILES: Oc1cc(-c2ncc3c(N4CCCOCC4)nc(OC[C@@]45CCCN4C[C@H](F)C5)nc3c2F)c2ccccc2c1] | 4-(8-Fluoro-2- (((2R,7aS)-2- fluorotetrahydro- 1H-pyrrolizin- 7a(5H)- yl)methoxy)-4- (1,4-oxazepan-4- yl)pyrido[4,3- d]pyrimidin-7- yl)naphthalen-2- ol | 2,2,2- trifluoro- acetate | Step 3: 4- (4,4,5,5- tetramethyl- 1,3,2- dioxaborolan-2- yl)-2-naphthol (CAS#: 2043962- 01-0, ChemBridge Corporation) | Step 3: Pd(PPh₃)₄ and Cs₂CO₃ were used. |
| 15 | 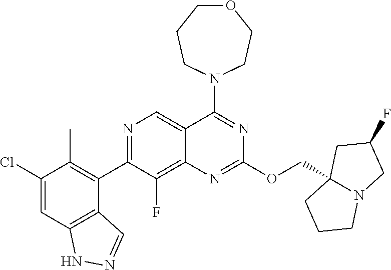 [SMILES: Cc1c(Cl)cc2[nH]ncc2c1-c1ncc2c(N3CCCOCC3)nc(OC[C@@]34CCCN3C[C@H](F)C4)nc2c1F] | 4-(7-(6-Chloro-5- methyl-1H- indazol-4-yl)-8- fluoro-2- (((2R,7aS)-2- fluorotetrahydro- 1H-pyrrolizin- 7a(5H)- yl)methoxy)pyrido [4,3- d]pyrimidin-4- yl)-1,4- oxazepane | 2,2,2- trifluoro- acetate | Step 3: 6-chloro- 5-methyl-1- (tetrahydro-2H- pyran-2-yl)-4- (4,4,5,5- tetramethyl- 1,3,2- dioxaborolan-2- yl)-1H-indazole (CAS#: 2374152- 83-5, PharmaBlock) | Step 3: Pd(dtbpf)Cl₂ were used. |
| 16 | 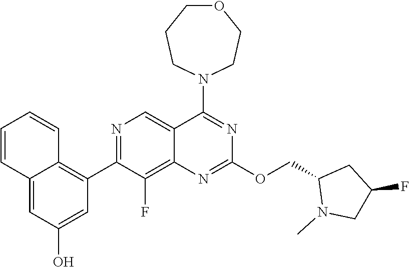 [SMILES: CN1C[C@H](F)C[C@H]1COc1nc(N2CCCOCC2)c2cnc(-c3cc(O)cc4ccccc34)c(F)c2n1] | 4-(8-Fluoro-2- (((2S,4R)-4- fluoro-1- methylpyrrolidin- 2-yl)methoxy)-4- (1,4-oxazepan-4- yl)pyrido[4,3- d]pyrimidin-7- yl)naphthalen-2- ol | 2,2,2- trifluoro- acetate | Step 2: ((2S,4R)- 4-fluoro-1- methylpyrrolidin- 2-yl)methanol (CAS#: 2206737- 78-0, eNovation) Step 3: 4- (4,4,5,5- tetramethyl- 1,3,2- dioxaborolan-2- yl)-2-naphthol (CAS#: 2043962- 01-0, ChemBridge Corporation) | Step 3: Pd(PPh₃)₄ and Cs₂CO₃ were used. |
| 17 | 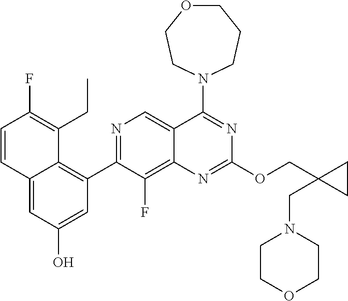 [SMILES: CCc1c(F)ccc2cc(O)cc(-c3ncc4c(N5CCCOCC5)nc(OCC5(CN6CCOCC6)CC5)nc4c3F)c12] | 5-Ethyl-6-fluoro- 4-(8-fluoro-2-((1- (morpholinomethyl) cyclopropyl) methoxy)-4-(1,4- oxazepan-4- yl)pyrido[4,3- d]pyrimidin-7- yl)naphthalen-2- ol | Bis(2, 2,2- trifluoro- acetate) | Step 2: (1- (morpholinomethyl) cyclopropyl) methanol (CAS#: 1267956-65-9, Combi Block) | Step 2: DBACO (0.2 eq.), Cs₂CO₃ (3 eq.) was used. |
| 18 | 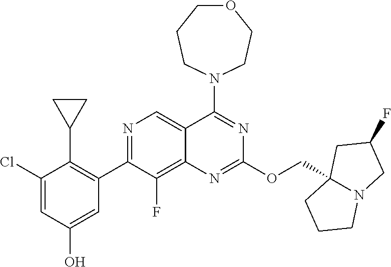 [SMILES: Oc1cc(Cl)c(C2CC2)c(-c2ncc3c(N4CCCOCC4)nc(OC[C@@]45CCCN4C[C@H](F)C5)nc3c2F)c1] | 3-Chloro-4- cyclopropyl-5-(8- fluoro-2- (((2R,7aS)-2- fluorotetrahydro- 1H-pyrrolizin- 7a(5H)- yl)methoxy)-4- (1,4-oxazepan-4- yl)pyrido[4,3- d]pyrimidin-7- yl)phenol | Free base | Step 3: 2-(3- chloro-2- cyclopropyl-5- (methoxymethoxy) phenyl)- 4,4,5,5- tetramethyl- 1,3,2- dioxaborolane (CAS#: 2621936- 26-1, LabNetwork) | |
| 19 | 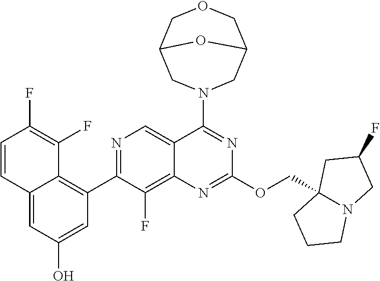 [SMILES: Oc1cc(-c2ncc3c(N4CC5COCC(C4)O5)nc(OC[C@@]45CCCN4C[C@H](F)C5)nc3c2F)c2c(F)c(F)ccc2c1] | 4-(4-((1R,5S)- 3,9-Dioxa-7- azabicyclo[3.3.1] nonan-7-yl)-8- fluoro-2- (((2R,7aS)-2- fluorotetrahydro- 1H-pyrrolizin- 7a(5H)- yl)methoxy)pyrido [4,3- d]pyrimidin-7- yl)-5,6- difluoronaphthalen- 2-ol | Bis (2,2,2- trifluoro- acetate) | Step 1: 3,9- dioxa-7- azabicyclo[3.3.1] nonane hydrochloride (CAS#: 1803587- 96-3, Enamine) Step 3: 2-(7,8- difluoro-3- (methoxymethoxy) naphthalen-1- yl)-4,4,5,5- tetramethyl- 1,3,2- dioxaborolane (CAS#: 2621935- 35-9, LabNetwork) | Step 2: DABCO (0.2 eq.), Cs₂CO₃ (3 eq.) was used. |
| 20 | 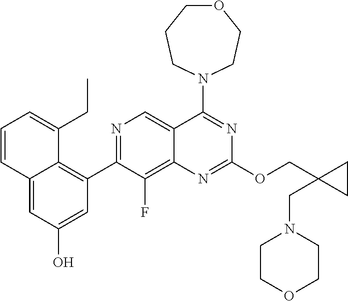 [SMILES: CCc1cccc2cc(O)cc(-c3ncc4c(N5CCCOCC5)nc(OCC5(CN6CCOCC6)CC5)nc4c3F)c12] | 5-Ethyl-4-(8- fluoro-2-((1- (morpholinomethyl) cyclopropyl) methoxy)-4-(1,4- oxazepan-4- yl)pyrido[4,3- d]pyrimidin-7- yl)naphthalen-2- ol | Bis (2,2,2- trifluoro- acetate) | Step 2: (1- (morpholinomethyl) cyclopropyl) methanol (CAS#: 1267956-65-9, Combi Block) Step 3: 2-(8- ethyl-3- (methoxymethoxy) naphthalen-1- yl)-4,4,5,5- tetramethyl- 1,3,2- dioxaborolane (CAS#: 2621932- 60-1, LabNetwork) | Step 2: DABCO (0.2 eq.), Cs₂CO₃ (3 eq.) was used. |
| 21 | 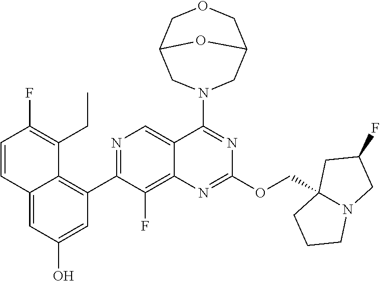 [SMILES: CCc1c(F)ccc2cc(O)cc(-c3ncc4c(N5CC6COCC(C5)O6)nc(OC[C@@]56CCCN5C[C@H](F)C6)nc4c3F)c12] | 4-(4-((1R,5S)- 3,9-Dioxa-7- azabicyclo[3.3.1] nonan-7-yl)-8- fluoro-2- (((2R,7aS)-2- fluorotetrahydro- 1H-pyrrolizin- 7a(5H)- yl)methoxy)pyrido [4,3- d]pyrimidin-7- yl)-5-ethyl-6- fluoronaphthalen- 2-ol | Bis (2,2,2- trifluoro- acetate | Step 1: 3,9- dioxa-7- azabicyclo[3.3.1] nonane hydrochloride (CAS#: 1803587- 96-3, Enamine) | Step 2: DABCO (0.2 eq.), Cs₂CO₃ (3 eq.) was used |
| 22 | 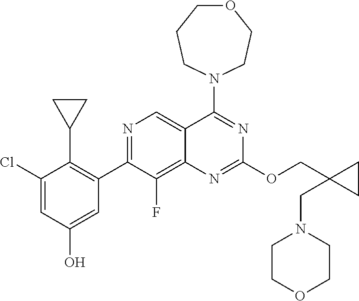 [SMILES: Oc1cc(Cl)c(C2CC2)c(-c2ncc3c(N4CCCOCC4)nc(OCC4(CN5CCOCC5)CC4)nc3c2F)c1] | 3-Chloro-4- cyclopropyl-5-(8- fluoro-2-((1- (morpholinomethyl) cyclopropyl) methoxy)-4-(1,4- oxazepan-4- yl)pyrido[4,3- d]pyrimidin-7- yl)phenol | Bis (2,2,2- trifluoro- acetate) | Step 2: (1- (morpholinomethyl) cyclopropyl) methanol (CAS#: 1267956-65-9, Combi Blocks) Step 3: 2-(3- chloro-2- cyclopropyl-5- (methoxymethoxy) phenyl)- 4,4,5,5- tetramethyl- 1,3,2- dioxaborolane (CAS#: 2621936- 26-1, LabNetwork) | Step 2: DABCO (0.2 eq.), Cs₂CO₃ (3 eq.) was used. |
| 23 | 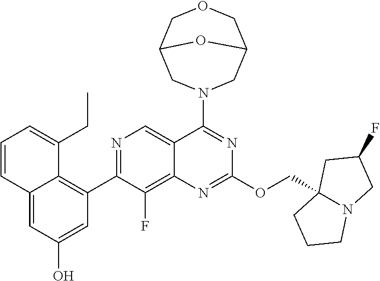 [SMILES: CCc1cccc2cc(O)cc(-c3ncc4c(N5CC6COCC(C5)O6)nc(OC[C@@]56CCCN5C[C@H](F)C6)nc4c3F)c12] | 4-(4-((1R,5S)- 3,9-Dioxa-7- azabicyclo[3.3.1] nonan-7-yl)-8- fluoro-2- (((2R,7aS)-2- fluorotetrahydro- 1H-pyrrolizin- 7a(5H)- yl)methoxy)pyrido [4,3- d]pyrimidin-7- yl)-5- ethylnaphthalen- 2-ol | Bis (2,2,2- trifluoro- acetate) | Step 1: 3,9- dioxa-7- azabicyclo[3.3.1] nonane hydrochloride (CAS#: 1803587- 96-3, Enamine) Step 3: 2-(8- ethyl-3- (methoxymethoxy) naphthalen-1- yl)-4,4,5,5- tetramethyl- 1,3,2- dioxaborolane (CAS#: 2621932- 60-1, LabNetwork) | Step 2: DABCO (0.2 eq.), Cs₂CO₃ (3 eq.) was used |
| 24 | 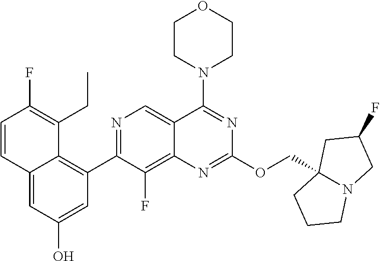 [SMILES: CCc1c(F)ccc2cc(O)cc(-c3ncc4c(N5CCOCC5)nc(OC[C@@]56CCCN5C[C@H](F)C6)nc4c3F)c12] | 5-Ethyl-6-fluoro- 4-(8-fluoro-2- (((2R,7aS)-2- fluorotetrahydro- 1H-pyrrolizin- 7a(5H)- yl)methoxy)-4- morpholinopyrido [4,3- d]pyrimidin-7- yl)naphthalen-2- ol | 2,2,2- trifluoro- acetate | Step 1: morpholine (CAS#: 110-91- 8, Sigma-Aldrich Corporation) | Step 2: DABCO (0.2 eq.), Cs₂CO₃ (3 eq.) was used |
| 25 | 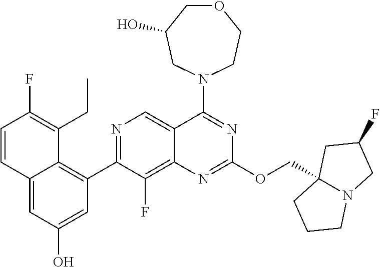 [SMILES: CCc1c(F)ccc2cc(O)cc(-c3ncc4c(N5CCOC[C@@H](O)C5)nc(OC[C@@]56CCCN5C[C@H](F)C6)nc4c3F)c12] | (S)-4-(7-(8-Ethyl- 7-fluoro-3- hydroxynaphthalen- 1-yl)-8-fluoro- 2-(((2R,7aS)-2- fluorotetrahydro- 1H-pyrrolizin- 7a(5H)- yl)methoxy)pyrido [4,3- d]pyrimidin-4- yl)-1,4-oxazepan- 6-ol | 2,2,2- trifluoro- acetate | Step 1: (S)-1,4- oxazepan-6-ol (CAS#: 1373232- 31-5, J&W Pharmlab) | |
| 26 | 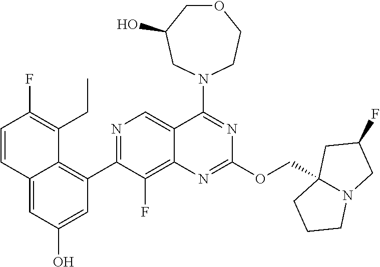 [SMILES: CCc1c(F)ccc2cc(O)cc(-c3ncc4c(N5CCOC[C@H](O)C5)nc(OC[C@@]56CCCN5C[C@H](F)C6)nc4c3F)c12] | (R)-4-(7-(8- Ethyl-7-fluoro-3- hydroxynaphthalen- 1-yl)-8-fluoro- 2-(((2R,7aS)-2- fluorotetrahydro- 1H-pyrrolizin- 7a(5H)- yl)methoxy)pyrido [4,3- d]pyrimidin-4- yl)-1,4-oxazepan- 6-ol | 2,2,2- trifluoro- acetate | Step 1: (R)-1,4- oxazepan-6-ol (CAS#: 1022915- 33-8, J&W Pharmlab) | |
| 27 | 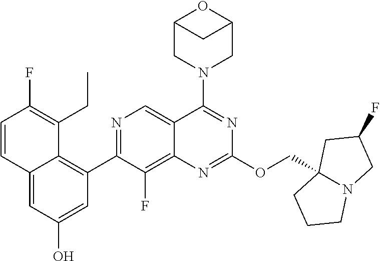 [SMILES: CCc1c(F)ccc2cc(O)cc(-c3ncc4c(N5CC6CC(C5)O6)nc(OC[C@@]56CCCN5C[C@H](F)C6)nc4c3F)c12] | 4-(4-((1R,5S)-6- Oxa-3- azabicyclo[3.1.1] heptan-3-yl)-8- fluoro-2- (((2R,7aS)-2- fluorotetrahydro- 1H-pyrrolizin- 7a(5H)- yl)methoxy)pyrido [4,3- d]pyrimidin-7- yl)-5-ethyl-6- fluoronaphthalen- 2-ol | 2,2,2- trifluoro- acetate | Step 1: 6-oxa-3- azabicyclo[3.1.1] heptane hydrochloride (CAS#: 1414958- 33-0, Combi Blocks Inc.) | Step 2: DABCO (0.2 eq.), Cs₂CO₃ (3 eq.) was used |
| 28 | 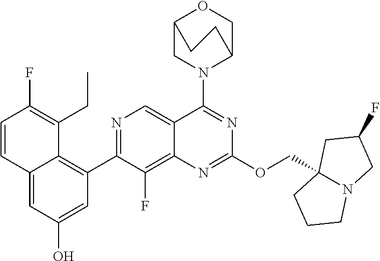 [SMILES: CCc1c(F)ccc2cc(O)cc(-c3ncc4c(N5CC6CCC5CO6)nc(OC[C@@]56CCCN5C[C@H](F)C6)nc4c3F)c12] | 4-(4-((1S,4R)-2- Oxa-5- azabicyclo[2.2.2] octan-5-yl)-8- fluoro-2- (((2R,7aS)-2- fluorotetrahydro- 1H-pyrrolizin- 7a(5H)- yl)methoxy)pyrido [4,3- d]pyrimidin-7- yl)-5-ethyl-6- fluoronaphthalen- 2-ol | 2,2,2- trifluoro- acetate | Step 1: 2-oxa-5- azabicyclo[2.2.2] octane hemioxalate (CAS#: 1523606- 41-8, Advanced ChemBlocks Inc.) | |
| 29 | 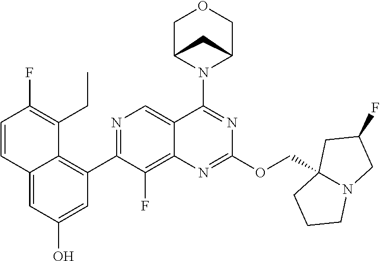 [SMILES: CCc1c(F)ccc2cc(O)cc(-c3ncc4c(N5[C@@H]6COC[C@H]5C6)nc(OC[C@@]56CCCN5C[C@H](F)C6)nc4c3F)c12] | 4-(4-(3-oxa-6- azabicyclo[3.1.1] heptan-6-yl)-8- fluoro-2- (((2R,7aS)-2- fluorotetrahydro- 1H-pyrrolizin- 7a(5H)- yl)methoxy)pyrido [4,3- d]pyrimidin-7- yl)-5-ethyl-6- fluoronaphthalen- 2-ol | 2,2,2- trifluoro- aceate | Step 1: 3-oxa-6- azabicyclo[3.1.1] heptane hydrochloride (CAS#: 1860028- 23-4, Angel Pharmatech Ltd.) | None |
| 30 | 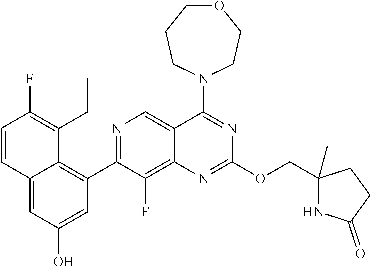 [SMILES: CCc1c(F)ccc2cc(O)cc(-c3ncc4c(N5CCCOCC5)nc(OCC5(C)CCC(=O)N5)nc4c3F)c12] | 5-(((7-(8-ethyl-7- fluoro-3- hydroxynaphthalen- 1-yl)-8-fluoro- 4-(1,4-oxazepan- 4-yl)pyrido[4,3- d]pyrimidin-2- yl)oxy)methyl)- 5- methylpyrrolidin- 2-one | 2,2,2- trifluoro- aceate | Step 2: 5- (hydroxymethyl)- 5- methylpyrrolidin- 2-one (CAS#: 1159991-46-4, Enamine) | Step 2: NaH and THF were used. |
| 31 | 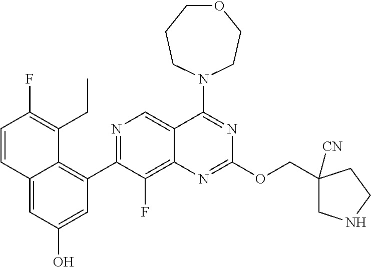 [SMILES: CCc1c(F)ccc2cc(O)cc(-c3ncc4c(N5CCCOCC5)nc(OCC5(C#N)CCNC5)nc4c3F)c12] | 3-(((7-(8-ethyl-7- fluoro-3- hydroxynaphthalen- 1-yl)-8-fluoro- 4-(1,4-oxazepan- 4-yl)pyrido[4,3- d]pyrimidin-2- yl)oxy)methyl) pyrrolidine-3- carbonitrile | bis (2,2,2- trifluoro- aceate) | Step 2: tert-butyl 3-cyano-3- (hydroxymethyl) pyrrolidine-1- carboxylate (CAS#: 1374656- 61-7, PharmaBlock) | Step 2: NaH and THF were used. |
| 32 | 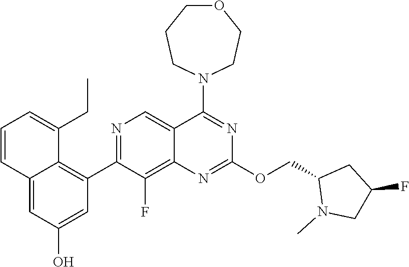 [SMILES: CCc1cccc2cc(O)cc(-c3ncc4c(N5CCCOCC5)nc(OC[C@@H]5C[C@@H](F)CN5C)nc4c3F)c12] | 5-Ethyl-4-(8- fluoro-2- (((2S,4R)-4- fluoro-1- methylpyrrolidin- 2-yl)methoxy)-4- (1,4-oxazepan-4- yl)pyrido[4,3- d]pyrimidin-7- yl)naphthalen-2- ol | Free base | Step 2: ((2S,4R)- 4-fluoro-1- methylpyrrolidin- 2-yl)methanol (CAS#: 2206737- 78-0, eNovation) Step 3: 2-(8- ethyl-3- (methoxymethoxy) naphthalen-1- yl)-4,4,5,5- tetramethyl- 1,3,2- dioxaborolane (CAS#: 2621932- 60-1, LabNetwork) | After Step 4, the fractions were washed with aq. NaHCO₃ solution to free base the product. |
| 33 | 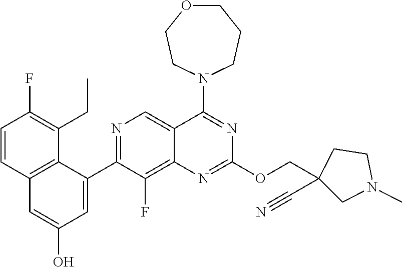 [SMILES: CCc1c(F)ccc2cc(O)cc(-c3ncc4c(N5CCCOCC5)nc(OCC5(C#N)CCN(C)C5)nc4c3F)c12] | 3-(((7-(8-ethyl-7- fluoro-3- hydroxynaphthalen- 1-yl)-8-fluoro- 4-(1,4-oxazepan- 4-yl)pyrido[4,3- d]pyrimidin-2- yl)oxy)methyl)- 1- methylpyrrolidine- 3-carbonitrile | bis (2,2,2- trifluoro- aceate) | Step 1: 1,4- oxazepane (CAS#: 5638-60- 8, Oakwood Products, Inc.) Step 2: Step 2: tert-butyl 3- cyano-3- (hydroxymethyl) pyrrolidine-1- carboxylate (CAS#: 1374656- 61-7, PharmaBlock) | Additional step after Step 4. Details included below. |
| 78 | 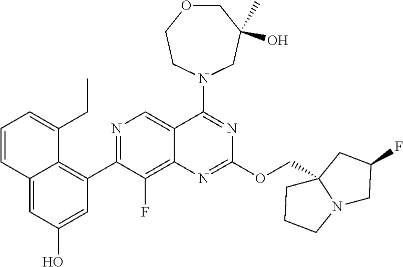 [SMILES: CCc1cccc2cc(O)cc(-c3ncc4c(N5CCOC[C@@](C)(O)C5)nc(OC[C@@]56CCCN5C[C@H](F)C6)nc4c3F)c12] | (S)-4-(7-(8-Ethyl- 3- hydroxynaphthalen- 1-yl)-8-fluoro- 2-(((2R,7aS)-2- fluorotetrahydro- 1H-pyrrolizin- 7a(5H)- yl)methoxy)pyrido [4,3- d]pyrimidin-4- yl)-6-methyl-1,4- oxazepan-6-ol | 2,2,2- trifluoro- aceate | Step 1: Intermediate J1 | |
| 79 | 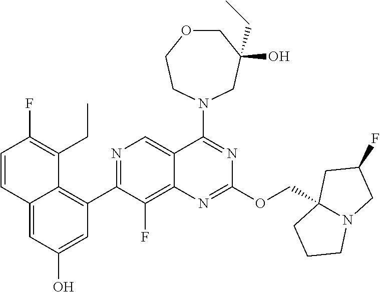 | (S)-6-Ethyl-4-(7- (8-ethyl-7-fluoro- 3- hydroxynaphthalen- 1-yl)-8-fluoro- 2-(((2R,7aS)-2- fluorotetrahydro- 1H-pyrrolizin- 7a(5H)- yl)methoxy)pyrido [4,3- d]pyrimidin-4- yl)-1,4-oxazepan- 6-o1 | 2,2,2- trifluoro- aceate | Step 1: Intermediate M1 | Steps 1 and 2 were performed in one pot |
| 80 | 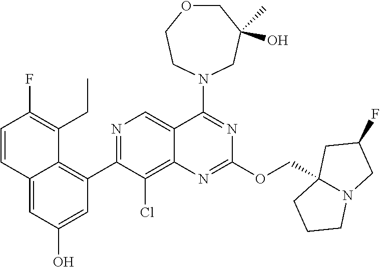 [SMILES: CCc1c(F)ccc2cc(O)cc(-c3ncc4c(N5CCOC[C@@](C)(O)C5)nc(OC[C@@]56CCCN5C[C@H](F)C6)nc4c3Cl)c12] | (S)-4-(8-Chloro- 7-(8-ethyl-7- fluoro-3- hydroxynaphthalen- 1-yl)-2- (((2R,7aS)-2- fluorotetrahydro- 1H-pyrrolizin- 7a(5H)- yl)methoxy)pyrido [4,3- d]pyrimidin-4- yl)-6-methyl-1,4- oxazepan-6-ol | 2,2,2- trifluoro- aceate | Step 1: 6- Methyl-1,4- oxazepan-6-ol hydrochloride (CAS#: 1823315- 50-9, Pharmablock, Inc.) and 2,4,7,8- tetrachloropyrido [4,3-d]pyrimidine (CAS#: 2454396- 63-3, LabNetwork) | Chiral separation after Step 1 (Column: ChiralPak AD, 2 × 25 cm 5 μm Mobile phase: 45% MeOH w/ 0.2% TEA Flowrate: 65 mL/min. Yield: 1082 mg sample was submitted to generate 475 mg of peak 1 with an ee of >99% and 456 mg of peak 2 with an ee of >99%, peak 1 was used) Step 2: reaction was performed with cesium carbonate, DBACO in a mixture of THF and DMF at 30° C. |

### Additional Step for Example 12

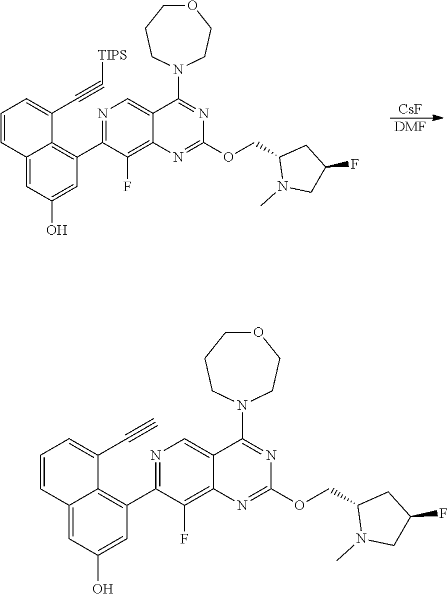

To a 10-mL round-bottomed flask was added 4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)-5-((triisopropylsilyl)ethynyl)naphthalen-2-ol (14 mg, 0.02 mmol) in DMF (0.2 mL). At 0° C., cesium fluoride (15 mg, 0.10 mmol, Sigma-Aldrich Corporation) was added. The reaction was stirred overnight. The crude mixture was purified by reverse phase HPLC to yield 5-ethynyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol as 2,2,2-trifluoroacetate and as brown solid (4.0 mg, 6.1 mol, 30% yield).

### Additional Step for Example 33

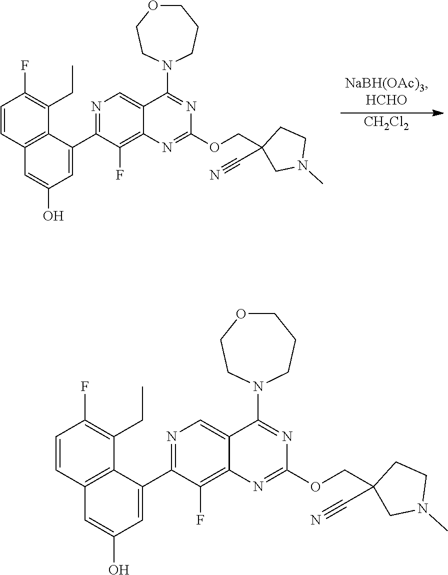 [SMILES: CCc1c(F)ccc2cc(O)cc(-c3ncc4c(N5CCCOCC5)nc(OCC5(C#N)CCN(C)C5)nc4c3F)c12 ; "CH2Cl2" ; CCc1c(F)ccc2cc(O)cc(-c3ncc4c(N5CCCOCC5)nc(OCC5(C#N)CCN(C)C5)nc4c3F)c12]

To a vial was added 3-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)pyrrolidine-3-carbonitrile (34 mg, 0.06 mmol) and formaldehyde, 37% solution (58 mg, 53 μL, 0.72 mmol, Sigma-Aldrich Corporation) in dichloromethane (0.3 mL) at 0° C. To the reaction mixture was added 1 drop of acetic acid and the reaction was stirred for 10 min at 0° C. Then, sodium triacetoxyborohydride (38 mg, 0.18 mmol, Sigma-Aldrich Corporation) was added at 0° C. and the reaction was stirred for 2 h at rt. Upon completion, the solution was transferred to a separatory funnel and extracted three times with ethyl acetate. The combined organic layers were dried over Na₂SO₄. The resulting solution was filtered, and concentrated in vacuo to afford the crude product. The resulting crude was purified via reverse phase HPLC to provide 3-(((7-(8-ethyl-7-fluoro-3-hydroxynaphthalen-1-yl)-8-fluoro-4-(1,4-oxazepan-4-yl)pyrido[4,3-d]pyrimidin-2-yl)oxy)methyl)-1-methylpyrrolidine-3-carbonitrile as bis(2,2,2-trifluoroacetate) and as light-yellow solid (15 mg, 0.02 mmol, 31% yield).

TABLE 4
Analytical Data for Examples 2 to 33, and 78-80.

| | MS | |
|---|---|---|
| Ex. | m/z (ESI) | |
| # | (M + H)⁺ | ¹H NMR |
| 2 | 576.2 | ¹H NMR (400 MHz, METHANOL-d₄) δ ppm 9.22 (s, 1 H) 7.59- |
| | | 7.72 (m, 1 H) 7.36-7.43 (m, 1 H) 7.33 (d, J = 2.72 Hz, 1 H) |
| | | 7.19 (d, J = 6.69 Hz, 1 H) 7.02-7.08 (m, 1 H) 5.46-5.70 (m, 1 |
| | | H) 4.63-4.78 (m, 2 H) 4.26-4.36 (m, 4 H) 4.05-4.10 (m, 2 |
| | | H) 3.84-4.02 (m, 5 H) 3.42-3.59 (m, 1 H) 2.54-2.82 (m, 2 H) |
| | | 2.11-2.50 (m, 8 H) 0.92 (td, J = 7.42, 1.46 Hz, 3 H). |
| 3 | 584.0 | ¹H NMR (400 MHz, METHANOL-d₄) δ ppm 9.38 (s, 1 H) 7.64- |
| | | 7.76 (m, 1 H) 7.37-7.58 (m, 3 H) 5.50-5.75 (m, 1 H) 4.89 (d, |
| | | J = 9.41 Hz, 2 H) 4.40-4.53 (m, 4 H) 3.83-4.19 (m, 7 H) 3.43- |
| | | 3.56 (m, 1 H) 2.59-2.86 (m, 2 H) 2.46-2.57 (m, 1 H) 2.18- |
| | | 2.46 (m, 5 H). |
| 4 | 596.2 | ¹H NMR (400 MHz, DMSO-d6): δ ppm 9.12 (s, 1 H), 7.78 (dd, |
| | | J = 9.1, 6.0 Hz, 1 H), 7.32-7.40 (m, 2 H), 7.02 (d, J = 2.7 Hz, 1 |
| | | H), 5.46-5.69 (m, 1 H), 4.54-4.68 (m, 2 H), 4.17-4.35 (m, 4 |
| | | H), 3.69-3.96 (m, 5 H), 3.25-3.38 (m, 2 H), 2.94-3.00 (m, 3 |
| | | H), 2.34 (br dd, J = 3.8, 1.9 Hz, 2 H), 2.01-2.27 (m, 5 H), 0.74 |
| | | (t, J = 7.4 Hz, 3 H). |
| 5 | 610.3 | ¹H NMR (400 MHz, METHANOL-d₄) δ ppm 9.23 (s, 1 H), 7.70 |
| | | (dd, J = 9.1, 5.7 Hz, 1 H), 7.34 (d, J = 2.7 Hz, 1 H), 7.27 (t, J-9.4 |
| | | Hz, 1 H), 7.09 (d, J = 2.7 Hz, 1 H), 5.46-5.71 (m, 1 H), 4.71 (dd, |
| | | J = 14.0, 4.2 Hz, 2 H), 4.25-4.48 (m, 4 H), 3.80-4.12 (m, 3 H), |
| | | 3.43-3.56 (m, 1 H), 3.18 (t, J = 5.7 Hz, 2 H), 2.54-2.85 (m, 4 |
| | | H), 2.29-2.53 (m, 6 H), 2.11-2.27 (m, 2 H), 0.82 (t, J = 7.2 Hz, |
| | | 3 H). |
| 6 | 606.0 | ¹H NMR (400 MHz, METHANOL-d₄) δ ppm 9.18 (s, 1 H) 7.66- |
| | | 7.75 (m, 1 H) 7.34 (d, J = 2.72 Hz, 1 H) 7.23-7.31 (m, 1 H) |
| | | 7.05-7.10 (m, 1 H) 5.40-5.73 (m, 1 H) 5.15-5.27 (m, 2 H) |
| | | 4.63-4.73 (m, 2 H) 3.89 (s, 7 H) 3.45-3.56 (m, 1 H) 2.54- |
| | | 2.82 (m, 2 H) 2.32-2.54 (m, 4 H) 2.10-2.30 (m, 6 H) 0.82 (s, 3 |
| | | H). |
| 7 | 612.2 | ¹H NMR (400 MHz, DMSO-d₆) δ ppm 10.68 (br s, 1 H) 9.78- |
| | | 10.00 (br s, 1 H) 9.26 (s, 1 H) 7.64-7.74 (m, 1 H) 7.34-7.45 |
| | | (m, 1 H) 7.31 (d, J = 2.72 Hz, 1 H) 7.15 (d, J = 6.90 Hz, 1 H) 6.94- |
| | | 7.02 (m, 1 H) 5.48-5.71 (m, 1 H) 4.56-4.92 (m, 4 H) 4.17- |
| | | 4.37 (m, 4 H) 4.02-4.17 (m, 2 H) 3.75-3.94 (m, 4 H) 3.27- |
| | | 3.39 (m, 1 H) 1.95-2.42 (m, 7 H) 0.83 (t, J = 7.42 Hz, 3 H). |
| 8 | 558.1 | ¹H NMR (400 MHz, METHANOL-d₄) δ ppm 9.24 (s, 1 H) 7.61- |
| | | 7.70 (m, 1 H) 7.34-7.47 (m, 2 H) 7.28 (d, J = 2.30 Hz, 1 H) |
| | | 5.42-5.61 (m, 1 H) 4.95-5.06 (m, 1 H) 4.70-4.79 (m, 1 H) |
| | | 4.24-4.38 (m, 5 H) 4.04-4.10 (m, 1 H) 3.98-4.16 (m, 2 H) |
| | | 3.83-3.92 (m, 2 H) 3.61-3.77 (m, 1 H) 3.21 (s, 3 H) 2.63- |
| | | 2.77 (m, 1 H) 2.33-2.55 (m, 1 H) 2.17-2.28 (m, 2 H). |
| 9 | 568.1 | ¹H NMR (400 MHz, METHANOL-d₄) δ ppm 9.24 (s, 1 H) 7.66- |
| | | 7.75 (m, 1 H) 7.23-7.38 (m, 2 H) 7.08 (d, J = 2.51 Hz, 1 H) |
| | | 5.40-5.61 (m, 1 H) 4.94-5.04 (m, 1 H) 4.70-4.78 (m, 1 H) |
| | | 4.25-4.35 (m, 5 H) 3.98-4.18 (m, 3 H) 3.89 (t, J = 5.23 Hz, 2 |
| | | H) 3.63-3.78 (m, 1 H) 3.20 (s, 3 H) 2.62-2.78 (m, 1 H) 2.34- |
| | | 2.56 (m, 2 H) 2.16-2.28 (m, 3 H) 0.83 (t, J = 7.32 Hz, 3 H). |
| 10 | 588.2 | ¹H NMR (400 MHz, DMSO-d₆) δ ppm 10.59-10.81 (br s, 1 H) |
| | | 9.73-10.00 (br s, 1 H) 9.25 (s, 1 H) 7.68 (d, J-7.94 Hz, 1 H) |
| | | 7.34-7.43 (m, 1 H) 7.26-7.32 (m, 1 H) 7.10-7.18 (m, 1 H) |
| | | 6.93-7.01 (m, 1 H) 5.46-5.71 (m, 1 H) 4.70-4.82 (m, 1 H) |
| | | 4.55-4.67 (m, 2 H) 4.40-4.49 (m, 2 H) 3.64-3.89 (m, 5 H) |
| | | 3.27-3.39 (m, 1 H) 2.55 (s, 2 H) 1.89-2.40 (m, 9 H) 0.83 (t, |
| | | J = 7.42 Hz, 3 H). |
| 11 | 622.1 | ¹H NMR (400 MHz, DMSO-d₆) δ ppm 10.63-10.85 (m, 1 H) |
| | | 9.33 (s, 1 H) 7.73-7.83 (m, 1 H) 7.30-7.40 (m, 2 H) 6.99- |
| | | 7.05 (m, 1 H) 5.47-5.69 (m, 1 H) 4.53-4.70 (m, 3 H) 4.33- |
| | | 4.44 (m, 1 H) 4.27 (t, J = 5.33 Hz, 1 H) 4.00-4.13 (m, 2 H) 3.80- |
| | | 3.98 (m, 7 H) 3.27-3.38 (m, 2 H) 1.97-2.46 (m, 7 H) 0.74 (t, |
| | | J = 7.32 Hz, 3 H). |
| 12 | 545.6 | ¹H NMR (400 MHz, METHANOL-d₄) δ ppm 9.18 (s, 1 H), 7.85 |
| | | (d, J = 8.2 Hz, 1 H), 7.54 (d, J = 6.9 Hz, 1 H), 7.35-7.45 (m, 2 H), |
| | | 7.20 (dd, J = 2.6, 0.9 Hz, 1 H), 5.54-5.61 (m, 1 H), 4.99 (dt, |
| | | J = 13.2, 2.5 Hz, 1 H), 4.75 (ddd, J = 13.2, 5.9, 2.9 Hz, 1 H), 4.20- |
| | | 4.40 (m, 5 H), 3.99-4.16 (m, 3 H), 3.82-3.96 (m, 2 H), 3.59- |
| | | 3.79 (m, 1 H), 3.20 (s, 3 H), 3.11 (s, 1 H), 2.68-2.76 (m, 1 H), |
| | | 2.21-2.26 (m, 3 H). |
| 13 | 530.2 | ¹H NMR (400 MHz, METHANOL-d₄) δ ppm 9.22 (s, 1 H), |
| | | 7.73- 7.90 (m, 1 H), 7.50-7.62 (m, 1 H), 7.37-7.50 |
| | | (m, 1 H), 7.07-7.37 (m, 3 H), 4.21-4.44 (m, 6 H), 4.00-4.15 |
| | | (m, 2 H), 3.79-3.96 (m, 2 H), 3.10-3.25 |
| | | (m, 2 H), 2.60-2.91 (m, 2 H), 1.68-2.32 (m, 10 H). |
| 14 | 548.6 | ¹H NMR (400 MHz, METHANOL-d₄) δ ppm 9.29 |
| | | (s, 1 H), 7.78 (s, 1 H), 7.40-7.61 (m, 2 H), 7.10-7.35 |
| | | (m, 3 H), 5.31-5.90 (m, 1 H), 4.71 (d, J = 18.2 Hz, |
| | | 2 H), 4.32 (s, 4 H), 3.77-4.13 (m, 7 H), |
| | | 3.42-3.58 (m, 1 H), 2.52-2.84 (m, 2 H), 2.30-2.52 |
| | | (m, 3 H), 2.13-2.29 (m, 3 H). |
| 15 | 572.2 | ¹H NMR (400 MHz, METHANOL-d₄) δ ppm 9.32 (s, 1 H), 7.59- |
| | | 7.92 (m, 2 H), 5.52-5.94 (m, 1H), 4.68-4.73 (m, 2 H), 4.20- |
| | | 4.52 (m, 4 H), 3.73-4.12 (m, 7 H), 3.40-3.56 (m, 1 H), 2.29- |
| | | 2.92 (m, 8 H), 2.11-2.26 (m, 3 H). |
| 16 | 522.2 | ¹H NMR (400 MHz, METHANOL-d₄) δ ppm 8.87-9.43 (m, 1 |
| | | H), 7.66-8.02 (m, 1 H), 7.51-7.61 (m, 1 H), 7.37-7.51 (m, 1 |
| | | H), 7.01-7.37 (m, 3 H), 5.30-5.93 (m, 1 H), 4.94-5.11 (m, 1 |
| | | H), 4.62-4.82 (m, 1 H), 4.29 (br dd, J = 9.1, 5.2 Hz, 5 H), 4.07 |
| | | (br d, J = 5.0 Hz, 3 H), 3.87 (s, 2 H), 3.54-3.78 (m, 1 H), 3.21 (s, |
| | | 3 H), 2.62-2.80 (m, 1 H), 2.30-2.59 (m, 1 H), 2.04-2.30 (m, 2 |
| | | H). |
| 17 | 606.0 | ¹H NMR (400 MHz, METHANOL-d₄) δ ppm 9.15-9.23 (m, 1 |
| | | H), 7.64-7.73 (m, 1 H), 7.31-7.34 (m, 1 H), 7.22-7.29 (m, 1 |
| | | H), 7.04-7.08 (m, 1 H), 4.44-4.55 (m, 2 H), 4.21-4.33 (m, 4 |
| | | H), 3.61-4.13 (m, 11 H), 3.35-3.40 (m, 2 H), 3.06-3.25 (m, 1 |
| | | H), 2.40-2.56 (m, 1 H), 2.11-2.30 (m, 3 H), 0.98-1.04 (m, 2 |
| | | H), 0.86-0.93 (m, 2 H), 0.77-0.84 (m, 3 H). |
| 18 | 572.2 | ¹H NMR (400 MHz, METHANOL-d₄) δ ppm 9.26 (s, 1 H), 7.09 |
| | | (d, J = 2.5 Hz, 1 H), 6.92 (d, J = 2.5 Hz, 1 H), 5.34-5.54 (m, 1 H), |
| | | 4.32-4.45 (m, 6 H), 4.15 (dd, J = 5.5, 4.1 Hz, 2 H), 3.94-4.00 |
| | | (m, 2 H), 3.27-3.39 (m, 3 H), 3.15 (s, 1 H), 2.22-2.49 (m, 5 |
| | | H), 2.05-2.22 (m, 2 H), 1.94-2.04 (m, 2 H), 0.75 (br d, J = 7.9 |
| | | Hz, 2 H), 0.20 (br d, J = 4.8 Hz, 2 H). |
| 19 | 612.1 | ¹H NMR (400 MHz, METHANOL-d₄) δ ppm 9.18-9.23 (m, 1 |
| | | H), 7.60-7.66 (m, 1 H), 7.37-7.44 (m, 1 H), 7.33-7.36 (m, 1 |
| | | H), 7.25-7.28 (m, 1 H), 5.48-5.66 (m, 1 H), 4.96-5.04 (m, 2 |
| | | H), 4.69 (br s, 2 H), 3.83-4.16 (m, 11 H), 3.43-3.53 (m, 1 H), |
| | | 2.66-2.79 (m, 1 H), 2.53-2.64 (m, 1 H), 2.29-2.48 (m, 3 H), |
| | | 2.10-2.24 (m, 1 H). |
| 20 | 588.1 | ¹H NMR (400 MHz, METHANOL-d₄) δ ppm 9.18 (s, 1 H), 7.62- |
| | | 7.67 (m, 1 H), 7.34-7.40 (m, 1 H), 7.29-7.33 (m, 1 H), 7.15- |
| | | 7.20 (m, 1 H), 7.01-7.05 (m, 1 H), 4.46-4.51 (m, 2 H), 4.22- |
| | | 4.32 (m, 4 H), 3.63-4.15 (m, 11 H), 3.35-3.39 (m, 2 H), 2.14- |
| | | 2.43 (m, 4 H), 0.86-1.02 (m, 8 H). |
| 21 | 622.3 | ¹H NMR (400 MHz, METHANOL-d₄) δ ppm 9.16-9.25 (m, 1 |
| | | H), 7.64-7.73 (m, 1 H), 7.31-7.34 (m, 1 H), 7.22-7.29 (m, 1 |
| | | H), 7.06-7.09 (m, 1 H), 5.47-5.68 (m, 1 H), 4.93-5.04 (m, 2 |
| | | H), 4.63-4.70 (m, 1 H), 3.85-4.20 (m, 11 H), 3.42-3.54 (m, 1 |
| | | H), 2.67-2.80 (m, 1 H), 2.65 (br s, 6 H), 2.10-2.27 (m, 2 H), |
| | | 0.80 (br d, J = 1.5 Hz, 3 H). |
| 22 | 584.1 | ¹H NMR (400 MHz, METHANOL-d₄) δ ppm 9.19 (s, 1 H), 6.99 |
| | | (s, 1 H), 6.80-6.82 (m, 1 H), 4.46-4.55 (m, 2 H), 4.21-4.33 |
| | | (m, 5 H), 3.74 (br s, 11 H), 3.38 (s, 2 H), 2.13-2.27 (m, 2 H), |
| | | 1.79-1.93 (m, 1 H), 0.96-1.06 (m, 2 H), 0.86-0.93 (m, 2 H), |
| | | 0.58-0.70 (m, 2 H), 0.01-0.18 (m, 2 H). |
| 23 | 604.2 | ¹H NMR (400 MHz, METHANOL-d₄) δ ppm 9.15-9.20 (m, 1 |
| | | H), 7.61-7.67 (m, 1 H), 7.33-7.40 (m, 1 H), 7.29-7.32 (m, 1 |
| | | H), 7.14-7.20 (m, 1 H), 7.01-7.05 (m, 1 H), 5.48-5.67 (m, 1 |
| | | H), 4.94-5.04 (m, 2 H), 4.69 (br s, 2 H), 3.86-4.17 (m, 11 H), |
| | | 3.43-3.53 (m, 1 H), 2.66-2.79 (m, 1 H), 2.53-2.64 (m, 1 H), |
| | | 2.10-2.49 (m, 6 H), 0.84-0.94 (m, 3 H). |
| 24 | 580.3 | ¹H NMR (400 MHz, METHANOL-d₄) δ ppm 9.15-9.21 (m, 1 |
| | | H), 7.70 (dd, J = 9.1, 5.7 Hz, 1 H), 7.34 (d, J = 2.7 Hz, 1 H), 7.25- |
| | | 7.30 (m, 1 H), 7.07 (d, J = 2.7 Hz, 1 H), 5.47-5.73 (m, 1 H), 4.63- |
| | | 4.73 (m, 2 H), 4.18-4.26 (m, 4 H), 3.82-4.10 (m, 7 H), 3.45- |
| | | 3.56 (m, 1 H), 2.54-2.81 (m, 2 H), 2.33-2.53 (m, 4 H), 2.10- |
| | | 2.30 (m, 2 H), 0.82 (t, J = 7.0 Hz, 3 H), 0.54-0.55 (m, 1 H). |
| 25 | 610.2 | ¹H NMR (400 MHz, METHANOL-d₄) δ ppm 8.53-8.68 (m, 1 |
| | | H), 6.77-6.94 (m, 1 H), 6.44-6.57 (m, 2 H), 6.15-6.27 (m, 1 |
| | | H), 4.71-4.92 (m, 1 H), 3.43 (br d, J = 6.1 Hz, 4 H), 3.26-3.33 |
| | | (m, 2 H), 3.02-3.20 (m, 6 H), 2.65-2.74 (m, 2 H), 1.50-1.73 |
| | | (m, 6 H), 1.39 (br dd, J = 4.3, 2.2 Hz, 3 H),-0.04-0.06 (m, 3 H). |
| 26 | 610.2 | ¹H NMR (400 MHz, METHANOL-d₄) δ ppm 8.51-8.74 (m, 1 |
| | | H), 6.82-6.95 (m, 1 H), 6.53 (br d, J = 2.5 Hz, 2 H), 6.18-6.31 |
| | | (m, 1 H), 4.69-4.84 (m, 1 H), 3.36-3.68 (m, 5 H), 2.82-3.35 |
| | | (m, 7 H), 2.63-2.76 (m, 1 H), 1.86 (s, 4 H), 1.25-1.71 (m, 6 |
| | | H),-0.19-0.07 (m, 3 H). |
| 27 | 592.2 | ¹H NMR (400 MHz, METHANOL-d₄) δ ppm 9.54 (s, 1 H), 7.71 |
| | | (dd, J = 9.0, 5.9 Hz, 1 H), 7.34 (d, J = 2.7 Hz, 1 H), 7.28 (t, J = 9.4 |
| | | Hz, 1 H), 7.08 (d, J = 2.7 Hz, 1 H), 5.46-5.70 (m, 1 H), 4.87 (br |
| | | d, J = 6.5 Hz, 2 H), 4.66-4.73 (m, 2 H), 4.46-4.56 (m, 2 H), |
| | | 4.26-4.45 (m, 2 H), 3.83-4.16 (m, 3 H), 3.45-3.56 (m, 1 H), |
| | | 3.35-3.43 (m, 1 H), 2.68-2.83 (m, 1 H), 2.30-2.63 (m, 5 H), |
| | | 2.21 (td, J = 7.2, 3.0 Hz, 2 H), 2.03 (d, J = 9.2 Hz, 1 H), 0.82 (t, |
| | | J = 6.9 Hz, 3 H). |
| 28 | 606.3 | ¹H NMR (400 MHz, METHANOL-d₄) δ ppm 9.26-9.40 (m, 1 |
| | | H), 7.70 (dd, J = 9.0, 5.9 Hz, 1 H), 7.34 (d, J = 2.5 Hz, 1 H), 7.28 |
| | | (t, J = 9.4 Hz, 1 H), 7.02-7.11 (m, 1 H), 5.50-5.69 (m, 1 H), |
| | | 5.16 (br s, 1 H), 4.60-4.69 (m, 2 H), 4.14-4.39 (m, 4 H), 3.92 |
| | | (br s, 3 H), 3.50 (br s, 1 H), 2.71-2.81 (m, 1 H), 2.10-2.65 (m, |
| | | 11 H), 1.83-1.98 (m, 1 H), 0.76-0.88 (m, 3 H). |
| 29 | 592.3 | ¹H NMR (400 MHz, METHANOL-d₄) δ ppm 9.01 (s, 1 H), 7.70 |
| | | (dd, J = 9.1, 6.0 Hz, 1 H), 7.34 (d, J = 2.5 Hz, 1 H), 7.27 (t, J = 9.4 |
| | | Hz, 1 H), 7.07 (d, J = 2.5 Hz, 1 H), 5.48-5.71 (m, 1 H), 4.83- |
| | | 4.92 (m, 1 H), 4.59-4.77 (m, 4 H), 3.81-4.25 (m, 6 H), 3.44- |
| | | 3.56 (m, 1 H), 3.03-3.15 (m, 1 H), 2.68-2.85 (m, 1 H), 2.52- |
| | | 2.65 (m, 1 H), 2.30-2.51 (m, 4 H), 2.10-2.28 (m, 3 H), 0.81 (t, |
| | | J = 7.3 Hz, 3 H). |
| 30 | 564.1 | ¹H NMR (400 MHz, METHANOL-d₄) δ ppm 9.18-9.23 (m, 1 |
| | | H), 7.64-7.73 (m, 1 H), 7.31-7.35 (m, 1 H), 7.22-7.30 (m, 1 |
| | | H), 7.05-7.12 (m, 1 H), 4.43-4.52 (m, 2 H), 4.26-4.36 (m, 4 |
| | | H), 4.03-4.12 (m, 2 H), 3.83-3.92 (m, 2 H), 2.15-2.64 (m, 7 |
| | | H), 1.91-2.07 (m, 1 H), 1.43 (s, 3 H), 1.34-1.40 (m, 1 H), 0.76- |
| | | 0.88 (m, 3 H). |
| 31 | 561.1 | ¹H NMR (400 MHz, METHANOL-d₄) δ ppm 9.21 (s, 1 H), 7.65- |
| | | 7.73 (m, 1 H), 7.30-7.34 (m, 1 H), 7.22-7.29 (m, 1 H), 7.03- |
| | | 7.09 (m, 1 H), 4.81-4.85 (m, 1 H), 4.75 (br s, 2 H), 4.26-4.35 |
| | | (m, 4 H), 4.04-4.11 (m, 2 H), 3.85-3.93 (m, 3 H), 3.73-3.78 |
| | | (m, 1 H), 3.56-3.70 (m, 2 H), 2.66-2.75 (m, 1 H), 2.42-2.58 |
| | | (m, 2 H), 2.14-2.26 (m, 3 H), 0.81 (s, 3 H). |
| 32 | 550.1 | ¹H NMR (400 MHz, METHANOL-d₄) δ ppm 9.15 (s, 1 H) 7.60- |
| | | 7.69 (m, 1 H) 7.34-7.42 (m, 1 H) 7.31 (d, J = 2.51 Hz, 1 H) |
| | | 7.18 (d, J = 6.90 Hz, 1 H) 7.02-7.07 (m, 1 H) 5.10-5.33 (m, 1 |
| | | H) 4.56 (d, J = 5.02 Hz, 2 H) 4.21-4.32 (m, 4 H) 4.03-4.11 (m, |
| | | 2 H) 3.82-3.92 (m, 2 H) 3.47-3.71 (m, 2 H) 3.17-3.27 (m, 1 |
| | | H) 2.65-2.79 (m, 1 H) 2.60 (s, 3 H) 2.16-2.44 (m, 5 H) 1.96- |
| | | 2.15 (m, 1 H) 1.25-1.35 (m, 3 H). |
| 33 | 575.2 | ¹H NMR (400 MHz, METHANOL-d₄) δ ppm 9.11-9.26 (m, 1 |
| | | H), 7.61-7.73 (m, 1 H), 7.30-7.40 (m, 1 H), 7.22-7.29 (m, 1 |
| | | H), 7.03-7.11 (m, 1 H), 4.71 (s, 1 H), 4.21-4.37 (m, 4 H), 3.82- |
| | | 4.10 (m, 6 H), 3.57-3.79 (m, 2 H), 3.05-3.10 (m, 3 H), 2.73- |
| | | 2.87 (m, 1 H), 2.58-2.70 (m, 2 H), 2.42-2.56 (m, 1 H), 2.14- |
| | | 2.30 (m, 3 H), 0.78-0.85 (m, 3 H). |
| 78 | 605.9 | ¹H NMR (400 MHz, METHANOL-d₄) δ ppm 9.56-9.77 (m, 1 |
| | | H), 7.62-7.74 (m, 1 H), 7.30-7.43 (m, 2 H), 7.17-7.23 (m, 1 |
| | | H), 7.01-7.10 (m, 1 H), 5.49-5.75 (m, 1 H), 4.55-4.73 (m, 4 |
| | | H), 4.16-4.29 (m, 1 H), 3.82-4.12 (m, 6 H), 3.65-3.75 (m, 2 |
| | | H), 3.43-3.55 (m, 1 H), 2.53-2.87 (m, 2 H), 2.14-2.49 (m, 6 |
| | | H), 1.31 (d, J = 1.3 Hz, 3 H), 0.82-1.01 (m, 3 H). |
| 79 | 638.2 | ¹H NMR (400 MHz, METHANOL-d₄) δ ppm 9.53-9.79 (m, 1 |
| | | H), 7.61-7.87 (m, 1 H), 7.24-7.43 (m, 2 H), 6.94-7.17 (m, 1 |
| | | H), 5.37-5.81 (m, 1 H), 4.53-4.82 (m, 4 H), 4.18-4.30 (m, 1 |
| | | H), 3.63-4.10 (m, 7 H), 3.42-3.57 (m, 1 H), 2.31-2.79 (m, 6 |
| | | H), 2.10-2.29 (m, 2 H), 1.46-1.79 (m, 2 H), 1.07 (s, 3 H), 0.68- |
| | | 0.91 (m, 3 H). |
| 80 | 640.0 | ¹H NMR (400 MHz, METHANOL-d₄) δ ppm 9.72-9.86 (m, 1 |
| | | H), 7.65-7.75 (m, 1 H), 7.31-7.34 (m, 1 H), 7.24-7.31 (m, 1 |
| | | H), 6.98-7.09 (m, 1 H), 5.47-5.71 (m, 1 H), 4.68-4.81 (m, 2 |
| | | H), 4.48-4.67 (m, 2 H), 4.18-4.28 (m, 1 H), 4.01-4.15 (m, 2 |
| | | H), 3.85-4.00 (m, 4 H), 3.64-3.78 (m, 2 H), 3.45-3.56 (m, 1 |
| | | H), 2.55-2.81 (m, 2 H), 2.43-2.53 (m, 1 H), 2.25-2.42 (m, 4 |
| | | H), 2.08-2.24 (m, 1 H), 1.30 (d, J = 1.2 Hz, 3 H), 0.86 (s, 3 H). |

### 4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholine 1-oxide (Example 34)

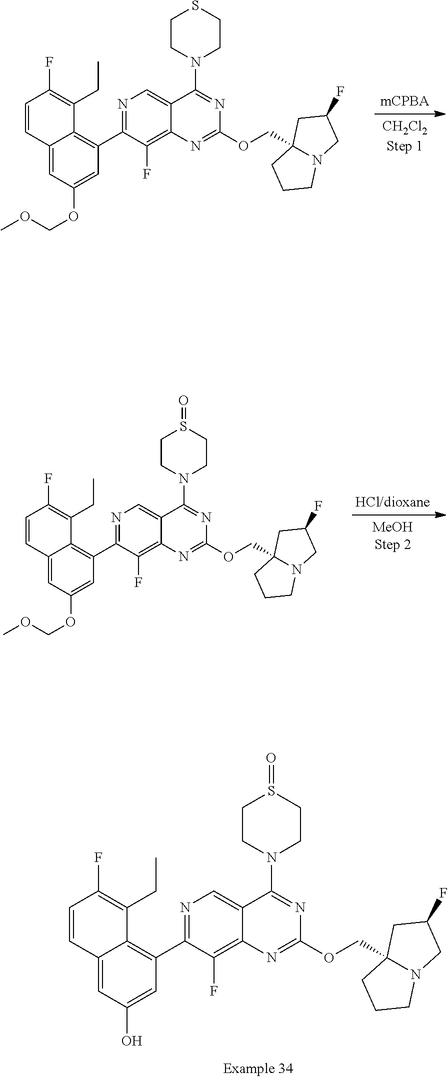 [SMILES: CCc1c(F)ccc2cc(OCOC)cc(-c3ncc4c(N5CCSCC5)nc(OC[C@@]56CCCN5C[C@H](F)C6)nc4c3F)c12 ; CCc1c(F)ccc2cc(OCOC)cc(-c3ncc4c(N5CCS(=O)CC5)nc(OC[C@@]56CCCN5C[C@H](F)C6)nc4c3F)c12 ; CCc1c(F)ccc2cc(O)cc(-c3ncc4c(N5CCS(=O)CC5)nc(OC[C@@]56CCCN5C[C@H](F)C6)nc4c3F)c12]

### Example 34

Step 1: 4-(7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholine 1-oxide. To a 20-mL vial were added 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholine (0.10 g, 0.16 mmol) and DCM (3.1 mL). To the resulting solution was added mCPBA (70.1 mg, 0.31 mmol), and the reaction mixture was allowed to stir at rt for 1 h. Upon completion, the reaction was concentrated under reduced pressure and the residue was purified by column chromatography on silica gel, eluting with 0-100% 3:1 EtOAc/EtOH blend in heptane with 2% triethylamine additive to give 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholine 1-oxide (21 mg, 0.031 mmol, 20% yield) as yellow solid. m/z (ESI): 656.2 (M+H)⁺.

Step 2: 4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholine 1-oxide. Synthesized in an analogous manner to Example 1. The product was isolated as a TFA salt. m/z (ESI): 612.2 (M+H)⁺. ¹H NMR (400 MHz, DMSO-d₆): δ ppm 9.21 (s, 1H), 7.78 (dd, J=9.1, 6.0 Hz, 1H), 7.31-7.41 (m, 2H), 7.02 (d, J=2.7 Hz, 1H), 5.47-5.71 (m, 1H), 4.51-4.70 (m, 4H), 4.20-4.32 (m, 2H), 3.68-3.93 (m, 4H), 2.97-3.30 (m, 6H), 2.08-2.40 (m, 7H), 0.74 (t, J=7.4 Hz, 3H). 4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1 λ⁶-thiomorpholine 1-oxide (Example 35)

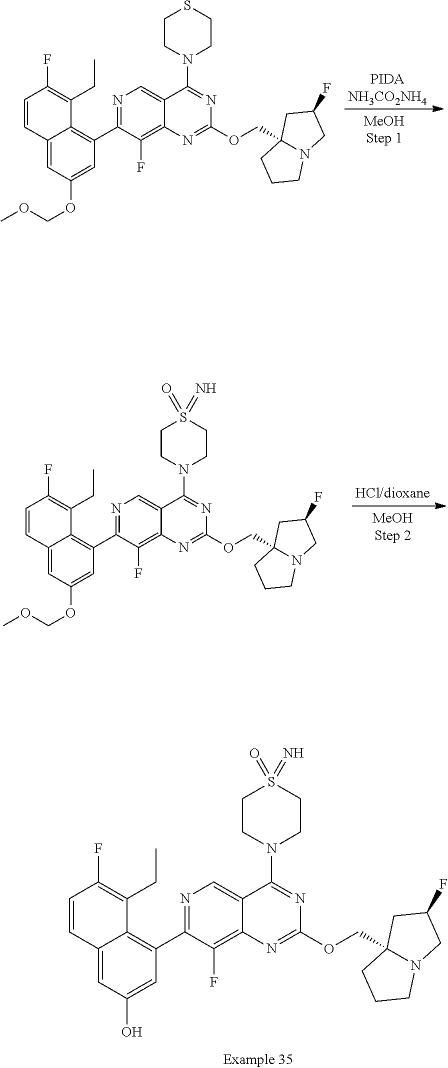 [SMILES: CCc1c(F)ccc2cc(OCOC)cc(-c3ncc4c(N5CCSCC5)nc(OC[C@@]56CCCN5C[C@H](F)C6)nc4c3F)c12 ; CCc1c(F)ccc2cc(OCOC)cc(-c3ncc4c(N5CCS(=N)(=O)CC5)nc(OC[C@@]56CCCN5C[C@H](F)C6)nc4c3F)c12 ; CCc1c(F)ccc2cc(O)cc(-c3ncc4c(N5CCS(=N)(=O)CC5)nc(OC[C@@]56CCCN5C[C@H](F)C6)nc4c3F)c12]

Step 1: 4-(7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1 λ⁶-thiomorpholine 1-oxide. To an 8-mL vial were added 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholine (0.10 g, 0.16 mmol) and methanol (3.1 mL). To the resulting solution were added ammonium carbamate (24 mg, 0.31 mmol) and iodobenzene diacetate (0.13 g, 0.39 mmol) and the mixture was allowed to stir at rt for 2 h. Upon completion, the reaction was concentrated under reduced pressure and was purified by column chromatography on silica gel, eluting with 0-100% 3:1 EtOAc/EtOH blend in heptane with 2% triethylamine additive in heptane to give 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1λ⁶-thiomorpholine 1-oxide (77 mg, 0.115 mmol, 73% yield) as yellow solid. m/z (ESI): 671.25 (M+H)⁺.

Step 2: 4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1 λ⁶-thiomorpholine 1-oxide. Synthesized in an analogous manner to Example 1. The product was isolated as TFA salt. m/z (ESI): 627.20 (M+H)⁺. ¹H NMR (400 MHz, DMSO-d₆): δ ppm 9.21 (s, 1H), 7.78 (dd, J=9.0, 6.1 Hz, 1H), 7.31-7.41 (m, 2H), 7.02 (d, J=2.5 Hz, 1H), 5.48-5.70 (m, 1H), 4.60-4.72 (m, 2H), 4.44-4.57 (m, 2H), 4.08-4.26 (m, 3H), 3.82-3.84 (m, 2H), 3.26-3.55 (m, 7H), 1.96-2.40 (m, 8H), 0.74 (t, J=7.3 Hz, 3H).

### 4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1-oxide (Example 36) and 4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1,1-dioxide (Example 37)

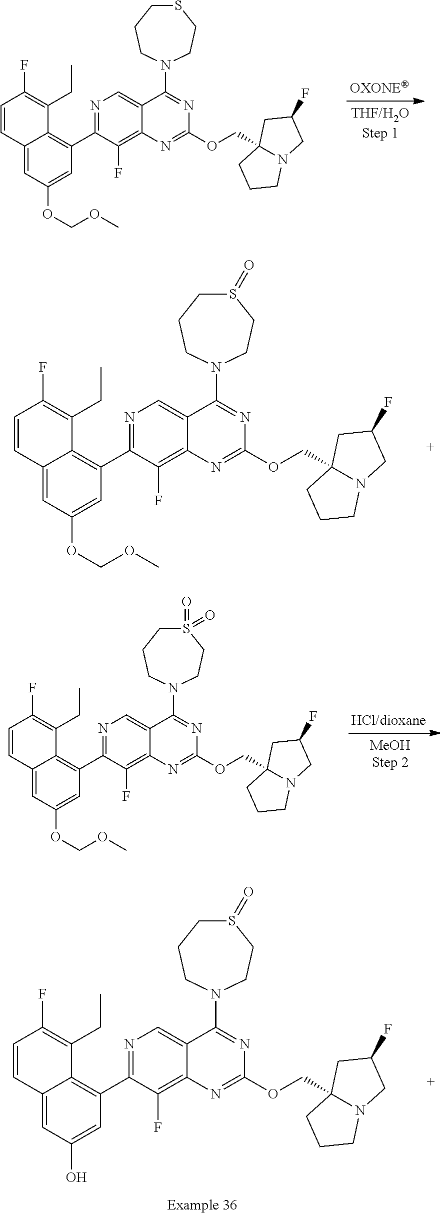 [SMILES: CCc1c(F)ccc2cc(OCOC)cc(-c3ncc4c(N5CCCSCC5)nc(OC[C@@]56CCCN5C[C@H](F)C6)nc4c3F)c12 ; CCc1c(F)ccc2cc(OCOC)cc(-c3ncc4c(N5CCCS(=O)CC5)nc(OC[C@@]56CCCN5C[C@H](F)C6)nc4c3F)c12 ; CCc1c(F)ccc2cc(OCOC)cc(-c3ncc4c(N5CCCS(=O)(=O)CC5)nc(OC[C@@]56CCCN5C[C@H](F)C6)nc4c3F)c12 ; CCc1c(F)ccc2cc(O)cc(-c3ncc4c(N5CCCS(=O)CC5)nc(OC[C@@]56CCCN5C[C@H](F)C6)nc4c3F)c12]

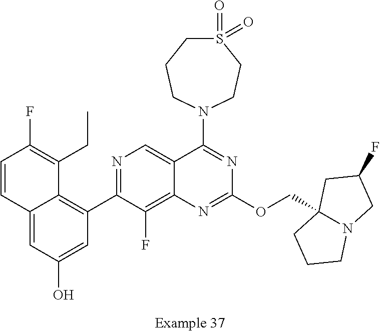 [SMILES: CCc1c(F)ccc2cc(O)cc(-c3ncc4c(N5CCCS(=O)(=O)CC5)nc(OC[C@@]56CCCN5C[C@H](F)C6)nc4c3F)c12]

Step 1: 4-(7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1-oxide and 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1,1-dioxide. To a 20-mL vial were added 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane (54 mg, 0.083 mmol), tetrahydrofuran (6.3 mL), and water (0.06 mL). The resulting solution was cooled to 0° C. and OXONE®, monopersulfate (51 mg, 0.083 mmol) was added, and the reaction mixture was allowed to stir at 0° C. After 45 min, the reaction mixture was allowed to warm to 23° C. After an additional 19 h, 10% aq. Na₂S₂O₃ (3 mL) was added and the mixture was stirred vigorously for 5 min. The aqueous layer was extracted with CH₂Cl₂ (2×5 mL), and the combined organics were dried with anhydrous Na₂SO₄ and concentrated to dryness to afford a crude mixture of 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1-oxide and 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1,1-dioxide as light-yellow solid. m/z (ESI): 670.2, 686.1 (M+H)⁺.

Step 2: 4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1-oxide and 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1,1-dioxide. Synthesized in an analogous manner to Example 1. Products were isolated as TFA salts. 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1-oxide. m/z (ESI, +ve ion): 626.1 (M+H)⁺. ¹H NMR (400 MHz, METHANOL-d₄) δ ppm 9.29 (s, 1H), 7.70 (dd, J=9.0, 5.9 Hz, 1H), 7.34 (d, J=2.5 Hz, 1H), 7.28 (t, J=9.4 Hz, 1H), 7.07 (dd, J=12.3, 2.5 Hz, 1H), 5.46-5.73 (m, 1H), 4.62-4.71 (m, 1H), 4.31-4.58 (m, 3H), 4.15-4.27 (m, 1H), 3.82-4.11 (m, 3H), 3.34-3.58 (m, 4H), 2.83-3.00 (m, 2H), 2.13-2.81 (m, 10H), 0.71-0.91 (m, 3H). 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-thiazepane 1,1-dioxide. m/z (ESI): 642.0 (M+H)⁺. ¹H NMR (400 MHz, METHANOL-d₄) δ ppm 9.27 (s, 1H), 7.71 (dd, J=9.0, 5.9 Hz, 1H), 7.34 (d, J=2.5 Hz, 1H), 7.28 (t, J=9.4 Hz, 1H), 7.08 (d, J=2.7 Hz, 1H), 5.46-5.73 (m, 1H), 4.61-4.75 (m, 2H), 4.31-4.52 (m, 4H), 3.84-4.14 (m, 3H), 3.76 (t, J=5.5 Hz, 2H), 3.37-3.57 (m, 3H), 2.29-2.83 (m, 8H), 2.22 (ddd, J=14.0, 7.0, 4.0 Hz, 2H), 0.76-0.88 (m, 3H).

### 4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-imino-1 λ⁶,4-thiazepane 1-oxide (Example 38)

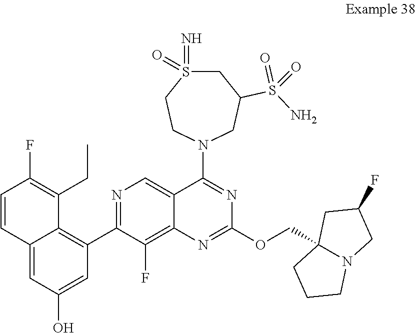 [SMILES: CCc1c(F)ccc2cc(O)cc(-c3ncc4c(N5CCS(=N)(=O)CC(S(N)(=O)=O)C5)nc(OC[C@@]56CCCN5C[C@H](F)C6)nc4c3F)c12]

Synthesized in an analogous manner to Example 35. The product was isolated as TFA salt. m/z (ESI): 641.1 (M+H)⁺. ¹H NMR (400 MHz, METHANOL-d₄) δ ppm 9.27 (s, 1H), 7.71 (dd, J=9.0, 5.9 Hz, 1H), 7.34 (d, J=2.5 Hz, 1H), 7.28 (t, J=9.4 Hz, 1H), 7.07 (d, J=2.3 Hz, 1H), 5.49-5.78 (m, 1H), 4.79-4.98 (m, 1H), 3.67-4.61 (m, 12H), 3.41-3.56 (m, 1H), 2.32-2.72 (m, 8H), 2.11-2.28 (m, 2H), 0.81 (t, J=7.3 Hz, 3H).

### 5-Ethyl-6-fluoro-4-(8-fluoro-2-(((2R,7aS)-2-fluorotetrahydro-1H-pyrrolizin-7a(5H)-yl)methoxy)-4-((S)-6-methoxy-1,4-oxazepan-4-yl)pyrido[4,3-d]pyrimidin-7-yl)naphthalen-2-ol (Example 39)

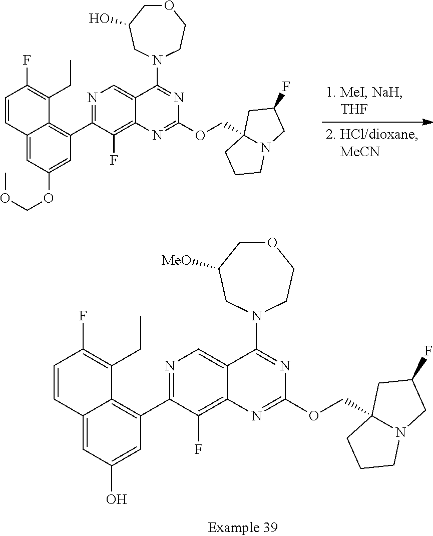 [SMILES: CCc1c(F)ccc2cc(OCOC)cc(-c3ncc4c(N5CCOC[C@@H](O)C5)nc(OC[C@@]56CCCN5C[C@H](F)C6)nc4c3F)c12 ; CCc1c(F)ccc2cc(O)cc(-c3ncc4c(N5CCOC[C@@H](OC)C5)nc(OC[C@@]56CCCN5C[C@H](F)C6)nc4c3F)c12]

To a 20-mL vial was added (S)-4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol (31 mg, 0.05 mmol, synthesized en route to Example 25) and sodium hydride (5.8 mg, 0.14 mmol, TCI America) in tetrahydrofuran (1.0 mL) at 0° C. The reaction was stirred at 0° C. for 20 min. Next, iodomethane (14 mg, 6.0 μL, 0.10 mmol, Sigma-Aldrich Corporation) was added and the reaction was warmed to rt. After 1 h the reaction mixture was diluted with water and extracted with CH₂Cl₂. The organic extract was dried over MgSO₄. The solution was filtered and concentrated in vacuo to give the crude material as light-yellow solid, which was added to a 20-mL vial. HCl in dioxane (4 M, 0.3 mL, 1.20 mmol, Sigma-Aldrich Corporation) and acetonitrile (0.9 mL) were added at 0° C. After 30 min, the solvent was removed under reduced pressure. The crude material was purified by reverse-phase HPLC to provide 5-ethyl-6-fluoro-4-(8-fluoro-2-(((2R,7aS)-2-fluorotetrahydro-1H-pyrrolizin-7a(5H)-yl)methoxy)-4-((S)-6-methoxy-1,4-oxazepan-4-yl)pyrido[4,3-d]pyrimidin-7-yl)naphthalen-2-ol as light-yellow solid (14 mg, 0.02 mmol, 40% yield). m/z (ESI): 624.3 (M+H)⁺. ¹H NMR (400 MHz, METHANOL-d₄) δ ppm 9.35-9.43 (m, 1H), 7.70 (dd, J=9.2, 5.9 Hz, 1H), 7.34 (d, J=2.7 Hz, 1H), 7.24-7.31 (m, 1H), 7.08 (s, 1H), 5.46-5.71 (m, 1H), 4.72 (dd, J=9.2, 2.1 Hz, 2H), 4.32-4.48 (m, 3H), 3.97-4.27 (m, 4H), 3.82-3.96 (m, 5H), 3.49 (d, J=4.6 Hz, 3H), 2.69-2.81 (m, 1H), 2.31-2.67 (m, 6H), 2.13-2.28 (m, 2H), 0.75-0.87 (m, 3H).

### 5-Ethyl-6-fluoro-4-(8-fluoro-2-(((2R,7aS)-2-fluorotetrahydro-1H-pyrrolizin-7a(5H)-yl)methoxy)-4-(2-(hydroxymethyl)thiomorpholino)pyrido[4,3-d]pyrimidin-7-yl)naphthalen-2-ol (Example 40)

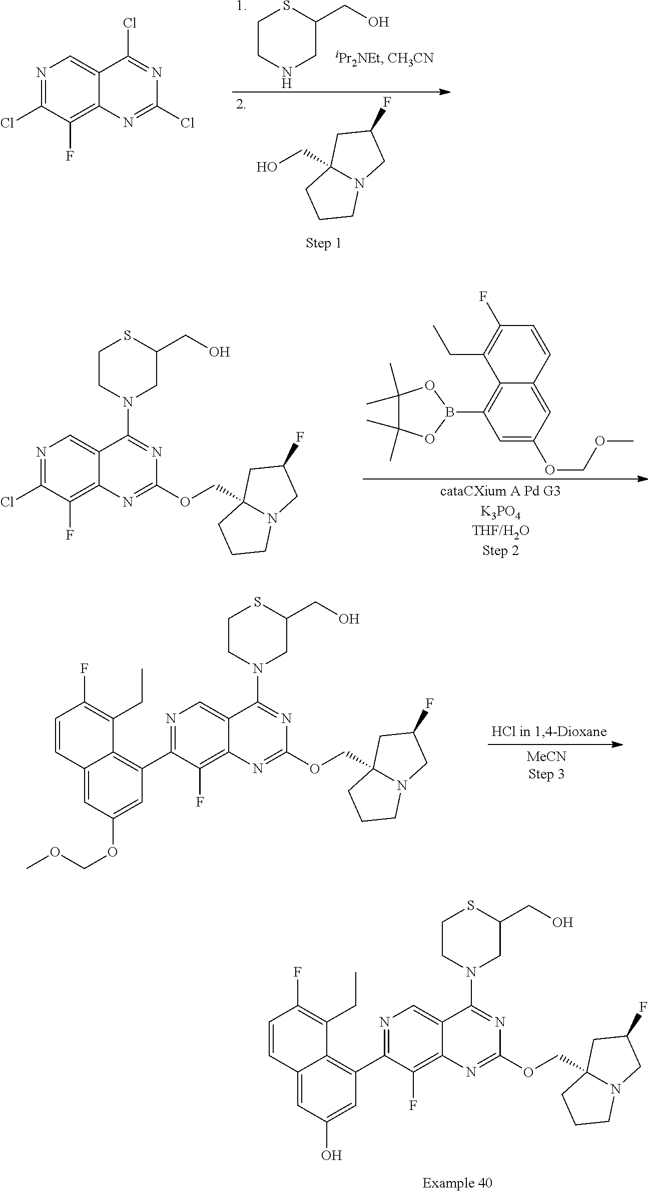

Step 1: (4-(7-Chloro-8-fluoro-2-(((2R,7aS)-2-fluorotetrahydro-1H-pyrrolizin-7a(5H)-yl)methoxy)pyrido[4,3-d]pyrimidin-4-yl)thiomorpholin-2-yl)methanol. To a 40-mL vial were added 2,4,7-trichloro-8-fluoropyrido[4,3-d]pyrimidine (0.46 g, 1.80 mmol, LabNetwork), thiomorpholin-2-ylmethanol (0.24 g, 1.80 mmol, Intermediate E), and acetonitrile (7.0 mL). The resulting suspension was cooled to −40° C. and DIPEA (1.16 g, 1.6 mL, 9.01 mmol, Aldrich) was added. The resulting mixture was warmed to 23° C. over 30 min. Upon completion, ((2R,7aS)-2-fluorotetrahydro-1H-pyrrolizin-7a(5H)-yl)methanol (0.57 g, 3.60 mmol, LabNetwork) was added and the mixture was heated to 80° C. for 16 h. The mixture was purified by column chromatographed on silica gel, eluting with a gradient of 0-30% MeOH in DCM to give (4-(7-chloro-8-fluoro-2-(((2R,7aS)-2-fluorotetrahydro-1H-pyrrolizin-7a(5H)-yl)methoxy)pyrido[4,3-d]pyrimidin-4-yl)thiomorpholin-2-yl)methanol (0.85 g, 1.79 mmol, 99% yield) as orange solid. m/z (ESI): 472.2 (M+H)⁺.

Step 2: (4-(7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholin-2-yl)methanol. A 1 dram vial was charged with (4-(7-chloro-8-fluoro-2-(((2R,7aS)-2-fluorotetrahydro-1H-pyrrolizin-7a(5H)-yl)methoxy)pyrido[4,3-d]pyrimidin-4-yl)thiomorpholin-2-yl)methanol (0.30 g, 0.64 mmol), 2-(8-ethyl-7-fluoro-3-(methoxymethoxy)naphthalen-1-yl)-4,4,5,5-tetramethyl-1,3,2-dioxaborolane (0.34 g, 0.95 mmol, LabNetwork), potassium phosphate (0.41 g, 1.91 mmol, Sigma Aldrich corporation), and cataCXium A Pd G3 (93 mg, 0.13 mmol, Sigma Aldrich Corporation). The vial was purged with nitrogen and the reactants were suspended in degassed tetrahydrofuran (5.8 mL) and water (0.6 mL). The reaction was then sealed and heated to 70° C. After stirring overnight, the reaction was cooled to rt and concentrated under reduced pressure to afford a crude black oil. The oil was then purified by column chromatography on silica gel, eluting with a gradient of 0-30% MeOH in DCM to provide (4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholin-2-yl)methanol as orange oil. Yield was not determined. m/z (ESI): 670.3 (M+H)⁺.

Step 3: 5-Ethyl-6-fluoro-4-(8-fluoro-2-(((2R,7aS)-2-fluorotetrahydro-1H-pyrrolizin-7a(5H)-yl)methoxy)-4-(2-(hydroxymethyl)thiomorpholino)pyrido[4,3-d]pyrimidin-7-yl)naphthalen-2-ol. The above (4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)thiomorpholin-2-yl)methanol was dissolved in MeCN (3.0 mL) and HCl in 1,4-dioxane (4 M, 4.0 mL, 15.89 mmol, Sigma-Aldrich Corporation) was added. The reaction was then stirred at rt for 1 h. The reaction was concentrated under reduced pressure and purified by reverse phase HPLC to provide 5-ethyl-6-fluoro-4-(8-fluoro-2-(((2R,7aS)-2-fluorotetrahydro-1H-pyrrolizin-7a(5H)-yl)methoxy)-4-(2-(hydroxymethyl)thiomorpholino)pyrido[4,3-d]pyrimidin-7-yl)naphthalen-2-ol bis(2,2,2-trifluoroacetate) (0.13 g, 0.16 mmol, 24% yield) as light yellow solid. m/z (ESI, +ve ion): 626.1 (M+H)⁺. ¹H NMR (400 MHz, DMSO-d₆): δ ppm 10.58-10.90 (m, 1H), 9.20 (d, J=1.3 Hz, 1H), 7.78 (dd, J=9.0, 6.1 Hz, 1H), 7.31-7.41 (m, 2H), 7.03 (dd, J=5.9, 2.7 Hz, 1H), 5.49-5.67 (m, 1H), 4.34-4.79 (m, 8H), 4.03 (br dd, J=13.6, 7.9 Hz, 1H), 3.75-3.95 (m, 4H), 3.50-3.68 (m, 2H), 3.18-3.37 (m, 2H), 2.89-3.10 (m, 2H), 2.53-2.70 (m, 1H), 2.31-2.42 (m, 2H), 2.02-2.24 (m, 4H), 0.74 (t, J=7.3 Hz, 3H).

TABLE 5
Examples 41 to 53, prepared in an Analogous Manner to Example 40.

| Ex. | | | | | Method |
|---|---|---|---|---|---|
| # | Structure | Name | Salt Code | Reagents | Change |
| 41 | 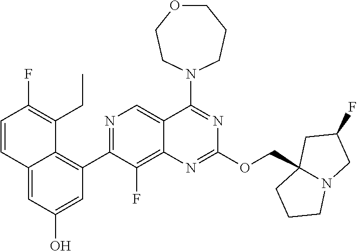 [SMILES: CCc1c(F)ccc2cc(O)cc(-c3ncc4c(N5CCCOCC5)nc(OC[C@]56CCCN5C[C@H](F)C6)nc4c3F)c12] | 5-ethyl-6-fluoro-4- (8-fluoro-2-(((2R, 7aR)-2-fluorotetra- hydro-1H-pyrrol- izin-7a(5H)-yl)- methoxy)-4-(1,4- oxazepan-4-yl)pyr- ido[4,3-d]pyrimi- din-7-yl)naphtha- len-2-ol | 2,2,2- trifluoro- acetate | Step 1: ((2R, 7aR)-2-Fluoro- hexahydro- 1H-pyrrolizin- 7a-yl)methanol hyrdochloride (CAS#: 2454490-66-3, WuXi App Tec Co. Ltd.) | Step 3: MeOH was used. |
| 42 | 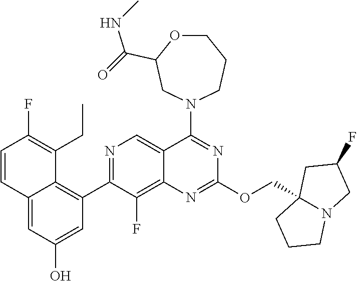 [SMILES: CCc1c(F)ccc2cc(O)cc(-c3ncc4c(N5CCCOC(C(=O)NC)C5)nc(OC[C@@]56CCCN5C[C@H](F)C6)nc4c3F)c12] | 4-(7-(8-Ethyl-7- fluoro-3-hydroxy- naphthalen-1-yl)- 8-fluoro-2- (((2R,7aS)-2- fluorotetrahydro- 1H-pyrrolizin- 7a(5H)-yl)meth- oxy)pyrido[4,3- d]pyrimidin-4-yl)- N-methyl-1,4- oxazepane-2- carboxamide Isomer 1 | bis(2,2,2- trifluoro- acetate) | Step 1: N- methyl-1,4- oxazepane-2- carboxamide (Intermediate C1) | Chiral separation after Step 1. Details included below. |
| 43 | 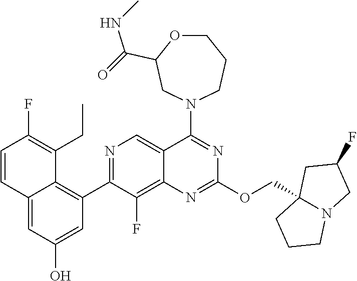 [SMILES: CCc1c(F)ccc2cc(O)cc(-c3ncc4c(N5CCCOC(C(=O)NC)C5)nc(OC[C@@]56CCCN5C[C@H](F)C6)nc4c3F)c12] | 4-(7-(8-Ethyl-7- fluoro-3-hydroxy- naphthalen-1-yl)- 8-fluoro-2- (((2R,7aS)-2- fluorotetrahydro- 1H-pyrrolizin- 7a(5H)-yl)meth- oxy)pyrido[4,3- d]pyrimidin-4-yl)- N-methyl-1,4- oxazepane-2- carboxamide Isomer 2 | bis(2,2,2- trifluoro- acetate) | Step 1: N- methyl-1,4- oxazepane-2- carboxamide (Intermediate C1) | Chiral separation after Step 1. Details included below. |
| 44 | 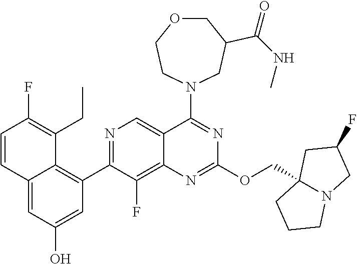 [SMILES: CCc1c(F)ccc2cc(O)cc(-c3ncc4c(N5CCOCC(C(=O)NC)C5)nc(OC[C@@]56CCCN5C[C@H](F)C6)nc4c3F)c12] | 4-(7-(8-Ethyl-7- fluoro-3-hydroxy- naphthalen-1-yl)-8- fluoro-2-(((2R,7aS)- 2-fluorotetrahydro- 1H-pyrrolizin- 7a(5H)-yl)meth- oxy)pyrido[4,3- d]pyrimidin-4-yl)- N-methyl-1,4- oxazepane-6- carboxamide Isomer 1 | bis(2,2,2- trifluoro- acetate) | Step 1: N- methyl-1,4- oxazepane-6- carboxamide (Intermediate C4) | Chiral separation after Step 1. Details included below. |
| 45 | 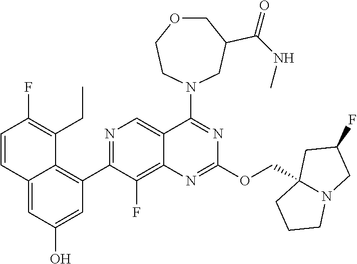 [SMILES: CCc1c(F)ccc2cc(O)cc(-c3ncc4c(N5CCOCC(C(=O)NC)C5)nc(OC[C@@]56CCCN5C[C@H](F)C6)nc4c3F)c12] | 4-(7-(8-Ethyl-7- fluoro-3-hydroxy- naphthalen-1-yl)-8- fluoro-2-(((2R,7aS)- 2-fluorotetrahydro- 1H-pyrrolizin- 7a(5H)-yl)meth- oxy)pyrido[4,3-d] pyrimidin-4-yl)- N-methyl-1,4- oxazepane-6- carboxamide Isomer 2 | bis(2,2,2- trifluoro- acetate) | Step 1: N- methyl-1,4- oxazepane-6- carboxamide (Intermediate C4) | Chiral separation after Step 1. Details included below. |
| 46 | 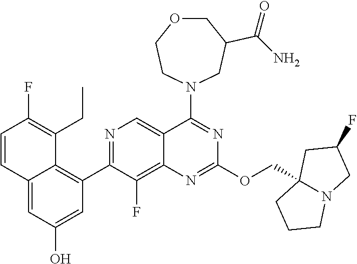 [SMILES: CCc1c(F)ccc2cc(O)cc(-c3ncc4c(N5CCOCC(C(N)=O)C5)nc(OC[C@@]56CCCN5C[C@H](F)C6)nc4c3F)c12] | 4-(7-(8-Ethyl-7- fluoro-3-hydroxy- naphthalen-1-yl)-8- fluoro-2-(((2R,7aS)- 2-fluorotetrahydro- 1H-pyrrolizin- 7a(5H)-yl)meth- oxy)pyrido[4,3-d] pyrimidin-4-yl)- 1,4-oxazepane-6- carboxamide Isomer 1 | bis(2,2,2- trifluoro- acetate) | Step 1: 1,4- oxazepane-6- carboxamide (Intermediate C3) | Chiral separation after Step 1. Details included below. |
| 47 | 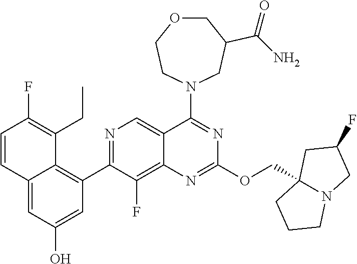 [SMILES: CCc1c(F)ccc2cc(O)cc(-c3ncc4c(N5CCOCC(C(N)=O)C5)nc(OC[C@@]56CCCN5C[C@H](F)C6)nc4c3F)c12] | 4-(7-(8-Ethyl-7- fluoro-3-hydroxy- naphthalen-1-yl)- 8-fluoro-2- (((2R,7aS)-2- fluorotetrahydro- 1H-pyrrolizin- 7a(5H)-yl)meth- oxy)pyrido[4,3-d] pyrimidin-4-yl)- 1,4-oxazepane-6- carboxamide Isomer 2 | bis(2,2,2- trifluoro- acetate) | Step 1: 1,4- oxazepane-6- carboxamide (Intermediate C3) | Chiral separation after Step 1. Details included below. |
| 48 | 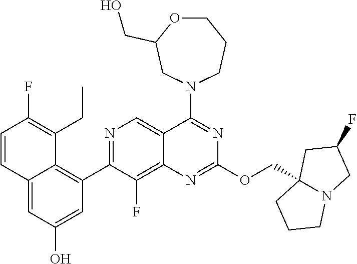 | 5-Ethyl-6-fluoro-4- (8-fluoro-2-(((2R, 7aS)-2-fluorotetra- hydro-1H-pyrrol- izin-7a(5H)-yl)- methoxy)-4-(2- (hydroxymethyl)- 1,4-oxazepan-4- yl)pyrido[4,3-d] pyrimidin-7-yl)- naphthalen-2-ol Isomer 1 | bis(2,2,2- trifluoro- acetate) | Step 1: (1,4- oxazepan-2- yl)methanol (Intermediate D1) | Chiral separation after Step 1. Details included below. |
| 49 | 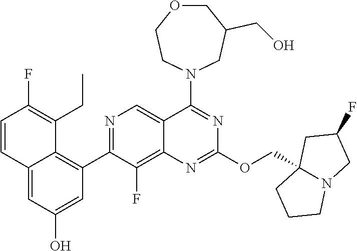 | 5-Ethyl-6-fluoro-4- (8-fluoro-2-(((2R, 7aS)-2-fluorotetra- hydro-1H-pyrrol- izin-7a(5H)-yl)- methoxy)-4-((S)-6- (hydroxymethyl)- 1,4-oxazepan-4-yl)- pyrido[4,3-d]pyr- imidin-7-yl)naph- thalen-2-ol Isomer 1 | bis(2,2,2- trifluoro- acetate) | Step 1: (1,4- oxazepan-6- yl)methanol (Intermediate D2) | Chiral separation after Step 1. Details included below. |
| 50 | 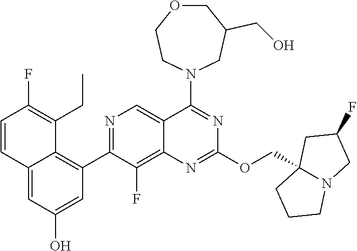 | 5-Ethyl-6-fluoro-4- (8-fluoro-2-(((2R, 7aS)-2-fluorotetra- hydro-1H-pyrrol- izin-7a(5H)-yl)- methoxy)-4-((R)- 6-(hydroxymeth- yl)-1,4-oxazepan- 4-yl)pyrido[4,3-d] pyrimidin-7-yl)- naphthalen-2-ol Isomer 2 | bis(2,2,2- trifluoro- acetate) | Step 1: (1,4- oxazepan-6- yl)methanol (Intermediate D2) | Chiral separation after Step 1. Details included below. |
| 51 | 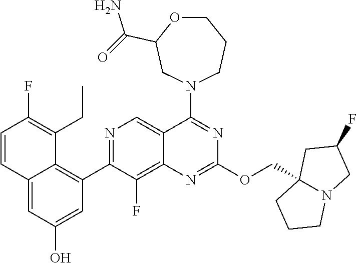 [SMILES: CCc1c(F)ccc2cc(O)cc(-c3ncc4c(N5CCCOC(C(N)=O)C5)nc(OC[C@@]56CCCN5C[C@H](F)C6)nc4c3F)c12] | 4-(7-(8-Ethyl-7- fluoro-3-hydroxy- naphthalen-1-yl)- 8-fluoro-2-(((2R, 7aS)-2-fluorotetra- hydro-1H-pyrrol- zin-7a(5H)-yl)- methoxy)pyrido [4,3-d]pyrimidin- 4-yl)-1,4- oxazepane-2- carboxamide | bis(2,2,2- trifluoro- acetate) | Step 1: 1,4- oxazepane-2- carboxamide (Intermediate C2) | |
| 52 | 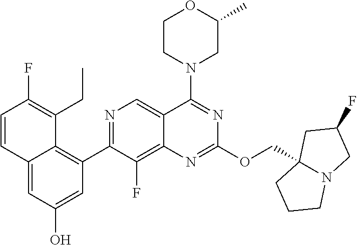 [SMILES: CCc1c(F)ccc2cc(O)cc(-c3ncc4c(N5CCO[C@H](C)C5)nc(OC[C@@]56CCCN5C[C@H](F)C6)nc4c3F)c12] | 5-Ethyl-6-fluoro- 4-(8-fluoro-2- (((2R,7aS)-2- fluorotetrahydro- 1H-pyrrolizin- 7a(5H)-yl)meth- oxy)-4-((R)-2- methylmorpho- lino)-pyrido[4,3-d] pyrimidin-7-yl)- naphthalen-2-ol | Free base | Step 1: (R)-2- methylmorph- oline hydro- chloride (CAS#: 168038-14-0, Advanced ChemBlocks, Inc.) | After Step 3, product was washed with aq. NaHCO₃ solution to free base. |
| 53 | 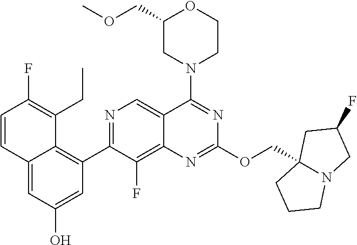 [SMILES: CCc1c(F)ccc2cc(O)cc(-c3ncc4c(N5CCO[C@@H](COC)C5)nc(OC[C@@]56CCCN5C[C@H](F)C6)nc4c3F)c12] | 5-Ethyl-6-fluoro-4- (8-fluoro-2-(((2R, 7aS)-2-fluorotetra- hydro-1H-pyrrol- izin-7a(5H)-yl)- methoxy)-4-((R)- 2-(methoxymeth- yl)morpholino)pyr- ido[4,3-d]pyrimi- din-7-yl)naph- thalen-2-ol | Free base | Step 1: (S)-3- (methoxymeth- yl)morpholine hydrochloride (CAS#: 218594-76-4, Ark Pharma) | Step 1: DBACO (0.2 eq.), Cs₂CO₃ (3 eq.) were used. After Step 3, product was washed with aq. NaHCO₃ solution to free base. |

TABLE 6
SFC Conditions for Chiral Separation.

| Separation | Conditions | Peak to Ex.# |
|---|---|---|
| 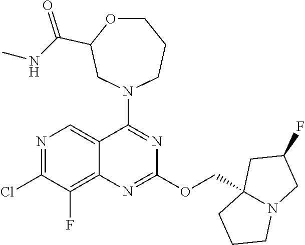 [SMILES: CNC(=O)C1CN(c2nc(OC[C@@]34CCCN3C[C@H](F)C4)nc3c(F)c(Cl)ncc23)CCCO1] | Column: Chiralcel OX (21 × 150 mm 5 μm) Mobile phase: 50% iPrOH w/ 0.2% TEA Flowrate: 120 mL/min. Yield: 600 mg sample submitted to generate 112.9 mg of peak 1 with an ee of >99% and 104.8 mg of peak 2 with an ee of >99% | Peak 1: Ex. 42 Peak 2: Ex. 43 |
| 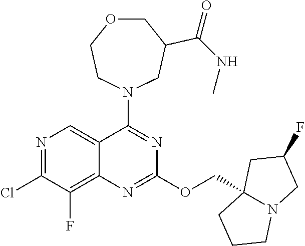 [SMILES: CNC(=O)C1COCCN(c2nc(OC[C@@]34CCCN3C[C@H](F)C4)nc3c(F)c(Cl)ncc23)C1] | Column: ChiralPak IF (2 × 25 cm 5 μm) Mobile phase: 40% iPrOH w/ 0.2% TEA Flowrate: 80 mL/min. Yield: 550 mg sample submitted to generate 71.1 mg of peak 1 with an ee of 99% and 70.8 mg of peak 2 with an ee of 97%. | Peak 1: Ex. 44 Peak 2: Ex. 45 |
| 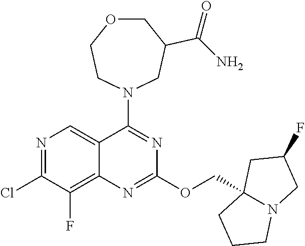 [SMILES: NC(=O)C1COCCN(c2nc(OC[C@@]34CCCN3C[C@H](F)C4)nc3c(F)c(Cl)ncc23)C1] | Column: (S,S) Whelk-01 (21 × 250 mm 5μm) Mobile phase: 45% MeOH w/ 0.2% TEA Flowrate: 80 mL/min Yield: 600 mg of sample submitted to generate 215.7 mg of peak 1 with an ee of >99% and 72.8 mg of peak 2 with an ee of >96%. | Peak 1: Ex. 46 Peak 2: Ex. 47 |
| 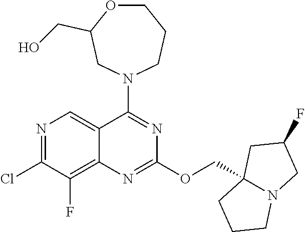 [SMILES: OCC1CN(c2nc(OC[C@@]34CCCN3C[C@H](F)C4)nc3c(F)c(Cl)ncc23)CCCO1] | Column: Chiralcel OD (2 × 25 cm 5 μm) Mobile phase 30% MeOH w/ 0.2% DEA Flowrate: 80 mL/min. Yield: 1200 mg of substrate submitted to generate 259 mg of peak 1 with an ee of 99% and 409 mg of peak 2 with an ee of 95%. | Peak 2: Ex. 48 |
| 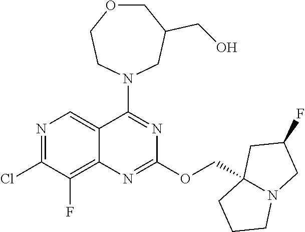 [SMILES: OCC1COCCN(c2nc(OC[C@@]34CCCN3C[C@H](F)C4)nc3c(F)c(Cl)ncc23)C1] | Column: ChiralPak IF (2 × 25 cm 5 μm) Mobile phase: 40% MeOH w/ 0.2% DEA Flowrate: 80 mL/min. Yield: 730 mg of sample submitted to generate 109 mg of peak 1 with an ee of 93% and 170.8 mg of peak 2 with an ee of 90%. | Peak 1: Ex. 49 Peak 2: Ex. 50 |

TABLE 7
Analytical Data for Examples 41 to 53.

| | MS | |
|---|---|---|
| Ex. | m/z (ESI): | |
| # | (M + H)⁺ | ¹H NMR |
| 41 | 594.0 | ¹H NMR (400 MHz, METHANOL-d₄) δ ppm 9.23 (s, 1 H), 7.66-7.77 |
| | | (m, 1 H), 7.32-7.37 (m, 1 H), 7.23-7.31 (m, 1 H), 7.06-7.11 (m, 1 H), |
| | | 5.45-5.63 (m, 1 H), 4.71-4.76 (m, 1 H), 4.24-4.36 (m, 4 H), 3.99- |
| | | 4.12 (m, 3 H), 3.85-3.92 (m, 2 H), 3.67-3.79 (m, 1 H), 3.53-3.61 (m, |
| | | 1 H), 3.44-3.52 (m, 2 H), 2.64-2.80 (m, 1 H), 2.38-2.57 (m, 3 H), |
| | | 2.22 (br s, 6 H), 0.79-0.88 (m, 3 H). |
| 42 | 651.1 | ¹H NMR (400 MHz, METHANOL-d₄): δ ppm 9.27 (s, 1 H), 7.69 (dd, |
| | | J = 9.0, 5.7 Hz, 1 H), 7.32 (d, J = 2.5 Hz, 1 H), 7.26 (t, J = 9.3 Hz, 1 H), |
| | | 7.05 (dd, J = 9.7, 2.5 Hz, 1 H), 5.50-5.71 (m, 1 H), 4.88-5.07 (m, 2 H), |
| | | 4.70 (d, J = 12.6 Hz, 1 H), 4.32-4.49 (m, 2 H), 4.17-4.31 (m, 2 H), 3.86- |
| | | 4.13 (m, 4 H), 3.63-3.79 (m, 1 H), 3.43-3.56 (m, 1 H), 2.78 (d, J = 2.1 |
| | | Hz, 3 H), 2.09-2.72 (m, 10 H), 0.80 (dt, J = 17.8, 7.5 Hz, 3 H). |
| 43 | 651.1 | ¹H NMR (400 MHz, METHANOL-d₄) δ ppm 9.27 (s, 1 H), 7.69 (dd, |
| | | J = 8.9, 5.8 Hz, 1 H), 7.32 (d, J = 2.5 Hz, 1 H), 7.26 (t, J = 9.3 Hz, 1 H), |
| | | 7.05 (dd, J = 12.2, 2.5 Hz, 1 H), 5.47-5.70 (m, 1 H), 4.99 (ddd, J = 19.7, |
| | | 14.6, 2.2 Hz, 1 H), 4.73-4.83 (m, 2 H), 4.33-4.48 (m, 2 H), 4.16-4.31 |
| | | (m, 2 H), 3.85-4.13 (m, 4 H), 3.70 (dt, J = 17.8, 11.1 Hz, 1 H), 3.43- |
| | | 3.55 (m, 1 H), 2.58-2.83 (m, 5 H), 2.13-2.52 (m, 8 H), 0.80 (dt, |
| | | J = 11.4, 7.4 Hz, 3 H). |
| 44 | 651.1 | ¹H NMR (400 MHz, METHANOL-d₄): δ ppm 9.21-9.31 (m, 1 H), 7.71 |
| | | (dd, J = 8.9, 5.8 Hz, 1 H), 7.34 (d, J = 2.5 Hz, 1 H), 7.28 (t, J = 9.4 Hz, 1 H), |
| | | 7.08 (dd, J = 5.7, 2.4 Hz, 1 H), 5.48-5.71 (m, 1 H), 4.63-4.79 (m, 3 H), |
| | | 3.85-4.46 (m, 10 H), 3.46-3.59 (m, 1 H), 3.20 (dt, J = 8.6, 4.5 Hz, 1 H), |
| | | 2.78 (s, 3 H), 2.12-2.71 (m, 8 H), 0.82 (dt, J = 13.0, 7.3 Hz, 3 H). |
| 45 | 651.1 | ¹H NMR (400 MHz, METHANOL-d₄): δ ppm 9.24 (d, J = 2.1 Hz, 1 H), |
| | | 7.71 (dd, J = 9.0, 5.9 Hz, 1 H), 7.34 (d, J = 2.7 Hz, 1 H), 7.28 (t, J = 9.3 Hz, |
| | | 1 H), 7.08 (dd, J = 5.1, 2.8 Hz, 1 H), 5.50-5.72 (m, 1 H), 4.61-4.78 (m, |
| | | 3 H), 4.33-4.47 (m, 1 H), 3.98-4.32 (m, 6 H), 3.82-3.97 (m, 3 H), |
| | | 3.44-3.56 (m, 1 H), 3.13-3.25 (m, 1 H), 2.56-2.83 (m, 5 H), 2.32- |
| | | 2.53 (m, 4 H), 2.12-2.29 (m, 2 H), 0.82 (dt, J = 18.0, 7.5 Hz, 3 H). |
| 46 | 637.1 | 1H NMR (400 MHz, METHANOL-d₄): δ ppm 9.25 (d, J-3.1 Hz, 1 H), |
| | | 7.71 (dd, J = 9.0, 5.9 Hz, 1 H), 7.34 (d, J = 2.7 Hz, 1 H), 7.28 (t, J = 9.4 Hz, |
| | | 1 H), 7.08 (dd, J = 6.6, 2.5 Hz, 1 H), 5.49-5.70 (m, 1 H), 4.64-4.80 (m, |
| | | 3 H), 4.32-4.46 (m, 1 H), 3.84-4.30 (m, 9 H), 3.44-3.57 (m, 1 H), |
| | | 3.17-3.29 (m, 1 H), 2.15-2.80 (m, 8 H), 0.75-0.89 (m, 3 H). |
| 47 | 637.1 | ¹H NMR (400 MHz, METHANOL-d₄): δ ppm 9.23-9.33 (m, 1 H), 7.71 |
| | | (dd, J = 9.0, 5.9 Hz, 1 H), 7.34 (d, J = 2.7 Hz, 1 H), 7.28 (t, J-9.3 Hz, 1 H), |
| | | 7.05-7.11 (m, 1 H), 5.46-5.69 (m, 1 H), 3.76-4.82 (m, 13 H), 3.45- |
| | | 3.57 (m, 1 H), 3.19-3.31 (m, 1 H), 2.35-2.86 (m, 6 H), 2.11-2.30 (m, |
| | | 2 H), 0.73-0.90 (m, 3 H). |
| 48 | 624.1 | ¹H NMR (400 MHz, METHANOL-d₄): δ ppm 9.27 (d, J = 1.7 Hz, 1 H), |
| | | 7.70 (dd, J = 9.0, 5.9 Hz, 1 H), 7.34 (d, J = 2.7 Hz, 1 H), 7.28 (t, J = 9.4 Hz, |
| | | 1 H), 7.08 (dd, J = 8.2, 2.5 Hz, 1 H), 5.49-5.71 (m, 1 H), 4.65-4.75 (m, |
| | | 3 H), 3.64-4.43 (m, 11 H), 3.45-3.56 (m, 1 H), 2.34-2.81 (m, 7 H), |
| | | 2.13-2.27 (m, 3 H), 0.83 (dt, J = 11.8, 7.4 Hz, 3 H). |
| 49 | 624.1 | 1H NMR (400 MHz, METHANOL-d₄): δ ppm 9.24-9.34 (m, 1 H), 7.70 |
| | | (dd, J = 9.0, 5.9 Hz, 1 H), 7.34 (d, J = 2.7 Hz, 1 H), 7.28 (t, J-9.4 Hz, 1 H), |
| | | 7.08 (dd, J = 9.0, 2.5 Hz, 1 H), 5.48-5.73 (m, 1 H), 4.75 (s, 2 H), 4.40- |
| | | 4.54 (m, 1 H), 3.85-4.22 (m, 8 H), 3.44-3.71 (m, 4 H), 1.95-2.82 (m, |
| | | 10 H), 0.82 (dt, J = 12.4, 7.4 Hz, 3 H). |
| 50 | 624.1 | ¹H NMR (400 MHz, METHANOL-d₄): δ ppm 9.29 (d, J = 2.1 Hz, 1 H), |
| | | 7.70 (dd, J = 9.0, 5.9 Hz, 1 H), 7.34 (d, J = 2.7 Hz, 1 H), 7.28 (t, J = 9.3 Hz, |
| | | 1 H), 7.08 (dd, J = 7.6, 2.4 Hz, 1 H), 5.50-5.71 (m, 1 H), 4.62-4.74 (m, |
| | | 2 H), 4.36-4.56 (m, 1 H), 3.39-4.27 (m, 12 H), 1.99-2.81 (m, 10 H), |
| | | 0.76-0.89 (m, 3 H). |
| 51 | 637.1 | ¹H NMR (400 MHz, METHANOL-d₄): δ ppm 8.92-9.32 (m, 1 H), 7.66- |
| | | 7.75 (m, 1 H), 7.32-7.37 (m, 1 H), 7.24-7.31 (m, 1 H), 7.01-7.11 |
| | | (m, 1 H), 5.44-5.74 (m, 1 H), 4.79-5.04 (m, 3 H), 3.66-4.45 (m, 8 H), |
| | | 3.42-3.57 (m, 1 H), 2.11-2.89 (m, 10 H), 1.80-2.01 (m, 1 H), 0.74- |
| | | 0.89 (m, 3 H). |
| 52 | 594.3 | ¹H NMR (400 MHz, DMSO-d₆) δ ppm 9.88 (s, 1 H), 9.15 (s, 1 H), 7.77 |
| | | (dd, J = 9.1, 6.0 Hz, 1 H), 7.28-7.40 (m, 2 H), 7.03 (d, J = 2.3 Hz, 1 H), |
| | | 5.15-5.44 (m, 1 H), 4.37-4.54 (m, 2 H), 4.12-4.22 (m, 1 H), 4.04- |
| | | 4.12 (m, 1 H), 3.98 (br d, J = 11.9 Hz, 1 H), 3.67-3.85 (m, 2 H), 3.44- |
| | | 3.60 (m, 1 H), 2.93-3.25 (m, 4 H), 2.78-2.89 (m, 1 H), 2.28-2.43 (m, |
| | | 1 H), 1.96-2.23 (m, 4 H), 1.71-1.91 (m, 3 H), 1.20 (dd, J-6.2, 2.8 Hz, |
| | | 3 H), 0.74 (t, J = 7.3 Hz, 3 H). |
| 53 | 624.3 | ¹H NMR (400 MHz, DMSO-d₆) δ ppm 9.88 (s, 1 H), 9.14 (s, 1 H), 7.76 |
| | | (dd, J-9.0, 6.1 Hz, 1 H), 7.29-7.40 (m, 2 H), 7.03 (d, J = 2.5 Hz, 1 H), |
| | | 5.17-5.42 (m, 1 H), 4.35-4.51 (m, 2 H), 4.19 (dd, J = 10.5, 5.9 Hz, 1 |
| | | H), 4.09 (dd, J = 10.6, 2.0 Hz, 1 H), 3.95-4.05 (m, 1 H), 3.86 (br dd, |
| | | J-6.6, 3.7 Hz, 1 H), 3.75 (td, J = 11.3, 2.2 Hz, 1 H), 3.43-3.62 (m, 3 H), |
| | | 3.33-3.42 (m, 1 H), 3.31 (d, J = 1.0 Hz, 3 H), 2.98-3.17 (m, 3 H), 2.80- |
| | | 2.89 (m, 1 H), 2.27-2.41 (m, 1 H), 2.11-2.25 (m, 2 H), 2.05-2.11 (m, |
| | | 1 H), 1.95-2.05 (m, 1 H), 1.71-1.93 (m, 3 H), 0.74 (t, J = 7.4 Hz, 3 H). |

### 5-Ethyl-6-fluoro-4-(8-fluoro-2-(((2R,7aS)-2-fluorotetrahydro-1H-pyrrolizin-7a(5H1)-yl)methoxy)-4-((R)-5-methyl-1,4-oxazepan-4-yl)pyrido[4,3-d]pyrimidin-7-yl)naphthalen-2-ol (Example 54)

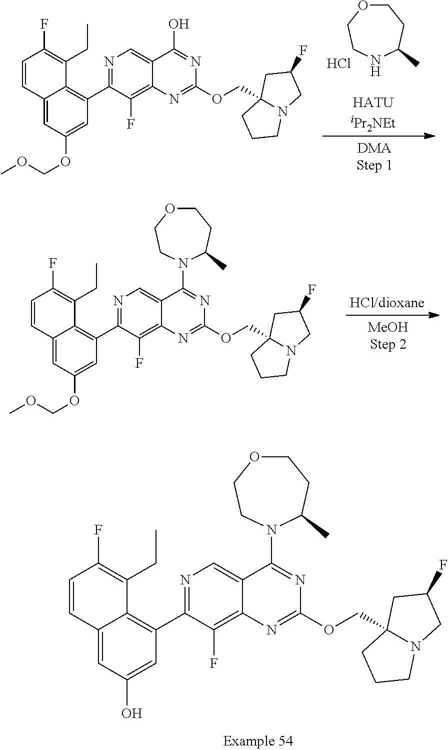

Step 1: (R)-4-(7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-5-methyl-1,4-oxazepane. To a stirred solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (18 mg, 0.03 mmol, Intermediate A) in N,N-dimethylacetamide (0.20 mL) was added DIPEA (21 mg, 0.03 mL, 0.16 mmol, Sigma-Aldrich Corporation) and HATU (49 mg, 0.13 mmol, Combi-Blocks Inc.). The resulting mixture was stirred at rt for 5 min. (R)-5-Methyl-1,4-oxazepane hydrochloride (7.4 mg, 0.05 mmol, Enamine) in N,N-dimethylacetamide (0.20 mL) was added and the reaction was stirred at rt for 1 h. The reaction mixture was purified by reverse phase HPLC to afford (R)-4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-5-methyl-1,4-oxazepane (11 mg, 0.02 mmol, 52% yield) as yellow solid which was used directly in the next step. m/z (ESI, +ve ion): 652.2 (M+H)⁺.

Step 2: 5-Ethyl-6-fluoro-4-(8-fluoro-2-(((2R,7aS)-2-fluorotetrahydro-1H-pyrrolizin-7a(5H)-yl)methoxy)-4-((R)-5-methyl-1,4-oxazepan-4-yl)pyrido[4,3-d]pyrimidin-7-yl)naphthalen-2-ol. (R)-4-(7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-5-methyl-1,4-oxazepane (11 mg, 0.02 mmol) was dissolved in acetonitrile (0.50 mL). HCl Solution (4.0 M in dioxane, 0.04 mL, 0.17 mmol, Sigma-Aldrich Corporation) was added and the reaction mixture was stirred at rt for 0.5 h. The reaction mixture was purified by reverse phase HPLC to afford 5-ethyl-6-fluoro-4-(8-fluoro-2-(((2R,7aS)-2-fluorotetrahydro-1H-pyrrolizin-7a(5H)-yl)methoxy)-4-((R)-5-methyl-1,4-oxazepan-4-yl)pyrido[4,3-d]pyrimidin-7-yl)naphthalen-2-ol as bis(2,2,2-trifluoroacetate) and as yellow solid (11 mg, 0.01 mmol, 79% yield). m/z (ESI): 608.0 (M+H. ¹H NMR (400 MHz, METHANOL-d₄) δ ppm 9.11 (d, J=5.4 Hz, 1H), 7.71 (dd, J=9.2, 5.9 Hz, 1H), 7.25-7.35 (m, 2H), 7.07 (dd, J=17.1, 2.5 Hz, 1H), 5.47-5.70 (m, 1H), 4.88-4.99 (m, 1H), 4.71-4.76 (m, 1H), 4.54-4.69 (m, 2H), 4.18 (brt, J=10.0 Hz, 1H), 3.89-4.07 (m, 5H), 3.76-3.86 (m, 1H), 3.41-3.54 (m, 2H), 2.11-2.79 (m, 10H), 1.59 (d, J=6.3 Hz, 3H), 0.83 (q, J=7.1 Hz, 3H).

TABLE 8
Examples 55 to 72, prepared in an analogous manner to Example 54.

| Ex. | | | | | Method |
|---|---|---|---|---|---|
| # | Structure | Name | Salt Form | Reagent | Change |
| 55 | 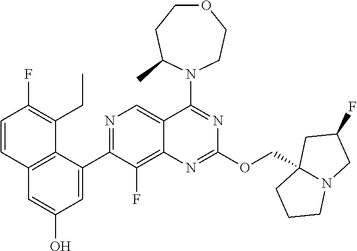 | 5-Ethyl-6-fluoro-4- (8-fluoro-2-(((2R, 7aS)-2-fluorotetra- hydro-1H-pyrrol- izin-7a(5H)-yl)- methoxy)-4-((S)- 5-methyl-1,4- oxazepan-4-yl)- pyrido[4,3-d] pyrimidin-7-yl)- naphthalen-2-ol | 2,2,2- trifluoro- aceate | Step 1: (S)-5- methyl-1,4- oxazepane hydrochloride (CAS#: 2694057-41-3, Enamine) | |
| 56 | 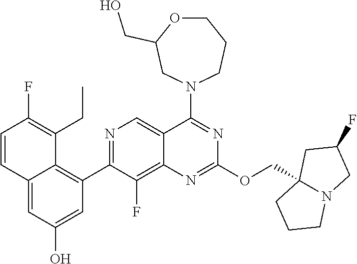 | 5-Ethyl-6-fluoro-4- (8-fluoro-2-(((2R, 7aS)-2-fluorotetra- hydro-1H-pyrrol- izin-7a(5H)-yl)- methoxy)-4-(2- (hydroxymethyl)- 1,4-oxazepan-4- yl)pyrido[4,3-d] pyrimidin-7-yl)- naphthalen-2-ol | bis(2,2,2- trifluoro- acetate) | Step 1: (1,4- oxazepan-2- yl)methanol (CAS#: 1207254-23-6, Aurum Pharmatech) | |
| 57 | 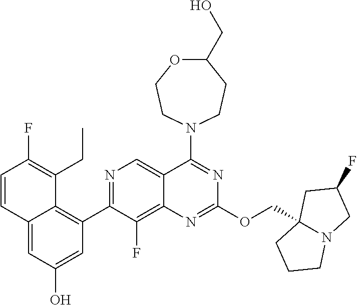 [SMILES: CCc1c(F)ccc2cc(O)cc(-c3ncc4c(N5CCOC(CO)CC5)nc(OC[C@@]56CCCN5C[C@H](F)C6)nc4c3F)c12] | 5-Ethyl-6-fluoro-4- (8-fluoro-2-(((2R, 7aS)-2-fluorotetra- hydro-1H-pyrrolizin- 7a(5H)-yl)methoxy)- 4-(7-(hydroxymeth- yl)-1,4-oxazepan-4- yl)pyrido[4,3-d] pyrimidin-7-yl)- naphthalen-2-ol | bis(2,2,2- trifluoro- acetate) | Step 1: (1,4- oxazepan-7- yl)methanol (CAS#: 627860-89-3, Aurum Pharmatech) | |
| 58 | 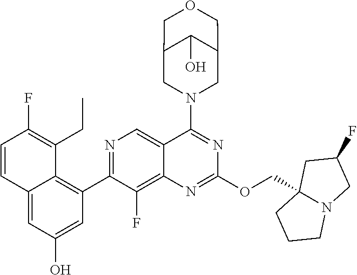 | 7-(7-(8-Ethyl-7- fluoro-3-hydroxy- naphthalen-1-yl)-8- fluoro-2-(((2R,7aS)- 2-fluorotetrahydro- 1H-pyrrolizin- 7a(5H)-yl)meth- oxy)pyrido[4,3-d] pyrimidin-4-yl)-3- oxa-7-azabicyclo [3.3.1]nonan-9-ol | bis(2,2,2- trifluoro- acetate) | Step 1: 3-oxa- 7-azabicyclo [3.3.1]nonan- 9-ol (Intermediate F) | |
| 59 | 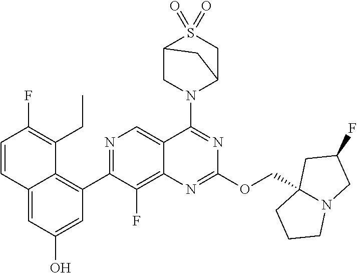 [SMILES: CCc1c(F)ccc2cc(O)cc(-c3ncc4c(N5CC6CC5CS6(=O)=O)nc(OC[C@@]56CCCN5C[C@H](F)C6)nc4c3F)c12] | 5-(7-(8-Ethyl-7- fluoro-3-hydroxy- naphthalen-1-yl)-8- fluoro-2-(((2R,7aS)- 2-fluorotetrahydro- 1H-pyrrolizin- 7a(5H)-yl)meth- oxy)pyrido[4,3-d] pyrimidin-4-yl)-2- thia-5-azabicyclo [2.2.1]heptane 2,2- dioxide | bis(2,2,2- trifluoro- acetate) | Step 1: 2-thia- 5-azabicyclo [2.2.1]heptane 2,2-dioxide (CAS#: 1312812-72-8, Ambeed) | |
| 60 | 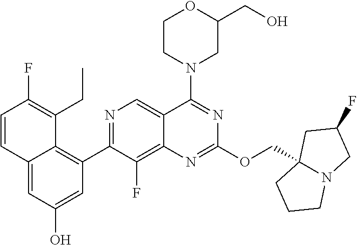 [SMILES: CCc1c(F)ccc2cc(O)cc(-c3ncc4c(N5CCOC(CO)C5)nc(OC[C@@]56CCCN5C[C@H](F)C6)nc4c3F)c12] | 5-Ethyl-6-fluoro-4- (8-fluoro-2-(((2R, 7aS)-2-fluorotetra- hydro-1H-pyrrol- izin-7a(5H)-yl)- methoxy)-4-(2- (hydroxymethyl)- morpholino)pyrido [4,3-d]pyrimidin-7- yl)naphthalen-2-ol | 2,2,2- trifluoro- acetate | Step 1: 2- (((tert-butyl- dimethylsilyl)- oxy)methyl) morpholine (Intermediate G) | Before Step 2, TBAF/ THF (1M) was used to remove the TBS group. |
| 61 | 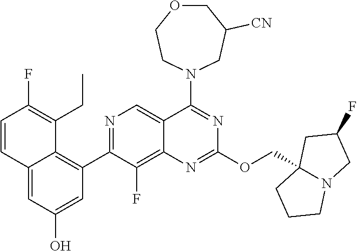 [SMILES: CCc1c(F)ccc2cc(O)cc(-c3ncc4c(N5CCOCC(C#N)C5)nc(OC[C@@]56CCCN5C[C@H](F)C6)nc4c3F)c12] | 4-(7-(8-Ethyl-7- fluoro-3-hydroxy- naphthalen-1-yl)-8- fluoro-2-(((2R,7aS)- 2-fluorotetrahydro- 1H-pyrrolizin- 7a(5H)-yl)meth- oxy)-pyrido[4,3-d] pyrimidin-4-yl)- 1,4-oxazepane-6- carbonitrile | Free base | Step 1: 1,4- oxazepane-6- carbonitrile (Intermediate H) | Purifica- tion per- formed with 0.1% formic acid in H₂O and MeCN as mobile phase, XSelect column (19 × 100 mm, 5 μm) |
| 62 | 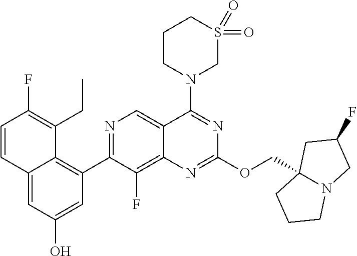 [SMILES: CCc1c(F)ccc2cc(O)cc(-c3ncc4c(N5CCCS(=O)(=O)C5)nc(OC[C@@]56CCCN5C[C@H](F)C6)nc4c3F)c12] | 3-(7-(8-Ethyl-7- fluoro-3-hydroxy- naphthalen-1-yl)-8- fluoro-2-(((2R,7aS)- 2-fluorotetrahydro- 1H-pyrrolizin- 7a(5H)-yl)meth- oxy)pyrido[4,3-d] pyrimidin-4-yl)- 1,3-thiazinane 1,1-dioxide | 2,2,2- trifluoro- aceate | Step 1: 1-λ6,3- thiazinane-1,1- dione hydro- chloride (CAS#: 2059999-55-0, Enamine) | |
| 63 | 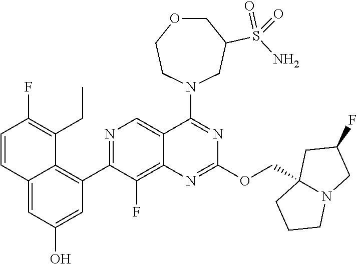 [SMILES: CCc1c(F)ccc2cc(O)cc(-c3ncc4c(N5CCOCC(S(N)(=O)=O)C5)nc(OC[C@@]56CCCN5C[C@H](F)C6)nc4c3F)c12] | 4-(7-(8-Ethyl-7- fluoro-3-hydroxy- naphthalen-1-yl)-8- fluoro-2-(((2R,7aS)- 2-fluorotetrahydro- 1H-pyrrolizin- 7a(5H)-yl)meth- oxy)pyrido[4,3-d] pyrimidin-4-yl)- 1,4-oxazepane-6- sulfonamide | 2,2,2- trifluoro- aceate | Step 1: 1,4- oxazepane-6- sulfonamide hydrochloride (CAS#: 2694729-07-0, Chemspace) | |
| 64 | 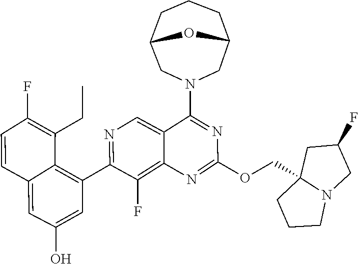 | 4-(4-((1R,5S)-9- oxa-3-azabicyclo [3.3.1]nonan-3-yl)- 8-fluoro-2-(((2R, 7aS)-2-fluorotetra- hydro-1H-pyrrolizin- 7a(5H)-yl)methoxy)- pyrido[4,3-d]pyrim- idin-7-yl)-5-ethyl- 6-fluoronaphthalen- 2-ol | Formate | Step 1: 9-oxa- 3-azabicyclo [3.3.1]nonane hydrochloride (CAS#: 1947317-74-9, Enamine) | |
| 65 | 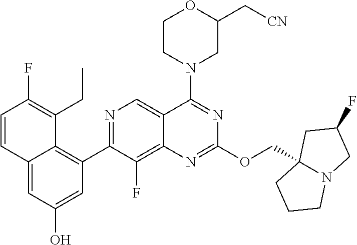 | 2-(4-(7-(8-Ethyl-7- fluoro-3-hydroxy- naphthalen-1-yl)-8- fluoro-2-(((2R,7aS)- 2-fluorotetrahydro- 1H-pyrrolizin- 7a(5H)-yl)methoxy)- pyrido[4,3-d]pyrim- idin-4-yl)morpholin- 2-yl)acetonitrile | Formate | Step 1: 2- (morpholin-2- yl)acetonitrile hydrochloride (CAS#: 1242465-38-8, WuXi) | |
| 66 | 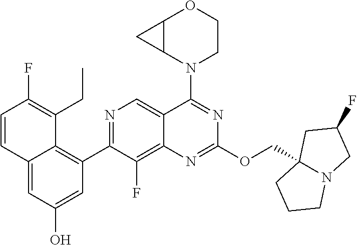 [SMILES: CCc1c(F)ccc2cc(O)cc(-c3ncc4c(N5CCOC6CC65)nc(OC[C@@]56CCCN5C[C@H](F)C6)nc4c3F)c12] | 4-(4-((1R,6S)-2-oxa- 5-azabicyclo[4.1.0] heptan-5-yl)-8- fluoro-2-(((2R,7aS)- 2-fluorotetrahydro- 1H-pyrrolizin- 7a(5H)-yl)methoxy)- pyrido[4,3-d]pyrim- idin-7-yl)-5-ethyl- 6-fluoronaphthalen- 2-ol | 2,2,2- trifluoro- aceate | Step 1: 2-oxa- 5-azabicyclo [4.1.0]heptane hydrochloride (CAS#: 1354952-28-5, Enamine) | |
| 67 | 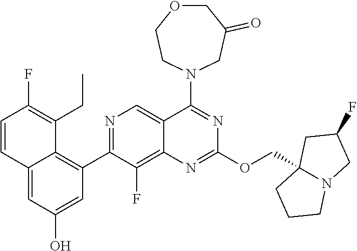 | 4-(7-(8-ethyl-7- fluoro-3-hydroxy- naphthalen-1-yl)-8- fluoro-2-(((2R,7aS)- 2-fluorotetrahydro- 1H-pyrrolizin- 7a(5H)-yl)methoxy)- pyrido[4,3-d]pyrim- idin-4-yl)-1,4- oxazepan-6-one | 2,2,2- trifluoro- aceate | Step 1: 1,4- oxazepan-6- one hydro- chloride (CAS#: 2306265-53-0, AA Blocks) | |
| 68 | 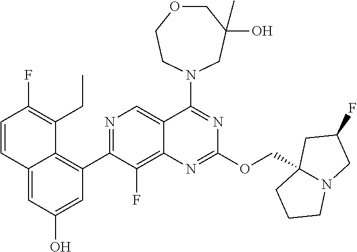 | 4-(7-(8-ethyl-7- fluoro-3-hydroxy- naphthalen-1-yl)-8- fluoro-2-(((2R,7aS)- 2-fluorotetrahydro- 1H-pyrrolizin- 7a(5H)-yl)methoxy)- pyrido[4,3-d]pyrim- idin-4-yl)-6-methyl- 1,4-oxazepan-6-ol Isomer 1 | 2,2,2- trifluoro- aceate | Step 1: 6- methyl-1,4- oxazepan-6-ol hydrochloride (Intermediate J1) | |
| 69 | 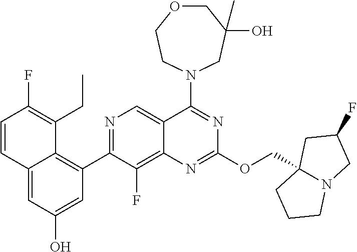 [SMILES: CCc1c(F)ccc2cc(O)cc(-c3ncc4c(N5CCOCC(C)(O)C5)nc(OC[C@@]56CCCN5C[C@H](F)C6)nc4c3F)c12] | 4-(7-(8-ethyl-7- fluoro-3-hydroxy- naphthalen-1-yl)-8- fluoro-2-(((2R,7aS)- 2-fluorotetrahydro- 1H-pyrrolizin- 7a(5H)-yl)methoxy)- pyrido[4,3-d]pyrim- idin-4-yl)-6-methyl- 1,4-oxazepan-6-ol Isomer 2 | 2,2,2- trifluoro- aceate | Step 1: 6- methyl-1,4- oxazepan-6-ol hydrochloride (Intermediate J2) | |
| 70 | 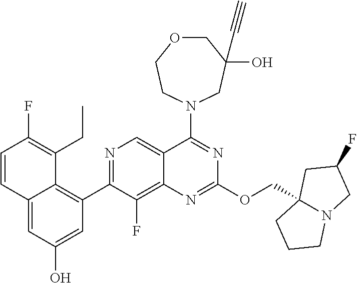 [SMILES: C#CC1(O)COCCN(c2nc(OC[C@@]34CCCN3C[C@H](F)C4)nc3c(F)c(-c4cc(O)cc5ccc(F)c(CC)c45)ncc23)C1] | 4-(7-(8-ethyl-7- fluoro-3-hydroxy- naphthalen-1-yl)-8- fluoro-2-(((2R,7aS)- 2-fluorotetrahydro- 1H-pyrrolizin- 7a(5H)-yl)methoxy)- pyrido[4,3-d]pyrim- idin-4-yl)-6-ethynyl- 1,4-oxazepan-6-ol | 2,2,2- trifluoro- aceate | Step 1: 6-((tri- methylsilyl) ethynyl)-1,4- oxazepan-6-ol hydrochloride (Intermediate K) | Additional step after Step 2. Procedure included below. |
| 71 | 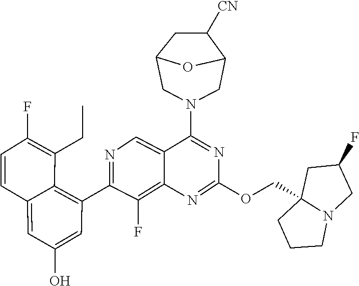 [SMILES: CCc1c(F)ccc2cc(O)cc(-c3ncc4c(N5CC6CC(C#N)C(C5)O6)nc(OC[C@@]56CCCN5C[C@H](F)C6)nc4c3F)c12] | 3-(7-(8-ethyl-7- fluoro-3-(methoxy- methoxy)naphthalen- 1-yl)-8-fluoro-2- (((2R,7aS)-2-fluoro- tetrahydro-1H- pyrrolizin-7a(5H)- yl)methoxy)pyrido [4,3-d]pyrimidin- 4-yl)-8-oxa-3-aza- bicyclo[3.2.1]octane- 6-carbonitrile Isomer 1 | 2,2,2- trifluoro- aceate | Step 1: 8-oxa- 3-azabicyclo [3.2.1]octane- 6-carbonitrile hydrochloride (Intermediate I) | |
| 72 | 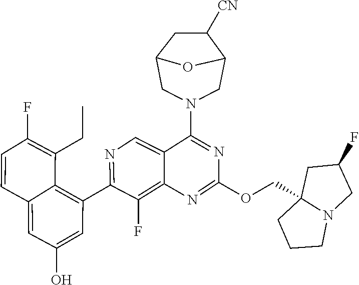 [SMILES: CCc1c(F)ccc2cc(O)cc(-c3ncc4c(N5CC6CC(C#N)C(C5)O6)nc(OC[C@@]56CCCN5C[C@H](F)C6)nc4c3F)c12] | 3-(7-(8-ethyl-7- fluoro-3-(methoxy- methoxy)naphthalen- 1-yl)-8-fluoro-2- (((2R,7aS)-2-fluoro- tetrahydro-1H-pyr- rolizin-7a(5H)-yl)- methoxy)pyrido[4,3- d]pyrimidin-4-yl)- 8-oxa-3-azabicyclo [3.2.1]octane-6- carbonitrile Isomer 2 | 2,2,2- trifluoro- aceate | Step 1: 8-oxa- azabicyclo [3.2.1]octane- 6-carbonitrile hydrochloride (Intermediate I) | |

### Additional Step for Example 70

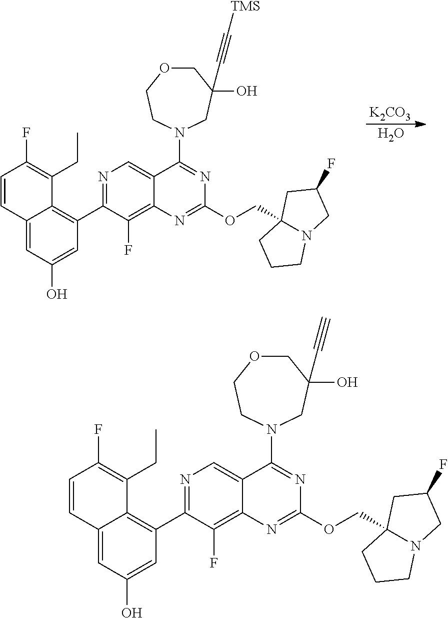 [SMILES: CCc1c(F)ccc2cc(O)cc(-c3ncc4c(N5CCOCC(O)(C#C[Si](C)(C)C)C5)nc(OC[C@@]56CCCN5C[C@H](F)C6)nc4c3F)c12 ; "K2CO3" ; O ; C#CC1(O)COCCN(c2nc(OC[C@@]34CCCN3C[C@H](F)C4)nc3c(F)c(-c4cc(O)cc5ccc(F)c(CC)c45)ncc23)C1]

4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-((trimethylsilyl)ethynyl)-1,4-oxazepan-6-ol (64 mg, 0.09 mmol) was dissolved in methanol and treated with potassium carbonate (25 mg, 0.18 mmol, Sigma-Aldrich Corporation) in water (1.0 mL). The reaction was stirred for 3 h. The material was purified by reverse phase HPLC to give 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-ethynyl-1,4-oxazepan-6-ol as 2,2,2-trifluoroacetate and as yellow solid (8 mg, 11 mol, 12% yield).

TABLE 9
Analytical Data for Examples 55 to 72.

| | MS | |
|---|---|---|
| | m/z (ESI): | |
| Ex. # | (M+H)* | ¹H NMR |
| 55 | 608.0 | ¹H NMR (400 MHz, METHANOL-d₄) δ ppm 9.11 (d, J = 5.2 Hz, 1 H), |
| | | 7.70 (dd, J = 9.2, 5.9 Hz, 1 H), 7.34 (d, J = 2.7 Hz, 1 H), 7.28 (t, J = 9.3 |
| | | Hz, 1 H), 7.08 (dd, J = 17.6, 2.5 Hz, 1 H), 5.47-5.73 (m, 1 H), 4.84- |
| | | 4.97 (m, 1 H), 4.68-4.73 (m, 2 H), 4.54-4.64 (m, 1 H), 4.13-4.21 |
| | | (m, 1 H), 3.88-4.11 (m, 5 H), 3.75-3.86 (m, 1 H), 3.43-3.53 (m, 2 |
| | | H), 2.60-2.88 (m, 2 H), 2.33-2.57 (m, 5 H), 2.07-2.29 (m, 3 H), |
| | | 1.59 (d, J-6.3 Hz, 3 H), 0.82 (dt, J = 10.5, 7.5 Hz, 3 H). |
| 56 | 624.3 | ¹H NMR (400 MHz, METHANOL-d₄): δ ppm 9.27 (d, J = 1.7 Hz, 1 H), |
| | | 7.70 (dd, J = 9.2, 5.9 Hz, 1 H), 7.34 (d, J = 2.7 Hz, 1 H), 7.28 (t, J = 9.4 |
| | | Hz, 1 H), 7.08 (dd, J = 7.8, 2.6 Hz, 1 H), 5.42-5.74 (m, 1 H), 4.63- |
| | | 4.77 (m, 3 H), 3.55-4.43 (m, 11 H), 3.42-3.53 (m, 1 H), 2.14-2.84 |
| | | (m, 10 H), 0.74-0.92 (m, 3 H). |
| 57 | 624.3 | ¹H NMR (400 MHz, METHANOL-d₄): δ ppm 8.98-9.32 (m, 1 H), |
| | | 7.70 (dd, J = 9.1, 5.7 Hz, 1 H), 7.34 (d, J = 2.5 Hz, 1 H), 7.28 (t, J = 9.4 |
| | | Hz, 1 H), 7.08 (dd, J = 6.3, 2.5 Hz, 1 H), 5.46-5.78 (m, 1 H), 4.63- |
| | | 4.76 (m, 2 H), 3.63-4.59 (m, 10 H), 3.41-3.61 (m, 3 H), 1.87-2.78 |
| | | (m, 10 H), 0.70-0.91 (m, 3 H). |
| 58 | 636.1 | ¹H NMR (400 MHz, DMSO-d₆): δ ppm 10.64-10.80 (m, 1 H), 9.70- |
| | | 10.11 (m, 1 H), 9.19-9.30 (m, 1 H), 7.77 (dd, J = 9.1, 6.2 Hz, 1 H), |
| | | 7.30-7.40 (m, 2 H), 7.05 (d, J-2.3 Hz, 1 H), 5.48-5.72 (m, 1 H), |
| | | 4.70-5.10 (m, 2 H), 4.60 (s, 2 H), 4.00 (br d, J = 8.2 Hz, 6 H), 3.57- |
| | | 3.71 (m, 5 H), 3.33 (br dd, J = 4.9, 2.8 Hz, 1 H), 2.07-2.43 (m, 7 H), |
| | | 1.78-1.93 (m, 2 H), 0.73 (t, J = 7.4 Hz, 3 H). |
| 59 | 640.2 | ¹H NMR (400 MHz, DMSO-d₆): δ ppm 10.58-10.81 (m, 1 H), 9.75- |
| | | 10.14 (m, 1 H), 9.29 (br s, 1 H), 7.78 (dd, J = 8.9, 6.0 Hz, 1 H), 7.33- |
| | | 7.40 (m, 2 H), 7.03 (d, J = 2.5 Hz, 1 H), 5.43-5.72 (m, 2 H), 4.61- |
| | | 4.70 (m, 2 H), 4.25-4.43 (m, 2 H), 3.77-3.96 (m, 4 H), 3.24-3.38 |
| | | (m, 2 H), 2.52-2.75 (m, 3 H), 1.96-2.42 (m, 7 H), 0.74 (dt, J = 12.0, |
| | | 7.4 Hz, 3 H). |
| 60 | 610.0 | ¹H NMR (400 MHz, METHANOL-d₄) δ ppm 9.20 (s, 1 H), 7.70 (dd, |
| | | J-9.2, 5.9 Hz, 1 H), 7.34 (d, J = 2.7 Hz, 1 H), 7.27 (t, J = 9.3 Hz, 1 H), |
| | | 7.07 (d, J = 2.5 Hz, 1 H), 5.51-5.68 (m, 1 H), 4.70 (br s, 3 H), 4.09- |
| | | 4.17 (m, 1 H), 3.64-4.01 (m, 8 H), 3.51 (br d, J = 5.6 Hz, 2 H), 3.11- |
| | | 3.19 (m, 2 H), 2.55-2.80 (m, 2 H), 2.33-2.53 (m, 4 H), 2.16-2.29 |
| | | (m, 2 H), 0.82 (t, J = 7.4 Hz, 3 H). |
| 61 | 619.2 | ¹H NMR (400 MHz, METHANOL-d₄) δ ppm 9.24 (s, 1 H), 7.70 (dd, |
| | | J = 9.1, 5.7 Hz, 1 H), 7.33 (d, J = 2.5 Hz, 1 H), 7.27 (t, J = 9.4 Hz, 1 H), |
| | | 7.08 (dd, J = 9.1, 2.6 Hz, 1 H), 5.34-5.54 (m, 1 H), 4.56 (s, 5 H), 4.28- |
| | | 4.38 (m, 2 H), 3.98-4.26 (m, 4 H), 3.55 (br s, 4 H), 3.17-3.26 (m, 1 |
| | | H), 2.32-2.60 (m, 3 H), 2.01-2.31 (m, 5 H), 0.82 (br s, 3 H). |
| 62 | 628.3 | ¹H NMR (400 MHz, METHANOL-d₄) δ ppm 9.25 (s, 1 H), 7.71 (dd, |
| | | J = 9.0, 5.9 Hz, 1 H), 7.34 (d, J = 2.7 Hz, 1 H), 7.28 (t, J-9.4 Hz, 1 H), |
| | | 7.09 (d, J = 2.5 Hz, 1 H), 5.50-5.71 (m, 1 H), 5.47 (br s, 2 H), 4.65- |
| | | 4.76 (m, 2 H), 4.24-4.42 (m, 2 H), 3.93 (br d, J = 15.9 Hz, 3 H), 3.50 |
| | | (br t, J = 5.7 Hz, 3 H), 2.71-2.90 (m, 1 H), 2.70 (s, 1 H), 2.68-2.76 |
| | | (m, 1 H), 2.54-2.70 (m, 1 H), 2.30-2.54 (m, 4 H), 2.28-2.33 (m, 1 |
| | | H), 2.13-2.27 (m, 2 H), 0.81 (t, J = 7.3 Hz, 3 H). |
| 63 | 673.0 | ¹H NMR (400 MHz, METHANOL-d₄) δ ppm 9.03-9.30 (m, 1 H), |
| | | 7.66-7.74 (m, 1 H), 7.32-7.36 (m, 1 H), 7.24-7.31 (m, 1 H), 7.01- |
| | | 7.11 (m, 1 H), 5.50-5.74 (m, 1 H), 5.10-5.44 (m, 1 H), 4.83-5.00 |
| | | (m, 1 H), 4.60-4.74 (m, 1 H), 4.35-4.49 (m, 1 H), 3.75-4.33 (m, 8 |
| | | H), 3.43-3.58 (m, 1 H), 3.09-3.28 (m, 1 H), 2.06-2.86 (m, 8 H), |
| | | 0.74-0.93 (m, 3 H). |
| 64 | 620.2 | ¹H NMR (500 MHz, DMSO-d₆) δ ppm 10.42-10.74 (m, 1 H), 9.21- |
| | | 9.24 (m, 1 H), 7.78 (dd, J-9.1, 6.1 Hz, 1 H), 7.33-7.38 (m, 2 H), 7.03 |
| | | (d, J = 2.3 Hz, 1 H), 5.49-5.66 (m, 1 H), 4.57-4.71 (m, 4 H), 4.03- |
| | | 4.15 (m, 2 H), 3.70-4.01 (m, 6 H), 3.27-3.36 (m, 1 H), 2.30-2.40 |
| | | (m, 2 H), 2.11-2.24 (m, 3 H), 1.90-2.11 (m, 3 H), 1.73-1.88 (m, 3 |
| | | H), 1.46-1.58 (m, 1 H), 0.70-0.75 (m, 3 H). |
| 65 | 619.2 | ¹H NMR (500 MHz, DMSO-d₆) δ ppm 8.15 (s, 1 H), 7.77 (dd, J = 9.1, |
| | | 6.1 Hz, 1 H), 7.31-7.37 (m, 2 H), 7.00-7.02 (m, 1 H), 5.18-5.41 (m, |
| | | 1 H), 4.51 (br d, J = 13.1 Hz, 1 H), 4.37-4.44 (m, 1 H), 4.17 (ddd, |
| | | J = 10.4, 6.4, 4.0 Hz, 1 H), 3.95-4.10 (m, 3 H), 3.79-3.88 (m, 1 H), |
| | | 3.47-3.57 (m, 1 H), 3.22-3.43 (m, 1 H), 2.98-3.17 (m, 4 H), 2.80- |
| | | 2.98 (m, 2 H), 2.31-2.40 (m, 1 H), 1.97-2.18 (m, 4 H), 1.74-1.91 |
| | | (m, 3 H), 0.73 (t, J-7.3 Hz, 3 H). |
| 66 | 592.3 | ¹H NMR (400 MHz, METHANOL-d₄) δ ppm 9.81-9.98 (m, 1 H), |
| | | 7.64-7.80 (m, 1 H), 7.35 (d, J = 2.7 Hz, 1 H), 7.26-7.31 (m, 1 H), |
| | | 7.18-7.18 (m, 1 H), 7.09 (d, J = 2.5 Hz, 1 H), 6.88-6.88 (m, 1 H), |
| | | 5.46-5.74 (m, 1 H), 4.64-4.72 (m, 2 H), 4.20-4.30 (m, 1 H), 4.09- |
| | | 4.14 (m, 1 H), 3.89-3.95 (m, 2 H), 3.68-3.76 (m, 1 H), 3.49-3.54 |
| | | (m, 1 H), 2.32-2.52 (m, 6 H), 2.16-2.28 (m, 3 H), 1.30-1.43 (m, 2 |
| | | H), 1.18 (br d, J = 3.1 Hz, 1 H), 0.82 (br d, J = 2.1 Hz, 3 H). |
| 67 | 608.2 | ¹H NMR (400 MHz, METHANOL-d₄) δ ppm 9.25-9.65 (m, 1 H), |
| | | 7.49-7.89 (m, 1 H), 7.21-7.47 (m, 2 H), 7.00-7.14 (m, 1 H), 5.43- |
| | | 5.85 (m, 1 H), 4.46-4.74 (m, 5 H), 4.17-4.38 (m, 2 H), 3.97-4.17 |
| | | (m, 3 H), 3.65-3.97 (m, 3 H), 3.40-3.56 (m, 1 H), 2.31-2.86 (m, 6 |
| | | H), 2.12-2.27 (m, 2 H), 0.57-0.97 (m, 3 H). |
| 68 | 624.2 | ¹H NMR (400 MHz, METHANOL-d₄) δ ppm 9.52-9.91 (m, 1 H), |
| | | 7.57-7.81 (m, 1 H), 7.18-7.45 (m, 2 H), 6.92-7.16 (m, 1 H), 5.50- |
| | | 5.83 (m, 1 H), 4.55-4.72 (m, 3 H), 4.17-4.30 (m, 1 H), 3.86-4.11 |
| | | (m, 6 H), 3.60-3.79 (m, 2 H), 3.44-3.57 (m, 1 H), 2.11-2.88 (m, 8 |
| | | H), 1.18-1.55 (m, 3 H), 0.50-0.94 (m, 3 H). |
| 69 | 624.2 | ¹H NMR (400 MHz, METHANOL-d₄) δ ppm 9.50-9.81 (m, 1 H), |
| | | 7.55-7.94 (m, 1 H), 7.18-7.43 (m, 2 H), 6.90-7.14 (m, 1 H), 5.43- |
| | | 5.93 (m, 1 H), 4.47-4.75 (m, 4 H), 3.88-4.31 (m, 7 H), 3.61-3.78 |
| | | (m, 2 H), 3.40-3.56 (m, 1 H), 2.13-2.87 (m, 8 H), 1.31 (d, J = 1.9 Hz, |
| | | 3 H), 0.39-0.90 (m, 3 H). |
| 70 | 634.2 | ¹H NMR (400 MHz, METHANOL-d₄) δ ppm 9.35-9.81 (m, 1 H), |
| | | 7.70 (dd, J = 9.0, 5.9 Hz, 1 H), 7.23-7.50 (m, 2 H), 7.02-7.12 (m, 1 |
| | | H), 5.27-5.82 (m, 1 H), 4.64-4.75 (m, 2 H), 4.33-4.56 (m, 2 H), |
| | | 3.78-4.32 (m, 8 H), 3.43-3.62 (m, 1 H), 3.04-3.12 (m, 1 H), 2.02- |
| | | 2.83 (m, 8 H), 1.87-1.99 (m, 1 H), 0.61-0.97 (m, 3 H). |
| 71 | 631.0 | ¹H NMR (400 MHz, METHANOL-d₄) δ ppm 9.17 (s, 1 H) 7.65-7.74 |
| | | (m, 1 H) 7.34 (d, J-2.72 Hz, 1 H) 7.24-7.31 (m, 1 H) 7.05 (s, 1 H) |
| | | 5.41-5.73 (m, 1 H) 4.80-4.83 (m, 1 H) 4.65-4.76 (m, 4 H) 4.47- |
| | | 4.63 (m, 1 H) 3.76-4.12 (m, 5 H) 3.45-3.56 (m, 1 H) 3.36-3.44 (m, |
| | | 1 H) 2.55-2.84 (m, 2 H) 2.39 (br d, J-5.43 Hz, 6 H) 2.14-2.26 (m, 2 |
| | | H) 0.74-0.86 (m, 3 H). |
| 72 | 631.0 | ¹H NMR (400 MHz, METHANOL-d₄) δ ppm 9.17-9.28 (m, 1 H) |
| | | 7.69 (dd, J = 9.09, 5.54 Hz, 1 H) 7.34 (d, J = 2.72 Hz, 1 H) 7.21-7.30 |
| | | (m, 1 H) 7.03-7.12 (m, 1 H) 5.49-5.69 (m, 1 H) 4.83 (s, 2 H) 4.64- |
| | | 4.76 (m, 4 H) 3.72-4.24 (m, 5 H) 3.35-3.56 (m, 2 H) 2.52-2.84 (m, |
| | | 3 H) 2.30-2.52 (m, 4 H) 2.02-2.28 (m, 3 H) 0.74-0.86 (m, 3 H). |

### 6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol (Example 73)

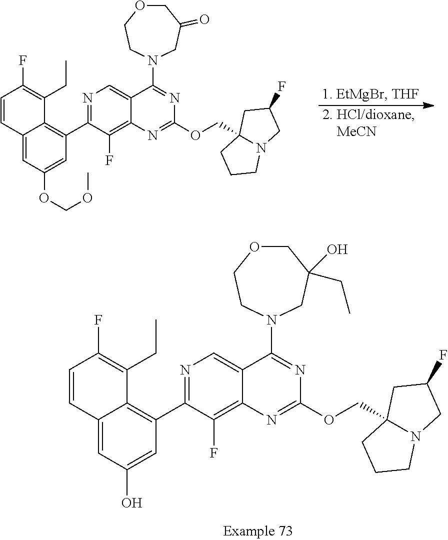 [SMILES: CCc1c(F)ccc2cc(OCOC)cc(-c3ncc4c(N5CCOCC(=O)C5)nc(OC[C@@]56CCCN5C[C@H](F)C6)nc4c3F)c12 ; "1. EtMgBr, THF" ; "2. HCl/dioxane,    MeCN" ; CCc1c(F)ccc2cc(O)cc(-c3ncc4c(N5CCOCC(O)(CC)C5)nc(OC[C@@]56CCCN5C[C@H](F)C6)nc4c3F)c12]

To a 10-mL round-bottomed flask was added 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one (20 mg, 0.03 mmol, synthesized en route to Example 67) in THF (0.6 mL). The reaction was cooled to 0° C. before ethylmagnesium bromide (1 M in THF, 0.09 mL, 0.09 mmol, Sigma-Aldrich Corporation) was added. The reaction was stirred for 1 h. The crude material was quenched with MeOH and acetic acid. The crude mixture was purified by column chromatography on silica gel, eluting with a gradient of 0-100% 3:1 EtOAc/EtOH with 1% TEA in heptane, to provide 6-ethyl-4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol, which was dissolved in 2 mL of acetonitrile. The mixture was cooled to 0° C. before HCl (4 M in dioxane, 0.4 mL) was added. The reaction was stirred for 1 h. The mixture was concentrated under reduced pressure and then purified by reverse phase HPLC to give 6-ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol as 2,2,2-trifluoroacetate and as off-white solid (8.0 mg, 0.01 mmol, 35% yield). m/z (ESI): 638.2 (M+H)⁺. ¹H NMR (400 MHz, METHANOL-d₄) δ ppm 9.57-9.87 (m, 1H), 7.60-8.01 (m, 1H), 7.17-7.45 (m, 2H), 6.86-7.14 (m, 1H), 5.45-5.83 (m, 1H), 4.42-4.81 (m, 4H), 3.61-4.34 (m, 8H), 3.47-3.58 (m, 1H), 2.31-2.82 (m, 6H), 2.11-2.27 (m, 2H), 1.46-1.80 (m, 2H), 1.00-1.19 (m, 2H), 0.72-0.93 (m, 3H).

### 6-Cyclopropyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol (Example 74)

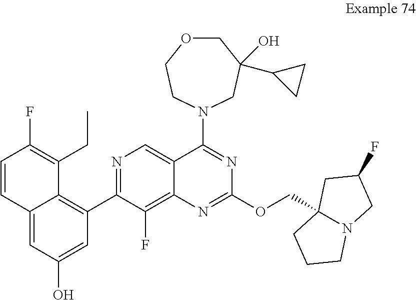 [SMILES: CCc1c(F)ccc2cc(O)cc(-c3ncc4c(N5CCOCC(O)(C6CC6)C5)nc(OC[C@@]56CCCN5C[C@H](F)C6)nc4c3F)c12]

Synthesized in an analogous manner to Example 73, using cyclopropylmagnesium bromide solution in THF. The product was isolated as TFA salt. m/z (ESI): 650.2 (M+H)⁺. ¹H NMR (400 MHz, METHANOL-d₄) δ ppm 9.56-9.79 (m, 1H), 7.51-7.86 (m, 1H), 7.18-7.44 (m, 2H), 6.94-7.12 (m, 1H), 5.22-6.00 (m, 1H), 4.56-4.74 (m, 4H), 4.18-4.34 (m, 1H), 3.80-4.11 (m, 7H), 3.64-3.80 (m, 1H), 3.46-3.59 (m, 1H), 2.05-2.91 (m, 8H), 0.91-1.12 (m, 1H), 0.84 (s, 3H), 0.51-0.69 (m, 2H), 0.33-0.50 (m, 2H).

### 4-(4-(2-(Difluoromethyl)morpholino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol (Example 75)

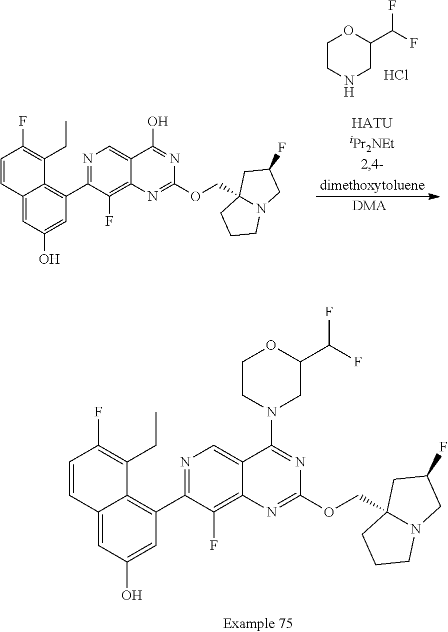

7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (20 mg, 0.04 mmol, Intermediate B) was dissolved in N,N-dimethylacetamide (0.50 mL). HATU (60 mg, 0.16 mmol, Combi-Blocks Inc.), DIPEA (25 mg, 34 μL, 0.20 mmol, Sigma-Aldrich Corporation), 2,4-dimethoxytoluene (12 mg, 12 μL, 0.08 mmol, Aurum Pharmatech LLC) and 2-(difluoromethyl)morpholin-4-ium chloride (11 mg, 0.06 mmol, Enamine) were added and the mixture was stirred at rt overnight. Water (0.5 mL) was then added and the mixture was stirred overnight. The crude reaction mixture was purified with 0.1% formic acid in H₂O and MeCN as mobile phase, XSelect column (19×100 mm, 5 μm), MS mode: ESI⁺ to yield 4-(4-(2-(difluoromethyl)morpholino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol (5.3 mg, 7.8 mol, 20% yield) as formate. m/z (ESI): 630.2 (M+H)⁺. ¹H NMR (500 MHz, DMSO-d₆) δ ppm 9.95 (s, 1H), 9.24 (s, 1H), 7.77 (dd, J=6.4, 3.1 Hz, 2H), 7.34 (d, J=2.8 Hz, 3H), 7.00-7.03 (m, 1H), 7.01 (d, J=2.3 Hz, 1H), 6.10-6.37 (m, 1H), 5.45-5.65 (m, 1H), 4.42-4.69 (m, 5H), 4.06-4.17 (m, 3H), 3.52-3.90 (m, 5H), 2.01-2.39 (m, 7H), 0.71-0.75 (m, 4H).

### 4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)morpholine-2-carboxamide (Example 76)

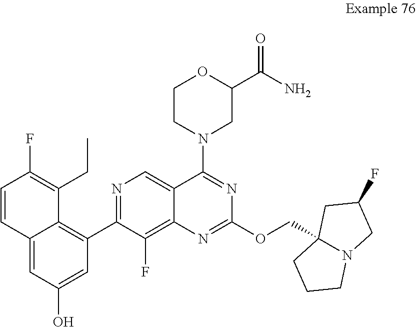 [SMILES: CCc1c(F)ccc2cc(O)cc(-c3ncc4c(N5CCOC(C(N)=O)C5)nc(OC[C@@]56CCCN5C[C@H](F)C6)nc4c3F)c12]

Synthesized in an analogous manner to Example 75, using morpholine-2-carboxamide (CAS #: 135072-13-8, Enamine). Purification was performed with 0.1% TFA in H₂O and MeCN as mobile phase, XSelect column (19×100 mm, 5 μm). m/z (ESI): 623.2 (M+H)⁺. ¹H NMR (500 MHz, DMSO-d₆) δ ppm 9.29 (d, J=1.3 Hz, 1H), 7.75 (ddt, J=6.4, 3.3, 1.0, 1.0 Hz, 1H), 7.43-7.52 (m, 2H), 7.33-7.40 (m, 2H), 7.00-7.04 (m, 1H), 5.47-5.64 (m, 1H), 4.54-4.69 (m, 3H), 4.21-4.37 (m, 2H), 4.04-4.12 (m, 1H), 3.61-3.93 (m, 6H), 2.00-2.39 (m, 6H), 0.73 (t, J=7.4 Hz, 3H).

### 5-Ethyl-6-fluoro-4-(8-fluoro-2-((1-(hydroxymethyl)cyclopropyl)methoxy)-4-(1,4-oxazepan-4-yl)pyrido[4,3-d]pyrimidin-7-yl)naphthalen-2-ol (Example 77)

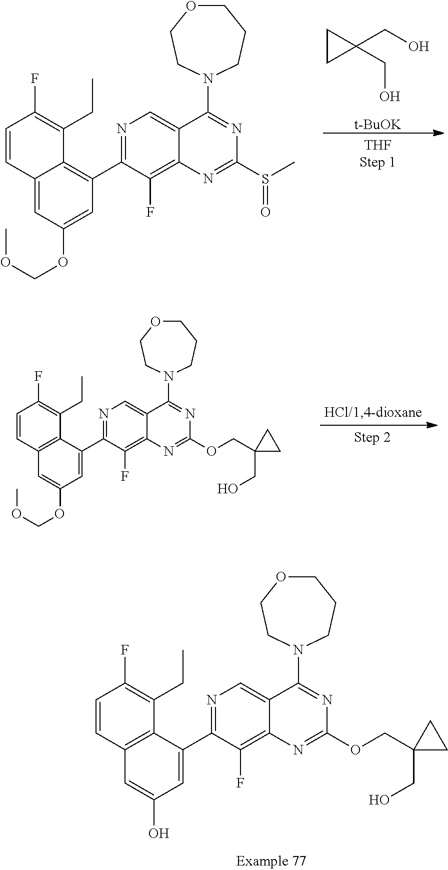 [SMILES: "t-BuOK" ; CCc1c(F)ccc2cc(OCOC)cc(-c3ncc4c(N5CCCOCC5)nc(S(C)=O)nc4c3F)c12 ; OCC1(CO)CC1 ; CCc1c(F)ccc2cc(OCOC)cc(-c3ncc4c(N5CCCOCC5)nc(OCC5(CO)CC5)nc4c3F)c12 ; CCc1c(F)ccc2cc(O)cc(-c3ncc4c(N5CCCOCC5)nc(OCC5(CO)CC5)nc4c3F)c12]

Step 1: (1-(((7-(8-Ethyl-7-fluoro-3-(methoxymethoxy)naphthalen-1-yl)-8-fluoro-4-(1,4-oxazepan-4-yl)pyrido[4,3-d]pyrimidin-2-yl)oxy)methyl)cyclopropyl)methanol. To a 20 mL vial was added [1-(hydroxymethyl)cyclopropyl]methanol (11 mg, 0.11 mmol, Enamine) and potassium tert-butoxide (16 mg, 16 μL, 0.14 mmol, AK Scientific, Inc.) in tetrahydrofuran (0.3 mL) at 0° C. The reaction was stirred for 10 min. Then, 4-(7-(8-ethyl-7-fluoro-3-(methoxymethoxy)naphthalen-1-yl)-8-fluoro-2-(methylsulfinyl)pyrido[4,3-d]pyrimidin-4-yl)-1,4-oxazepane (50 mg, 0.09 mmol, Intermediate L) was transferred and the reaction was stirred at 0° C. until completion. The reaction was carefully quenched with water. The solution was transferred to a separatory funnel and extracted three times with ethyl acetate. The combined organic layers were dried over Na₂SO₄. The resulting solution was filtered, and concentrated in vacuo to afford the crude product. The resulting crude was purified by column chromatography on silica gel, eluting with a gradient of 5-10% MeOH (with 10% 2 M NH₃) in DCM to afford (1-(((7-(8-ethyl-7-fluoro-3-(methoxymethoxy)naphthalen-1-yl)-8-fluoro-4-(1,4-oxazepan-4-yl)pyrido[4,3-d]pyrimidin-2-yl)oxy)methyl)cyclopropyl)methanol (31 mg, 0.05 mmol, 58% yield) as yellow solid. m/z (ESI): 581.0 (M+H)⁺.

Step 2: 5-Ethyl-6-fluoro-4-(8-fluoro-2-((1-(hydroxymethyl)cyclopropyl)methoxy)-4-(1,4-oxazepan-4-yl)pyrido[4,3-d]pyrimidin-7-yl)naphthalen-2-ol. (1-(((7-(8-Ethyl-7-fluoro-3-(methoxymethoxy)naphthalen-1-yl)-8-fluoro-4-(1,4-oxazepan-4-yl)pyrido[4,3-d]pyrimidin-2-yl)oxy)methyl)cyclopropyl)methanol (31 mg, 0.05 mmol) was dissolved in MeCN (2 mL) and HCl in dioxane (4 M, 0.23 mL, 0.92 mmol, Sigma-Aldrich Corporation) was added. The reaction was stirred at 0° C. Upon completion, the reaction was cooled to room temperature and concentrated under reduced pressure to provide a crude mixture. The crude product was then purified by reverse phase HPLC to provide 5-ethyl-6-fluoro-4-(8-fluoro-2-((1-(hydroxymethyl)cyclopropyl)methoxy)-4-(1,4-oxazepan-4-yl)pyrido[4,3-d]pyrimidin-7-yl)naphthalen-2-ol (7.0 mg, 0.01 mmol, 14% yield) as light yellow solid. m/z (ESI): 537.2 (M+H)⁺. ¹H NMR (600 MHz, DMSO-d₆) δ ppm 9.55-9.62 (m, 1H), 8.18 (dd, J=9.0, 5.9 Hz, 1H), 7.73-7.78 (m, 2H), 7.44 (d, J=2.6 Hz, 1H), 5.06 (t, J=5.6 Hz, 1H), 4.68-4.76 (m, 2H), 4.52-4.62 (m, 4H), 4.36 (t, J=4.7 Hz, 2H), 4.13-4.19 (m, 2H), 3.77-3.84 (m, 3H), 2.92 (dt, J=3.5, 1.9 Hz, 1H), 2.52-2.83 (m, 2H), 1.15 (t, J=7.4 Hz, 3H), 0.90-0.99 (m, 4H).

### Biological Evaluation

Provided in this section is the biological evaluation of the specific examples provided herein.

### KRAS G12D TR-FRET Assay

Compounds of interest were prepared in a dose-response titration in DMSO, and 80 nL were added via Labcyte Echo to each well of a 384-well plate (Perkin Elmer 6008280). The His-tagged KRAS G12D protein (Amgen) was diluted to 20 nM in Assay Buffer (20 mM HEPES, pH 7.4, 10 mM MgCl₂, 50 mM NaCl, 0.1% BSA, 0.01% Tween-20, 10 M GDP) and 2 uL was added to the appropriate wells of the 384-well plate. The plate was incubated for 30 minutes at room temperature. Biotinylated KRPep-2d substrate (Amgen) was diluted to 20 nM in Assay Buffer and 2 L was added to all wells and incubated for 1 hour at room temperature. Detection Reagent (0.4 nM LANCE Eu-W1024 Anti-6xHis (Perkin Elmer AD0401), 5 nM streptavidin-d² (Cisbio 610SADLA)) was prepared in Assay Buffer, then 4 μL was added to the plate and incubated for 1 hour at room temperature. Plates were read using PerkinElmer EnVision (ex: 320 nm, em1: 665 nm, em2: 615 nm) and em1/em2 data was used to generate curve fits using a 4-parameter logistic model to calculate IC₅₀ values.

### KRAS G12D Coupled Nucleotide Exchange Assay

Purified GDP-bound KRAS protein (aa 1-169), containing both G12D and C118A amino acid substitutions and an N-terminal His-tag, was pre-incubated in assay buffer (25 mM HEPES pH 7.4, 10 mM MgCl₂, and 0.01% Triton X-100) with a compound dose-response titration for 2 hours. Following compound pre-incubation, purified SOS protein (aa 564-1049) and GTP (Roche 10106399001) were added to the assay wells and incubated for an additional 30 min. To determine the extent of inhibition of SOS-mediated nucleotide exchange, purified GST-tagged cRAF (aa 1-149), nickel chelate AlphaLISA acceptor beads (PerkinElmer AL108R), and AlphaScreen glutathione donor beads (PerkinElmer 6765302) were added to the assay wells and incubated for 10 minutes. The assay plates were then read on a PerkinElmer EnVision Multilabel Reader, using AlphaScreen® technology, and data were analyzed using a 4-parameter logistic model to calculate IC₅₀ values.

### Phospho-ERK1/2 MSD Assay

AsPC-1 (ATCC® CRL-1682™) cells were cultured in RPMI 1640 Medium (ThermoFisher Scientific 11875093) containing 10% fetal bovine serum (ThermoFisher Scientific 16000044) and 1× penicillin-streptomycin-glutamine (ThermoFisher Scientific 10378016). Sixteen hours prior to compound treatment, AsPC-1 cells were seeded in 96-well cell culture plates at a density of 25,000 cells/well and incubated at 37° C., 5% CO₂. A compound dose-response titration was diluted in growth media, added to appropriate wells of a cell culture plate, and then incubated at 37° C., 5% CO₂ for 2 hours. Following compound treatment, cells were washed with ice-cold Dulbecco's phosphate-buffered saline, no Ca²⁺ or Mg²⁺ (ThermoFisher Scientific 14190144), and then lysed in RIPA buffer (50 mM Tris-HCl pH 7.5, 1% Igepal, 0.50 sodium deoxycholate, 150 mM NaCl, and 0.50 sodium dodecyl sulfate) containing protease inhibitors (Roche 4693132001) and phosphatase inhibitors (Roche 4906837001). Phosphorylation of ERK1/2 in compound-treated lysates was assayed using Phospho-ERK1/2 Whole Cell Lysate kits (Meso Scale Discovery K151DWD) according to the manufacturer's protocol. Assay plates were read on a Meso Scale Discovery Sector Imager 6000, and data were analyzed using a 4-parameter logistic model to calculate IC₅₀ values.

TABLE 10
Biochemical and cellular activity of examples.

| | KRAS G12D | KRAS G12D | 2 h p-ERK |
|---|---|---|---|
| Ex. | Binding | Coupled Exchange | (AsPC-1 cells), |
| # | IC₅₀ (μM) | IC₅₀ (μM) | IC₅₀ (μM) |
| 1 | 0.003 | 0.002 | 0.016 |
| 2 | 0.001 | 0.001 | 0.033 |
| 3 | 0.002 | 0.001 | 0.047 |
| 4 | 0.004 | 0.005 | 0.486 |
| 5 | 0.002 | 0.002 | 0.088 |
| 6 | 0.069 | 0.03 | 1.77 |
| 7 | 0.002 | 0.001 | 0.046 |
| 8 | 0.005 | 0.004 | 0.103 |
| 9 | 0.008 | 0.004 | 0.353 |
| 10 | 0.008 | 0.006 | 0.277 |
| 11 | 0.039 | 0.02 | >10 |
| 12 | 0.004 | 0.005 | 0.131 |
| 13 | 0.011 | 0.005 | 4.18 |
| 14 | 0.011 | 0.006 | 0.794 |
| 15 | 0.036 | 0.025 | 4.51 |
| 16 | 0.073 | 0.05 | 4.83 |
| 17 | 0.003 | 0.002 | 0.189 |
| 18 | 0.002 | 0.002 | 0.162 |
| 19 | 0.009 | 0.005 | 1.1 |
| 20 | 0.005 | 0.005 | 0.336 |
| 21 | 0.011 | 0.007 | 0.621 |
| 22 | 0.011 | 0.01 | 1.29 |
| 23 | 0.029 | 0.014 | 1.15 |
| 24 | 0.011 | 0.004 | 0.586 |
| 25 | 0.003 | 0.004 | 0.292 |
| 26 | 0.017 | 0.01 | 2.27 |
| 27 | 0.062 | 0.037 | 2.28 |
| 28 | 0.079 | 0.038 | 1.77 |
| 29 | 0.012 | 0.007 | NT |
| 30 | 0.037 | 0.018 | NT |
| 31 | 0.011 | 0.015 | NT |
| 32 | 0.013 | 0.004 | 0.966 |
| 33 | 0.027 | 0.034 | NT |
| 34 | 0.001 | 0.002 | 0.393 |
| 35 | 0.057 | 0.039 | >10 |
| 36 | 0.001 | 0.002 | 0.651 |
| 37 | 0.011 | 0.008 | 2.66 |
| 38 | 0.021 | 0.011 | >10 |
| 39 | 0.005 | 0.004 | 0.171 |
| 40 | 0.006 | 0.006 | NT |
| 41 | 0.002 | 0.002 | 0.035 |
| 42 | 0.002 | 0.004 | NT |
| 43 | 0.249 | 0.33 | NT |
| 44 | 0.026 | 0.029 | NT |
| 45 | 0.002 | 0.004 | 0.427 |
| 46 | 0.017 | 0.012 | NT |
| 47 | 0.003 | 0.006 | 1.47 |
| 48 | 0.01 | 0.012 | 1.05 |
| 49 | 0.013 | 0.013 | 0.889 |
| 50 | 0 | 0.001 | 0.07 |
| 51 | 0.006 | 0.01 | 6.23 |
| 52 | 0.033 | 0.031 | 1.28 |
| 53 | 0.039 | 0.026 | 1.24 |
| 54 | 0.007 | 0.009 | NT |
| 55 | 0.007 | 0.004 | NT |
| 56 | 0.003 | 0.007 | 0.081 |
| 57 | 0.013 | 0.018 | 5.95 |
| 58 | 0.014 | 0.011 | NT |
| 59 | 0.014 | 0.009 | 3.63 |
| 60 | 0.015 | 0.009 | NT |
| 61 | 0.003 | 0.003 | 0.193 |
| 62 | 0.001 | 0.002 | 0.07 |
| 63 | 0.005 | 0.005 | 1.38 |
| 64 | 0.044 | 0.03 | NT |
| 65 | 0.043 | 0.024 | NT |
| 66 | 0.015 | 0.016 | 0.592 |
| 67 | 0.002 | 0.002 | 0.072 |
| 68 | 0.024 | 0.03 | NT |
| 69 | 0.002 | 0.002 | 0.004 |
| 70 | 0.001 | 0.001 | 0.643 |
| 71 | 0.012 | 0.01 | NT |
| 72 | 0.006 | 0.008 | 0.308 |
| 73 | 0.003 | 0.006 | 0.063 |
| 74 | 0.004 | 0.004 | 0.436 |
| 75 | 0.052 | 0.029 | NT |
| 76 | 0.006 | 0.005 | 3.33 |
| 77 | 0.041 | 0.033 | NT |
| 78 | 0.001 | 0.001 | 0.013 |
| 79 | 0.001 | 0.001 | 0.024 |
| 80 | 0.004 | 0.002 | 0.024 |

NT: not tested.

### REFERENCES

All references, for example, a scientific publication or patent application publication, cited herein are incorporated herein by reference in their entirety and for all purposes to the same extent as if each reference was specifically and individually indicated to be incorporated by reference in its entirety for all purposes.

What is claimed is:

1. A compound of formula (I): 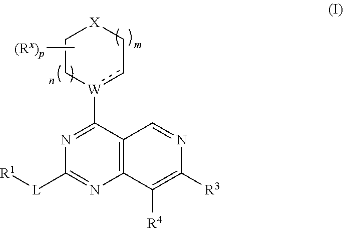
or a pharmaceutically acceptable salt of said compound, wherein; [symbol]
is a single bond or a double bond;
W is C, CH or N, wherein when W is CH or N, [symbol] is a single bond;
X is O, S, S(O), S(O)(NR^{z}) or S(O)₂;
n is 0, 1, 2, or 3;
m is 0, 1, 2 or 3;
p is 0, 1, 2 or 3;
each R^{x} is hydroxyl, halogen, oxo, cyano, —N(R^{z})₂, C₁₋₄ alkyl, C₁₋₄ alkoxy, C₁₋₄ haloalkyl, C₁₋₄ haloalkoxy, C₃₋₆ cycloalkyl, 5-7 membered heteroaryl, -T-R^{y} or two R^{x} taken together with adjacent carbon atoms can form a C₃₋₇ cycloalkyl or 5-7 membered heterocycloalkyl, wherein each C₃₋₇ cycloalkyl or 5-7 membered heterocycloalkyl is further substituted with 0-3 occurrences of R^{y} or two R^{x} taken together can form a bridged ring where the bridge is selected from one of the following: —C₁₋₄ alkylene, —C₁₋₄ alkylene-O—C₁₋₄ alkylene-, —O—, —S— or —C₁₋₄ alkylene-S—C₁₋₄ alkylene- and wherein each C₁₋₄ alkylene is further substituted with 0-2 occurrences of R^{y};
L is C₁₋₆ alkylene, —O—C₁₋₆ alkylene, —S—C₁₋₆ alkylene, NR^{z}, O or S, wherein each C₁₋₆ alkylene, —O—C₁₋₆ alkylene and —S—C₁₋₆ alkylene chain is substituted with 0-2 occurrences of R²;
R¹ is hydroxyl, aryl, heteroaryl, C₃₋₈ cycloalkyl or heterocycloalkyl substituted with 0-3 occurrences of R⁵;
R² is halogen, hydroxyl, C₁₋₄ alkyl or two R² on the same or adjacent carbon atoms can be taken together to form a C₃₋₇ cycloalkyl;
R³ is aryl or heteroaryl substituted with 0-3 occurrences of R⁶;
R⁴ is hydrogen, hydroxyl, halogen, C₁₋₄ alkyl, C₁₋₄ alkoxy, C₁₋₄ haloalkyl, C₂₋₄ alkenyl, C₂₋₄ alkynyl, C₃₋₇ cycloalkyl or cyano;
each R⁵ is halogen, oxo, hydroxyl, amino, cyano or C₁₋₄ alkyl;
each R⁶ is halogen, hydroxyl, cyano, —N(R^{z})₂, C₁₋₄ alkyl, C₁₋₄ alkoxy, C₁₋₄ haloalkyl, C₁₋₄ haloalkoxy, C₂₋₄ alkynyl or C₃₋₆ cycloalkyl;
T is C₁₋₄ alkylene, —S(O)₂—, —C(O)—, —C₁₋₄ alkylene-C(O)—, C₁₋₄ alkylene-S(O)₂— or —S—;
R^{y} is halogen, C₁₋₄ alkyl, C₁₋₄ haloalkyl, C₁₋₄ alkoxy, hydroxyl, cyano or —N(R)₂; and
R_{z} is hydrogen or C₁₋₄ alkyl, wherein when
X is O, R³ is aryl substituted with one occurrence of R⁶ and R⁶ is hydroxyl, then p is not zero.

2. The compound of claim 1, wherein when R³ is 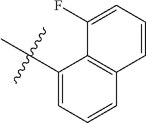 [SMILES: *c1cccc2cccc(F)c12]
and R⁴ is fluorine, then 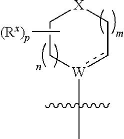
is not

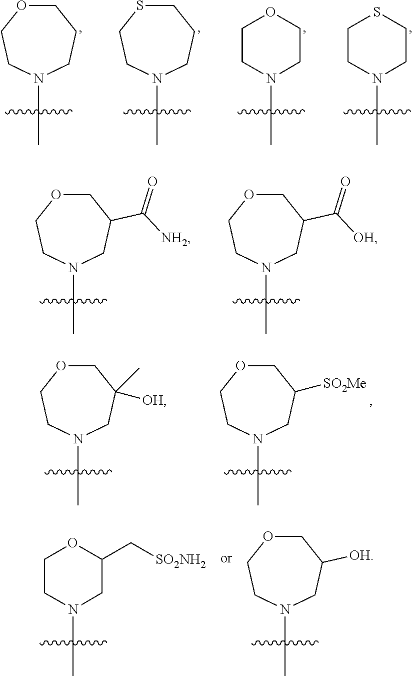

3. The compound of claim 1, wherein when R³ is 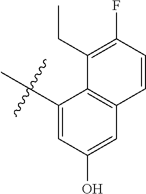 [SMILES: *c1cc(O)cc2ccc(F)c(CC)c12]
and R⁴ is fluorine, then 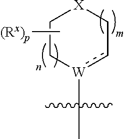
is not

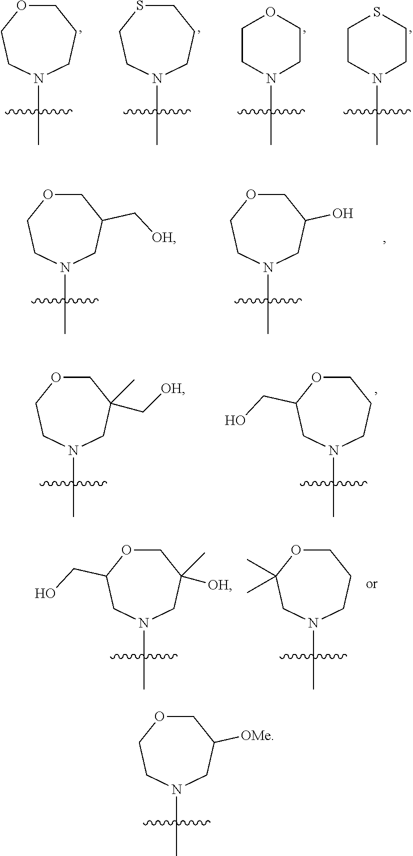

4. The compound of claim 1, wherein when R³ is 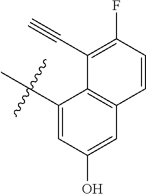 [SMILES: *c1cc(O)cc2ccc(F)c(C#C)c12]
and R⁴ is fluorine, then 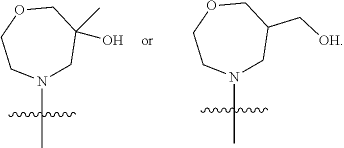
is not

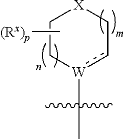

5. The compound of claim 1, wherein when R³ is 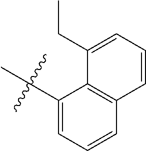 [SMILES: *c1cccc2cccc(CC)c12]
and R⁴ is fluorine, then 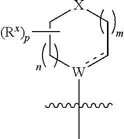
is not

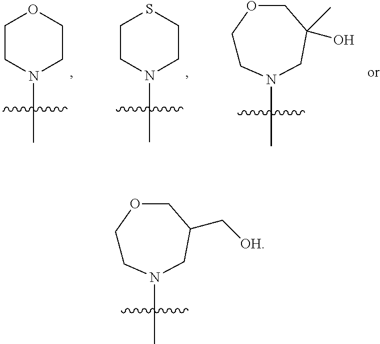

6. The compound of claim 1, wherein when R³ is 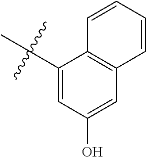 [SMILES: *c1cc(O)cc2ccccc12]
and R⁴ is fluorine, then 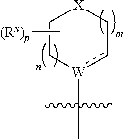
is not

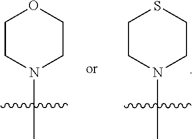 [SMILES: *N1CCOCC1 ; *N1CCSCC1]

7. The compound of claim 1, wherein when R³ is 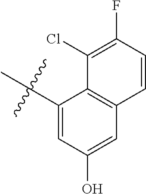 [SMILES: *c1cc(O)cc2ccc(F)c(Cl)c12]
and R⁴ is fluorine, then 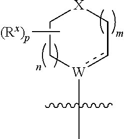
is not

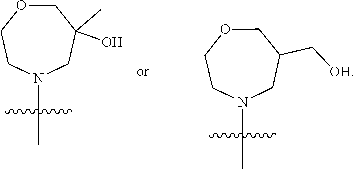

8. The compound of claim 1, wherein when R³ is 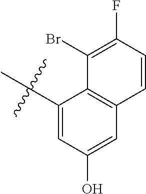 [SMILES: *c1cc(O)cc2ccc(F)c(Br)c12]
and R⁴ is fluorine, then 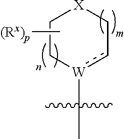
is not

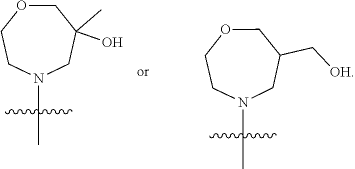

9. The compound of claim 1, wherein when R³ is 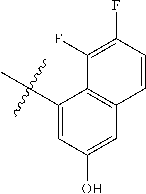 [SMILES: *c1cc(O)cc2ccc(F)c(F)c12]
and R⁴ is fluorine, then 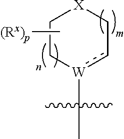
is not

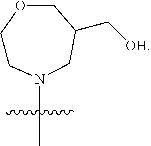 [SMILES: *N1CCOCC(CO)C1]

10. The compound of claim 1, wherein when R³ is 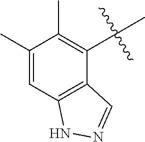 [SMILES: *c1c(C)c(C)cc2[nH]ncc12]
and R⁴ is fluorine, then 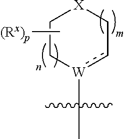
is not

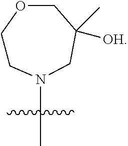 [SMILES: *N1CCOCC(C)(O)C1]

11. The compound of claims 1-10, wherein L is —O—C₁₋₆ alkylene (e.g., —O-methylene-, —O-ethylene- or —O-n-propylene) substituted with 0-2 occurrences of R².

12. The compound of claim 11, wherein L is —O-ethylene or —O-n-propylene substituted with 0-2 occurrences of R².

13. The compound of claim 12, wherein R¹ is hydroxyl or heterocycloalkyl substituted with 0-3 occurrences of R⁵.

14. The compound of any of claims 1-10, wherein R⁵ is halogen, cyano, C₁₋₄ alkyl or oxo.

15. The compound of claim 14, wherein -L-R¹ is

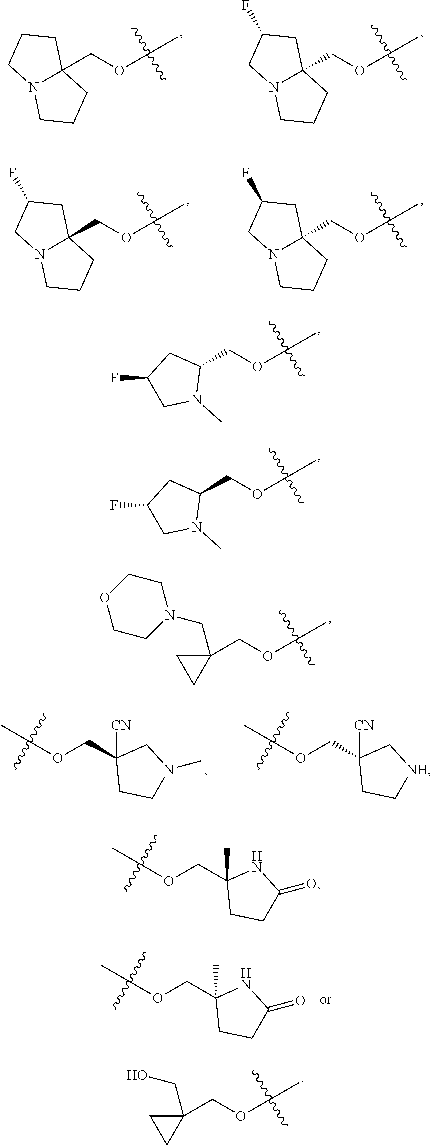

16. The compound of claim 15, wherein -L-R¹ is

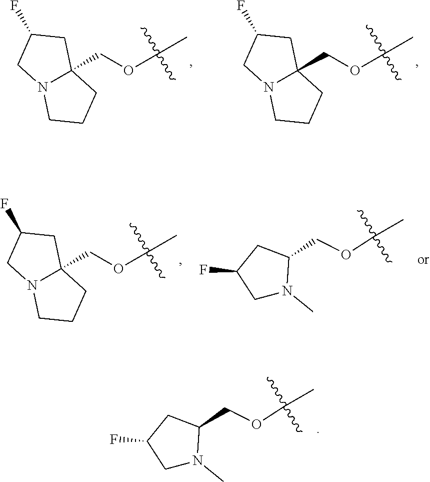

17. The compound of any of claims 1-10, wherein R³ is aryl substituted with 0-3 occurrences of R⁶.

18. The compound of claim 17, wherein R³ is phenyl or naphthyl substituted with 0-3 occurrences of R⁶.

19. The compound of claim 18, wherein R³ is heteroaryl substituted with 0-3 occurrences of R⁶.

20. The compound of claim 18, wherein R⁶ is hydroxyl, halogen, C₁₋₄ alkyl, C₁₋₄ haloalkyl, C₂₋₄ alkynyl, C₃₋₆ cycloalkyl or —N(R^{z})₂.

21. The compound of claim 18, wherein R⁶ is hydroxyl, methyl, ethyl, trifluoromethyl, difluoromethyl, ethynyl, fluorine, chlorine, cyclopropyl or —NH₂.

22. The compound of claim 18, wherein R³ is

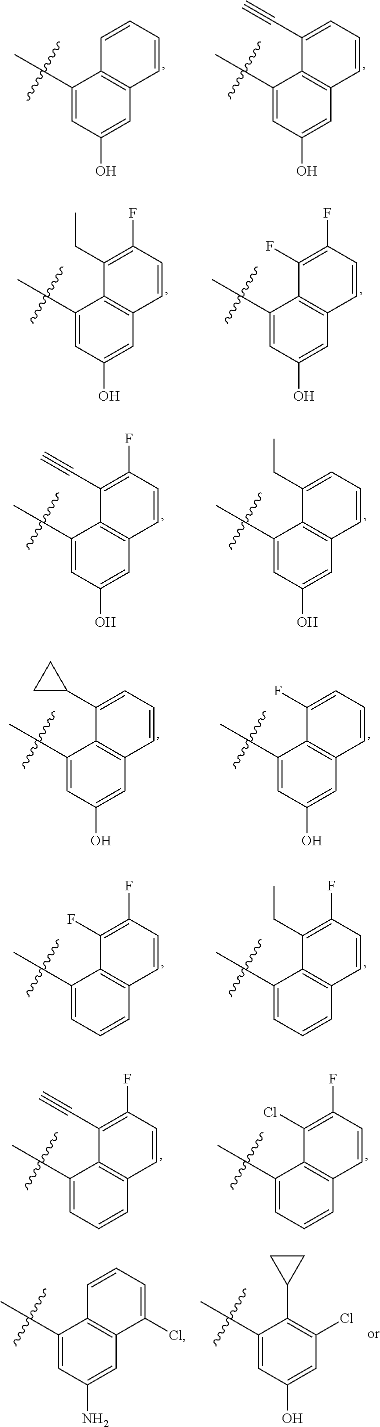

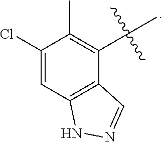 [SMILES: *c1c(C)c(Cl)cc2[nH]ncc12]

23. The compound of claim 22, wherein R³ is

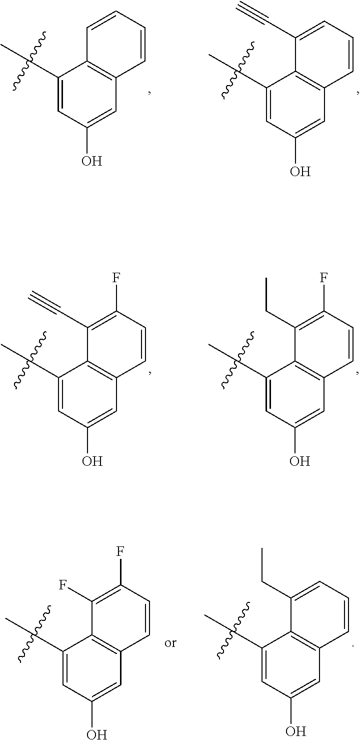 [SMILES: *c1cc(O)cc2ccccc12 ; *c1cc(O)cc2cccc(C#C)c12 ; *c1cc(O)cc2ccc(F)c(C#C)c12 ; *c1cc(O)cc2ccc(F)c(CC)c12 ; *c1cc(O)cc2ccc(F)c(F)c12 ; *c1cc(O)cc2cccc(CC)c12]

24. The compound of any of claims 1-10, wherein W is N and [symbol] is a single bond.

25. The compound of any of claims 1-10, wherein X is S, S(O)₂, S(O) or S(O)(NR^{z}).

26. The compound of claim 25, wherein p is 0, 1 or 2.

27. The compound of claim 25, wherein each R^{x} is -T-Ry or two R^{x} are taken together to form a bridged ring wherein the bridge is —C₁₋₄ alkylene further substituted with 0-2 occurrences of R^{y}.

28. The compound of claim 27, wherein each R^{x} is —CH₂OH or two R^{x} are taken together to form a bridged ring wherein the bridge is methylene or ethylene further substituted with 0-2 occurrences of R^{y}.

29. The compound of claim 25, wherein 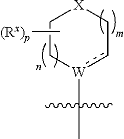
is

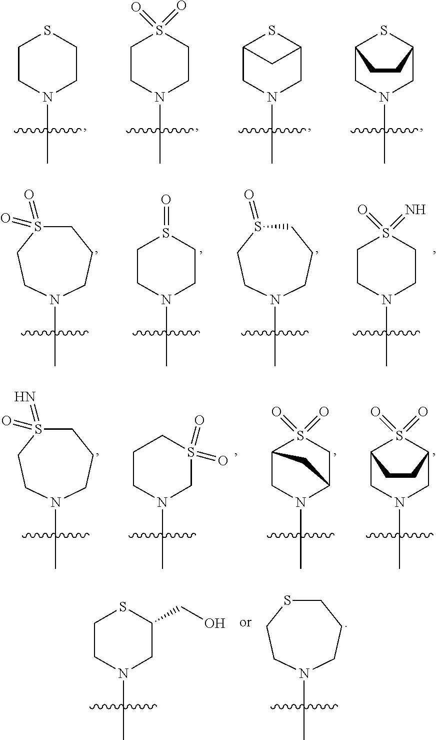

30. The compound of any of claims 1-10, wherein X is O.

31. The compound of claim 30, wherein n is 1 and m is 1.

32. The compound of claim 31, wherein each R^{x} is C₁₋₄ alkyl, C₁₋₄ haloalkyl, oxo or -T-R^{y} or two R^{x} taken together form a bridged ring wherein the bridge is selected from —C₁₋₄ alkylene further substituted with 0-2 occurrences of R^{y} or two R^{x} taken together with adjacent carbon atoms to form a C₃₋₇ cycloalkyl further substituted with 0-3 occurrences of R^{y}.

33. The compound of claim 32, wherein each R^{x} is methyl, difluoromethyl, —CH₂CN, CH₂OH, —C(O)NH₂ or —CH₂OMe or two R^{x} taken together form a bridged ring wherein the bridge is selected from methylene or ethylene further substituted with 0-2 occurrences of R^{y} or two R^{x} taken together with adjacent carbon atoms form a cyclopropyl further substituted with 0-3 occurrences of R^{y}.

34. The compound of claim 31, wherein 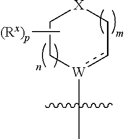
is

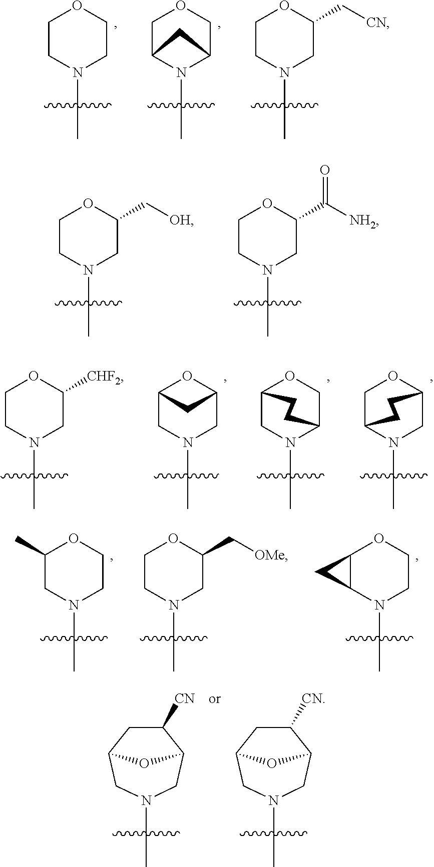

35. The compound of claim 30, wherein n is 1 and m is 2 or n is 2 and m is 2.

36. The compound of claim 35, wherein each R^{x} is oxo, C₁₋₄ alkyl, C₁₋₄ alkoxy, C₂₋₄ alkynyl, C₃₋₆ cycloalkyl, hydroxy, halogen, cyano or -T-R^{y} or two R^{x} taken together form a bridged ring wherein the bridge is —O— or —C₁₋₄ alkylene wherein the —C₁₋₄ alkylene is further substituted with 0-2 occurrences of R^{y}.

37. The compound of claim 36, wherein each R^{x} is methyl, ethyl, ethynyl, fluorine, cyclopropyl, cyano, oxo, hydroxy, methoxy, —C(O)N(H)(Me), —C(O)NH₂, —CH₂OH or —SO₂NH₂ or two R^{x} taken together form a bridged ring wherein the bridge is —O— or methylene wherein methylene is further substituted with 0-2 occurrences of R^{y}.

38. The compound of claim 35, wherein 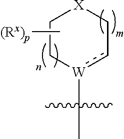
is

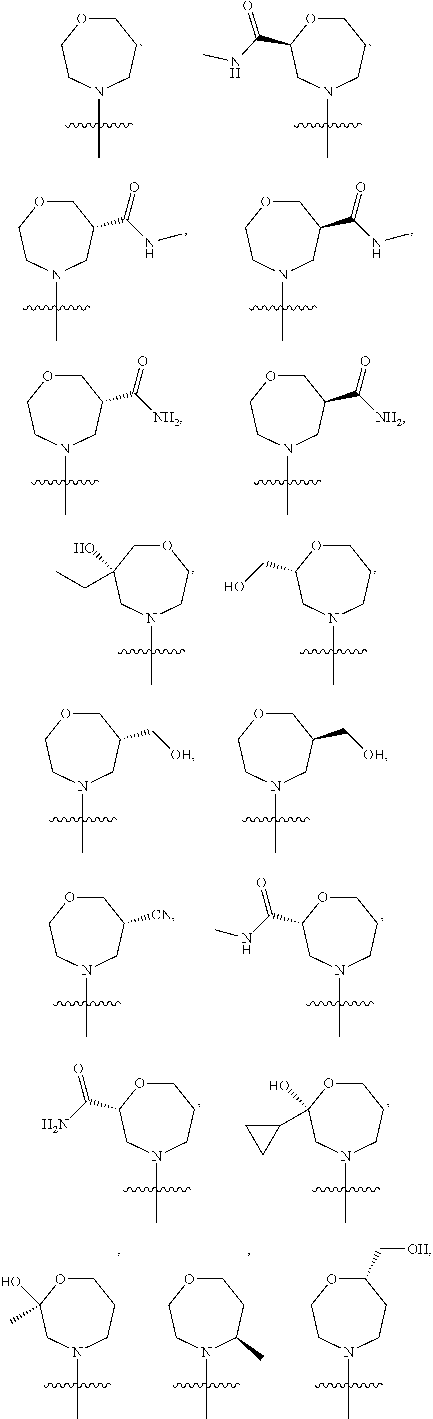

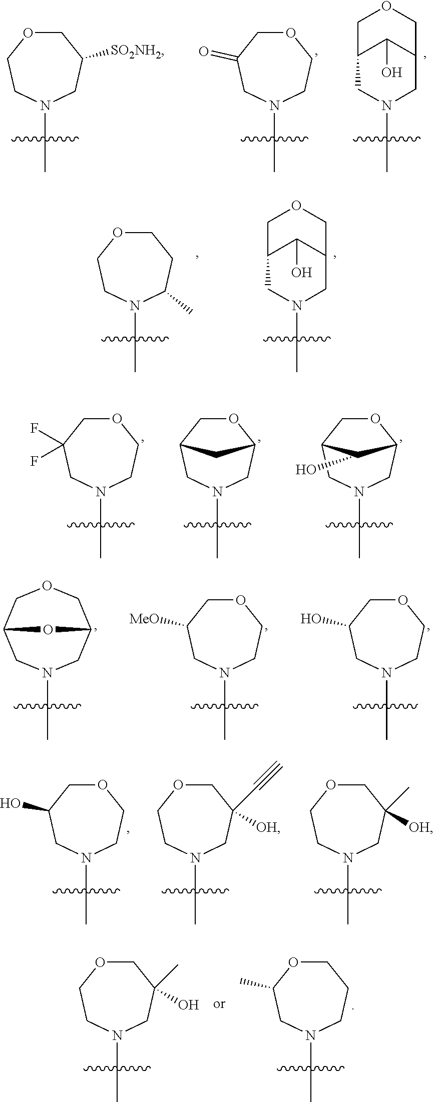

39. The compound of any one of claims 1-10, wherein R⁴ is C₁₋₄ alkyl, C₁₋₄ alkoxy, hydroxyl, halogen or C₁₋₄ haloalkyl.

40. The compound of claim 39, wherein R⁴ is C₁₋₄ alkyl, hydroxyl or halogen.

41. The compound of claim 1, wherein the compound is selected from one of the following compounds:
5-Ethyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-ethyl-6-fluoro-4-(8-fluoro-2-(((2R,7aR)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide;
4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one;
5-Ethyl-6-fluoro-4-(8-fluoro-2-(((2R,7aS)-2-fluorotetrahydro-1H-pyrrolizin-7a(5H)-yl)methoxy)-4-(1,4-thiazepan-4-yl)pyrido[4,3-d]pyrimidin-7-yl)naphthalen-2-ol;
5,6-Difluoro-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
5-Ethynyl-4-(8-fluoro-2-(((2S,4R)-4-fluoro-1-methylpyrrolidin-2-yl)methoxy)-4-(1,4-oxazepan-4-yl)pyrido[4,3-d]pyrimidin-7-yl)naphthalen-2-ol;
3-Chloro-4-cyclopropyl-5-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)phenol;
5-Ethyl-6-fluoro-4-(8-fluoro-2-(((2R,7aS)-2-fluorotetrahydro-1H-pyrrolizin-7a(5H)-yl)methoxy)-4-((S)-6-methoxy-1,4-oxazepan-4-yl)pyrido[4,3-d]pyrimidin-7-yl)naphthalen-2-ol;
5-Ethyl-6-fluoro-4-(8-fluoro-2-((1-(morpholinomethyl)cyclopropyl)methoxy)-4-(1,4-oxazepan-4-yl)pyrido[4,3-d]pyrimidin-7-yl)naphthalen-2-ol;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carbonitrile;
4-(4-(6-Oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol; or
(S)-4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol.

42. The compound of claim 1, wherein the compound is selected from one of the following compounds:
5-Ethyl-6-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-Ethyl-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
5-ethyl-6-fluoro-4-(8-fluoro-2-(((2R,7aR)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
4-(4-(6,6-Difluoro-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol;
5,6-Difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)naphthalen-2-ol;
6-Ethyl-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol;
5-Ethyl-6-fluoro-4-(8-fluoro-2-(((2R,7aS)-2-fluorotetrahydro-1H-pyrrolizin-7a(5H)-yl)methoxy)-4-((R)-6-(hydroxymethyl)-1,4-oxazepan-4-yl)pyrido[4,3-d]pyrimidin-7-yl)naphthalen-2-ol (Isomer 2);
3-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,3-thiazinane 1,1-dioxide;
or 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-one.

43. A pharmaceutical composition comprising the compound according to any one of claims 1-42 or a pharmaceutically acceptable salt of said compound, and a pharmaceutically acceptable excipient.

44. A compound according to any one of claims 1-42, or a tautomer thereof, or a pharmaceutically acceptable salt of said compound, or the pharmaceutical composition according to claim 43 for use as a medicament.

45. A compound according to any one of claims 1-42 or a pharmaceutically acceptable salt thereof, or the pharmaceutical composition according to claim 43 for use in treating cancer.

46. A compound according to any one of claims 1-42 or a pharmaceutically acceptable salt thereof, or the pharmaceutical composition according to claim 43 for use in treating cancer, wherein one or more cells express KRAS G12D mutant protein.

47. The compound or pharmaceutical composition for use of claims 45 or 45, wherein the cancer is pancreatic cancer, colorectal cancer, non-small cell lung cancer, small bowel cancer, appendiceal cancer, cancer of unknown primary, endometrial cancer, mixed cancer types, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

48. A use of the compound according to any one of claims 1-42 or a pharmaceutically acceptable salt thereof, or the pharmaceutical composition according to claim 43 in the preparation of a medicament for treating cancer.

49. A use of the compound according to any one of claims 1-42 or a pharmaceutically acceptable salt thereof, or the pharmaceutical composition according to claim 43 in the preparation of a medicament for treating cancer, wherein one or more cells express KRAS G12D mutant protein.

50. The use according to claim 48 or 49, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

51. A method of treating cancer in a subject in need thereof, the method comprising administering to the subject a therapeutically effective amount of the compound according to any one of to any one of claims 1-42 or a pharmaceutically acceptable salt thereof or a pharmaceutical composition according to claim 43.

52. A method of treating cancer in a subject in need thereof, the method comprising administering to the subject a therapeutically effective amount of the compound according to any one of to any one of claims 1-42 or a pharmaceutically acceptable salt thereof or a pharmaceutical composition according to claim 43, wherein one or more cells express KRAS G12D mutant protein.

53. The method according to claim 51 or 52, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

54. The method according to claim 51 or 52, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

55. The method according to claim 54, wherein the cancer is non-small cell lung cancer.

56. The method according to claim 54, wherein the cancer is colorectal cancer.

57. The method according to claim 54, wherein the cancer is pancreatic cancer.

58. The method according to anyone of claims 51-57, wherein the subject has a cancer that was determined to have one or more cells expressing the KRAS G12D mutant protein prior to administration of the compound or a pharmaceutically acceptable salt thereof.
